(19) United States
(12) Patent Application Publication
(10) Pub. No.: US 2017/0037480 A1
(43) Pub. Date: Feb. 9, 2017

(54) HSF1 IN TUMOR STROMA

(71) Applicant: Whitehead Institute for Biomedical Research, Cambridge, MA (US)

(72) Inventors: Ruth Scherz-Shouval, Brookline, MA (US); Susan Lindquist, Chestnut Hill, MA (US); Luke J. Whitesell, Somerville, MA (US); Sandro Santagata, Jamaica Plain, MA (US); Marc Mendillo, Cambridge, MA (US)

(21) Appl. No.: 15/303,397

(22) PCT Filed: Apr. 13, 2015
(86) PCT No.: PCT/US15/25610

§ 371 Date: Oct. 11, 2016

Related U.S. Application Data
(60) Provisional application No. 61/978,796, filed on Apr. 11, 2014.

(51) Int. Cl. C12Q 1/68; G01N 33/50; G01N 33/574

(52) U.S. Cl. CPC C12Q 1/6886; G01N 33/57415; G01N 33/57423; G01N 33/57407; G01N 33/5011; C12Q 2600/158; C12Q 2600/118; C12Q 2600/106; G01N 2800/52; G01N 2333/4703

(57) ABSTRACT

In some aspects, the invention relates to Heat Shock Protein-1 (HSF1) gene and HSF1 gene products in tumor stroma. In some aspects, the invention provides methods of tumor prognosis, treatment-specific prediction, or treatment selection, the methods comprising measuring the level of HSF1 expression or HSF1 activation in a sample obtained from the tumor that comprises tumor-associated stromal cells. In some aspects, the invention relates to the discovery that increased HSF1 expression and increased HSF1 activation in tumor-associated stromal cells correlate with poor outcome in cancer. In some embodiments, the methods comprise measuring HSF1 expression or activation specifically in tumor-associated stromal cells. In some embodiments, the methods comprise measuring HSF1 expression or activation specifically in tumor-associated stromal cells and specifically in cancer cells. In some embodiments HSF1 expression or activation is measured using an antibody that specifically binds to HSF1. In some embodiments HSF1 expression or activation is measured by measuring expression of genes that are regulated by HSF1 in tumor-associated stromal cells. In some aspects, the invention relates to inhibiting HSF1 in tumor-associated stromal cells as an approach to cancer therapy.

### RELATED APPLICATIONS

This application claims the benefit of U.S. Provisional Application No. 61/978,796, filed Apr. 11, 2014. The entire teachings of the above application(s) are incorporated herein by reference.

### BACKGROUND

Cancer cells in a tumor mass are surrounded by a variety of other cell types, including immune cells, fibroblasts and endothelial cells as well as extracellular matrix (ECM) components. Taken together, these comprise the tumor microenvironment. Cells of the tumor microenvironment contribute to hallmarks of cancer, and their co-evolution with cancer cells plays a key role in tumor formation and progression (Bissell and Hines, 2011; Hanahan and Coussens, 2012; Hanahan and Weinberg, 2011). In the majority of carcinomas, the most abundant cells in the tumor microenvironment are the CAFs, cancer-associated fibroblasts, (Hanahan and Coussens, 2012; Hanahan and Weinberg, 2011; Kalluri and Zeisberg, 2006).

CAFs include myofibroblasts and various variants of normal tissue-derived fibroblasts that are recruited by the tumor. CAFs contribute to diverse processes driving malignant progression including cancer cell proliferation, angiogenesis, invasion, metastasis and drug-resistance (Erez et al., 2010; Kalluri and Zeisberg, 2006; Olumi et al., 1999; Orimo et al., 2005; Straussman et al., 2012; Wilson et al., 2012). CAFs support cancer cells in a non-cell-autonomous manner through secretion of ECM molecules, chemokines and cytokines (such as stromal-derived factor 1 (SDF1), and IL6) and growth factors (such as transforming growth factor β (TGFβ), hepatocyte growth factor (HGF) and fibroblast growth factor (FGF)) (Kalluri and Zeisberg, 2006; Lu et al., 2012; Moskovits et al., 2006; Newman et al., 2011; Orimo et al., 2005; Pickup et al., 2013; Siegel and Massague, 2003; Spaeth et al., 2009; Tomasek et al., 2002). The secretion of cytokines also feeds back to promote the fibroblast-to-CAF transition, through autocrine TGFβ and SDF1 signaling (Kojima et al., 2010).

Despite accumulating evidence for the non-cell-autonomous effects of CAFs on cancer cells, little is known about the factors that drive the reprogramming of stromal cells within tumors.

### SUMMARY

In some aspects, the invention provides diagnostic methods based at least in part on measuring HSF1 expression and/or activation in stromal cells.

In some aspects, the invention provides prognostic methods based at least in part on measuring HSF1 expression and/or activation in tumor-associated stromal cells.

In some aspects, the invention provides a method of assessing the prognosis of a subject in need of prognosis for a tumor: comprising: measuring the level of HSF1 expression and/or activation in a sample comprising tumor-associated stromal cells obtained from the tumor; and comparing the level of HSF1 expression and/or activation in the tumor-associated stromal cells with a control level of HSF1 expression and/or activation of HSF1, wherein an increased level of HSF1 expression and/or activation in tumor-associated stromal cells of a tumor as compared to a control level of HSF1 expression and/or activation is correlated with poor outcome, thereby assessing the prognosis of the subject. In some embodiments a higher level of HSF1 expression and/or activation in tumor-associated stromal cells of a tumor as compared to a control level indicates that the prognosis of the subject is poor, and a lower or similar level of HSF1 expression and/or activation in tumor-associated stromal cells of a tumor as compared to a control level indicates that the prognosis of the subject is more favorable, e.g., that the prognosis is good. In some embodiments the level of HSF1 expression and/or activation is measured specifically in tumor-associated stromal cells. In some embodiments the level of HSF1 expression and/or activation is measured specifically in cancer-associated fibroblasts. In some embodiments the level of HSF1 expression and/or activation is measured specifically in tumor-associated stromal cells and is also measured specifically in cancer cells. In some embodiments the level of HSF1 expression and/or activation is measured specifically in cancer-associated fibroblasts and is also measured specifically in cancer cells. In some embodiments the level of HSF1 expression and/or activation as compared with a control level is measured specifically in tumor-associated stromal cells, and the method further comprises: measuring the level of HSF1 expression and/or activation specifically in cancer cells in the sample; comparing the level of HSF1 expression and/or HSF1 activation in the cancer cells with a control level of HSF1 expression and/or activation, wherein a lower or similar level of HSF1 expression and/or activation in cancer cells of the tumor as compared to a control level is indicative of a better prognosis than if the level of HSF1 expression and/or activation in cancer cells of the tumor is higher than the control level; and refining the prognosis based on the results of the comparison. In some embodiments a prognosis is for overall survival. In some embodiments a prognosis is for disease-free survival. In some embodiments a prognosis is for progression-free survival.

In some embodiments, a prognostic method further comprises selecting a treatment regimen for the subject based at least in part on the prognosis; and subjecting the subject to the selected treatment regimen. In some embodiments the method comprises determining that the subject has a poor prognosis and the method further comprises subjecting the subject to a relatively intensive treatment regimen based at least in part on the prognosis. In some embodiments the treatment regimen comprises administering an anticancer agent or radiotherapy to the subject. In some embodiments the treatment regimen comprises administering adjuvant chemotherapy or radiotherapy to the subject based at least in part on the prognosis.

In some aspects the invention provides a method of diagnosing a tumor in a subject comprising: measuring the level of HSF1 expression and/or activation in a sample comprising stromal cells obtained from a location in the subject's body that is suspected of harboring a tumor; and comparing the level of HSF1 expression and/or activation in the stromal cells with a control level of HSF1 expression and/or activation, wherein a higher level of HSF1 expression and/or activation in the stromal cells as compared to a control level of HSF1 expression and/or activation is indicative of the presence of a tumor. In some embodiments the stromal cells comprise fibroblasts.

In some aspects the invention provides a method for providing treatment-specific predictive information relating to a tumor, the method comprising: measuring the level of HSF1 expression and/or activation in a sample comprising tumor-associated stromal cells obtained from the tumor; and comparing the level of HSF1 expression and/or activation in the tumor-associated stromal cells with a control level of HSF1 expression and/or activation, wherein a higher level of HSF1 expression and/or activation in tumor-associated stromal cells of a tumor as compared to a control level of HSF1 expression and/or activation is correlated with tumor sensitivity or resistance to a treatment, thereby providing treatment-specific predictive information. In some embodiments a higher level of HSF1 expression and/or activation in tumor-associated stromal cells as compared to a control level indicates that the tumor has an increased likelihood of being sensitive to HSF1 inhibition.

In some aspects the invention provides a method of determining whether a subject with a tumor is a suitable candidate for treatment with an HSF1 inhibitor comprising: measuring the level of HSF1 expression and/or activation in a tumor sample comprising tumor-associated stromal cells obtained from the tumor; and comparing the level of HSF1 expression and/or activation in the tumor-associated stromal cells with a control level of HSF1 expression and/or activation of HSF1, wherein a higher level of HSF1 expression and/or activation in tumor-associated stromal cells of a tumor as compared to a control level of HSF1 expression and/or activation is indicative that the subject is a suitable candidate for treatment with an HSF1 inhibitor.

In some aspects the invention provides a method of predicting the likelihood that a tumor will be sensitive to an HSF1 inhibitor, the method comprising: measuring the level of HSF1 expression and/or activation in a tumor sample comprising tumor-associated stromal cells obtained from the tumor; and comparing the level of HSF1 expression and/or activation in the tumor-associated stromal cells with a control level of HSF1 expression and/or activation of HSF1, wherein a higher level of HSF1 expression and/or activation in tumor-associated stromal cells of a tumor as compared to a control level of HSF1 expression and/or activation is indicative that the tumor has an increased likelihood of being sensitive to the HSF inhibitor.

In some aspects the invention provides a method for tumor diagnosis, prognosis, treatment-specific prediction, or treatment selection comprising: measuring the level of HSF1 expression and/or activation in a sample comprising tumor-associated stromal cells obtained from a subject in need of diagnosis, prognosis, treatment-specific prediction, or treatment selection for a tumor; and scoring the sample based on the level of HSF1 expression and/or activation in the tumor-associated stromal cells, wherein the score provides diagnostic, prognostic, treatment-specific predictive, or treatment selection information.

In some embodiments of any aspect the level of HSF1 expression and/or activation is measured specifically in tumor-associated stromal cells. In some embodiments of any aspect, the tumor-associated stromal cells comprise or consist of cancer-associated fibroblasts.

In some embodiments of any aspect the tumor is a carcinoma. In some embodiments of any aspect the tumor is an adenocarcinoma. In some aspects of any embodiment the tumor is a Stage I tumor. In some aspects of any embodiment the tumor is a solid tumor. In some aspects of any embodiment the tumor is an epithelial tumor. In some aspects of any embodiment the tumor is a carcinoma. In some aspects of any embodiment the tumor is a breast, lung, skin, esophageal, colon, gastric, or prostate tumor.

In some embodiments of any aspect measuring the level of HSF1 expression comprises determining the level of an HSF1 gene product, e.g., an HSF1 mRNA or HSF1 polypeptide. In some embodiments measuring the level of HSF1 expression and/or activation comprises detecting HSF1 polypeptide using an antibody that binds to HSF1 polypeptide. In some embodiments the method comprises: contacting a sample comprising tumor-associated stromal cells or tumor stromal tissue obtained from the subject with an antibody that binds specifically to HSF1; and detecting the level of antibody binding to the sample thereby measuring the level of HSF1 expression. In some embodiments the method comprises: contacting a sample comprising tumor-associated stromal cells or tumor stromal tissue obtained from the subject with an antibody that binds specifically to HSF1; and detecting the level of antibody binding to cell nuclei in the sample thereby measuring the level of HSF1 activation. In some embodiments the method comprises contacting a sample comprising tumor-associated stromal cells or tumor stromal tissue obtained from the subject with a first antibody that binds specifically to HSF1 and a second antibody that binds to tumor-associated stromal cells; and detecting binding of the first antibody to cells in the sample to which the second antibody binds, thereby detecting HSF1 specifically in tumor-associated stromal cells. In some embodiments the sample comprises tumor stromal tissue, and determining the level of expression of HSF1 comprises performing immunohistochemistry (IHC) on the tissue sample. In some embodiments determining the level of HSF1 activation comprises determining the localization of HSF1 polypeptide in cells, wherein nuclear localization is indicative of HSF1 activation. In some embodiments measuring the level of HSF1 expression and/or activation comprises measuring the expression of one or more genes that are regulated by HSF1 in tumor-associated stromal cells; and comparing the level of expression of the one or more genes with a control level, wherein an increased level of expression of the one or more genes is indicative of increased HSF1 expression and/or activation.

In some embodiments of any of the methods for tumor diagnosis, prognosis, treatment-specific prediction, or treatment selection, measuring the level of HSF1 expression and/or activation in tumor-associated stromal cells comprises measuring the level of HSF1 activity in tumor-associated stromal cells by measuring the level of expression of one or more HSF1-regulated genes selected from the genes listed in Table D, e.g., at least 5, 10, 20, 30, or 40 genes listed in Table D. In some embodiments an increased level of expression of the gene(s) in a sample as compared to a control level indicates that the sample was obtained from a tumor and/or indicates that a tumor from which the sample was obtained is an aggressive tumor as compared with a tumor in which HSF1 activity in tumor-associated stromal cells is not increased. In some embodiments an increased level of expression of the gene(s) in a sample as compared to a control level indicates that a subject with a tumor from whom the sample was obtained has an increased likelihood of poor outcome as compared to a subject with a tumor that does not have an increased level of expression of the gene(s). In some embodiments an increased level of expression of the gene(s) in a sample as compared to a control level indicates that a subject with a tumor from which the sample was obtained is a suitable candidate for treatment with a proteostasis modulator, e.g., an HSF1 inhibitor, as compared to the case in which a tumor does not have an increased level of expression of the gene(s).

In some embodiments of any of the methods for tumor diagnosis, prognosis, treatment-specific prediction, or treatment selection, measuring the level of HSF1 expression and/or activation further comprises (in addition to measuring the level of HSF1 expression and/or activation in tumor-associated stromal cells, measuring the level of HSF1 activity in cancer cells by measuring the level of expression of one or more HSF1-regulated genes selected from the genes listed in Table A1, Table A2, Table A3, Table B, or Table C. In some embodiments the method comprises measuring expression of at least 5, 10, 20, 30, 40, 50, 60, 70, 80, 90, 100, 110, 120, 130, 140, or 150 genes listed in Table B. In some embodiments the method comprises measuring expression of at least 5, 10, 20, 30, 40, or 50 genes listed in Table B. In some embodiments an increased level of expression of one or more HSF1-regulated genes selected from the genes the expression of which are increased by HSF-1 and are listed in Table A1, Table A2, Table A3, Table B, or Table C in a tumor sample as compared to a control level, in combination with an increased level of expression of one or more genes listed in Table D in tumor-associated stromal cells from the same tumor, indicates that the tumor is an aggressive tumor as compared with a tumor in which HSF1 activity in tumor-associated stromal cells is not increased or as compared with a tumor in which HSF1 activity in cancer cells is not increased or is increased.

As described herein, any of the methods may be applied to subsets of the genes listed in the relevant tables or lists. A subset of a set can consist of any one or more members of the set in any combination. In some embodiments a subset has fewer members than the set of which it is a subset. In some embodiments a subset consists of up to 20%, up to 30%, up to 40%, up to 50%, up to 60%, up to 70%, up to 80%, or up to 90% of the genes listed in the relevant table or list.

In some embodiments of any of the methods for tumor classification, diagnosis, prognosis, treatment-specific prediction, or treatment selection, measuring the level of HSF1 expression and/or activation in tumor-associated stromal cells and in cancer cells comprises measuring the level of HSF1 activity in tumor-associated stromal cells by measuring the level of expression of one or more HSF1-regulated genes selected from the genes listed in Table D and measuring the level of HSF1 activity in cancer cells by measuring expression of one or more HSF1-regulated genes selected from the genes listed in Table A-1, Table A-2, Table A-3, Table B, or Table C. For example, in some embodiments of any of the methods for tumor classification, diagnosis, prognosis, treatment-specific prediction, or treatment selection, measuring the level of HSF1 expression and/or activation in tumor-associated stromal cells and in cancer cells comprises measuring the level of HSF1 activity in tumor-associated stromal cells by measuring the level of expression of one or more HSF1-regulated genes selected from the genes listed in Table D (e.g., at least 70%, at least 75%, or at least 80% of the genes listed in Table D) and measuring the level of HSF1 activity in cancer cells by measuring expression of one or more HSF1-regulated genes selected from the genes listed in Table C (e.g., at least 70%, at least 75%, or at least 80% of the genes listed in Table C). In some embodiments the measurements are made on a tumor sample that comprises tumor-associated stromal cells and cancer cells. In some embodiments the expression of one or more cancer-stroma normalization genes, e.g., one or more genes listed in Table E, in the sample is also measured (e.g., at least 70%, at least 75%, or at least 80% of the genes listed in Table E). The expression level of the one or more cancer-stroma normalization genes may be used to normalize the expression levels of the HSF1-regulated genes to account for the fact that the sample may contain a variable proportion of tumor-associated stromal cells and cancer cells.

In some aspects the invention provides a method of identifying a candidate anti-cancer agent comprising: contacting tumor-associated stromal cells with a test agent; measuring HSF1 expression and/or activation in the tumor-associated stromal cells; comparing the level of HSF1 expression and/or activation with a control level; and identifying the test agent as a candidate anti-cancer agent if the level of HSF1 expression and/or activation measured is lower than the control level.

In some embodiments of any of the methods, a control sample for comparison with tumor-associated stromal cells may comprise normal stromal cells or tissue, e.g., normal stromal cells or tissue found in the same organ or tissue as that from which a tumor arose or in which a tumor is present. A control level for comparison with a level measured in tumor-associated stromal cells may be a level measured in normal stromal cells or tissue, e.g., normal stromal cells or tissue found in the same organ or tissue as that from which a tumor arose or in which a tumor is present. For example, HSF1 expression and/or activation in cancer-associated fibroblasts may be compared with HSF1 expression and/or activation in normal fibroblasts. In some embodiments of any of the methods a control level is a level in fibroblasts present in normal tissue of the same type or origin as that from which a tumor arose or is present. In some embodiments the normal stromal cells may be in the same tissue section as tumor tissue but are within normal, non-neoplastic tissue. In some embodiments of any of the methods involving measuring HSF1 expression and/or activation in tumor cells, a control level of HSF1 expression and/or HSF1 activation for comparison with a level of HSF1 expression and/or HSF1 activation in tumor cells is a level measured in normal cells or tissue, e.g., normal cells or tissue of the same type or origin as that from which a tumor arose or is present.

In some embodiments, any of the methods may comprise providing or obtaining a sample comprising tumor-associated stromal cells. In some embodiments the sample further comprises cancer cells. In some embodiments a separate sample that comprises cancer cells may be provided or obtained. In some embodiments, any of the methods may comprise providing a subject in need of tumor diagnosis, prognosis, treatment-specific predictive information, or treatment selection.

In some embodiments, any of the methods may further comprise assessing at least one additional cancer biomarker. The at least one additional cancer biomarker is typically a gene or gene product (e.g., mRNA or protein) whose expression, activation, localization, or activity, correlates with the presence or absence of cancer, with cancer aggressiveness, with cancer outcome, cancer prognosis, or treatment-specific cancer outcome. The cancer biomarker(s) can be selected based on the tumor type.

In some embodiments, any of the methods can further comprise selecting or administering a therapeutic regimen based at least in part on results of assessing the level of HSF1 expression and/or activation in tumor-associated stromal cells. In some aspects, the invention provides a method comprising selecting or administering a treatment to a subject in need of treatment for a tumor, wherein the treatment is selected based at least in part on an assessment of the level of HSF1 expression and/or activation in a sample comprising tumor-associated stromal cells obtained from the tumor. In some embodiments a method comprises selecting or administering a more intensive monitoring and/or therapy regimen if a tumor (or sample obtained therefrom) is classified as having an increased likelihood of being aggressive, if a tumor or subject is classified as having an increased likelihood of having a poor outcome, or if a subject is classified as having a poor prognosis. For example, in some embodiments a method comprises selecting or administering adjuvant therapy (e.g., adjuvant chemotherapy or adjuvant radiation) if a tumor (or sample obtained therefrom) is classified as having an increased likelihood of being aggressive, if a tumor or subject is classified as having an increased likelihood of having a poor outcome, or if a subject is classified as having a poor prognosis. In some embodiments a method comprises selecting or administering an HSF1 inhibitor if the level of HSF1 expression or the level of HSF1 activation is increased in tumor-associated stromal cells. In some embodiments a method comprises selecting or administering an HSF1 inhibitor if the level of HSF1 expression or the level of HSF1 activation is increased in tumor-associated stromal cells and in cancer cells. In some embodiments a method comprises selecting or administering a proteostasis modulator if the level of HSF1 expression or the level of HSF1 activation is increased in tumor-associated stromal cells and in cancer cells. In some embodiments a method comprises selecting or administering a proteostasis modulator if the level of HSF1 expression or the level of HSF1 activation is increased in tumor-associated stromal cells and in cancer cells.

In some aspects, the invention provides a kit that comprises at least one agent of use to measure the level of HSF1 expression or HSF1 activation in a sample, e.g., an agent that specifically binds to an HSF1 gene product (e.g., HSF1 mRNA or HSF1 protein). The agent may be, e.g., an antibody, or a nucleic acid. In some embodiments the agent is validated for use in measuring HSF1 expression and/or activation in tumor-associated stromal cells, in that results of an assay using the agent have been shown to correlate with cancer outcome or treatment efficacy of at least one specific treatment. In some embodiments the agent is an antibody useful for performing IHC to detect HSF1 in tumor-associated stromal cells.

In some aspects, the invention provides a kit that comprises at least one reagent of use to measure the level of HSF1 activity by measuring expression of one or more HSF1-regulated genes. In some embodiments the kit comprises probes and/or primers suitable for measuring expression of at least 5, 10, 20, 30, or 40 genes listed in Table D. In some embodiments the kit further comprises probes and/or primers suitable for measuring expression of at least 5, 10, 20, 30, 40, or 50 genes listed in Table C, or at least 5, 10, 20, 30, 40, 50, 60, 70, 80, 90, 100, 110, 120, 130, 140, or 150 genes listed in Table B. In some embodiments the kit further comprises probes and/or primers suitable for measuring expression of at least 2, 4, 6, 8, 10, 12, 14, or 16 genes listed in Table E.

Certain conventional techniques and concepts of cell biology, cell culture, molecular biology, microbiology, recombinant nucleic acid (e.g., DNA) technology, immunology, etc., which are within the skill and knowledge of those of ordinary skill in the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005; Buchwalow, I. and Böcker, W. (2010) Immunohistochemistry: Basics and Methods, Methods in Molecular Medicine, Springer) Lodish H, et al. (2007). Molecular cell biology (6th ed.). New York: W.H. Freeman and CO. Further information on cancer and treatment thereof may be found in Cancer: Principles and Practice of Oncology (V. T. De Vita et al., eds., J.B. Lippincott Company, 8^{th} ed., 2008 or 9^{th} ed., 2011) and Weinberg, R A, The Biology of Cancer, Garland Science, 2^{nd} ed. 2013. All patents, patent applications, books, journal articles, databases, websites, and other publications mentioned herein are incorporated herein by reference in their entirety. In the event of a conflict or inconsistency with the specification, the specification shall control. Applicants reserve the right to amend the specification based on any of the incorporated references and/or to correct obvious errors. None of the content of the incorporated references shall limit the invention.

### BRIEF DESCRIPTION OF THE DRAWINGS

FIGS. 1A-1C show HSF1 activation in cancer-associated fibroblasts within human tumors. (A) Tissue sections of breast resection specimens from 12 patients encompassing both invasive ductal carcinoma and neighboring normal breast lobules (in the same section) were immunostained with anti-HSF1 antibodies (brown signal, upper panels) or co-stained with anti-HSF1 and anti-SMA (pink) antibodies (lower panels). Representative images are shown. Arrows indicate HSF1-positive CAFs in the left panels, and HSF1-negative normal fibroblasts in the lower right panel. C and S indicate cancer- or stroma-rich regions, respectively. For the normal tissue, E and F indicate regions rich with epithelial cells or fibroblasts, respectively. (B) Pie charts depict the distribution of relative nuclear HSF1 staining intensity in the stroma amongst 12 breast resection specimens with matching controls. For each specimen, 4 regions of tumor or normal tissue were evaluated. The statistical significance of the differences between normal and tumor was assessed using repeated-measures ANOVA (p=4e-13), as well as paired t-tests, followed by Bonferroni correction (p<0.01). (C) Representative images of tumor sections from patients with the indicated types of cancer co-stained for HSF1 (brown) and SMA (pink). C and S indicate cancer- or stroma-rich regions, respectively. See also Fig. S1.

FIGS. 2A-2B illustrate that stromal Hsf1 status alters tumor progression and histology in human breast xenografts. MCF7 breast cancer cells alone or mixed with WT or Hsf1 null primary MEFs were injected subcutaneously into NOD-scid mice. The experiment was repeated twice, with 4 mice per group in each experiment. (A) Tumor growth was monitored by caliper measurements twice weekly. The mean tumor volume (total 8 tumors per treatment group, error bars, SEM) is shown. The distribution of individual measurements is shown in the lower panels, in scatter plots for days 22 and 38 post injection. Error bars, SEM. *p<0.05, **p<0.01. (B) Mice were sacrificed when tumor burden reached size limit and the tumors were excised, formalin-fixed and sections stained with hematoxylin & eosin (H&E, upper panels) or Masson's trichrome stain (lower panels). All images collected at the same magnification. Scale bar=50 μm. See also Figs. S2A-S2B.

FIGS. 3A-3F show HSF1 in fibroblasts supports cancer cell growth by activating gene expression programs both in cancer cells and in fibroblasts. (A-B) WT or Hsf1 null immortalized MEFs were plated at near confluency, allowed to adhere and treated with 10 g/ml mitomycin C. D2A1 mouse mammary tumor cells stably expressing dsRed were seeded on top of the MEFs (1:5 ratio of D2A1:MEFs), and allowed to grow for 72 h-96 h, after which cancer cells were either visualized by fluorescent microscopy (A) or trypsinized and quantitated by flow cytometry (B). The mean of 3 independent experiments is shown. Error bars, SEM ** p<0.005. (C-D) Total RNA was purified from duplicate cultures of D2A1 cancer cells grown with or without WT or Hsf1 null MEFs and sorted as described above. RNA was hybridized to Agilent microarrays, and relative gene expression levels were analyzed using cluster 3.0. For each gene, expression in D2A1 cells without co-culture was set to 1, and the relative change in expression upon co-culture with WT or Hsf1 null MEFs was calculated. (C) Overlap of genes differentially expressed in D2A1 cancer cells in the presence of WT or Hsf1 null MEFs is depicted. (D) Heat-map depicting fold change in mRNA levels of genes differentially expressed in D2A1 cells grown in co-culture with WT versus Hsf1 null fibroblasts. Duplicate samples are shown for each condition. Gene ontology (GO) enrichment was calculated using GOrilla software. The most significantly enriched processes are shown to the right of the panel. Groups a and c correspond to groups a and c in panel (C). (E) WT or Hsf1 null MEFs were plated in duplicate, near confluency and allowed to adhere for 24 h after which they were co-cultured with dsRed-marked D2A1 cells as described in (A). After 72-96 h, cultures were trypsinized, sorted and mRNA was extracted and hybridized to Agilent microarrays. MEFs cultured without D2A1 cells and processed in the same manner served as controls. Expression data was analyzed using cluster 3.0. Genes differentially expressed under the conditions tested were clustered into 4 groups according to expression pattern as shown. GO enrichment was calculated using GOrilla software and the most significantly enriched processes are shown to the right of the panel. (F) Gene set enrichment analysis of genes upregulated in WT vs Hsf1 null MEFs co-cultured with cancer cells (groups 1 and 4 in panel E). Enrichment was calculated for the indicated gene sets, and is presented as normalized enrichment score (NES). Statistically significant enrichment (false discovery rate (FDR) q-value <0.05) is shown in red, non-significant enrichment is shown in gray. See also Figs. S3A-S3D.

FIGS. 4A-4F show that TGFβ and SDF1 mediate the support of cancer cell growth by stromal HSF1. (A) The relative expression of Sdf1, Tgfβ1 and Tgfβ2 in WT or Hsf1 null immortalized MEFs was measured by quantitative PCR. mRNA expression levels were normalized to the house keeping gene Gapdh. The mean of 3 independent experiments is shown. Error bars, SEM. *p<0.05, **p<0.01. (B-C) WT or Hsf1 null immortalized MEFs were plated and treated with mitomycin C as in FIG. 3A. D2A1 cells marked with dsRed were then seeded on top of the MEFs in the presence or absence of 10 ng/ml TGFβ1 and 100 ng/ml SDF1. After 96 h, cells were either visualized by fluorescent microscopy (B) or trypsinized and quantitated by flow cytometry (C). The percentage of cancer cells in co-culture is presented. The experiment was repeated 3 times, in triplicate. Representative results of one experiment are shown as the mean+/−SEM. *p<0.05. (D) Immortalized WT or Hsf1 null mitomycin-treated MEFs were pretreated, or not, with LY2109761 for 30 minutes before D2A1 cells marked with dsRed were seeded on top. Cultures were continued for 72 h, with daily supplementation of LY2109761 (or not, as control), and then analyzed as in (C). The experiment was repeated 3 times, in triplicates. Results are expressed as the mean relative number of cancer cells, normalized to the non-drug treated co-cultures with WT MEFs. Error bars, SEM; *p<0.05, **p<0.01. (E) Immortalized WT MEFs stably expressing shRNA hairpins targeting Smad2 (shSmad2) or GFP (shGFP) were co-cultured with D2A1 cells, treated and analyzed as in (C). The percentage of cancer cells in the co-culture is presented. (F) Chromatin immunoprecipitation (IP) was performed with anti-HSF1 antibodies using material prepared from MCF7 tumor xenografts. Normal rat-IgG served as a negative IP control. IPs were analyzed by qPCR with primers targeting potential heat shock elements in mouse Sdf1 and Tgfβ2. Primers targeting an intergenic region in the mouse DNA, not expected to be amplified, were used as a negative control. The experiment was repeated twice and tumors from 3 mice were used for each experiment. Representative results from one experiment are shown as mean+/−SEM, *p<0.05. See also Figs. S4A-S4F.

FIGS. 5A-5D show that increased HSF1 activation in the stroma is associated with decreased survival in breast cancer patients. (A-C) Analysis of Hsf1 mRNA expression levels in the stroma of 53 breast cancer patients from (Finak et al., 2008). (A) The association between Hsf1 expression and tumor grade is presented in a box & whiskers plot. *p<0.05 (B) Kaplan-Meier (KM) analysis of patients stratified by Hsf1 expression. (C) The correlation between Hsf1 expression level and HER2 status is presented in a box & whiskers plot. *p<0.05 (D) Breast cancer resections from 46 early-stage patients were stained with anti-HSF1 antibodies and scored in a blinded manner for HSF1 protein activation (relative nuclear staining intensity in the stroma) by immunohistochemistry. Association of stromal HSF1 activation with disease-free survival was assessed by KM analysis. See also Figs. S5A-S5E.

FIGS. 6A-6D show increased HSF1 activation in the stroma is associated with decreased survival in lung cancer patients. (A) Lung cancer resections from 5 patients were stained with anti-HSF1 (brown), anti-SMA (brown) or a combination of both antibodies (HSF1 in brown; SMA in red). Representative images are shown. Scale bar=20 μm (B-C) Lung cancer resections from 72 patients with Stage I disease were stained with anti-HSF1 antibodies and relative nuclear HSF1 intensity in the stromal cells and in the cancer cells were scored in a blinded manner. (B) HSF1 stromal scores are correlated with disease-free survival by KM analysis. (C) KM analysis of disease-free survival for patients with concordant high or low HSF1 scores in both stromal cells and cancer cells. (D) Stromal HSF1 levels in KRAS mutant tumors (n=18) from the lung cancer cohort correlate with disease-free survival by KM analysis. See also Figs. S6A-S6D.

Fig. S1 shows cancer-associated fibroblasts are the predominant HSF1-positive cell type in human breast cancer stroma. Tissue sections from breast tumor resection specimens of 12 patients with invasive ductal carcinoma were stained with hematoxylin & eosin (H&E, upper left panel) or immunostained with the following antibodies: anti-CD45 (Leukocyte Common Antigen; LCA for leukocytes), anti-CD31 (for endothelial cells), anti-HSF1 and anti-SMA (all single stains in brown) or a combination of anti-HSF1 (brown) and anti-SMA (pink) antibodies. Representative images are shown at the same magnification for all panels. Scale bar, 100 μm. C and S indicate cancer- or stroma-rich regions, respectively.

Figs. S2A-S2B show nuclear HSF1 levels are increased in mouse cancer-associated fibroblasts recruited into xenografts by human MCF7 cancer cells. (A) MCF7 breast cancer cells were injected subcutaneously into NOD-scid mice. Mice were sacrificed when tumor burden reached size limit and the tumors were excised, formalin-fixed and sections stained with H&E, anti-SMA (brown), or anti-HSF1 (brown) or co-stained with anti-HSF1 (brown) and anti-SMA (pink). Scale bar for all images, 50 μM. (B) Tumors arising from MCF7 cells co-injected with Hsf1 null MEFs contain HSF1-negative (injected) as well as HSF1-positive (host) stromal cells. MCF7 breast cancer cells alone or mixed with WT or Hsf1 null primary MEFs were injected subcutaneously into NOD-scid mice. Mice were sacrificed when tumor burden reached size limit and the tumors were excised, formalin-fixed and sections stained with anti-HSF1 antibodies (brown). The experiment was repeated twice, with 4 mice per group in each experiment. Typical fields are shown in the upper panels. Stromal rich fields chosen on the basis of H&E staining are shown in the middle row of panels to demonstrate HSF1 expression in the stroma. The boxed areas in these images are presented at higher magnification in the bottom panels. Scale bar, 50 μM.

Figs. S3A-S3D illustrate that Hsf1 null fibroblasts show a reduced ability to support cancer cell accumulation in co-culture as compared to WT fibroblasts. (A) WT or Hsf1 null MEFs were plated at near confluency, allowed to adhere and treated with 10 μg/mL mitomycin C. D2A1 mouse mammary tumor cells stably expressing fluorescent dsRed protein were then seeded on top of the MEFs (1:5 ratio of D2A1:MEFs), and allowed to grow for 72 h-96 h, after which co-cultures were visualized by fluorescence microscopy (upper panels) or phase contrast microscopy (lower panels). All images are presented at the same magnification. (B) MCF7 human breast cancer cells stably expressing GFP were seeded on top of MEF feeder layers, allowed to grow and visualized by fluorescence microscopy as in (A). (C) HCC38 human breast cancer cells stably expressing GFP (left) or 4T7 mouse breast cancer cells stably expressing dsRed (right) were seeded on top of MEF feeder layers and allowed to grow for 72 h. Co-cultures were then trypsinized and quantitated by flow cytometry. The percentage of cancer cells in the co-culture is presented. The mean of 3 independent experiments is shown +/−SEM. *p<0.05, **p<0.01. (D) Short-term loss of HSF1 in fibroblasts impairs their ability to support the growth of cancer cells in co-culture. Left panel: Immortalized Bi-Tet-Hsf1 MEFs were pre-treated with doxycycline for 5d (Dox) or left untreated (Cont). They were then plated and treated with mitomycin C after which dsRed-marked D2A1 cells were plated on top as in (C) and allowed to grow for 72 h in the continued presence (Dox) or absence (Cont) of Dox. Co-cultures were analyzed as in (C). The percentage of cancer cells in the co-culture is presented. The experiment was repeated 3 times, in quadruplicate. Representative results of one experiment are shown as the mean+/−SEM. *p<0.05. Right panel: Immortalized Bi-Tet-Hsf1 MEFs treated with Dox as described above (or left untreated) were incubated for 72 h, without co-culture, after which lysates were prepared and the relative HSF1 protein level was determined by western blot analysis. Tubulin was blotted as a loading control.

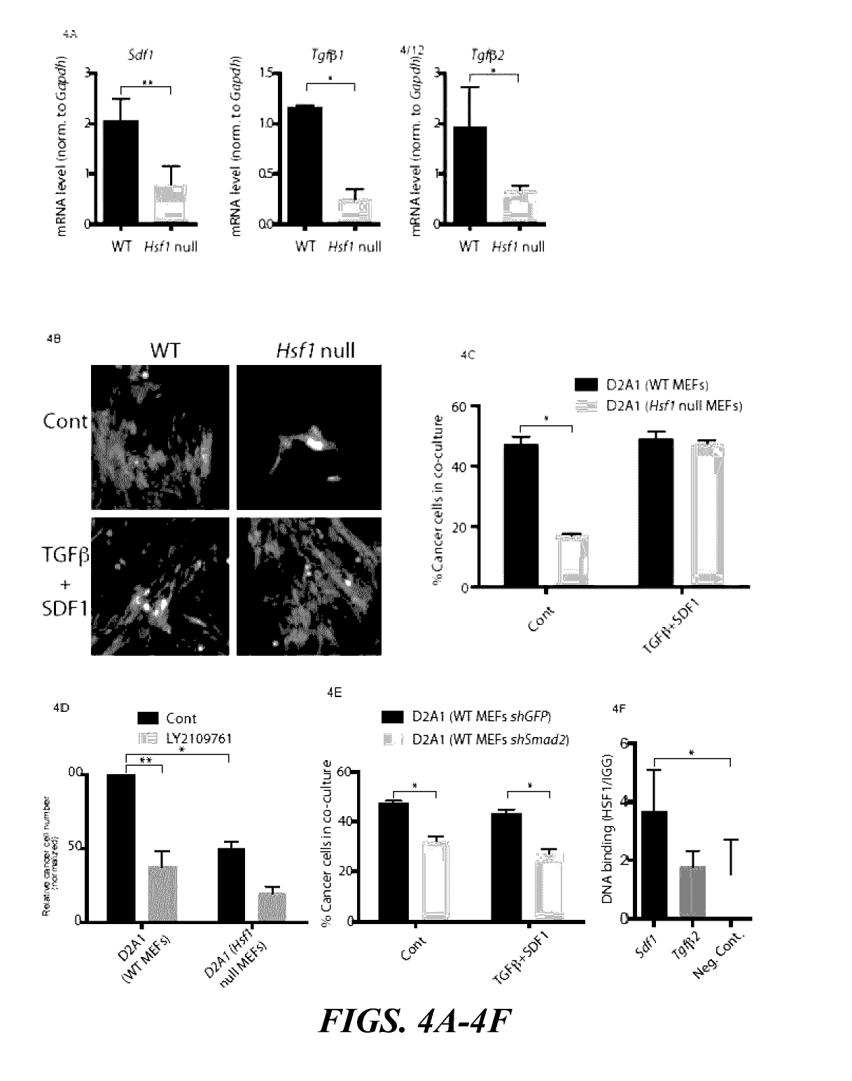

Figs. S4A-S4F show additional results. (A) Tgfβ and Sdf1 mRNA levels are regulated by HSF1 in primary MEFs. The relative expression of Sdf1, Tgfβ1 and Tgfβ2 in three separate sets of WT or Hsf1 null MEFs (each derived from a different pregnancy) was measured by quantitative PCR. mRNA expression levels were normalized to the house keeping gene Gapdh. The experiment was repeated 3 times (each time with a different set of primary MEFs), in triplicate. Representative results are shown as the mean+/−SEM. *p<0.05, **p<0.01, ***p<0.001. (B) TGFβ1 and SDF1 in combination, but not separately, are sufficient to restore cancer cell accumulation in the presence of Hsf1 null MEFs. WT or Hsf1 null immortalized MEFs were plated and treated with mitomycin C. D2A1 cells marked with dsRed were then seeded on top of the MEFs in the presence or absence of 10 ng/ml TGFβ1, 100 ng/ml SDF1 or a combination of both. After 96 h, cells were trypsinized and quantitated by flow cytometry. The percentage of cancer cells in the co-culture is presented. The experiment was repeated 3 times, in triplicate. Representative results of one experiment are shown as the mean+/−SEM. *p<0.05. (C) The TGFβ receptor inhibitor LY2109761 does not directly inhibit the growth or survival of D2A1 cancer cells. Cells were treated, or not, with LY2109761 (1 μM) and allowed to grow for 72 h with daily re-addition of the inhibitor (or not, as control). Relative cell number was measured by luminescence using Cell-Titer Glo reagent (Promega). The experiment was repeated 3 times, in triplicate. The mean+/−SEM of 3 experiments is shown. (D) Knock down of Smad2 in D2A1 cells doesn't affect their growth in co-culture with MEFs. dsRed-marked D2A cells stably expressing shRNA hairpins that target Smad2 (shSmad2) or GFP (shGFP) were seeded on a feeder layer of WT immortalized MEFs pretreated with mitomycin C. After growth for 96 h, cells were trypsinized and quantitated by flow cytometry. The experiment was repeated 3 times, in triplicate. Representative results of one experiment are shown as the mean+/−SEM. (E) Verification of Smad2 knockdown. Relative levels of SMAD2 in WT immortalized MEFs (left two panels) or D2A1 cells (right two panels) were measured by immunoblotting lysates prepared from cells stably expressing shRNA hairpins targeting Smad2 (shSmad2) or GFP (shGFP). Tubulin was blotted as a loading control. (F) Validation of species-specificity of the primers used for detection of HSF1 binding to mouse-Sdf1 and Tgfβ2. qPCR was performed using total DNA extracts prepared from human and mouse cells in a manner similar to that used for ChIP-PCR experiments (FIG. 4F). The primers used were the same as those used in FIG. 4F. Primers for a human gene (Dihydrofolate Reductase, Dhfr) were used as positive control (human pos. cont).

Figs. S5A-S5E show breast cancer results. (A-B) Stromal Hsf1 mRNA levels do not correlate with estrogen receptor (ER) or progesterone receptor (PR) status of breast cancer samples. Primary data for 53 breast cancer patients derived from a previous study reported by (Finak et al., 2008). (A) The association between Hsf1 expression and ER status is presented in a box & whiskers plot (see Extended Experimental Procedures). p=ns. (B) The correlation between Hsf1 expression and PR status is presented in a box & whiskers plot. p=ns. (C-E) Activation of HSF1 in stromal cells and cancer cells is associated with poor patient outcome in breast cancer. Breast cancer resections from 46 early-stage patients were immunostained with anti-HSF1 antibodies and scored in a blinded manner for HSF1 protein activation (relative nuclear staining intensity) in the stroma and in the cancer cells. (C) Association of stromal HSF1 activation with overall survival was assessed by KM analysis. (D) Association of cancer-cell HSF1 status with overall survival was assessed by KM analysis. (E) Association of cancer-cell HSF1 status with disease-free survival was assessed by KM analysis.

Figs. S6A-S6D show lung cancer results. (A) Hsf1 null fibroblasts support lung cancer cell accumulation in co-culture poorly, as compared to WT fibroblasts. A549 (left) or H1703 (right) non-small lung cancer cells were seeded on a feeder layer of WT or Hsf1 null immortalized MEFs pre-treated with mitomycin C. After 72 h, cells were trypsinized and quantitated by flow cytometry. The percentage of cancer cells in the co-culture is presented. The experiment was repeated 3 times, in triplicate. Means+/−SEM of 3 experiments are shown. *p<0.05. (B) Increased HSF1 activation in the cancer cells tends to correlate with reduced disease-free survival in lung cancer. Lung cancer resections from 72 patients with Stage I disease were stained with anti-HSF1 antibodies and relative nuclear HSF1 intensity in the cancer cells was scored in a blinded manner. Association of cancer-cell HSF1 status with disease-free survival was assessed by KM analysis. A trend towards association between high HSF1 and shortened survival is seen, but does not reach statistical significance. (C) KRAS mutations are significantly associated with poor patient outcome. Potential association of tumor KRAS mutational status with disease-free survival in our lung cancer cohort was assessed by KM analysis. (D) EGFR mutational status does not correlate with patient outcome. Potential association of tumor EGFR mutational status with disease-free survival in our lung cancer cohort was assessed by KM analysis.

### DETAILED DESCRIPTION OF CERTAIN EMBODIMENTS

### Glossary

For convenience, certain terms employed herein are collected below. It should be understood that any description of a term or concept below or elsewhere herein may be applied wherever such term or concept appears herein.

The term “antibody” refers to an immunoglobulin, whether natural or wholly or partially synthetically produced. An antibody may be a member of any immunoglobulin class, including any of the mammalian, e.g., human, classes: IgG, IgM, IgA, IgD, and IgE, or subclasses thereof, and may be an antibody fragment, in various embodiments of the invention. An antibody can originate from any of a variety of vertebrate (e.g., mammalian or avian) organisms, e.g., mouse, rat, rabbit, hamster, goat, chicken, human, etc. As used herein, the term “antibody fragment” refers to a derivative of an antibody which contains less than a complete antibody. In general, an antibody fragment retains at least a significant portion of the full-length antibody's specific binding ability. Examples of antibody fragments include, but are not limited to, Fab, Fab′, F(ab′)2, scFv, Fv, dsFv diabody, Fd fragments, and domain antibodies. Standard methods of antibody identification and production known in the art can be used to produce an antibody that binds to a polypeptide of interest. In some embodiments, an antibody is a monoclonal antibody. Monoclonal antibodies can be identified and produced, e.g., using hybridoma technology or recombinant nucleic acid technology (e.g., phage or yeast display). In some embodiments, an antibody is a chimeric or humanized or fully human antibody. In some embodiments, an antibody is a polyclonal antibody. In some embodiments an antibody is affinity purified. It will be appreciated that certain antibodies, e.g., recombinantly produced antibodies, can comprise a heterologous sequence not derived from naturally occurring antibodies, such as an epitope tags. In some embodiments an antibody further has a detectable label attached (e.g., covalently attached) thereto (e.g., the label can comprise a radioisotope, fluorescent compound, enzyme, hapten).

“Cancer” is generally used interchangeably with “tumor” herein and encompasses pre-invasive and invasive neoplastic growths comprising abnormally proliferating cells, including malignant solid tumors (carcinomas, sarcomas) and including hematologic malignancies such as leukemias in which there may be no detectable solid tumor mass. As used herein, the term cancer includes, but is not limited to, the following types of cancer: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer such as oral squamous cell carcinoma; ovarian cancer including ovarian cancer arising from epithelial cells, stromal cells, germ cells and mesenchymal cells; pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. “Carcinoma” as used herein, refers to a cancer arising or believed to have arisen from epithelial cells, e.g., cells of the cancer possess various molecular, cellular, and/or histological characteristics typical of epithelial cells. A tumor may be classified according to the TNM Classification of Malignant Tumours (TNM) (Sobin L H, et al., eds. TNM Classification of Malignant Tumors, 7th ed. Wiley-Blackwell, Oxford 2009.

The term “diagnostic method” generally refers to a method that provides information regarding the identity of a disease or condition that affects a subject or whether a subject is suffering from a disease or disorder of interest, such as cancer. For example, a diagnostic method may determine that a subject is suffering from a disease or condition of interest or may identify a disease or condition that affects a subject or may identify a subject suffering from a disease or condition of interest.

The term “prognostic method”, generally refers to a method that provides information regarding the likely course or outcome of a disease regardless of treatment or across treatments (e.g., after adjusting for treatment variables or assuming that a subject receives standard of care treatment). A prognostic method may comprise classifying a subject or sample obtained from a subject into one of multiple categories, wherein the categories correlate with different likelihoods that a subject will experience a particular outcome. For example, categories can be low risk and high risk, wherein subjects in the low risk category have a lower likelihood of experiencing a poor outcome (e.g., within a given time period such as 5 years or 10 years) than do subjects in the high risk category. A poor outcome could be, for example, disease progression, disease recurrence, or death attributable to the disease.

The term “treatment-specific predictive method” generally refers to a method that provides information regarding the likely effect of a specified treatment, e.g., that can be used to predict whether a subject is likely to benefit from the treatment or to predict which subjects in a group will be likely or most likely to benefit from the treatment. It will be understood that a treatment-specific predictive method may be specific to a single treatment or to a class of treatments (e.g., a class of treatments having the same or a similar mechanism of action or that act on the same biological process, pathway or molecular target, etc.). A treatment-specific predictive method may comprise classifying a subject or sample obtained from a subject into one of multiple categories, wherein the categories correlate with different likelihoods that a subject will benefit from a specified treatment. For example, categories can be low likelihood and high likelihood, wherein subjects in the low likelihood category have a lower likelihood of benefiting from the treatment than do subjects in the high likelihood category. In some embodiments, a benefit is increased survival, increased progression-free survival, or decreased likelihood of recurrence. In some embodiments, a “suitable candidate for treatment” with a specified agent refers to a subject for whom there is a reasonable likelihood that the subject would benefit from administration of the agent, e.g., the tumor has one or more characteristics that correlate with a beneficial effect resulting from administration of the agent as compared with, e.g., no treatment or as compared with a standard treatment. In some embodiments, a “suitable candidate for treatment” with an agent refers to a subject for whom there is a reasonable likelihood that the subject would benefit from administration of the agent in combination with (i.e., in addition to) one or more other therapeutic interventions, e.g., the tumor has one or more characteristics that correlate with a beneficial effect from treatment with the agent and the other therapeutic interventions as compared with treatment with the other therapeutic interventions only. In some embodiments, a suitable candidate for treatment with an agent is a subject for whom there is a reasonable likelihood that the subject would benefit from addition of the agent to a standard regimen for treatment of cancer. See, e.g., De Vita, et al., supra for non-limiting discussion of standard regimens for treatment of cancer.

“Expression” refers to the cellular processes involved in producing RNA and protein such as, but not limited to, transcription, RNA processing, and translation.

As used herein, the term “gene product” (also referred to as a “gene expression product”) encompasses products resulting from expression of a gene, such as RNA transcribed from a gene and polypeptides arising from translation of mRNA. RNA transcribed from a gene can be non-coding RNA or coding RNA (e.g., mRNA). It will be appreciated that gene products may undergo processing or modification by a cell. For example, RNA transcripts may be spliced, polyadenylated, etc., prior to mRNA translation, and/or polypeptides may undergo co-translational or post-translational processing such as removal of secretion signal sequences or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Exemplary databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species due to natural allelic variation. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations frequently do not alter the encoded amino acid sequence, although DNA polymorphisms that lead to changes in the amino acid sequences of the encoded proteins can exist. It will also be understood that multiple isoforms of certain proteins encoded by the same gene may exist as a result of alternative RNA splicing or editing. In general, where aspects of the invention relate to a gene or gene product it should be understood that embodiments relating to such isoforms or allelic variants are encompassed unless indicated otherwise. For example, in general, allelic variants and most isoforms would be detectable using the same reagents (e.g., antibodies, probes, etc.) and methods.

“Isolated”, in general, means 1) separated from at least some of the components with which it is usually associated in nature; 2) prepared or purified by a process that involves the hand of man; and/or 3) not occurring in nature, e.g., present in an artificial environment.

“Nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments a nucleic acid comprises standard nucleosides (abbreviated A, G, C, T, U). In other embodiments a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos, in various embodiments. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, antisense, primer, or probe molecules may be used in various embodiments of the invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., relative to RNA or DNA of the same length and strandedness. A nucleic acid may comprise a detectable label, e.g., a fluorescent dye, radioactive atom, etc. “Oligonucleotide” refers to a relatively short nucleic acid, e.g., typically between about 4 and about 100 nucleotides long. Where reference is made herein to a polynucleotide, it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence, if presented herein, is presented in a 5′ to 3′ direction unless otherwise indicated.

“Proteostasis” refers to controlling the concentration, conformation (e.g., folding), binding interactions (quaternary structure), and subcellular location of the proteins within a cell, often through mechanisms such as transcriptional and/or translational changes, chaperone-assisted folding and disaggregation, or controlled protein degradation. Proteostasis can be thought of as a network comprising multiple distinguishable pathways (“proteostasis pathways”) that may interact with and influence each other. Proteostasis pathways include, e.g., the heat shock response, the ubiquitination-proteasome degradation pathway, and the unfolded protein response (UPR). “Proteostasis modulator” refers to an agent that modulates one or more proteostasis pathways. Proteostasis modulators include HSF1 inhibitors, HSP90 inhibitors, and proteasome inhibitors. “Proteasome inhibitor” refers to an agent that inhibits activity of the proteasome or inhibits synthesis of a proteasome component. Proteasome inhibitors include, e.g., a variety of peptidic and non-peptidic agents that bind reversibly to the proteasome, bind covalently to the active site of the proteasome, or bind to the proteasome outside the active site (sometimes termed “allosteric inhibitors”) (Ruschak A M, et al., J Natl Cancer Inst. (2011) 103(13): 1007-17). A number of proteasome inhibitors have shown promise in the treatment of cancer, including bortezomib (Velcade®) and carfilzomib (both approved by the US FDA), and various others under investigation. Exemplary proteasome inhibitors that have been tested in clinical trials in cancer include bortezomib, CEP-1 8770, MLN-9708, carfilzomib, ONX 0912, and NPI-0052 (salinosporamide A). HIV protease inhibitors such as nelvinavir also inhibit the proteasome. Other agents that inhibit the proteasome include chloroquine, 5-amino-8-hydroxyquinoline (5AHQ), disulfiram, tea polyphenols such as epigallocatechin-3-gallate, MG-132, PR-39, PS-I, PS-IX, and lactacystin. In some embodiments, a method of the invention is applied with regard to a proteasome inhibitor that has entered clinical development for, e.g., treatment of cancer.

“Polypeptide” refers to a polymer of amino acids. The terms “protein” and “polypeptide” are used interchangeably herein. A peptide is a relatively short polypeptide, typically between about 2 and 100 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity thereto. Exemplary modifications include phosphorylation, glycosylation, SUMOylation, acetylation, methylation, acylation, etc. In some embodiments, a polypeptide is modified by attachment of a linker useful for conjugating the polypeptide to or with another entity. Polypeptides may be present in or purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence, if presented herein, is presented in an N-terminal to C-terminal direction unless otherwise indicated.

A “sample” as used herein can be any biological specimen that contains cells, tissue, or cellular material (e.g., cell lysate or fraction thereof). Typically, a sample is obtained from (i.e., originates from, was initially removed from) a subject. Methods of obtaining such samples are known in the art and include, e.g., tissue biopsy such as excisional biopsy, incisional biopsy, or core biopsy; fine needle aspiration biopsy; brushings; lavage; or collecting body fluids such as blood, sputum, lymph, mucus, saliva, urine, etc., etc. In some embodiments, a sample contains at least some intact cells at the time it is removed from a subject and, in some embodiments, the sample retains at least some of the tissue microarchitecture. In some embodiments a sample is obtained from a tumor either prior to or after removal of the tumor from a subject. A sample may be subjected to one or more processing steps after having been obtained from a subject and/or may be split into one or more portions, which may entail removing or discarding part of the original sample. The portions of a sample may be considered to constitute a single sample unless otherwise indicated. It will be understood that the term “sample” encompasses such processed samples, portions of samples, etc., and such samples are still considered to have been obtained from the subject from whom the initial sample was removed. In many embodiments, a sample is obtained from an individual who has been diagnosed with cancer or is at increased risk of cancer, is suspected of having cancer, or is at risk of cancer recurrence. A sample used in a method of the present invention may have been procured directly from a subject, or indirectly by receiving the sample from one or more persons who procured the sample directly from the subject, e.g., by performing a biopsy or other procedure on the subject. A “tumor sample” is a sample that includes at least some cells, tissue, or cellular material obtained from a tumor. In general, a “sample” as used herein is typically a tumor sample or a sample obtained from tissue being evaluated for presence of a tumor.

The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, or 200 Da. In some embodiments, the small molecule is between about 500 kDa and about 1.0 kDa. In some embodiments, the small molecule is between about 1 kDa and about 1.5 kDa. In some embodiments, the small molecule is between about 1.5 kDa and about 1.9 kDa. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments a small molecule is an artificial (non-naturally occurring) molecule. In some embodiments, a small molecule is non-polymeric. In some embodiments, a small molecule is not an amino acid or protein. In some embodiments, a small molecule is not a nucleotide or nucleic acid. In some embodiments, a small molecule is not a saccharide. In some embodiments, the term “small molecule” excludes molecules that are ingredients found in standard tissue culture medium.

“Specific binding” generally refers to a physical association between a target molecule or complex (e.g., a polypeptide) and a binding agent such as an antibody or ligand. The association is typically dependent upon the presence of a particular structural feature of the target such as an antigenic determinant, epitope, binding pocket or cleft, recognized by the binding agent. For example, if an antibody is specific for epitope A, the presence of a polypeptide containing epitope A or the presence of free unlabeled A in a reaction containing both free labeled A and the binding molecule that binds thereto, will typically reduce the amount of labeled A that binds to the binding molecule. It is to be understood that specificity need not be absolute but generally refers to the context in which the binding occurs. For example, it is well known in the art that antibodies may in some instances cross-react with other epitopes in addition to those present in the target. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule such as HSF1). It is also to be understood that specificity may be evaluated in the context of additional factors such as the affinity of the binding agent for the target versus the affinity of the binding agent for other targets, e.g., competitors. If a binding agent exhibits a high affinity for a target molecule that it is desired to detect and low affinity for nontarget molecules, the antibody will likely be an acceptable reagent. Once the specificity of a binding molecule is established in one or more contexts, it may be employed in other contexts, e.g., similar contexts such as similar assays or assay conditions, without necessarily re-evaluating its specificity. In some embodiments specificity of an antibody can be tested by performing an appropriate assay on a sample expected to lack the target (e.g., a sample from cells in which the gene encoding the target has been disabled or effectively inhibited) and showing that the assay does not result in a signal significantly different to background.

“Subject” refers to any individual, e.g., any individual who has or may have cancer or is at risk of developing cancer or cancer recurrence. The subject is preferably a human or non-human animal, including but not limited to animals such as rodents (e.g., mice, rats, rabbits), cows, pigs, horses, chickens, cats, dogs, primates, etc., and is typically a mammal, and in many embodiments is a human. A subject may be referred to as a “patient”.

HSF1 in Tumor Stroma

HSF1 is a ubiquitously expressed transcription factor best known for its activation by heat (Pelham, 1982; Sakurai and Enoki, 2010; Shamovsky and Nudler, 2008). Recently it has been shown to play a fundamental role in tumor biology (Dai et al., 2012; Dai et al., 2007; Jin et al., 2011; Min et al., 2007). In a wide variety of human cancer cell lines, the depletion of HSF1 markedly reduces growth, survival and metastatic potential (Dai et al., 2007; Fang et al., 2012; Mendillo et al., 2012; Meng et al., 2010; Min et al., 2007; Santagata et al., 2012; Scott et al., 2011). Hsf1 null mice develop normally, but are profoundly resistant to tumorigenesis.

The transcriptional program that is activated by HSF1 in cancer cells is surprisingly different from the program activated by a classic heat-shock (Mendillo et al., 2012). In particular, it acts to support the malignant state by blunting apoptotic responses and promoting pathways that facilitate anabolic metabolism, protein folding, proliferation, invasion, and metastasis (Dai et al., 2012; Dai et al., 2007; Fang et al., 2012; Jin et al., 2011; Mendillo et al., 2012; Meng et al., 2010; Santagata et al., 2013; Santagata et al., 2012; Scott et al., 2011). In human patients, the activation of this program by HSF1 in cancer cells is strongly associated with disease progression in breast, colon, lung, and hepatocellular carcinomas (Fang et al., 2012; Mendillo et al., 2012; Santagata et al., 2011).

Tumor stroma refers to the non-neoplastic cells and connective tissue components of a tumor. Tumor stroma contains a variety of non-neoplastic cell types including fibroblasts, immune cells, and endothelial cells, collectively referred to as “tumor-associated stromal cells” as well as extracellular matrix. Tumor stroma plays a key role in promoting tumor growth and immune suppression. As described herein, Applicants have discovered that HSF1 plays an important role in subverting the normally repressive capacity of stroma and in converting it to a pro-tumorigenic state. HSF1 expression and activation are increased in tumor-associated stromal cells across a broad range of human tumor types, and increased HSF1 expression and activation in tumor-associated stromal cells serve as indicators of poor clinical outcome.

HSF1 activation in tumor-associated stromal cells has profound effects on the gene expression profiles both of the tumor-associated stromal cells themselves and on cancer cells with which they are associated. Compromising HSF1 expression in fibroblasts by genetic knockdown of HSF1 was found to significantly reduce the growth rate of tumors arising from cancer cells co-injected with such fibroblasts into immunocompromised mice as compared with the growth rate of tumors arising from cancer cells co-injected with wild type fibroblasts. Furthermore, tumors arising from cancer cells co-injected with Hsf1 null fibroblasts had a more differentiated, stromal-rich architecture, indicative of a less malignant phenotype. Gene expression analysis of cancer cells cultured with wild type or Hsf1 null fibroblasts demonstrated that activation of HSF1 in the stroma helps to reprogram cancer cells in at least two important ways, i.e., by causing upregulation of genes in cancer cells that enhance their malignant potential and downregulation of genes that would trigger host immune defense responses. Furthermore, HSF1 alters the basal phenotype of fibroblasts in culture and these alterations enhance the growth of cancer cells.

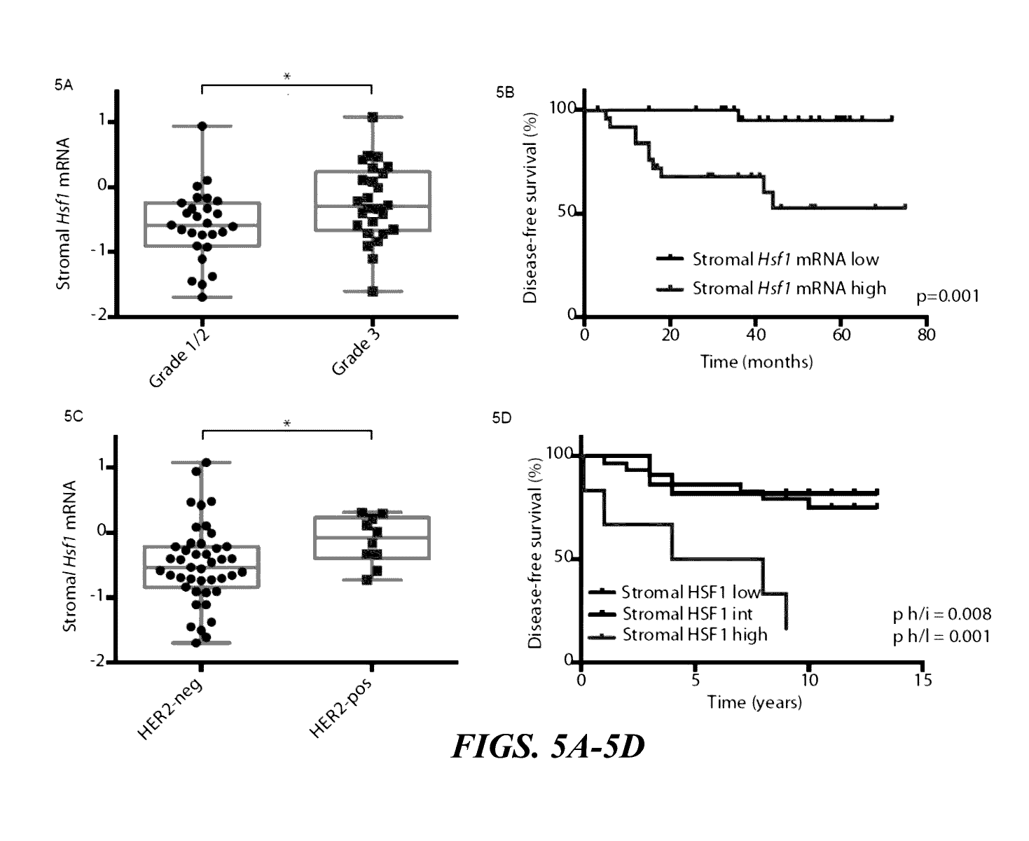

Applicants hypothesized that the increase in nuclear HSF1 in tumor-associated stromal cells might be associated with poor prognosis in human patients. Applicants found that high Hsf1 mRNA levels in tumor stroma samples collected from patients with primary breast tumors significantly correlated with increased tumor grade and poorer patient outcome (FIG. 5B). HSF1 activation was assessed at the protein level by immunohistochemistry (IHC) in a different breast cancer cohort by examining nuclear HSF1 staining. There was markedly reduced disease-free survival, as well as overall survival, in patients whose tumors had high stromal HSF1 activation (FIG. 5D and Figure S5C). In a multivariate model, stromal HSF1 was an independent, significant predictive factor in a multivariate model considering the independent contributions of HSF activation in cancer cells and also various clinicopathologic factors.

In samples from patients with stage I non-small cell lung adenocarcinoma, stromal HSF1 activation showed a significant correlation with poor patient outcome. Disease-free survival was significantly shorter in lung cancer patients whose tumors expressed either high or intermediate HSF1 activity in the stroma. As in the breast cancer cohort, stromal HSF1 activation was significantly and independently associated with disease-free survival. Stromal HSF1 was an independent predictor of progression-free survival in several multivariate models considering KRAS and EGFR mutational status as well as clinicopathologic factors. Applicants asked whether evaluation of HSF1 activation in both stromal cells and cancer cells could improve ability to predict patient outcome. Strikingly, there was not a single recurrence in any of the lung cancer patients that had low HSF1 activity in both the cancer cells and in the stroma over the course of followup.

For cancer cells to proliferate, invade and metastasize, they must recruit and reprogram non-malignant stromal cells to provide them with vital support functions. As described herein, the activation of HSF1 is a key factor in the transcriptional reprogramming of the stroma from a tumor-repressive environment to a supportive one. At least two central signaling pathways in the tumor microenvironment are empowered by HSF1—pathways mediated by TGFβ and by SDF1. HSF1 was found to be activated in the stroma of a wide variety of human cancers and this activation correlated strongly with poor outcome in both lung and breast cancer. In some aspects, the disclosure establishes a role for stromal HSF1 in tumor biology that is distinct, yet highly complementary to, its recently reported role in malignant cells. HSF1 has historically been characterized as a stress-activated transcription factor. In tumors, stromal and cancer cells alike must cope with a variety of potentially lethal challenges, including oxidative stress, nutrient-deprivation and protein misfolding. Yet neither the cancer-HSF1 program previously reported in malignant cells (Mendillo et al., 2012), nor the stromal-HSF1 program described herein is a simple reflection of these inevitable stresses.

The cancer-HSF1 program supports the malignant life style of cancer cells in a multitude of ways, including direct effects on cell cycle, DNA repair, anabolic metabolism and proliferation (Jin et al., 2011; Mendillo et al., 2012; Meng et al., 2010; Santagata et al., 2013). The stromal-HSF1 program, drives pathways that are of specific benefit to the malignant elements within the tumor. These pathways facilitate angiogenesis, ECM organization, adhesion and migration (Beck et al., 2008; Chang et al., 2004; Place et al., 2011; Wang et al., 2006). HSF1 is capable of driving highly divergent transcriptional programs depending on the cellular context. Without wishing to be bound by any theory, the recruitment of different transcriptional co-regulators, different underlying epigenetic states, and different modifications to HSF1 could all play a role. Indeed, HSF1 is known to undergo changes in phosphorylation at dozens of sites, as well as changes in acetylation and sumoylation (Akerfelt et al., 2010; Chou et al., 2012; Raychaudhuri et al., 2014). Given the diversity of tumors in which we observe the stromal activation of HSF1, the specific molecular mechanisms involved may encompass a highly diverse array of signaling pathways.

One feature of this program, described herein, is the way HSF1 responses are coordinated between cancer cells and stroma. Applicants found that TGFβ and SDF1 are two extracellular mediators of the HSF1 program in CAFs. While it was previously recognized that these proteins, when secreted by CAFs, enhanced the pro-tumorigenic phenotype (Kojima et al., 2010; Orimo et al., 2005), the factors responsible for their upregulation were not known. HSF1 has been shown to directly bind to heat-shock elements in the genes of several chemokines (Henderson and Kaiser, 2013; Maity et al., 2011) during heat shock. Conversely, HSF1 can be activated by exposure to cytokines such as TGFβ and IL-1β, in vitro (Sasaki et al., 2002). Taking these observations together, and without wishing to be bound by an theory, we suggest that reciprocal interactions between secreted cytokines and intracellular HSF1 programs that are normal responses to fever and infection have been co-opted by diverse cell types in tumors to fuel the malignant state.

The HSF1-dependent heat-shock response has traditionally been conceived as an internally-driven cellular response to proteotoxic stress. Remarkably, however, work in C. elegans has established that HSF1 can be activated in a non-cell-autonomous manner. Acute heat-stresses detected solely by thermosensory neurons can orchestrate HSF1-dependent heat-shock responses throughout the animal. This coordinated response benefits the organism as a whole (Prahlad et al., 2008). As described herein, in tumors, cancer cells induce the activation of HSF1 in the stroma, and this activation benefits the tumor as a whole (albeit to the detriment of the patient). But in addition to this, in stroma the HSF1-regulated program itself is non-cell-autonomous. It results in the secretion of factors that act to enhance the survival and proliferation of neighboring cancer cells. Without wishing to be bound by any theory, Applicants suggest that the interplay between HSF1 responses in cancer cells and stroma have their origins in ancient biological mechanisms that act to promote the survival of multicellular organisms in a non-cell-autonomous way.

The surprising biology of HSF1 responses in cancer and stromal cells of tumors have both diagnostic and therapeutic implications. From a diagnostic perspective, assessing HSF1 in both stromal and cancer cells may help to guide treatment choices in early stage cancers, especially lung cancer, where currently there are no reliable markers in use for gauging malignant potential other than tumor size. The increased surveillance of patients at high risk to develop lung cancer is creating an acute need for markers that can predict which early-stage tumors are most likely to progress, to avoid over-treatment and its associated morbidities. From a therapeutic perspective, the dependence of even the most robust cancers on the supporting stromal cells, and the relative genetic stability of the stroma, make HSF1 an attractive target for intervention in both cancer cells and the stroma. The formidable ability of advanced cancers to evolve resistance to therapy makes it attractive to target normal biological networks that have been co-opted to support malignancy.

In some aspects, the invention provides methods of classifying a sample comprising tumor-associated stromal cells with respect to cancer diagnosis (e.g., the presence or absence of cancer), cancer aggressiveness, cancer outcome, or cancer treatment selection, based at least in part on assessing the level of HSF1 expression, HSF1 activation, or both (i.e., HSF1 expression and/or activation), in tumor-associated stromal cells. In some aspects, the invention provides methods of cancer diagnosis, prognosis, or treatment-specific prediction, based at least in part on assessing the level of HSF1 expression, HSF1 activation, or both, in stromal cells of a sample, e.g., a tumor sample or suspected tumor sample. In some embodiments, the cancer is an adenocarcinoma. In some embodiments the cancer is a breast, lung, skin, esophageal, colon, gastric, or prostate tumor, e.g., a breast, lung, skin, esophageal, colon, gastric, or prostate adenocarcinoma. In some embodiments the lung tumor is a non small cell lung cancer (NSCLC). In some embodiments the tumor is a squamous cell carcinoma. In some embodiments the tumor is not a squamous cell carcinoma. In some embodiments the cancer is a sarcoma. In some embodiments a tumor is a Stage 1 tumor as defined in the TNM Classification of Malignant Tumours (2009), e.g., a Stage 1a or Stage 1b tumor. In some embodiments a tumor is a Stage II tumor as defined in the TNM Classification of Malignant Tumours (2009). It will be understood that results of an assay of HSF1 expression and/or HSF1 activation in tumor-associated stromal cells may be used in combination with results from other assays, or other information, to provide a sample classification, diagnosis, prognosis, or prediction relating to cancer, cancer outcome, or treatment response. Such combination methods are within the scope of the invention.

In some aspects, the invention relates to methods for classifying a sample comprising tumor-associated stromal cells according to the level of HSF1 expression and/or HSF1 activation in the tumor-associated stromal cells. In some aspects, the invention relates to methods for classifying a sample comprising tumor-associated stromal cells based at least in part on the level of HSF1 expression and/or HSF1 activation in the tumor-associated stromal cells. For purposes hereof, a method that comprises assessing HSF1 expression and/or assessing HSF1 activation in tumor-associated stromal cells may be referred to as an “HSF1-based method”. A procedure that is used to assess (detect, measure, determine, quantify) HSF1 expression and/or HSF1 activation may be referred to as an “HSF1-based assay”. An HSF1-based assay described herein may be performed to assess HSF1 expression and/or activation in tumor-associated stromal cells in or obtained from a tumor, e.g., in a sample comprising or derived at least in part from tumor-associated stromal cells. In some embodiments HSF1 expression and/or activation is assessed specifically in tumor-associated stromal cells as distinct from cancer cells. The tumor-associated stromal cells may be separated from or distinguished from cancer cells and/or other cells in the sample. In some embodiments HSF1 expression and/or activation may be assessed in a total cell population or sample that comprises tumor-associated stromal cells and cancer cells or cellular material wherein the tumor-associated stromal cells or cellular material constitute a significant proportion of total cells or cellular material in the sample. In some embodiments an HSF1-based assay may further comprise assessing HSF1 expression and/or assessing HSF1 activation in cancer cells from the same tumor as tumor-associated stromal cells in which HSF1 expression and/or activation is assessed.

In some embodiments of any aspect described herein, at least 10%, 20%, 30%, 40%, 50%, 60%, 70%, 80%, 90%, 95%, or more of the cells or cellular material are tumor-associated stromal cells or cellular material derived therefrom. In some embodiments of any aspect described herein, the tumor-associated stromal cells comprise or consist of cancer-associated fibroblasts. In some embodiments at least 10%, 20%, 30%, 40%, 50%, 60%, 70%, 80%, 90%, 95%, or more of the tumor-associated stromal cells are cancer-associated fibroblasts. In some embodiments at least 10%, 20%, 30%, 40%, 50%, 60%, 70%, 80%, 90%, 95%, or more of the cells are cancer-associated fibroblasts.

In some embodiments a tumor sample that is relatively enriched for stromal tissue versus neoplastic tissue may be used. Either HSF1 expression, HSF1 activation, or both, can be assessed in various embodiments. Certain assays such as IHC can be used to assess both expression and activation.

In some embodiments HSF1 expression and/or activation is assessed specifically in tumor-associated stromal cells and specifically in cancer cells of the same tumor. In some embodiments results of measuring HSF1 expression and/or activation in cancer cells are used to refine a diagnosis, classification, prognosis, or treatment selection that is based on HSF1 expression and/or activation in tumor-associated stromal cells. In some embodiments measuring HSF1 expression and/or activation in cancer cells and in tumor-associated stromal cells may provide diagnosis, classification, prognosis, or treatment selection information that has a higher likelihood of being correct than measuring HSF1 expression and/or activation in cancer cells only or in tumor-associated stromal cells only. For example, in some embodiments, if HSF1 expression and/or activation is determined to be absent or low both in tumor-associated stromal cells and in cancer cells of the tumor, one can predict with greater confidence that the patient will not have a poor outcome than if HSF1 expression and/or activation was determined in tumor-associated stromal cells and found to be absent or low but was not measured in cancer cells. In some embodiments a tumor may be classified into any of four groups: (1) cancer cell HSF1 absent or low, stromal cell HSF1 absent or low; (2) cancer cell HSF1 absent or low, stromal cell HSF1 high; (3) cancer cell HSF1 high, stromal cell HSF1 absent or low; (4) cancer cell HSF1 high, stromal cell HSF1 high. In some embodiments a subject having a tumor classified in group 1 has a more favorable prognosis than if the tumor were classified in group 2, 3, or 4.

In some embodiments, the level of HSF1 expression is assessed by determining the level of an HSF1 gene product. Thus in some embodiments, the invention relates to methods for classifying a tumor-associated stromal cell or sample comprising one or more tumor-associated stromal cells according to the level of an HSF1 gene product in the tumor-associated stromal cell or cells or sample. In some embodiments, the invention provides a method of classifying a sample, the method comprising steps of: (a) providing a sample obtained from a subject; and (b) assessing HSF1 expression in tumor-associated stromal cells the sample, wherein the level of HSF1 expression is correlated with a phenotypic characteristic, thereby classifying the sample with respect to the phenotypic characteristic. In some embodiments, the invention provides a method of classifying a sample, the method comprising steps of: (a) providing a sample obtained from a subject; and (b) determining the level of an HSF1 gene product in tumor-associated stromal cells of the sample, wherein the level of an HSF1 gene product is correlated with a phenotypic characteristic, thereby classifying the sample with respect to the phenotypic characteristic. In some embodiments the phenotypic characteristic is presence or absence of cancer. In some embodiments, the cancer is invasive cancer. In some embodiments the sample does not show evidence of invasive cancer, and the phenotypic characteristic is presence or absence of pre-invasive cancer (cancer in situ). In some embodiments the phenotypic characteristic is cancer prognosis. In some embodiments the phenotypic characteristic is predicted treatment outcome. In some embodiments the HSF1 gene product is HSF1 mRNA. In some embodiments the HSF1 gene product is HSF1 polypeptide.

In some aspects, the invention provides a method of classifying a sample, the method comprising: (a) determining the level of HSF1 expression and/or activation in a sample comprising stromal cells; (b) comparing the level of HSF1 expression and/or HSF1 activation with a control level; and (c) classifying the sample with respect to cancer diagnosis, wherein a greater (increased) level of HSF1 expression and/or activation in the stromal cells of the sample as compared with the control level is indicative of the presence of cancer. In some embodiments, a greater level of HSF1 expression and/or HSF1 activation in stromal cells of the sample than in normal stromal cells is indicative of the presence of in situ cancer in a sample that does not show evidence of invasive cancer.

In some aspects, the invention provides a method of classifying a sample, the method comprising: (a) determining the level of HSF1 expression or the level of HSF1 activation in a sample obtained from a tumor; (b) comparing the level of HSF1 expression or HSF1 activation with a control level of HSF1 gene expression or HSF1 activation; and (c) classifying the sample with respect to cancer prognosis, wherein a greater level of HSF1 gene expression or HSF activation in the sample obtained from the tumor as compared with the control level of HSF1 gene expression or HSF activation, respectively, is indicative that the sample originated from a tumor that belongs to a poor prognosis class. In some aspects, the invention provides a method of classifying a tumor, the method comprising: (a) determining the level of HSF1 expression and/or the level of HSF1 activation in a sample comprising tumor-associated stromal cells obtained from a tumor; (b) comparing the level of HSF1 expression and/or activation in said tumor-associated stromal cells with a control level of HSF1 gene expression or HSF1 activation; and (c) classifying the sample with respect to cancer prognosis, wherein a greater level of HSF1 gene expression or HSF activation in the tumor-associated stromal cells as compared with the control level of HSF gene expression or HSF1 activation, respectively, is indicative that the tumor belongs to a poor prognosis class.

In some aspects, the invention relates to methods for classifying a sample according to the level of HSF1 activation in tumor-associated stromal cells of the sample. As used herein, “HSF1 activation” refers the process in which HSF1 polypeptide is phosphorylated, trimerizes, and translocates to the nucleus, where it binds to DNA sequences and regulates expression of genes containing such sequences (e.g., in their promoter regions) (“HSF1-regulated genes”). In some embodiments, the invention is directed to a method of classifying a sample with respect to a phenotypic characteristic, the method comprising steps of: (a) providing a sample comprising tumor-associated stromal cells obtained from a subject; and (b) determining the level of activation of HSF1 polypeptide in the tumor-associated stromal cells, wherein the level of activation of an HSF1 polypeptide is correlated with a phenotypic characteristic, thereby classifying the sample with respect to the phenotypic characteristic. In some embodiments the sample does not show evidence of invasive cancer, and the phenotypic characteristic is presence or absence of pre-invasive cancer. In some embodiments the phenotypic characteristic is cancer prognosis. In some embodiments the phenotypic characteristic is predicted treatment outcome. In some embodiments, the level of HSF1 activation is assessed by determining the level of nuclear HSF1 in the sample. Thus in some embodiments the invention relates to methods for classifying a sample according to the level of nuclear HSF1 in tumor-associated stromal cells of the sample. In some embodiments, assessing the level of HSF1 activation comprises assessing HSF1 activity. In some embodiments, assessing the level of HSF1 activity comprises measuring expression of one or more genes that are regulated by HSF1 in tumor-associated stromal cells. In some embodiments, assessing the level of HSF1 activity comprises measuring binding of HSF1 to the promoter region of one or more HSF1-regulated genes.

In some aspects the invention provides a method of detecting a tumor-associated stromal cell comprising contacting a sample comprising a plurality of stromal cells with a reagent that specifically binds to HSF1 mRNA or HSF1 polypeptide and detecting increased binding of the reagent in at least one of said stromal cells as compared to a control level, thereby detecting a tumor-associated stromal cell.

In some aspects the invention provides a method of detecting a tumor-associated stromal cell comprising contacting a sample comprising stromal tissue with a reagent that specifically binds to HSF1 mRNA or HSF1 polypeptide and detecting increased binding of the reagent as compared to a control level, thereby detecting a tumor-associated stromal cell.

In some aspects of the invention, detection of increased HSF expression and/or activation in tumor-associated stromal cells is of use for diagnosis of cancer, e.g., for detection of cancer. According to certain methods of the invention, samples can be classified as belonging to (i.e., obtained from) an individual who has cancer or is likely to develop cancer. Without wishing to be bound by any theory, HSF1 expression and/or activation may become elevated in tumor-associated stromal cells during the in situ stage of malignant transformation, prior to invasion. Detection of elevated HSF expression and/or activation in tumor-associated stromal cells may be used for early diagnosis of cancer, e.g., for detection of cancer in situ. According to certain of the methods samples can be classified as belonging to (i.e., obtained from) an individual who has cancer in situ (CIS) or is likely to develop CIS or who has CIS and is likely to develop invasive cancer based at least in part on detecting increased HSF1 expression and/or activation in tumor-associated stromal cells.

In some embodiments, detection of increased HSF1 expression and/or activation in stromal cells indicates that a subject has an increased likelihood of having CIS or developing CIS than would be the case in the absence of increased HSF1 expression and/or activation in such cells. In some embodiments, detection of increased HSF1 expression and/or activation in a sample comprising or derived from stromal cells is of use to detect a CIS before it becomes detectable on physical examination or, in some embodiments, before it becomes detectable on imaging. In some embodiments, detection of increased HSF1 expression and/or activation in a sample comprising stromal cells may be used to help differentiate lesions that are malignant or that have significant potential to become invasive or metastasize from benign lesions. In accordance with certain embodiments of the invention, a lesion has an increased likelihood of being malignant or having significant potential to become invasive or metastasize if increased HSF1 expression and/or activation is detected in stromal cells of the sample than would be the case if increased HSF1 expression and/or activation is not detected in stromal cells. Detection of increased HSF1 expression and/or activation could, for example, indicate a need for additional or more frequent follow-up of the subject or for treatment of the subject from whom the sample was obtained. In some embodiments, detection of elevated HSF1 expression and/or activation in stromal cells is used together with one or more other indicators of dysplasia and/or neoplasia to detect the presence of CIS or to differentiate lesions that are malignant or that have significant potential to become invasive or metastasize from benign lesions. In some embodiments, detection of elevated HSF1 expression and/or activation may enable classification of a sample that could not be reliably classified (e.g., as high risk or low risk) using standard histopathologic criteria. It will be understood that whether a sample (or tumor from which the sample originated) has an increased level of HSF1 expression and/or HSF1 activation in stromal cells can be determined by comparing the sample with a suitable control.

In some aspects, the invention provides method of identifying CIS, comprising assessing HSF1 expression and/or activation in stromal cells of a tissue or cell sample, wherein the sample does not show evidence of invasive cancer, and wherein increased HSF1 expression and/or activation in stromal cells of the sample is indicative of CIS. In some aspects, the invention provides a method of predicting the likelihood that a subject will develop invasive cancer, comprising assessing HSF1 expression and/or activation of HSF1 in stromal cells of a tissue or cell sample obtained from the subject, wherein increased expression of HSF1 or increased activation of HSF1 in the stromal cells is indicative of an increased likelihood that the subject will develop invasive cancer. In some embodiments the stromal cells are tumor-associated stromal cells. In some aspects, the invention provides a method of method of diagnosing CIS in a subject, comprising assessing HSF1 expression and/or activation in stromal cells of a tissue or cell sample obtained from the subject, wherein the sample does not show evidence of invasive cancer, and wherein increased expression of HSF1 and/or increased activation of HSF1 in stromal cells in the sample indicates the presence of CIS in the subject.

In some embodiments, a method of identifying, detecting, or diagnosing cancer, e.g., cancer in situ, is applied to a sample obtained from a subject who is at increased risk of cancer (e.g., increased risk of developing cancer or having cancer) or is suspected of having cancer or is at risk of cancer recurrence. A subject at increased risk of cancer may be, e.g., a subject who has not been diagnosed with cancer but has an increased risk of developing cancer as compared with a control, who may be matched with regard to one or more demographic characteristics such as age, gender, etc. For example, the subject may have a risk at least 1.2, 1.5, 2, 3, 5, 10 or more times that of an age-matched control (e.g., of the same gender), in various embodiments of the invention. It will be understood that “age-matched” can refer to the same number of years of age as the subject or within the same age range as the subject (e.g., a range of 5 or 10 years). For example, a control may be up to 5 years older or younger than the subject. Determining whether a subject is considered “at increased risk” of cancer is within the skill of the ordinarily skilled medical practitioner. Any suitable test(s) and/or criteria can be used. For example, a subject may be considered “at increased risk” of developing cancer if any one or more of the following apply: (i) the subject has a mutation or genetic polymorphism that is associated with increased risk of developing or having cancer relative to other members of the general population not having such mutation or genetic polymorphism (e.g., certain mutations in the BRCA1 or BRCA2 genes are well known to be associated with increased risk of a variety of cancers, including breast cancer and ovarian cancer; mutations in tumor suppressor genes such as Rb or p53 can be associated with a variety of different cancer types); (ii) the subject has a gene or protein expression profile, and/or presence of particular substance(s) in a sample obtained from the subject (e.g., blood), that is/are associated with increased risk of developing or having cancer relative to other members of the general population not having such gene or protein expression profile, and/or substance(s) in a sample obtained from the subject; (iii) the subject has one or more risk factors such as having a family history of cancer, having been exposed to a tumor-promoting agent or carcinogen (e.g., a physical carcinogen, such as ultraviolet or ionizing radiation; a chemical carcinogen such as asbestos, tobacco components or other sources of smoke, aflatoxin, or arsenic; a biological carcinogen such as certain viruses or parasites), or has certain conditions such as chronic infection/inflammation that are correlated with increased risk of cancer; (iv) the subject is over a specified age, e.g., over 60 years of age, etc. In the case of breast cancer, a subject diagnosed as having lobular carcinoma in situ (LCIS) is at increased risk of developing cancer. A subject suspected of having cancer may be a subject who has one or more symptoms of cancer or who has had a diagnostic procedure performed that suggested or was at least consistent with the possible existence of cancer but was not definitive. A subject at risk of cancer recurrence can be any subject who has been treated for cancer such that the cancer was rendered undetectable as assessed, for example, by appropriate methods for cancer detection.

According to certain methods of the invention, a sample, tumor, or subject can be classified as belonging to a particular class of outcome based at least in part on the level of HSF1 expression and/or HSF1 activation in tumor-associated stromal cells. For example, in some embodiments, a sample, tumor, or subject can be classified as belonging to a high risk class (e.g., a class with a prognosis for a high likelihood of recurrence after treatment or a class with a prognosis for a high likelihood of discovery of metastasis post-diagnosis or a class with a poor prognosis for survival after treatment) or a low risk class (e.g., a class with a prognosis for a low likelihood of recurrence after treatment or a class with a prognosis for a low likelihood of discovery of metastasis post-diagnosis or a class with a good prognosis for survival after treatment). In some embodiments, survival after treatment is assessed 5 or 10 years after diagnosis, wherein increased expression of HSF1 and/or increased activation of HSF1 in tumor-associated stromal cells is predictive of decreased likelihood of survival at 5 years or 10 years post-diagnosis. In some embodiments, increased expression of HSF1 and/or increased activation of HSF1 in tumor-associated stromal cells is predictive of decreased mean (average) or median survival. In some embodiments survival is overall survival, wherein increased expression of HSF1 and/or increased activation of HSF1 in tumor-associated stromal cells is predictive of decreased overall survival (increased overall mortality). In some embodiments survival is disease-specific survival, wherein increased expression of HSF1 and/or increased activation of HSF1 in tumor-associated stromal cells is predictive of decreased disease-specific survival (i.e., increased disease-specific mortality), wherein “disease-specific” in the context of outcome, refers to considering only deaths due to cancer, e.g., breast cancer, lung cancer, or any other cancer of interest.

According to certain methods of the invention, a sample, tumor, or subject can be classified as belonging to a particular class with regard to tumor aggressiveness. For example, a sample or tumor can be classified into a more aggressive class or a less aggressive class or a subject can be classified as having a tumor that is more aggressive or less aggressive. “More aggressive” in this context means that the sample or tumor has one or more features that correlate with a poor outcome. A poor outcome may be, e.g., progression (e.g., after treatment), recurrence after treatment, or cancer-related mortality (e.g., within 5, 10, or 20 years after treatment). For example, a tumor classified as more aggressive may have an increased likelihood of having metastasized locally or to remote site(s) at the time of diagnosis, an increased likelihood of metastasizing or progressing locally (e.g., within a specified time period after diagnosis such as 1 year, 2 years, etc.), an increased likelihood of treatment resistance (e.g., a decreased likelihood of being eradicated or rendered undetectable by treatment). In some aspects, the invention provides a method of assessing the aggressiveness of a tumor, the method comprising: measuring the level of HSF1 expression and/or activation in tumor-associated stromal cells of the tumor, wherein if the level of HSF1 expression and/or activation in the tumor-associated stromal cells is increased, the tumor is classified as belonging to a more aggressive class. In some aspects, the invention provides a method of assessing the aggressiveness of a tumor, the method comprising: (a) measuring the level of HSF1 expression and/or activation in tumor-associated stromal cells of the tumor; (b) comparing the level of HSF1 expression and/or activation with a control level; and (c) assessing the aggressiveness of the tumor based at least in part on the result of step (b), wherein a greater level of HSF1 expression and/or activation in the tumor-associated stromal cells as compared with the control level is indicative of increased aggressiveness.

In some aspects, the invention provides a method of assessing the likelihood that a tumor has metastasized, the method comprising: (a) measuring the level of HSF1 expression and/or activation in tumor-associated stromal cells of the tumor; (b) comparing the level of HSF1 expression and/or activation with a control level, wherein a higher level of HSF1 expression and/or activation as compared with a control level is indicative of a greater likelihood that the tumor has metastasized. In some aspects, the invention provides a method of assessing the likelihood that a tumor will metastasize, the method comprising: (a) measuring the level of HSF1 expression and/or activation in tumor-associated stromal cells of the tumor; (b) comparing the level of HSF1 expression and/or activation with a control level, wherein a higher level of HSF1 expression and/or activation as compared with a control level is indicative of a greater likelihood that the tumor will metastasize.

In some embodiments an HSF1-based method of the invention may be useful for selecting a treatment regimen for a subject. For example, such results may be useful in determining whether a subject should receive, e.g., would likely benefit from, administration of one or more chemotherapeutic agents (chemotherapy), hormonal therapy, an anti-HER2 agent, or other treatment such as radiation. In some embodiments, “chemotherapeutic agent” refers to an anti-tumor agent that has cytotoxic or cytostatic properties and does not act primarily by interacting with (e.g., interfering with) a hormonal pathway that is specific or relatively specific to particular cell type(s). Exemplary chemotherapeutic agents include anti-metabolites, alkylating agents, microtubule stabilizers or microtubule assembly inhibitors (e.g., taxanes or vinca alkaloids), topoisomerase inhibitors, and DNA intercalators (e.g., anthracycline antibiotics). Such agents are frequently administered systemically. Often, multiple agents are administered. Exemplary treatment regimens for breast cancer include CMF (cyclophosphamide, methotrexate, and 5-FU), AC (doxorubicin and cyclophosphamide), and anthracycline-based regimens. Capecitabine is a prodrug, that is enzymatically converted to 5-fluorouracil following administration (e.g., in tumor tissue) and is a component of a number of breast cancer treatment regimens. Tegafur is another 5-FU prodrug, which may be administered together with uracil, a competitive inhibitor of dihydropyrimidine dehydrogenase. A “hormonal therapy” (also termed “endocrine therapy”) refers to an antitumor agent that acts primarily by interacting with the endocrine system, e.g., by interfering with a hormonal pathway that is active in a hormonally responsive tissue such as breast, prostate, or endometrium. Exemplary hormonal therapies include, e.g., drugs that inhibit the production or activity of hormones that would otherwise contribute to survival, proliferation, invasion, or other activities of cancer cells. For example, in the case of breast cancer, hormonal therapy can comprise an agent that inhibits ER signaling. The agent may interact with and inhibit the ER or inhibit estrogen biosynthesis. In some embodiments hormonal therapy comprises a selective estrogen receptor modulator (SERM) such as tamoxifen, raloxifene, or toremifene. It will be appreciated that SERMs can act as ER inhibitors (antagonists) in breast tissue but, depending on the agent, may act as activators (e.g., partial agonists) of the ER in certain other tissues (e.g., bone). It will also be understood that tamoxifen itself is a prodrug that has relatively little affinity for the ER but is metabolized into active metabolites such as 4-hydroxytamoxifen (afimoxifene) and N-desmethyl-4-hydroxytamoxifen (endoxifen). Such active metabolites may be used as ER inhibitors. In some embodiments, hormonal therapy comprises a selective estrogen receptor down-regulators (SERD) such as fulvestrant or CH4986399. In some embodiments hormonal therapy comprises an agent that inhibits estrogen biosynthesis. For example, estrogen deprivation can be achieved using inhibitors that block the last stage in the estrogen biosynthetic sequence, i.e., the conversion of androgens to estrogens by the enzyme aromatase (“aromatase inhibitors”). Aromatase inhibitors include, e.g., letrozole, anastrazole, and exemestane. In the case of prostate cancer, “hormonal therapy” can comprise administering an agent that interferes with androgen receptor (AR) signaling. For example, antiandrogens are drugs that bind to and inhibit the AR, blocking the growth- and survival-promoting effects of testosterone on certain prostate cancers. Examples include flutamide and bicalutamide. Analogs of gonadotropin-releasing hormone (GnRH) can be used to suppress production of estrogen and progesterone from the ovaries, or to suppress testosterone production from the testes. Leuprolide and goserelin are GnRH analogs which are used primarily for the treatment of hormone-responsive prostate cancer.

“Adjuvant therapy” refers to administration of one or more antitumor agents in connection with, e.g., following, local therapy such as surgery and/or radiation. Adjuvant therapy may be used, e.g., when a cancer appears to be largely or completely eradicated, but there is risk of recurrence. Such therapy may help eliminate residual cells at the site of the primary tumor and/or cells that have disseminated.

“Neoadjuvant therapy” refers to adjuvant therapy administered prior to local therapy, e.g., to shrink a primary tumor.

“Anti-HER2” therapy refers to administration of an antitumor agent that acts primarily by interacting with (e.g., interfering with) HER2. Such agents may be referred to as “anti-HER2” agents. Anti-HER2 agents include, e.g., monoclonal antibodies that bind to HER2, such as trastuzumab and pertuzumab, and various small molecule kinase inhibitors that bind to HER2 and inhibits its kinase activity. Pertuzumab is a recombinant, humanized monoclonal antibody that binds to the extracellular domain II, sterically blocking homo- and heterodimerization with other ERBB receptors, thus preventing signal transduction. In some embodiments, an anti-HER2 agent inhibits HER2 and at least one other member of the human epidermal growth factor receptor family. Examples of such agents include, e.g., dual EGFR (Erb-B1) and HER2 kinase inhibitors such as lapatinib and pan-ERBB kinase inhibitors such as neratinib. In some embodiments, an anti-tumor agent is an antibody-drug conjugate (ADC). For example, an anti-HER2 antibody can be conjugated to a cytotoxic agent. Cytotoxic agents useful for such purposes include, e.g., calicheamicins, auristatins, maytansinoids, and derivatives of CC 1065. For example, trastuzumab emtansine (T-DM1) is an antibody-drug conjugate ADC that combines intracellular delivery of the cytotoxic agent, DM1 (a derivative of maytansine) with the antitumor activity of trastuzumab.

In some embodiments, results of an HSF1-based assay may be useful for selecting an appropriate treatment regimen and/or for selecting the type or frequency of procedures to be used to monitor the subject for local or metastatic recurrence after therapy and/or the frequency with which such procedures are performed. For example, subjects classified as having a poor prognosis (being at high risk of poor outcome) may be treated and/or monitored more intensively than those classified as having a more favorable prognosis, e.g., a good prognosis. Thus any of the diagnostic, prognostic, or treatment-specific predictive methods can further comprise using information obtained from the assay to help in selecting a treatment or monitoring regimen for a subject suffering from cancer or at increased risk of cancer or at risk of cancer recurrence or in providing an estimate of the risk of poor outcome such as cancer related mortality or recurrence. The information may be used, for example, by a subject's health care provider in selecting a treatment or in treating a subject. A health care provider could also or alternatively use the information to provide a cancer patient with an accurate assessment of his or her prognosis. In some embodiments, a method of the invention can comprise making a treatment selection or administering a treatment based at least in part on the result of an HSF1-based assay. In some embodiments, a method of the invention can comprise selecting or administering more aggressive treatment to a subject, if the subject is determined to have a poor prognosis. In some embodiments, a method of the invention can comprise selecting or administering more aggressive treatment, if the subject is determined to have increased HSF1 expression or HSF1 activation in stromal cells, e.g., tumor-associated stromal cells. Often a “treatment” or “treatment regimen” refers to a course of treatment involving administration of an agent or use of a non-pharmacological therapy multiple times over a period of time, e.g., over weeks or months. A treatment can include one or more pharmacological agents (often referred to as “drugs” or “compounds”) and/or one or more non-pharmacological therapies such as radiation, surgery, etc. A treatment regimen can include the identity of agents to be administered to a subject and may include details such as the dose(s), dosing interval(s), number of courses, route of administration, etc. “Monitoring regimen” refers to repeated evaluation of a subject over time by a health care provider, typically separated in time by weeks, months, or years. The repeated evaluations can be on a regular or predetermined approximate schedule and are often performed with a view to determining whether a cancer has recurred or tracking the effect of a treatment on a tumor or subject.

“More aggressive” treatment (also referred to as “intensive” or “more intensive” treatment herein) can comprise, for example, (i) administration of chemotherapy in addition to, or instead of, hormonal therapy; (ii) administration of a dose of one or more agents (e.g., chemotherapeutic agent) that is at the higher end of the acceptable dosage range (e.g., a high dose rather than a medium or low dose, or a medium dose rather than a low dose) and/or administration of a number of doses or a number of courses at the higher end of the acceptable range and/or use of non-hormonal cytotoxic/cytostatic chemotherapy; (iii) administration of multiple agents rather than a single agent; (iv) administration of more, or more intense, radiation treatments; (v) administration of a greater number of agents in a combination therapy; (vi) use of adjuvant therapy; (vii) more extensive surgery, such as mastectomy rather than breast-conserving surgery such as lumpectomy. For example, a method can comprise (i) selecting that the subject not receive chemotherapy (e.g., adjuvant chemotherapy) if the tumor is considered to have a good prognosis; or (ii) selecting that the subject receive chemotherapy (e.g., adjuvant chemotherapy), or administering such chemotherapy, if the tumor is considered to have a poor prognosis. In some embodiments, a method of the invention can comprise selecting that a subject receives less aggressive treatment or administering such treatment, if the subject is determined to have a good prognosis. “Less aggressive” (also referred to as “less intensive”) treatment could entail, for example, using dose level or dose number at the lower end of the acceptable range, not administering adjuvant therapy, selecting a breast-conserving therapy rather than mastectomy, selecting hormonal therapy rather than non-hormonal cytotoxic/cytostatic chemotherapy, or simply monitoring the patient carefully. “More intensive” or “intensive” monitoring could include, for example, more frequent clinical and/or imaging examination of the subject or use of a more sensitive imaging technique rather than a less sensitive technique. “Administering” a treatment could include direct administration to a subject, instructing another individual to administer a treatment to the subject (which individual may be the subject themselves in the case of certain treatments), arranging for administration to a subject, prescribing a treatment for administration to a subject, and other activities resulting in administration of a treatment to a subject. “Selecting” a treatment or treatment regimen could include determining which among various treatment options is appropriate or most appropriate for a subject, recommending a treatment to a subject, or making a recommendation of a treatment for a subject to the subject's health care provider.

In some aspects, the invention provides a method of selecting a regimen for monitoring or treating a subject in need of treatment for cancer comprising: (a) measuring the level of HSF1 expression and/or activation in tumor-associated stromal cells obtained from the subject; and (b) selecting an intensive monitoring or treatment regimen if the level of HSF1 expression and/or HSF1 activation is increased in the tumor-associated stromal cells. In some aspects, the invention provides a method of selecting a regimen for monitoring or treating a subject in need of treatment for cancer, wherein said regimen is selected from among multiple options including at least one more intensive regimen and at least one less intensive regimen, the method comprising: (a) obtaining a classification of the subject, wherein the subject is classified into a high risk or a low risk group based at least in part on an assessment of the level of HSF1 expression or HSF1 activation in tumor-associated stromal cells obtained from the subject; and (b) selecting a more intensive regimen if the subject is classified as being in a high risk group or selecting a less intensive regimen if the subject is classified as being in a low risk group. In some aspects, the invention provides a method of monitoring or treating a subject in need of treatment for cancer comprising: (a) obtaining a classification of the subject, wherein the classification is based at least in part on an assessment of the level of HSF1 expression or HSF1 activation in tumor-associated stromal cells obtained from the subject; and (b) monitoring or treating the subject according to an intensive regimen if the subject is classified as being in a high risk group or monitoring or treating the subject with a less intensive regimen if the subject is classified as being in a low risk group. “Obtaining a classification” could comprise any means of ascertaining a classification such as performing an HSF1-based assay (or directing that an HSF1-based assay be performed) and assigning a classification based on the results, receiving results of an HSF1-based assay and assigning a classification using the results, receiving or reviewing a classification that was previously performed, etc.

In some embodiments a subject has been previously treated for the cancer, while in other embodiments the subject has not previously received treatment for the cancer. In some embodiments the previous treatment for a breast tumor is hormonal therapy such as tamoxifen or another anti-estrogen agent, e.g., another SERM.

In some embodiments, a subject falls within a selected age group or range, e.g., 40 years old or less, 50 years old or less, 55 years old or less, 60 years old or less, between 40 and 60 years of age, 40 years old or more, 50 years old or more, 55 years old or more, 60 years old or more, etc. Any age group or range may be selected in various embodiments of the invention, whether or not specifically mentioned here. In some embodiments, a female subject is pre-menopausal. In some embodiments, a female subject is post-menopausal.

In some embodiments a subject, e.g., a subject having or at risk of lung cancer or lung cancer recurrence, is a current smoker or former smoker. In some embodiments a subject, e.g., a subject having or at risk of developing lung cancer or lung cancer recurrence, is a non-smoker who has no or essentially no history of smoking.

In some embodiments, an HSF1-based method may be used to identify cancer patients that do not require adjuvant therapy, e.g., adjuvant hormonal therapy and/or adjuvant chemotherapy. For example, a prognostic method may identify patients that have a good prognosis and would be unlikely to experience clinically evident recurrence and/or metastasis even without adjuvant therapy. Since adjuvant therapy can cause significant side effects, it would be beneficial to avoid administering it to individuals whom it would not benefit. In some embodiments, an HSF1-based prognostic method of the invention may be used to identify cancer patients that have a poor prognosis (e.g., they are at high risk of recurrence and/or metastasis) and may therefore benefit from adjuvant therapy. In some embodiments, an HSF1-based prognostic method may be used to identify cancer patients that might not be considered at high risk of poor outcome based on other prognostic indicators (and may therefore not receive adjuvant therapy) but that are in fact at high risk of poor outcome, e.g., recurrence and/or metastasis. Such patients may therefore benefit from adjuvant therapy. In some embodiments, HSF1-based method may be used in a subject with cancer in whom an assessment of the tumor based on standard prognostic factors, e.g., standard staging criteria (e.g., TMN staging), histopathological grade, does not clearly place the subject into a high or low risk category for recurrence after local therapy (e.g., surgery) and/or for whom the likelihood of benefit from adjuvant therapy is unclear, as may be the case in various early stage cancers where, e.g., the cancer is small and has not detectably spread to regional lymph nodes or metastasized more remotely.

In some embodiments, an HSF1-based method may be used to provide prognostic information for a subject with a breast tumor that has one or more recognized clinicopathologic features and/or that falls into a particular class or category based on gene expression profiling. For example, breast cancers can be classified into molecular subtypes based on gene expression profiles, e.g., luminal A, luminal B, ERBB2-associated, basal-like, and normal-like (see, e.g., Serlie, T., et al., Proc Natl Acad Sci USA. (2001) 98(19):10869-74). Breast cancers can be classified based on a number of different clinicopathologic features such as histologic subtype (e.g., ductal; lobular; mixed), histologic grade (grade 1, 2, 3); estrogen receptor (ER) and/or progesterone receptor (PR) status (positive (+) or negative (−)), HER2 (ERBB2) expression status, and lymph node involvement. For example, the following breast cancer subtypes can be defined based on expression of estrogen receptor (ER) and human epidermal growth factor receptor 2 (HER2), e.g., as assessed by immunohistochemistry (IHC): (I) ER+, HER2+; (2) ER+, HER2; (3) ER−, HER2+; and (4) ER−, HER2−. The level of expression can be used to further divide these subtypes. Amplification of the HER2 locus can be assessed, e.g., using in situ hybridization (ISH), e.g., fluorescent in situ hybridization (FISH). In some embodiments, an HSF1-based method is applied to a tumor that is ER+. In some embodiments an HSF1-based method is applied to a tumor that is ER−. In some embodiments an HSF1-based method is applied to a tumor that is HER2+. In some embodiments an HSF1-based method is applied to a tumor that is HER2−. In some embodiments an HSF1-based method is applied to a tumor that is PR+. In some embodiments an HSF1-based method is applied to a tumor that is PR−. In some embodiments an HSF1-based method is applied to a tumor that is EGFR+. In some embodiments an HSF-based method is applied to a tumor that is EGFR−. It will be understood that these markers may be present or absent in any combination in various embodiments. For example, in some embodiments an HSF1-based method is applied to a tumor that is ER+/HER2+ or ER+/HER2− (each of which categories can include tumors that are PR+ or PR− and are EGFR+ or EGFR−). In some embodiments, the sample or tumor is not “triple negative”, i.e., the sample or tumor is negative for expression of ER, PR, and HER2.

In some embodiments a subject has DCIS. In some embodiments a subject has Stage I or Stage II breast cancer. In some embodiments a subject has Stage III breast cancer. In some embodiments, cancer stage is assigned using pathologic criteria, clinical criteria, or a combination of pathologic and clinical criteria.

In some embodiments a subject does not have detectable lymph node involvement, i.e., the subject is “lymph node negative” (LNN). For example, the subject may have be ER+/lymph node negative. The clinical management of subjects in this early stage group (e.g., treatment selection) is challenging with regard to identifying which small portion of the population will have a recurrence (e.g., following surgery) and could therefore benefit from more intensive monitoring and/or more aggressive treatment. In accordance with certain embodiments of the invention, a subject with ER+, LNN cancer that has increased HSF1 expression and/or activation in tumor-associated stromal cells is monitored and/or treated more intensively than if the cancer does not have increased HSF1 expression and/or activation in tumor-associated stromal cells.

In some embodiments, increased HSF1 expression and/or activation in tumor-associated stromal cells from an ER+ breast tumor identifies patients having ER+ tumors that may be resistant to hormonal therapy. Such patients may benefit from use of a more aggressive treatment regimen, e.g., chemotherapy in addition to, or instead of, hormonal therapy, or more extensive surgery.

It has been reported that while about half of all breast cancers are assigned histologic grade 1 or 3 status (with a low or high risk of recurrence, respectively), a substantial percentage of tumors (30%-60%) are classified as histologic grade 2, which is less informative for clinical decision making because of intermediate risk of recurrence (Sotiriou C, et al., J Natl Cancer Inst., 98(4):262-72, 2006). Improved prognostic methods could be of significant use in this setting, for example. In some embodiments, an HSF1-based method is applied to a tumor classified as histologic grade 2, e.g., to classify histologic grade 2 tumors into high and low risk groups. In some embodiments, an HSF1-based method is applied to a tumor classified as histologic grade 2, e.g., to classify histologic grade 2 tumors into higher and lower risk groups, wherein tumors that have increased HSF1 expression or HSF1 activation are classified into the higher risk group. Tumors that do not have increased HSF1 expression or HSF1 activation would be classified into the lower risk group.

In some embodiments, an HSF1-based assay is used to provide sample classification, diagnostic, prognostic, or treatment-predictive information pertaining to lung cancer, e.g., non-small cell lung cancer (NSCLS), such as a lung adenocarcinoma. In some embodiments, the lung cancer, e.g., lung adenocarcinoma, is a Stage I cancer (T1 N0 M0 or T2 N0 M0). In some embodiments the cancer is a Stage IA lung cancer (T1N0M0). In some embodiments the cancer is a Stage IB lung cancer (T1N0M0). In some embodiments, the lung cancer, e.g., lung adenocarcinoma, is a Stage II cancer. Stage I and II lung cancers are typically treated by surgical resection of the tumor. Although surgery can be curative, a significant fraction of patients develop recurrence or metastases. Such patients might benefit from adjuvant therapy (radiation and/or chemotherapy). However, the current standard staging system (TMN) cannot predict which stage I or II lung cancers will recur. Although studies have shown adjuvant chemotherapy to be of benefit in groups of patients with stage II lung cancer, its role in treating stage I lung cancer is unclear. Without wishing to be bound by any theory, the number of patients diagnosed with stage I or II lung cancer may increase significantly at least in part due to the increased use of imaging modalities such as computed tomography (CT) scans for screening purposes, e.g., in individuals who have a significant smoking history. It would be useful to be able to identify those patients with stage I or stage II cancer who are at increased likelihood of recurrence and may therefore be more likely to benefit from adjuvant chemotherapy. In some embodiments, an HSF1-based method is applied to classify a stage I or stage II lung tumor into a higher or lower risk group, wherein tumors that have increased (e.g., high or intermediate) HSF1 expression or HSF1 activation are classified into the higher risk group. Tumors that have absent or low HSF1 expression and/or activation in tumor-associated stromal cells are classified into the lower risk group. Subjects with tumors classified into the higher risk group have an increased likelihood of recurrence than subjects with tumors classified into the lower risk group and may benefit from adjuvant chemotherapy. Subjects with tumors classified into the lower risk group may be treated with surgery alone. Adjuvant chemotherapy for operable lung cancer frequently includes a platinum-based agent (e.g., cisplatin or carboplatin), optionally in combination with an anti-mitotic agent (e.g., an anti-microtubule agent) such as a taxane (e.g., paclitaxel (Taxol) or docetaxel (Taxotere)) or a vinca alkaloid such as vinblastine, vincristine, vindesine and vinorelbine. Other agents that may be administered as adjuvant chemotherapy in operable lung cancer, typically in combination with a platinum agent, include mitomycin, doxorubicin, or etoposide. Other adjuvant chemotherapy regiments include tegafur alone, uracil alone, a combination of tegafur and uracil, or a combination of tegafur and/or uracil with a platinum agent.

In some embodiments a subject has been previously treated for the cancer, while in other embodiments the subject has not previously received treatment for the cancer. In some embodiments, a subject falls within a selected age group or range, e.g., 40 years old or less, 50 years old or less, 55 years old or less, 60 years old or less, between 40 and 60 years of age, 40 years old or more, 50 years old or more, 55 years old or more, 60 years old or more, etc. Any age group or range may be selected in various embodiments of the invention, whether or not specifically mentioned here.

In some embodiments a subject, e.g., a subject having or at risk of lung cancer or lung cancer recurrence, is a current smoker or former smoker. In some embodiments a subject, e.g., a subject having or at risk of developing lung cancer or lung cancer recurrence, is a non-smoker who has no or essentially no history of smoking.

Any method of the invention that comprises assessing HSF1 expression or HSF1 activation or using the level of expression or activation of an HSF1 gene product may, in certain embodiments, further comprise assessing or using the level of expression, activation, or activity of one or more additional cancer biomarkers. In certain embodiments, the level of expression, activation, or activity of an HSF1 gene product is used in conjunction with the level of expression, activation, or activity of one or more additional cancer biomarkers in a method of providing diagnostic, prognostic, or treatment-specific predictive information. The additional cancer biomarker(s) may be selected based at least in part on the site in the body from which a sample was obtained or the suspected or known tissue of origin of a tumor. For example, in the case of suspected or known breast cancer, one or more breast cancer biomarkers may be assessed.

In some embodiments, an HSF1-based assay is used together with additional information, such as results of a second assay (or multiple assays) and/or clinicopathological information to provide diagnostic, prognostic, or treatment-predictive information pertaining to breast cancer. In some embodiments, such information comprises, e.g., subject age, tumor size, nodal involvement, tumor histologic grade, ER status, PR status, and/or HER2 status, menopausal status, etc.). In some embodiments, the additional information includes the PR status of the tumor. For example, a method can comprise determining the PR status of a tumor and, if the PR status is positive, classifying the tumor with respect to prognosis or treatment selection based on expression of HSF1 or activation of HSF1. In some embodiments, information from an HSF1-related assay is used together with a decision making or risk assessment tool such as the computer program Adjuvant! Online (https://www.adjuvantonline.com/index.jsp). The basic format of an early version of Adjuvant! was described in the article Ravdin, Siminoff, Davis, et al. JCO 19(4) 980-991, 2001. In some embodiments, the second assay is a gene expression profiling assay such as the MammaPrint® (Agendia BV, Amsterdam, the Netherlands), Oncotype DX™ (Genomic Health, Redwood City, Calif.), Celera Metastasis Score™ (Celera, Inc., Rockville, Md.), Breast BioClassifier (ARUP, Salt Lake City, Utah), Rotterdam signature 76-gene panel (Erasmus University Cancer Center, Rotterdam, The Netherlands), MapQuant Dx™ Genomic Grade test (Ipsogen, Stamford, Conn.), Invasiveness Gene Signature (OncoMed Pharmaceuticals, Redwood City, Calif.), NuvoSelect™ assay (Nuvera Biosciences, Woburn, Mass.), THEROS Breast Cancer IndexSM (BCI) (bioTheranostics, San Diego) that classifies tumors (e.g., into high or low risk groups) based on expression level of multiple genes using, e.g., a microarray or multiplex RT-polymerase chain reaction (PCR) assay. In some embodiments a lung tumor is assessed using an assay for detecting KRAS and/or EGFR mutations. In some embodiments a lung tumor is assessed using an assay for excision repair cross-complementation group 1 (ERCC1) and/or mutS homolog 2 (MSH2) expression, wherein low expression of either or both of said genes indicates a poorer prognosis and/or an increased likelihood of benefit from adjuvant therapy. The phrase “used together” with in regard to two or more assays means that the two or more assays are applied to a particular tumor. In some embodiments, the two or more assays are applied to the same sample (or a portion thereof) obtained from the tumor.

In some embodiments, an HSF1-based assay may be used together with a gene expression profile in which expression level of at least 1, at least 5, or at least 10 different genes (“classifier genes”) is used to classify a tumor. It will be understood that such gene expression profile assays may measure expression of control genes as well as classifier genes. In some embodiments an HSF1-based assay is used together with an H:I™ test (bioTheranostics, Carlsbad, Calif.), in which the ratio of expression of HOXB 13 and IL-17B genes is used to classify a tumor. In some embodiments, an HSF1-based assay is used together with an antibody-based assay, e.g., the ProEx™ Br (TriPath Oncology, Durham, N.C.), Mammostrat® (Applied Genomics, Inc., Huntsville, Ala.), ADH-5 (Atypical Ductal Hyperplasia) Breast marker antibody cocktail (Biocare Medical, Concord, Calif.), measurement of urokinase-like plasminogen activator (uPA) and/or its inhibitor plasminogen activator inhibitor 1 (PAI1), or a FISH-based test such as the eXaagenBC™ (eXagen Diagnostics, Inc., Albuquerque, N. Mex.). In some embodiments, an HSF1-based assay is used together with an assay that measures proliferation. For example, expression of a proliferation marker such as Ki67 (Yerushalmi et al., Lancet Oncol. (2010), 11(2):174-83) can be used.

An HSF1-based assay (e.g., any of the HSF1-based assays described herein) may be used together with another assay in any of a number of ways in various embodiments of the invention. For example, in some embodiments, if results of two tests are discordant (e.g., one test predicts that the subject is at high risk while the other predicts that the subject is at low risk), the subject may receive more aggressive therapeutic management than if both tests predict low risk. In some embodiments, if a result of a non-HSF1-based assay is inconclusive or indeterminate, an HSF1-based assay can be used to provide a diagnosis, prognosis, or predictive information. In some embodiments, one can have increased confidence if results of an HSF1-based assay and a second assay are in agreement. For example, if both tests indicate that the subject is at low risk, there can be increased confidence in the appropriateness of providing less aggressive therapeutic management, e.g., to not administer adjuvant chemotherapy, while if both tests indicate that the subject is at high risk, there can be increased confidence in the appropriateness of providing more aggressive therapeutic management.

In some embodiments, a method of the invention comprises providing treatment-specific predictive information relating to use of an HSF1 inhibitor to treat a subject with cancer, based at least in part on assessing the level of expression of HSF1 or activation of HSF1 in a sample obtained from the subject. In some embodiments, a sample can be classified as belonging to (i.e., obtained from) a subject with cancer who is a suitable candidate for treatment with an HSF1 inhibitor. For example, the invention provides a method of determining whether a subject with cancer is a suitable candidate for treatment with an HSF1 inhibitor comprising measuring the level of HSF1 expression and/or activation in a sample obtained from the subject comprising tumor-associated stromal cells, wherein an increased level of HSF1 expression or an increased level of HSF1 activation in the sample is indicative that the subject is a suitable candidate for treatment with an HSF inhibitor. In some embodiments, the invention provides a method of determining whether a subject with cancer is likely to benefit from treatment with an HSF1 inhibitor, comprising: measuring the level of HSF1 expression and/or activation in a sample obtained from the subject comprising tumor-associated stromal cells, wherein an increased level of HSF1 expression or an increased level of HSF1 activation in the sample is indicative that the subject is likely to benefit from treatment with an HSF1 inhibitor. In some embodiments, the invention provides a method of predicting the likelihood that a tumor will be sensitive to an HSF1 inhibitor, the method comprising: assessing the level of HSF1 expression or the level of HSF1 activation in a sample comprising tumor-associated stromal cells obtained from the tumor; wherein if the level of HSF1 expression or activation is increased, the tumor has an increased likelihood of being sensitive to the HSF1 inhibitor. A tumor is “sensitive” to a treatment if the subject experiences a partial or complete response or stabilization of disease following treatment. Response can be assessed, for example, by objective criteria such as anatomical tumor burden, as known in the art. In some embodiments, a response correlates with increased progression-free survival or increased overall survival. Thus in some embodiments, a tumor is sensitive to a treatment if administration of the treatment correlates with increased progression-free survival or increased overall survival.

In some embodiments, treatment with an HSF1 inhibitor comprises administering a HSF1 inhibitor to the subject in addition to a standard treatment regimen for treating the subject's cancer. It will be understood that the HSF1 inhibitor is typically administered in an effective amount in a suitable pharmaceutical composition that may comprise one or more pharmaceutically acceptable carriers. “Pharmaceutically acceptable carrier” refers to a diluent, excipient, or vehicle with which the therapeutically active agent is administered. An effective amount may be administered in one dose or multiple doses.

In some aspects, the invention encompasses the recognition that treatment of subjects without evidence of cancer (e.g., subjects at increased risk of cancer) with an HSF1 inhibitor may inhibit or reduce the likelihood that the subject will develop cancer. It should be noted that a subject may be a suitable candidate for treatment with an HSF1 inhibitor even if the cancer cells and/or tumor-associated stromal cells do not exhibit increased HSF1 expression and/or HSF1 activation. For example, subjects with early stage cancer that has not progressed to a state in which HSF1 expression and/or activation is increased may benefit from an HSF1 inhibitor. In some aspects, a method of treating a subject who has pre-invasive cancer comprises administering an HSF1 inhibitor to a subject with pre-invasive cancer. Such treatment may, for example, inhibit progression of the pre-invasive cancer to invasive cancer. In some aspects, a method of inhibiting recurrence of cancer in a subject comprises administering an HSF1 inhibitor to the subject. In some embodiments, the cancer is characterized by increased HSF1 expression or increased HSF1 activation.

In some aspects, the invention provides a method of inhibiting emergence of resistance to therapy in a subject with cancer, the method comprising administering a HSF1 inhibitor to the subject in combination with an additional therapy, thereby reducing the likelihood of resistance to the additional therapy. In some embodiments, the additional therapy is a chemotherapeutic agent. In some embodiments, the additional therapy is a hormonal agent. In some embodiments, the cancer is characterized by increased HSF1 expression and/or activation in tumor-associated stromal cells.

As used herein, an “HSF1 inhibitor” is an agent that inhibits expression or activity of HSF1. In some embodiments, an HSF1 inhibitor is an RNAi agent, e.g., a short interfering RNA (siRNA) or short hairpin RNA (shRNA) that, when present in a cell (e.g., as a result of exogenous introduction of an siRNA or intracellular expression of a shRNA) results in inhibition of HSF expression by RNA interference (e.g., by causing degradation or translational repression of mRNA encoding HSF1, mediated by the RNAi-induced silencing complex). Exemplary RNAi agents that inhibit HSF1 expression are disclosed, e.g., in PCT/EP2010/069917 (WO/2011/073326) or in reference 6. In some embodiments an HSF1 inhibitor may be an intrabody that binds to HSF1, or an agent such as a single chain antibody, aptamer, or dominant negative polypeptide that binds to HSF1, wherein the agent optionally comprises a moiety that allows it to gain entry into cells. For example, the agent may comprise a protein transduction domain that allows the agent to cross the plasma membrane or a ligand that binds to a cell surface receptor such that the agent is internalized, e.g., by endocytosis. In some embodiments the HSF1 inhibitor comprises a small molecule. In some embodiments the HSF 1 inhibitor comprises an agent that inhibits activation of HSF1. For example, the agent may at least in part block assembly of multimers, e.g., trimers, comprising HSF1. Suitable agents for inhibiting HSF1 may be identified using a variety of screening strategies. In some embodiments an HSF1 inhibitor may comprise an inhibitor of translation initiation. In some embodiments an HSF1 inhibitor may comprise a rocaglate. Exemplary rocaglates are described in U.S. Pat. No. 8,137,509, Santagata, et al. 2013, Rodrigo C M, et al. J Med Chem. (2012); 55:558, and/or in Roche S P, et al. Angew Chem Int Ed Engl. (2010); 49:6533.

In some embodiments the invention provides a method of identifying a candidate anti-cancer agent comprising: contacting tumor-associated stromal cells with a test agent; measuring HSF1 expression and/or activation in the tumor-associated stromal cells; comparing the level of HSF1 expression and/or activation with a control level; and identifying the test agent as a candidate anti-cancer agent if the level of HSF1 expression and/or activation measured is lower than the control level. In some embodiments the tumor-associated stromal cells are contacted with the test agent in vitro. In some embodiments the tumor-associated stromal cells are contacted with the test agent by administering the test agent to a subject having a tumor. In some embodiments the control level is a level of HSF1 expression and/or activation in tumor-associated stromal cells not contacted with the test agent. In some embodiments the tumor-associated stromal cells comprise cancer-associated fibroblasts. In some embodiments measuring the level of HSF1 expression comprises determining the level of an HSF1 gene product. In some embodiments the HSF1 gene product is an HSF1 mRNA. In some embodiments the HSF1 gene product is an HSF1 polypeptide. In some embodiments HSF1 expression and/or activation is assessed by measuring expression of a gene that is regulated by HSF1 in tumor-associated stromal cells, wherein decreased expression of the gene in the presence of the test agent is indicative that the test agent inhibits HSF1 expression and/or activation. In some embodiments the tumor-associated stromal cells may be co-cultured with cancer cells. In some embodiments the effect of a candidate agent identified according to the methods is tested on cancer cells.

Any of a wide variety of agents may be used as a test agent in various embodiments. For example, a test agent may be a small molecule, polypeptide, peptide, nucleic acid, oligonucleotide, lipid, carbohydrate, or hybrid molecule. In some embodiments an oligonucleotide comprises an siRNA, shRNA, antisense oligonucleotide, aptamer, or random oligonucleotide.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments a test agent is not an agent that is found in a cell culture medium known or used in the art, e.g., for culturing vertebrate, e.g., mammalian cells, e.g., an agent provided for purposes of culturing the cells, or, if the agent is found in a cell culture medium known or used in the art, the agent may be used at a different, e.g., higher, concentration when used as a test agent in a method or composition described herein.

In some aspects, the invention encompasses use of a method comprising assessing the level of HSF1 expression and/or activation in tumor-associated stromal cells as a “companion diagnostic” test to determine whether a subject is a suitable candidate for treatment with an HSF1 inhibitor. In some embodiments an HSF1 inhibitor may be approved (allowed to be sold commercially for treatment of humans or for veterinary purposes) by a government regulatory agency (such as the US FDA, the European Medicines Agency (EMA), or government agencies having similar authority over the approval of therapeutic agents in other jurisdictions) with the recommendation or requirement that the subject is determined to be a suitable candidate for treatment with the HSF1 inhibitor based at least in part on an HSF1-based assay. For example, the approval may be for an “indication” that includes the requirement that a subject or tumor sample be classified as having high levels or increased levels of HSF1 expression or HSF1 activation based on such assay. Such a requirement or recommendation may be included in the package insert provided with the agent. In some embodiments a particular method for detection or measurement of an HSF1 gene product or of HSF1 activation or a specific test reagent (e.g., an antibody that binds to HSF1 polypeptide or a probe that hybridizes to HSF1 mRNA) or kit may be specified. In some embodiments, the method, test reagent, or kit will have been used in a clinical trial whose results at least in part formed the basis for approval of the HSF1 inhibitor. In some embodiments, the method, test reagent, or kit will have been validated as providing results that correlate with outcome of treatment with the HSF1 inhibitor.

In some aspects, the invention provides a method of assessing efficacy of treatment of cancer comprising: (a) assessing the level of HSF1 expression and/or activation in stromal cells obtained from a subject that has been treated for cancer, wherein absence of increased HSF1 expression and/or activation in said stromal cells indicates effective treatment. In some embodiments, step (a) is repeated at one or more time points following treatment of the subject for cancer, wherein continued absence of increased HSF1 expression and/or continued absence of increased HSF1 activation of over time indicates effective treatment. The sample may be obtained, for example, from or close to the site of a cancer that was treated (e.g., from or near a site from which a tumor was removed).

In some aspects, the invention provides a method of assessing efficacy of treatment of cancer comprising: (a) assessing the level of HSF1 expression and/or activation in stromal cells obtained from a subject having cancer, and (b) repeating step (a) at one or more time points during treatment of the subject for cancer, wherein decreased HSF1 expression and/or or decreased HSF1 activation of over time in stromal cells indicates effective treatment. The sample may be obtained, for example, from or close to the site of a cancer being treated.

In some aspects, the invention provides a method of monitoring a subject for cancer recurrence comprising: (a) assessing the level of HSF1 expression and/or activation in stromal cells obtained from a subject that has been treated for cancer, wherein presence of increased HSF1 expression or increased HSF1 activation in the stromal cells indicates cancer recurrence. In some embodiments, step (a) is repeated at one or more time points following treatment of the subject for cancer. The sample may be obtained, for example, from or close to the site of a cancer that was treated (e.g., from or near a site from which a tumor was removed).

Certain aspects and embodiments of the invention are described herein mainly in regard to breast cancer or lung cancer. It will be understood that the invention encompasses embodiments in which products and processes described herein are applied in the context of tumors arising from other organs or tissues. One of ordinary skill in the art will recognize that certain details of the invention may be modified according, e.g., to the particular tumor type or organ interest. Such embodiments are within the scope of the invention.

It will be understood that many of the methods provided herein, e.g., methods of classification, may be described in terms of samples, tumors, or subjects and such descriptions maybe considered equivalent and freely interchangeable. For example, where reference is made herein to a method of classifying a sample, such method may be expressed as a method of classifying a tumor from which the sample was obtained or as a method of classifying a subject from which the sample originated in various embodiments. Similarly, where reference is made herein to assessing the level of HSF1 expression or HSF1 activation in a sample comprising tumor-associated stromal cells of a tumor, such method may be expressed as a method of assessing the level of HSF1 expression or HSF1 activation in tumor-associated stromal cells a tumor from which the sample was obtained in various embodiments. It will also be understood that a useful diagnostic, prognostic, or treatment-specific predictive method need not be completely accurate. For example, “predicting”, “predicting the likelihood”, and like terms, as used herein, do not imply or require the ability to predict with 100% accuracy and do not imply or require the ability to provide a numerical value for a likelihood (although such value may be provided). Instead, such terms typically refer to forecast of an increased or a decreased probability that a result, outcome, event, etc., of interest exists or will occur, e.g., when particular criteria or conditions exist, as compared with the probability that such result, outcome, or event, etc., exists or will occur when such criteria or conditions are not met.

Methods of Assessing HSF1 Expression or HSF1 Activation

HSF1 genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Exemplary databases include, e.g., GenBank, RefSeq, Gene, UniProtKB/SwissProt, UniProtKB/Trembl, and the like. The HSF1 gene has been assigned NCBI GeneID: 3297. The NCBI Reference Sequence accession numbers for human HSF1 mRNA and polypeptide are NM_005526 and NP_005517, respectively, and the human HSF1 polypeptide GenBank acc. no. is AAA52695.1. The human HSF1 gene is located on chromosome 8 (8q24.3), RefSeq accession number NC_000008.10. Sequences of other nucleic acids and polypeptides of interest herein, e.g., gene products of HSF1-regulated genes or cancer-stroma normalization genes such as those described herein, could also be readily obtained from such databases. Sequence information may be of use, for example, to generate reagents for detection of HSF1 gene products and/or reagents for detection of gene products of HSF1-regulated genes or cancer-stroma normalization genes.

In general, the level of HSF1 expression of HSF1 activation can be assessed using any of a variety of methods. In many embodiments, the level of HSF1 expression is assessed by determining the level of an HSF1 gene product in a sample comprising tumor-associated stromal cells. In some embodiments an HSF1 gene product comprises HSF1 mRNA. In general, any suitable method for measuring RNA can be used to measure the level of HSF1 mRNA in a sample. For example, methods based at least in part on hybridization and/or amplification can be used. Exemplary methods of use to detect mRNA include, e.g., in situ hybridization, Northern blots, microarray hybridization (e.g., using cDNA or oligonucleotide microarrays), reverse transcription PCR (e.g., real-time reverse transcription PCR), nanostring technology (see, e.g., Geiss, G., et al., Nature Biotechnology (2008), 26, 317-325; U.S. Ser. No. 09/898,743 (U.S. Pat. Pub. No. 20030013091) for exemplary discussion of nanostring technology and general description of probes of use in nanostring technology). A number of such methods include contacting a sample with one or more nucleic acid probe(s) or primer(s) comprising a sequence (e.g., at least 10 nucleotides in length, e.g., at least 12, 15, 20, or 25 nucleotides in length) substantially or perfectly complementary to a target RNA (e.g., HSF1 mRNA). The probe or primer is often detectably labeled using any of a variety of detectable labels. In many embodiments the sequence of the probe or primer is sufficiently complementary to HSF1 mRNA to allow the probe or primer to distinguish between HSF1 mRNA and most or essentially all (e.g., at least 99%, or more) transcripts from other genes in a mammalian cell, e.g., a human cell, under the conditions of an assay. In some embodiments, “substantially complementary” refers to at least 90% complementarity, e.g., at least 95%, 96%, 97%, 98%, or 99% complementarity. A probe or primer may also comprise sequences that are not complementary to HSF1 mRNA, so long as those sequences do not hybridize to other transcripts in a sample or interfere with hybridization to HSF1 mRNA under conditions of the assay. Such additional sequences may be used, for example, to immobilize the probe or primer to a support. A probe or primer may be labeled and/or attached to a support or may be in solution in various embodiments. A support may be a substantially planar support that may be made, for example, of glass or silicon, or a particulate support, e.g., an approximately spherical support such as a microparticle (also referred to as a “bead” or “microsphere”). In some embodiments, a sequencing-based approach such as serial analysis of gene expression (SAGE) (including variants thereof) or RNA-Sequencing (RNA-Seq) is used. RNA-Seq refers to the use of any of a variety of high throughput sequencing techniques to quantify RNA transcripts (see, e.g., Wang, Z., et al. Nature Reviews Genetics (2009), 10, 57-63). Other methods of use for detecting RNA include, e.g., electrochemical detection, bioluminescence-based methods, fluorescence-correlation spectroscopy, etc. It will be understood that certain methods that detect mRNA may, in some instances, also detect at least some pre-mRNA transcript(s), transcript processing intermediates, and degradation products of sufficient size. It will also be understood that a probe or primer may in some embodiments be substantially or perfectly complementary to a complement of HSF1 RNA.

In some embodiments an HSF1 gene product comprises HSF1 polypeptide. In general, any suitable method for measuring proteins can be used to measure the level of HSF1 polypeptide in a sample. In many embodiments, an immunological method or other affinity-based method is used. In general, immunological detection methods involve detecting specific antibody-antigen interactions in a sample such as a tissue section or cell sample. The sample is contacted with an antibody that binds to the target antigen of interest. The antibody is then detected using any of a variety of techniques. In some embodiments, the antibody that binds to the antigen (primary antibody) or a secondary antibody that binds to the primary antibody has been tagged or conjugated with a detectable label. In some embodiments a label-free detection method is used. A detectable label may be, for example, a fluorescent dye (e.g., a fluorescent small molecule) or quencher, colloidal metal, quantum dot, hapten, radioactive atom or isotope, or enzyme (e.g., peroxidase). It will be appreciated that a detectable label may be directly detectable or indirectly detectable. For example, a fluorescent dye would be directly detectable, whereas an enzyme may be indirectly detectable, e.g., the enzyme reacts with a substrate to generate a directly detectable signal. Numerous detectable labels and strategies that may be used for detection, e.g., immunological detection, are known in the art. Exemplary immunological detection methods include, e.g., immunohistochemistry (IHC); enzyme-linked immunosorbent assay (ELISA), bead-based assays such as the Luminex® assay platform (Invitrogen), flow cytometry, protein microarrays, surface plasmon resonance assays (e.g., using BiaCore technology), microcantilevers, immunoprecipitation, immunoblot (Western blot), etc. IHC generally refers to immunological detection of an antigen of interest (e.g., a cellular constituent) in a tissue sample such as a tissue section. As used herein, IHC is considered to encompass immunocytochemistry (ICC), which term generally refers to the immunological detection of a cellular constituent in isolated cells that essentially lack extracellular matrix components and tissue microarchitecture that would typically be present in a tissue sample. Traditional ELISA assays typically involve use of primary or secondary antibodies that are linked to an enzyme, which acts on a substrate to produce a detectable signal (e.g., production of a colored product) to indicate the presence of antigen or other analyte. IHC generally refers to the immunological detection of a tissue or cellular constituent in a tissue or cell sample comprising substantially intact (optionally permeabilized) cells. As used herein, the term “ELISA” also encompasses use of non-enzymatic reporters such as fluorogenic, electrochemiluminescent, or real-time PCR reporters that generate quantifiable signals. It will be appreciated that the term “ELISA” encompasses a number of variations such as “indirect”, “sandwich”, “competitive”, and “reverse” ELISA.

In some embodiments, e.g., wherein IHC is used for detecting HSF1, a sample is in the form of a tissue section, which may be a fixed or a fresh (e.g., fresh frozen) tissue section or cell smear in various embodiments. A sample, e.g., a tissue section, may be embedded, e.g., in paraffin or a synthetic resin or combination thereof. A sample, e.g., a tissue section, may be fixed using a suitable fixative such as a formalin-based fixative. The section may be a paraffin-embedded, formalin-fixed tissue section. A section may be deparaffinized (a process in which paraffin (or other substance in which the tissue section has been embedded) is removed (at least sufficiently to allow staining of a portion of the tissue section). To facilitate the immunological reaction of antibodies with antigens in fixed tissue or cells it may be helpful to unmask or “retrieve” the antigens through pretreatment of the sample. A variety of antigen retrieval procedures (sometimes called antigen recovery), can be used in IHC. Such methods can include, for example, applying heat (optionally with pressure) and/or treating with various proteolytic enzymes. Methods can include microwave oven irradiation, combined microwave oven irradiation and proteolytic enzyme digestion, pressure cooker heating, autoclave heating, water bath heating, steamer heating, high temperature incubator, etc. To reduce background staining in IHC, the sample may be incubated with a buffer that blocks the reactive sites to which the primary or secondary antibodies may otherwise bind. Common blocking buffers include, e.g., normal serum, non-fat dry milk, bovine serum albumin (BSA), or gelatin, and various commercial blocking buffers. The sample is then contacted with an antibody that specifically binds to the antigen whose detection is desired (e.g., HSF1 protein). After an appropriate period of time, unbound antibody is then removed (e.g., by washing) and antibody that remains bound to the sample is detected. After immunohistochemical staining, a second stain may be applied, e.g., to provide contrast that helps the primary stain stand out. Such a stain may be referred to as a “counterstain”. Such stains may show specificity for discrete cellular compartments or antigens or stain the whole cell. Examples of commonly used counterstains include, e.g., hematoxylin, Hoechst stain, or DAPI. The tissue section can be visualized using appropriate microscopy, e.g., light microscopy, fluorescence microscopy, etc. In some embodiments, automated imaging system with appropriate software to perform automated image analysis is used. In some embodiments parameters such as antibody dilution, incubation time, or other parameters are selected in order to increase or optimize detection of HSF1 in tumor-associated stromal cells.

In some embodiments, flow cytometry (optionally including cell sorting) is used to detect HSF1 expression. The use of flow cytometry would typically require the use of isolated cells substantially removed from the surrounding tissue microarchitecture, e.g., as a single cell suspension. HSF1 mRNA or polypeptide level may be assessed by contacting cells with a labeled probe that binds to HSF1 mRNA or a labeled antibody that binds to HSF1 protein, respectively, wherein said probe or antibody is appropriately labeled (e.g., with a fluorophore, quantum dot, or isotope) so as to be detectable by flow cytometry. In some embodiments, cell imaging can be used to detect HSF1. In some embodiments tumor-associated stromal cells are removed from a tumor sample. For example, laser capture microdissection may be used. In some embodiments cells are stained with an antibody that binds to a marker expressed by tumor-associated stromal cells. Such a marker may be used to specifically detect tumor-associated stromal cells, e.g., to distinguish them from cancer cells and/or from stromal cells not associated with a tumor and/or to determine the type of stromal cell. In some embodiments the marker is a marker of cancer-associated fibroblasts. In some embodiments the marker is smooth muscle actin. In some embodiments cancer-associated fibroblasts may be identified based at least in part on lack of expression of a marker of endothelial cells or leukocytes. In some embodiments stromal cells are distinguished from cancer cells based on cell morphology, size, location, or other indicators known in the art. Those of ordinary skill in the art are aware of appropriate methods to distinguish cancer cells from stromal cells and to distinguish between different types of stromal cells. One of ordinary skill in the art will be readily able to select appropriate portions of a sample on which to apply methods of the present invention. In some embodiments cells or a tissue section may be co-stained for HSF1 and for one or more markers of tumor-associated stromal cells, e.g., cancer-associated fibroblasts.

In some embodiments, an antibody for use in an immunological detection method, e.g., IHC, is monoclonal. In some embodiments an antibody is polyclonal. In some embodiments, an antibody is a preparation that comprises multiple monoclonal antibodies. In some embodiments, the monoclonal or polyclonal antibodies have been generated using the same portion of HSF1 (or full length HSF) as an immunogen or binding target. In some embodiments, an antibody is an anti-peptide antibody. In some embodiments, a monoclonal antibody preparation may comprise multiple distinct monoclonal antibodies generated using different portions of HSF1 as immunogens or binding targets. Many antibodies that specifically bind to HSF1 are commercially available and may be used in embodiments of the present invention. One of ordinary skill in the art would readily be able to generate additional antibodies suitable for use to detect HSF1 polypeptide using standard methods.

In some embodiments, a ligand that specifically binds to HSF1 but is not an antibody is used as an affinity reagent for detection of HSF1. For example, nucleic acid aptamers or certain non-naturally occurring polypeptides structurally unrelated to antibodies based on various protein scaffolds may be used as affinity reagents. Examples include, e.g., agents referred to in the art as affibodies, anticalins, adnectins, synbodies, etc. See, e.g., Gebauer, M. and Skerra, A., Current Opinion in Chemical Biology, (2009), 13(3): 245-255 or PCT/US2009/041570. In some embodiments an aptamer is used as an affinity reagent. The terms “affinity reagent” and “binding agent” are used interchangeably herein.

In some embodiments, a non-affinity based method is used to assess the level of HSF1 polypeptide or HSF1 activation. For example, mass spectrometry could be used to detect HSF1 or to specifically detect phosphorylated HSF1.

In some embodiments, an antibody (or other affinity reagent) or procedure for use to detect HSF1 (or HSF1 phosphorylated on serine 326) in tumor-associated stromal cells can be validated, if desired, by showing that the classification obtained using the antibody or procedure correlate with a phenotypic characteristic of interest such as presence or absence of CIS, cancer prognosis, or treatment outcome, in an appropriate set of samples. In some embodiments, an antibody or antibody preparation or a protocol or procedure for performing IHC may be validated for use in an inventive method by establishing that its use provides similar results to those obtained using RT-629-PABX (Thermo Scientific) and the procedures described in the Examples on an appropriate set of test samples. For example, an antibody or antibody preparation or a procedure may be validated by establishing that its use results in the same classification (concordant classification) of at least 80%, 85%, 90%, 95% or more of samples in an appropriate set of test samples as is obtained using the antibody preparation of RT-629-PABX. A set of test samples may be selected to include, e.g., at least 10, 20, 30, or more samples in each category in a classification scheme (e.g., “positive” and “negative” categories; categories of“no”, “low”, or “high” expression, scores of 1, 2, 3; etc.). Once a particular antibody or procedure is validated, it can be used to validate additional antibodies or procedures. Likewise, a probe, primer, microarray, or other reagent(s) or procedure(s) to detect HSF1 RNA can be validated, if desired, by showing that the classification obtained using the reagent or procedure correlates with a phenotypic characteristic of interest such as presence or absence of CIS, cancer prognosis, or treatment outcome, in an appropriate set of samples.

It will be understood that suitable controls and normalization procedures can be used to accurately quantify HSF1 expression and/or activation, where appropriate. For example, measured values can be normalized based on the expression of one or more RNAs or polypeptides whose expression is not correlated with a phenotypic characteristic of interest. In some embodiments, a measured value can be normalized to account for the fact that different samples may contain different proportions of a cell type of interest, e.g., tumor-associated stromal cells versus non-tumor associated cells. For example, in some embodiments, the percentage of non-tumor associated stromal cells may be assessed and the overall results adjusted to accurately reflect HSF1 mRNA or polypeptide level specifically in the cancer cells and tumor-associated stromal cells, or in the tumor-associated stromal cells or cancer cells alone. If a tissue section contains distinguishable (e.g., based on standard histopathological criteria), areas of tumor tissue and normal tissue the level of HSF1 expression or activation could be assessed in tumor-associated stromal cells in the area of tumor tissue, e.g., for purposes of comparison with a control level, which may optionally be the level measured in the normal tissue. In some embodiments the normal tissue is well separated from the tumor tissue. In some embodiments the HSF1 status of stromal cells present within the matrix of residual non-malignant elements present in the tissue section is assessed.

In certain embodiments of the invention the level of HSF1 mRNA or protein level is not measured or analyzed simply as a contributor to a cluster analysis, dendrogram, or hcatmap based on gene expression profiling in which expression at least 20; 50; 100; 500; 1,000, or more genes is assessed. In certain embodiments of the invention, e.g., if HSF1 mRNA or protein level is measured as part of such a gene expression profile, the level of HSF1 mRNA or protein is used to classify samples or tumors (e.g., for diagnostic, prognostic or treatment-specific predictive purposes) in a manner that is distinct from the manner in which the expression of many or most other genes in the gene expression profile are used. For example, the level of HSF1 mRNA or polypeptide may be used independently of most or all of the other measured expression levels or may be weighted more strongly than many or most other mRNAs in analyzing or using the results.

In some embodiments, HSF1 mRNA or polypeptide level is used together with levels of a set of no more than 10 other mRNAs or proteins that are selected for their utility for classification for diagnostic, prognostic, or predictive purposes in one or more types of cancer, such as breast cancer. For example, in the case of breast cancer, HSF1 mRNA or polypeptide levels can be used together with a measurement of estrogen receptor (ER), progesterone receptor (PR), or human epidermal growth factor receptor 2 (HER2) mRNA or polypeptide levels. In some embodiments, measurement of ER, PR, HER2 mRNA and/or other mRNA is performed using ISH. In some embodiments, measurement of ER, PR, HER2 polypeptide and/or other polypeptides is performed using IHC. In some embodiments such testing is performed in accordance with recommendations of the American Society of Clinical Oncology/College of American Pathologists Guideline Recommendations for Immunohistochemical Testing of Estrogen and Progesterone Receptors in Breast Cancer or the American Society of Clinical Oncology/College of American Pathologists Guideline Recommendations for Human Epidermal Growth Factor Receptor 2 Testing in Breast Cancer. In some embodiments such testing is performed according to recommendations of a commercially available kit, e.g., a kit approved by a governmental regulatory agency (e.g., the U.S. Food and Drug Administration) for use in clinical diagnostic, prognostic, or predictive purposes.

In general, the level of HSF1 activation can be assessed using any of a variety of methods in various embodiments of the invention. In some embodiments, the level of HSF1 activation is determined by detecting HSF1 polypeptide in cell nuclei, wherein nuclear localization of HSF1 polypeptide is indicative of HSF1 activation. HSF1 localization can be assessed, for example, using IHC, flow cytometry, FACS, etc. Alternately, or additionally, cell nuclei could be isolated and HSF1 polypeptide detected by immunoblot. In some embodiments, HSF1 nuclear localization could be assessed by staining for HSF1 protein, counterstaining with a dye that binds to a nuclear component such as DNA, and assessing co-localization of HSF1 and such nuclear component. Cell imaging can be used in some embodiments. It will be understood that “detecting” as used herein, can encompass applying a suitable detection procedure and obtaining a negative result, i.e., detecting a lack of expression or activation.

In some embodiments, the level of HSF1 activation is determined by determining the level of HSF1 phosphorylation, wherein HSF1 phosphorylation is indicative of HSF1 activation. In some embodiments, phosphorylation of HSF1 on serine 326 is determined as an indicator of HSF1 activation. Phosphorylation of HSF1 on serine 326 can be assessed, for example, using antibodies that bind specifically to HSF1 phosphorylated on serine 326. In some embodiments, a ratio of phosphorylated HSF1 to unphosphorylated HSF1 (on serine 326) is used as an indicator of HSF1 activation, with a higher ratio indicating more activation. Measurement of other post-translational modifications indicative of HSF1 activation could be used in various embodiments.

In some embodiments, the level of HSF1 activation in tumor-associated stromal cells is determined by measuring a gene expression profile of one or more genes whose expression is regulated by HSF1 in such cells, wherein increased expression of a gene that is positively regulated by HSF1 or decreased expression of a gene that is negatively regulated by HSF1 is indicative of HSF1 activation. In some embodiments a gene expression profile measures expression of at least 5 HSF1-regulated genes, e.g., between 5 and about 1,000 HSF1-regulated genes. In some embodiments measurement of expression of one or more genes that are not regulated by HSF1 in tumor-associated stromal cells is used as a control or for normalization purposes. In some embodiments measurement of expression of one or more genes that are not regulated by HSF1 in tumor-associated stromal cells may be disregarded. In some embodiments no more than 1%, 5%, 10%, 20%, 30%, 40%, or 50%, of measurements are of genes that are not regulated by HSF1 in tumor-associated stromal cells. In some embodiments, determining whether HSF1 is activated comprises comparing a gene expression profile obtained from a sample of interest with gene expression profile(s) obtained from one or more samples comprising tumor-associated stromal cells in which HSF1 is activated or is not activated. If the gene expression profile obtained from the sample clusters with or resembles the gene expression profile obtained from sample(s) in which HSF1 is activated, the sample of interest can be classified as exhibiting HSF1 activation. On the other hand, if the gene expression profile obtained from the sample of interest clusters with or resembles the gene expression profile obtained from sample(s) in which HSF1 is not activated, the sample of interest can be classified as not exhibiting HSF1 activation. Methods for clustering samples are well known in the art or assigning a sample to one of multiple clusters are well known in the art and include, e.g., hierarchical clustering, k-means clustering, and variants of these approaches. In some embodiments the level of HSF1 activation in tumor-associated stromal cells is determined by measuring the expression of one or more genes in cancer cells associated with such tumor-associated stromal cells, wherein the expression of said one or more genes in said cancer cells is regulated in a manner that depends on the activation of HSF1 in the associated stromal cells.

As described further in the Examples, Table S3 gene list G1 (hereinafter “Table S3-G1”), Table S3 gene list G2 (hereinafter “Table S3-G2”), Table S3 gene list G3 (hereinafter “Table S3-G3”), and Table S3 gene list G4 (hereinafter “Table S3-G4”) list genes that are regulated by HSF1 in tumor-associated stromal cells. In particular, as described in the Examples, Table S3-G1 and Table S3-G4 list genes that are upregulated (positively regulated) by HSF1 in tumor-associated stromal cells, and Table S3-G2 and Table S3-G3 list genes that are downregulated (negatively regulated) by HSF1 in tumor-associated stromal cells. Accordingly, in some embodiments described herein that relate to one or more HSF1-regulated genes, the genes may be selected from those listed in any of Tables S3-G1, S3-G2, S3-G3, and Table S3-G4.

In some embodiments that relate to one or more HSF1-regulated genes that are upregulated (positively regulated) by HSF1 in tumor-associated stromal cells, the genes may be selected from those listed in Tables S3-G1 and Table S3-G4 (i.e., any one or more of the genes may be listed in Table S3-G1 or Table S3-G4). For example, in some aspects measuring the level of HSF1 expression and/or activation comprises measuring the expression of one or more genes that are regulated by HSF1 in tumor-associated stromal cells and comparing the level of expression of the one or more genes with a control level, wherein an increased level of expression of the one or more genes is indicative of increased HSF1 expression and/or activation. In some embodiments one or more of the HSF1-regulated genes are selected from genes listed in Table S3-G1 or Table S3-G4 (i.e., any one or more of the genes may be listed in Table S3-G1 or Table S3-G4). The genes listed in Table S3-G1 may be referred to as HSF1-G1 genes. The genes listed in Table S3-G4 may be referred to as HSF1-G4 genes. The genes listed in Tables S3-G1 and S3-G4 (i.e., in either of those tables) may be referred to as HSF1 stromal signature set (HSF1-SSS) genes. In some embodiments of particular interest that relate to one or more HSF1-regulated genes that are upregulated (positively regulated) by HSF1 in tumor-associated stromal cells, the genes may be selected from those listed in Table D (Refined HSF1-SSS genes). The genes listed in Table D are upregulated by HSF1 in tumor-associated stromal cells; thus increased expression of genes listed in Table D (e.g., increased average expression level) is indicative of increased HSF1 activity in tumor-associated stromal cells. In some embodiments, wherein a subset of the genes in Table D is selected for measurement of expression, increased expression of such subset (e.g., increased average expression level) is indicative of increased HSF1 activity in tumor-associated stromal cells.

In some embodiments that relate to one or more HSF1-regulated genes that are downregulated (negatively regulated) by HSF1 in tumor-associated stromal cells, the gene(s) may be selected from those listed in Tables S3-G2 and Table S3-G3 (i.e., the gene may be listed in Table S3-G2 or Table S3-G3). For example, in some aspects measuring the level of HSF1 expression and/or activation comprises measuring the expression of one or more genes that are regulated by HSF1 in tumor-associated stromal cells and comparing the level of expression of the one or more genes with a control level, wherein a decreased level of expression of the one or more genes is indicative of increased HSF1 expression and/or activation. In some embodiments one or more of the HSF1-regulated genes are selected from genes listed in Table S3-G2 or Table S3-G3 (i.e., any one or more of the genes may be listed in Table S3-G2 or Table S3-G3). The genes listed in Table S3-G2 may be referred to as HSF1-G2 genes. The genes listed in Table S3-G3 may be referred to as HSF1-G3 genes.

In some embodiments of any of the methods that comprise measuring expression of one or more HSF1-G1 genes, the method may comprise measuring expression of at least 5, 10, 20, 50, 100, 200, 300, 400, 500, or more such genes. In some embodiments, expression of between 5 and 25, between 25 and 50, between 50 and 100, between 100 and 200, between 200 and 300, between 300 and 400, between 400 and 546 HSF1-G1 genes is measured. In some embodiments of any of the methods that comprise measuring expression of one or more HSF1-G2 genes, the method may comprise measuring expression of at least 5, 10, 20, 50, 100, 200, 300, 400, or more such genes. In some embodiments, expression of between 5 and 25, between 25 and 50, between 50 and 100, between 100 and 200, between 200 and 300, between 300 and 419 HSF1-G2 genes is measured. In some embodiments of any of the methods that comprise measuring expression of one or more HSF1-G3 genes, the method may comprise measuring expression of at least 5, 10, 20, 50, 100, or more such genes. In some embodiments, expression of between 5 and 25, between 25 and 50, between 50 and 100, between 100 and 144 HSF1-G3 genes is measured. In some embodiments of any of the methods that comprise measuring expression of one or more HSF1-G4 genes, the method may comprise measuring expression of at least 5, 10, 20, 50, 100, 200, 300, or more such genes. In some embodiments, expression of between 5 and 25, between 25 and 50, between 50 and 100, between 100 and 200, or between 200 and 325 HSF1-G4 genes is measured.

As described herein in some embodiments the level of HSF1 expression and/or the level of HSF1 activation is assessed in tumor-associated stromal cells and in cancer cells of the same tumor. It will be appreciated that the level of HSF1 expression and/or the level of HSF1 activation in tumor-associated stromal cells and/or in cancer cells may be measured using any of the methods for measuring HSF1 expression and/or HSF1 activation described herein. For example, in some embodiments, IHC is used to measure HSF1 expression and/or activation. In some embodiments, HSF1 protein and/or nuclear localization are measured using IHC. In some embodiments, in situ hybridization is used to measure HSF1 mRNA level. In some embodiments the level of HSF1 expression and/or activation is measured by measuring HSF1 activity. In some embodiments the level of HSF1 activity is measured by measuring expression of one or more genes that are regulated by HSF1. For example, in some embodiments, measuring HSF1 activity in cancer cells comprises measuring expression of one or more genes that are regulated by HSF1 in cancer cells, e.g., measuring expression of one or more genes that are part of the HSF1-dependent gene expression signature in cancer cells (see Mendillo, et al., 2012 (cited below) and/or PCT/US2013/039527 (WO/2013/166427)). For example, in some embodiments measuring HSF1 activity in cancer cells comprises measuring expression of at least 5, 10, 20, 50, 100, or 150 genes listed in Table A-1 hereof (HSF1 Cancer Signature Set (CSS) genes), Table A-2 hereof, Table A-3 hereof, or Table B hereof (Refined HSF1 Cancer Signature Set (CSS) genes). In some embodiments of any of the methods, measuring HSF1 activity in cancer cells comprises measuring expression of at least 200, 300, 350, 400, 450, or more genes listed in Table A-1. In some embodiments of any of the methods, measuring HSF1 activity in cancer cells comprises measuring expression of at least 200, 300 or more genes listed in Table A-2. In some embodiments of any of the methods, measuring HSF1 activity in cancer cells comprises measuring expression of at least 200 genes listed in Table A-3. The first 163 genes listed in Table A-3 (ABCA7-ZNF453) were positively associated with poor outcome. The last 44 genes listed in Table A-3 (AFF2-ZBTB20) were negatively associated with poor outcome, i.e., reduced expression of these genes correlated with poor outcome. It would be understood that when deriving an average expression level for a set of HSF1-regulated genes that includes genes whose expression level positively correlates with poor outcome (i.e., HSF1-regulated genes whose expression is upregulated by HSF1 such that increased expression correlates with poor outcome) and genes whose expression level negatively correlates with poor outcome (i.e., HSF1-regulated genes that are downregulated by HSF1 such that reduced expression correlates with poor outcome), those genes whose expression level positively correlates with poor outcome can be positively weighted and those genes whose expression level negatively correlates with poor outcome can be negatively weighted, so that a decrease in expression of a gene whose expression is downregulated by HSF1, such that decreased expression correlates with poor outcome, contributes positively to the average expression level of a set of HSF1-regulated genes.

In some embodiments measuring HSF1 activity in cancer cells comprises measuring expression of one or more genes in a subset of the Refined HSF1 Cancer Signature Set (CSS) genes, which subset is composed of genes of particular interest for measuring HSF1 activity in cancer cells. These genes are listed in Table C hereof. In some embodiments measuring HSF1 activity in cancer cells comprises measuring expression of at least 5, 10, 20, 30, 40, 45, or all 50 genes listed in Table C (HSF1 Cancer Signature Set (CSS) Genes Subset). In some embodiments, HSF1 activity is measured in cancer cells by measuring expression of the genes listed in Table C or a subset thereof, e.g., between 10 and 20, between 20 and 30, between 30 and 40, or between 40 and 50 of the genes listed in Table C. The genes listed in Table C are upregulated by HSF1 in cancer cells; thus increased expression of genes listed in Table C (e.g., increased average expression level) is indicative of increased HSF1 activity in cancer cells. In some embodiments, wherein a subset of the genes in Table C is selected for measurement of expression, increased expression of such subset (e.g., increased average expression level) is indicative of increased HSF1 activity in cancer cells. In some embodiments, the level of HSF1 activity in cancer cells measured by measuring expression of the genes listed in Table C or a subset thereof may be used independently of, or together with, measurement of HSF1 expression, activation, and/or activity in stromal cells from the same tumor for purposes of tumor classification, diagnosis, prognosis, treatment selection, and/or treatment-specific prediction.

Table A-1: HSF1 Cancer Signature Set (CSS) Genes

ABCC5, ABHD3, ACOT7, ADAMTS13, ADAT2, ADCK4, AGBL5, AHSA1, AK3L1, ALG10, ALOXE3, ANAPC2, ANG, ANGEL1, ANKRD13D, AOF2, APP, ASAH3L, ATF3, ATL3, ATP2C1, ATP6V1A, ATXN1, AZIN1, B3GALNT2, B3GNT1, BAG3, BAHD1, BANF1, BCL10, BCO2, BMF, BMS1, BRF2, BRMS1, C10orf4, C11orf2, C11orf68, C14orf112, C14orf133, C14orf43, C17orf75, C18orf25, C18orf55, C19orf33, C19orf6, C1orf160, C1orf172, C1orf182, C20orf19, C21orf7, C21orf70, C22orf16, C2orf37, C2orf67, C2orf7, C6orf108, C6orf150, C6orf211, C7orf55, C8orf37, C8orf73, C9orf156, CACYBP, CALM1, CAP2, CAV2, CBX3, CCDC109A, CCDC117, CCDC151, CCDC57, CCDC97, CCNL1, CCT3, CCT4, CCT5, CCT6A, CCT7, CCT8, CDC73, CDK3, CDKL3, CELSR1, CENPA, CENPT, CES2, CHD3, CHORDC1, CIAPIN1, CKS2, CLIP4, CLU, CMBL, CNN2, COASY, COMMD2, COPA, COPS7A, COQ9, CPSF1, CRELD1, CRY1, CRYZ, CSF3, CUEDC1, CUL4A, CYHR1, CYP24A1, D2HGDH, DARS, DEDD2, DGKE, DHX8, DNAJA1, DNAJA4, DNAJB, DNAJB4, DNAJB5, DNAJB6, DPY19L4, DRAP1, DTX2, DTX4, EARS2, EEF1G, EFCAB7, EIF1AD, EIF4A2, ENY2, EWSR1, FAM26B, FAM83E, FBXO15, FBXO31, FBXO45, FBXO47, FEM 1B, FGD6, FKBP4, FLJ21865, FLJ25404, FLJ35767, FRMD8, FRS3, FUT10, FXR1, GABRE, GALT, GCN5L2, GFM2, GLA, GNA15, GOLGA3, GPBP1, GPR4, GPR56, GPSN2, GPT, GRIFIN, GTF2F1, GTF3C3, GTPBP1, GUSB, HAO2, HEATR6, HEL308, HIST1H4H, HMHA1, HNRNPA2B1, HNRNPH3, HPS4, HSP90AA1, HSP90AB1, HSPA4, HSPA4L, HSPA6, HSPA8, HSPB1, HSPB9, HSPC152, HSPD1, HSPE1, HSPH1, HUS1, HYPK, IFNGR2, IFT122, IGHMBP2, IL11RA, IMP4, ITGB3BP, JMJD1B, JMJD6, JOSD1, JRK, KBTBD7, KCNIP3, KHK, KIAA0090, KIAA1737, KIAA1975, KIF21A, KIFC2, KILLIN, KLC1, KLF10, KLHL25, KLHL26, KNTC1, KPNA1, KREMEN2, LASP1, LCE1E, LMNB2, LOC124512, LOC134145, LOC26010, LOC653147, LRP12, LRRC27, LRRC59, LSM4, LTBP4, LY6K, LZIC, MAF1, MAP2K2, MAP7D1, MAT2A, MBD4, MBOAT2, MBOAT7, MDH2, MED23, METTL8, METTL9, MFSD3, MLL, MLL2, MLX, MMP11, MOBKL3, MORC4, MORF4L2, MRPL16, MRPL18, MRPL21, MRPL24, MRPL49, MRPS18C, MRPS23, MRPS6, MRTO4, MTCH1, MUL1, MUM1, MYL6, MYL6B, MYST2, N4BP2L2, NAT13, NBL1, NBN, NCOR1, NCSTN, NDOR1, NDRG1, NDUFA12, NECAP2, NEIL2, NGRN, NIBP, NMNAT1, NMT2, NOL1, NOP5/NOP58, NOSIP, NROB2, NSFL1C, NUDC, NUDCD1, NUF2, NUTF2, OPA3, OSGIN1, P4HA2, PABPC1, PAPOLA, PAQR4, PARD6B, PBLD, PCBD1, PCGF2, PCID2, PEX3, PFAS, PGAM5, PGK1, PIGL, PLEC1, PMEPA1, PMPCA, PMVK, PNRC2, POLD4, POLG, POLL, POLR2L, POLR3B, POLR3E, PPM1A, PRAF2, PRDX5, PRKCDBP, PRKCSH, PRKD2, PRRG2, PSMB3, PSMD3, PSPH, PTEN, PTGES3, PTOV1, PTP4A2, PUF60, RAB11B, RAB39, RABGAP1L, RANBP10, RANBP2, RANGAP1, RBM23, RBM25, REXO4, RHBDD2, RHBDD3, RMND1, RNASE4, RP11-529110.4, RPH3AL, RPL13, RPL18, RPL26L1, RPL29, RPS2, RPS5, RPS7, RRAD, RSRC2, S100A14, S100A16, SACM1L, SAPS1, SCFD1, SDCCAG10, SDCCAG3, SECISBP2, SEPW1, SERINC4, SERPINH1, SF3A3, SFRS10, SFRS12IP1, SFRS7, SH2D3A, SHARPIN, SHF, SLC25A45, SLC27A4, SLC45A4, SLC5A3, SLC9A1, SNAP23, SNX3, SOS1, SPATA21, SPECC1, SPHK2, SPR, SRRD, SSPO, ST13, STAT6, STIP1, STK40, STX16, STX18, STYXL1, SUGT1, SYNGR2, TAF7, TBC1D10B, TBC1D13, TBL3, TCP1, TCTN1, TESSP5, TIAL1, TIGD6, TINP1, TM2D3, TM9SF4, TMED3, TMEM203, TMEM66, TMEM95, TNP03, TPD52, TPD52L2, TPT1, TRAF3, TRAPPC3, TRIB3, TRIM41, TRIM52, TRIM7, TSEN34, TSNAXIP1, TSPAN4, TTC26, TYW3, UBB, UBC, UBE2B, UBE2D3, UBE21, UBE2O, UBFD1, UBL7, UBQLN1, UNC13D, USP30, USPL1, UTP11L, VAV1, VEZT, VIP, VRK3, WDR38, WDR45, WDR53, XPNPEP3, ZBTB25, ZCCHC2, ZFAND2A, ZNF180, ZNF207, ZNF250, ZNF337, ZNF34, ZNF467, ZNF473, ZNF526, ZSCAN22.

Table A-2: HSF1-CaSig2 Genes

ABCC1, ABCC5, ABCD3, ACBD6, ACD, ACOT7, AGBL5, AHSA1, AMOTL2, ANKMY2, AP4E1, ARID3B, ASNSD1, ATG16L1, ATL3, ATPBD4, AZIN1, BAG2, BANF1, BAX, BCAS4, BCL2L12, BMS1, BXDC2, BZW2, C12orf30, C14orf133, C18orf25, C18orf55, C19orf62, C1orf103, C21orf70, C2orf37, C3orf26, C6orf106, C7orf47, C9orf91, CACYBP, CAMTA1, CARS, CBX3, CCDC117, CCDC18, CCDC58, CCDC99, CCT3, CCT4, CCT5, CCT6A, CCT7, CCT8, CD3EAP, CD58, CD59, CDC42EP4, CDC6, CDK3, CDKN2A1PNL, CENPA, CHORDC1, CINP, CKAP2, CKS1B, CKS2, CLEC16A, CLIC4, COPS7B, CPSF3, CSNK1A1, CTCF, CTNNBL1, CYP2R1, CYR61, DAPK3, DCP1A, DGKE, DIDO1, DNAJA1, DNAJC21, DSN1, EARS2, EEF2, EFCAB7, EHD2, EIF1AD, EIF2B5, EIF3H, EIF6, ELAVL1, ENTPD6, ERCC1, EXT1, FAM122B, FAM55C, FAM83D, FAM96B, FAM98A, FKBP4, FLAD1, FLJ22222, FOXK2, FUT5, FXR1, GALNT2, GFM2, GNG5, GPBP1, GTF21RD1, GTF3C3, HNRNPA2B, HNRNPA3, HNRNPF, HNRNPUL1, HSP90AA1, HSP90AB1, HSPA4, HSPA8, HSPA9, HSPC152, HSPD1, HSPE1, HSPH1, HTATSF1, HYPK, ICT1, IGF2BP1, IGF2BP3, IPP, IRF3, ISY1, ITGB3BP, ITGB5, JMJD6, JTB, KIAA0146, KIAA0406, KIAA1303, KNTC1, KRT18, LAMC1, LCMT1, LIAS, LOC124512, LOC134145, LOC144097, LOC400506, LOH2CR1, LONP1, LSM10, LSM2, LSM4, LUC7L2, MANBAL, MAP2K2, MAP4K4, MAPRE1, MAT2A, MED1, MEPCE, METTL8, MFAP1, MLH1, MOCS2, MORF4L2, MPHOSPH10, MRPL13, MRPL18, MRPL44, MRPL48, MRPS28, MTBP, MTDH, MTHFD1L, MTMR12, MUM1, MYH9, MYL6, NARG1L, NAT13, NDUFV2, NKIRAS2, NKRF, NOB1, NSUN2, NT5DC1, NUDC, NUP93, NUTF2, NXT2, ORMDL1, PAPD5, PCGF3, PGK1, PGLS, PHTF1, PKNOX1, PLEKHH3, PMS1, PMS2, PNRC2, PPID, PRC1, PRDX6, PRKCSH, PRMT3, PRMT5, PRNPIP, PRPF6, PSPH, PTGES3, PTK2, PTPLAD1, PXDN, RAB22A, RAB5C, RAD51C, RAI14, RALY, RANBP3, RANGAP1, RBM23, RCC2, REXO4, RFC4, RHOF, RIC8A, RNF169, RPL13A, RPL19, RPL22, RPL39, RPS11, RPS21, RRAS, RUVBL1, S100A13, S100A16, SCAND1, SEC22B, SEC31A, SEC63, SECISBP2, SENP1, SEPSEC5, SERPINH1, SETD5, SF3B3, SFRS10, SFRS2, SFXN1, SH3KBP1, SHC1, SHISA5, SLC16A1, SLC35B2, SLC39A1, SLC3A2, SLC7A5, SMARCD2, SMS, SMYD5, SNAP23, SNAP29, SNAPIN, SNX5, SNX8, SOD1, SPR, SPRED2, SPTLC2, SRP68, ST13, STAG2, STAU1, STIP1, SUGT1, SYMPK, TAF12, TCP1, TDG, TEAD1, TH1L, TINP1, TM2D3, TMF1, TOMM34, TPD52L2, TRAF2, TRAF3, TRIP13, TSEN34, TTC4, TTC4, TTF2, TYW3, UBB, UBC, UBE2F, UBE2H, UBE2V1, UBFD1, UBQLN1, UBXD8, UHRF1, USPL1, UXT, VANGL1, WDR18, WDR70, WHSC1, XPNPEP3, XPO1, YY1, ZC3H18, ZC3HAV1, ZNF212, ZNF227, ZNF282, ZNF326, ZNF451, ZNF473, ZNHIT1, ZSCAN16.

Table A-3: HSF1-CaSig3

ABCA7, ACD, ACTN4, ACY1, ADCY9, ANTXR1, ASCC2, ATL3, ATP2C1, ATXN10, B3GALNT2, B3GNT1, B4GALT1, BAG2, BLVRB, BRMS1, C15orf63, C18orf55, C1orf172, C21orf70, C22orf15, C2orf18, C3orf64, CACNB2, CACYBP, CALM1, CARS, CCT5, CCT6A, CCT7, CDC6, CDC73, CDH23, CENPT, CHCHD6, CIAPIN1, CKS1B, CLIC4, CNDP2, COPA, CPSF3, CREG1, CTCF, CTNNBL1, CWC27, DGKE, DHRS12, EIF1AD, ELL, ERCC1, ESR2, EWSR1, EXT1, FAM96B, FAM98A, FCGR2A, GALNTL1, GNAS, GOLGA3, GOT1, GTF3C3, GTPBP1, HSPA4, HSPA6, HSPA8, HSPA9, HSPB1, ICT1, ING5, IRF3, ISY1, ITFG1, ITGB1BP1, IVNS1ABP, JMJD6, KCNC4, KIF21A, KPNA1, LDLR, LIAS, LONP1, LRRC59, LZIC, MAPK14, MBD4, METTL8, MFSD3, MMP11, MMP15, MORC4, MRPL21, MRPL44, MRPS23, MRTO4, MTDH, MTHFD1L, MUM1, MYLK3, NAA50, NCALD, NOB1, NOTCH2NL, NUDC, NUP93, NUTF2, OAZ1, PAFAH1B1, PARD6A, PDE4DIP, PDXK, PGK1, PHF20, PLA2G15, PLA2G6, PMPCA, PPID, PPME1, PPP1R16B, PRMT5, PSMB3, PSMD3, PTEN, PTPRS, RAD51C, RANBP10, RANGAP1, RORA, RPH3AL, RRAD, RTTN, SF3B2, SFRS7, SIRPB2, SLC12A4, SLC38A7, SMARCD2, SNAP29, SRP68, ST7L, STAU1, STIP1, TBC1D1, TGM2, TIAL1, TM7SF3, TM9SF4, TP63, TRIM16, TTC7A, UBE2D3, UBE2F, UBQLN1, VPS53, VRK3, WDR53, WNK1, WWC1, XPNPEP3, YIF1B, ZAN, ZC3H18, ZNF451, ZNF473, AFF2, ANKRD12, BCAN, BCO2, C10orf54, CHST3, COX16, EGFR, EPS15, FBLN1, FOXK2, FOXN3, GNAQ, GPR56, ITPR1, JUN, KIAA0182, LPP, LRRFIP1, LTBP4, LUZP1, MACF1, MAGI1, MAP3K13, MBP, MED23, MICAL2, NEDD4L, PDZD2, PPM1A, RAB2A, RGL1, SEC22B, SH3KBP1, SLCO3A1, SPG7, TEAD1, TNRC18, TPD52, TRIO, TYW1, UBE2I, XYLT1, ZBTB20.

Table B: Refined HSF1 Cancer Signature Set (CSS) Genes

ABCC5, AHNAK2, AHSA1, AK3L1, ATP2C1, ATP6V1A, AZIN1, BAIAP2, BCL10, C6orf106, C9orf3, CACYBP, CALM1, CARS, CBX3, CCNL1, CCT4, CCT5, CCT6A, CCT7, CDC25B, CDC73, CENPA, CES2, CHORDC1, CHST3, CKS2, CLIC4, CLPB, COL2A1, COPA, CORO1C, CPSF1, CRY1, CUL4A, CUX1, CYC1, DARS, DBN1, DNAJA1, DNAJB4, DNAJB6, DOCK4, DPY19L4, DVL1, EEF1D, EGFR, EMILIN1, EWSR1, FAM96B, FXR1, GALT, GIPC1, GNG7, GOLGA3, GPR56, HEATR6, HIST1H4H, HMGN4, HNRNPH3, HSP90AA1, HSPB1, HSPD1, HSPG2, HSPH1, HUS1, IGFBP7, IL1RAP, IMP4, JARID2, JMJD6, JOSD1, JRK, KIAA0090, KIAA0146, KIAA0406, KIAA1755, KLC1, KLHL25, KNTC1, KPNA1, KREMEN2, LDLR, LMNB2, LRP12, LRRC59, LTBP4, MAP4K4, MAP7D1, MBD4, MEGF6, MICAL2, MLX, MMP11, MRPL16, MRPL18, MTCH1, NARF, NCOR2, NDRG1, NMT2, NUDCD3, NUTF2, OPA3, P4HA2, PAPOLA, PAQR4, PDXK, PGK1, PMEPA1, POLR3B, PRKCA, PSMB3, PTGES3, PTK2, PUF60, PXDN, RAB5C, RBM25, REXO4, RFC4, RSRC2, SCHIP1, SF3B3, SFRS7, SLC2A1, SLC39A4, SLC5A3, SNX3, SPOCK1, STIP1, STK3, STX16, TBC1D13, TCP1, TPD52, TPD52L2, TSEN34, TTC26, UBE21, UBE2O, UPP1, UTP11L, WDR67, WNT2, ZCCHC2, ZNF207, ZNF250, ZNF337, ZNF473.

Table C: Refined HSF1 Cancer Signature Set (CSS) Genes Subset

ABCC5, AHSA1, ATP2C1, BCL10, C6orf106, CACYBP, CALM1, CBX3, CCT5, CCT6A, CDC73, CENPA, CKS2, CLPB, CORO1C, CUL4A, DARS, DBN1, DNAJB6, DPY19L4, EWSR1, GPR56, HIST1H4H, HMGN4, HSPH1, HUS1, JOSD1, KIAA0406, KNTC1, KPNA1, MAP4K4, MBD4, MLX, MMP11, MRPL16, MTCH1, NDRG1, NUTF2, P4HA2, PAPOLA, PGK1, POLR3B, SF3B3, SFRS7, SNX3, STX16, TPD52, TTC26, UBE2O, ZNF207.

In some aspects, described herein are cancer-stroma normalization genes and gene sets. As used herein, a “cancer-stroma normalization gene” refers to a gene whose expression level can be used itself or (more typically) together with the expression level of at least one other cancer-stroma normalization gene to determine the proportion of cancer cells versus tumor-associated stromal cells in a sample or to determine the proportion of a gene expression level of a gene of interest (i.e., a gene that is not a cancer-stroma normalization gene, e.g., an HSF1-regulated gene) that is attributable to cancer cells versus tumor-associated stromal cells in a sample. Measuring the expression level of one or more cancer-stroma normalization genes in a sample and the expression level of one or more genes of interest in the sample allows the extraction of individual cancer cell and tumor-associated stromal cell expression levels for the gene(s) of interest from a gene expression measurement obtained from the sample. Thus, a combined gene expression profile can be deconvoluted into a component attributable to tumor-associated stromal cells and a component attributable to cancer cells and/or normalized to indicate the expression level that would have been measured had the sample been composed entirely of tumor-associated stromal cells or entirely of cancer cells. In some embodiments, a cancer-stroma normalization gene is selected from the group consisting of: DSG2, DSP, ELF3, IRF6, MYO5B, MYO6, PTPLB, TRPS1 (the “Cancer High, Stroma Low” set). In some embodiments, a cancer-stroma normalization gene is selected from the group consisting of: BGN, CFH, LTBP2, MRC1, PECAM1, SLCO2B1, TCF4, WIPF1 (the “Stroma High, Cancer Low” set. In some embodiments, a cancer-stroma normalization gene set comprises or consists of at least 1, 2, 3, 4, 5, 6, 7, or all 8 genes selected from the Cancer High, Stroma Low set and at least 1, 2, 3, 4, 5, 6, 7, or all 8 genes selected from the Stroma High, Cancer Low set. In some embodiments a cancer-stroma normalization gene set comprises 2, 3, 4, 5, 6, 7, or 8 genes selected from each of the two sets. For example, in some embodiments a cancer-stroma normalization gene set comprises or consists of between 4 and 8 genes from the Stroma High, Cancer Low set and between 4 and 8 genes from the Cancer High, Stroma Low set. All subsets and combinations of subsets of genes from the Stroma High, Cancer Low set and the Cancer High, Stroma Low set are expressly disclosed.

In some aspects, described herein are sets of genes whose expression level in a sample comprising both cancer cells and cancer-associated stromal cells (e.g., a sample obtained from a tumor) allows measurement of HSF1 activity in either or both of these two groups of cells (i.e., individual measurement of HSF1 activity in cancer cells and/or an individual measurement of HSF1 activity in tumor-associated stromal cells) or an overall assessment HSF1 activity in the tumor. In some embodiments, such a gene set (which may be referred to as an “HSF1 combined tumor signature set”) comprises: (i) a set of genes that are regulated by HSF1 in tumor stromal cells; and (ii) a set of genes that are regulated by HSF1 in cancer cells. In some embodiments, an HSF1 combined tumor signature set is the HSF1 Combined Cancer-Stroma Signature Set (HSF1-CCSS Set), which is composed of the genes listed in Table C and Table D. In some embodiments described herein are HSF1 combined tumor signature sets comprising a subset of the genes listed in Table C and a subset of the genes listed in Table D, e.g., at least 1, at least 2, at least 3, at least 4, at least 5, at least 10, at least 15, at least 20, at least 25, at least 30, at least 35, or at least 40 genes from each of Tables C and D. In some embodiments an HSF1 combined tumor signature set comprises or consists of between 5 and 10, between 10 and 20, between 20 and 30, between 30 and 40, or between 40 and 50 genes from Table C and between 5 and 10, between 10 and 20, between 20 and 30, or between 30 and 42 genes from Table D. For example, in some embodiments an HSF1 combined tumor signature set comprises or consists of between 10 and 20 genes from each of Tables C and D. In some embodiments an HSF1 combined tumor signature set comprises or consists of between 20 and 30 genes from each of Tables C and D. In some embodiments an HSF1 combined tumor signature set comprises or consists of between 30 and 40 genes from each of Tables C and D. In some embodiments an HSF1 combined tumor signature set is augmented by including one or more cancer-stroma normalization genes or gene sets, such as the genes listed in Table E, or a subset thereof. Thus in some aspects, described herein is a gene set composed of the HSF1 Combined Cancer-Stroma Signature Set (or any subset thereof) and the genes listed in Table E (or any subset thereof). Exemplary subsets of the HSF1 Combined Cancer-Stroma Signature Set and exemplary subsets of the genes listed in Table E are described herein. All subsets and combinations of subsets are expressly disclosed. As described in Example 1, genes in Table E were identified by applying certain criteria to genes described in the Ma, et al. 2009, and/or Winslow, et al. 2015 datasets. It should be noted that other genes in these datasets meet these criteria and could be used as cancer-stroma normalization genes in addition to or instead of the genes listed in Table E in any of the methods or compositions described herein.

In some embodiments of any of the methods described herein, expression of between 5 and 10, between 10 and 25, between 25 and 50, between 50 and 100, between 100 and 150, or between 150 and 200 HSF-regulated genes is measured. In some embodiments, expression of between 200 and 300, between 300 and 400, between 400 and 500, between 500 and 600, between 600 and 750, or between 750 and 1000 HSF-regulated genes is measured. In some embodiments of particular interest, between 50 and 100 HSF1-regulated genes and at least 2, e.g., between 5 and 16, or between 10 and 16 cancer-stroma normalization genes are measured. A set of genes whose expression can be specifically measured by performing a particular assay or using a particular set of reagents may be referred to as target genes for that assay or set of reagents. In some embodiments, at least 50%, at least 60%, at least 70%, at least 80%, at least 85%, at least 90%, at least 95%, or more of the target genes are HSF-1 regulated genes. In some embodiments, at least 50%, at least 60%, at least 70%, at least 80%, at least 85%, at least 90%, at least 95%, or more of the target genes are HSF1-SSS genes. In some embodiments, at least 50%, at least 60%, at least 70%, at least 80%, or more of the target genes are HSF1-G1 genes. In some embodiments, at least 50%, at least 60%, at least 70%, at least 80%, or more of the target genes are HSF1-G4 genes. In some embodiments, at least 50%, at least 60%, at least 70%, at least 80%, at least 85%, at least 90%, at least 95%, or more of the target genes are either an HSF1-CSS gene (Table A) or an HSF1-SSS gene (gene lists G1 and G4). In some embodiments, at least 50%, at least 60%, at least 70%, at least 80%, at least 85%, at least 90%, at least 95%, or more of the target genes are either an HSF1-CSS gene (Table A) or a Refined HSF1-SSS gene (Table D). In some embodiments, at least 50%, at least 60%, at least 70%, at least 80%, at least 85%, at least 90%, at least 95%, or more of the target genes are either a Refined HSF1-CSS gene (Table B) or a Refined HSF1-SSS gene (Table D). In some embodiments, at least 50%, at least 60%, at least 70%, at least 80%, at least 85%, at least 90%, at least 95%, or more of the target genes are either a Refined HSF1-CSS gene listed in Table C or a Refined HSF1-SSS gene (Table D). In some embodiments the genes from Table D include at least 10, 11, 12, 13, 14, or all 15 genes from the “15 gene set” described in Example 10. In some embodiments the genes from Table D include at least 15, 16, 17, 18, 19, 20, 21, 22, 23, 24, or all 25 genes from the “25 gene set” described in Example 10. In some embodiments the genes from Table D include TGFB1 and CXCL12. In some embodiments one or more of the target genes are cancer-stroma normalization genes, e.g., genes listed in Table E. In some embodiments genes whose expression may be measured as part of an assay but whose expression level is not used in determining the level of HSF1 expression, activation, or activity and/or is not used to provide diagnostic, prognostic, treatment-specific predictive information or in treatment selection may be excluded from the set of genes which are considered to be target genes for that assay. For example, in some embodiments genes whose expression is measured as controls may be excluded. In some embodiments, genes whose expression is not regulated by HSF1 and that are not used as cancer-stroma normalization genes may be excluded. In certain embodiments the number of target genes is no more than 50. In certain embodiments the number of target genes is no more than 55. In certain embodiments the number of target genes is no more than 60. In certain embodiments the number of target genes is no more than 65. In certain embodiments the number of target genes is no more than 70. In certain embodiments the number of target genes is no more than 75. In certain embodiments the number of target genes is no more than 80. In certain embodiments the number of target genes is no more than 85. In certain embodiments the number of target genes is no more than 90. In certain embodiments the number of target genes is no more than 95. In certain embodiments the number of target genes is no more than 100.

In some aspects, the combined set of genes listed in Tables C, D, and E, or a subset of these genes, which may be any of the subset s described herein), is of particular interest in any of the methods described herein that comprise measuring HSF1 expression and/or activation in tumor-associated stromal cells and in tumor cells. For example, any of the methods described herein that comprise measuring HSF1 expression and/or activation in tumor-associated stromal cells, cancer cells, or both, may comprise measuring HSF1 activity in tumor-associated stromal cells, cancer cells, or both, by measuring expression of the genes listed in Tables C, D, and E, or a subset of these genes, which may be any of the subsets described herein.

In some embodiments the level of an HSF1 activity is expressed as an absolute level. In some embodiments the level of an HSF1 activity is expressed as a relative level. For example, activation or repression of a gene by HSF1 may be expressed as a fold-increase or fold-decrease in expression relative to a reference level.

In some embodiments in which expression of multiple HSF1-regulated genes in tumor-associated stromal cells is measured, an average expression level of such genes may be used as a measurement of HSF1 activity in tumor-associated stromal cells. Likewise, in some embodiments in which expression of multiple HSF1-regulated genes in cancer cells is measured, an average expression level of such genes may be used as a measurement of HSF1 activity in cancer cells. In some embodiments an average expression level may be an average fold difference relative to a reference level.

In some embodiments, expression of HSF1-regulated gene(s) is measured by detecting mRNA encoded by such gene(s). One of ordinary skill in the art appreciates that mRNA may be detected as cDNA after reverse transcription and that reverse transcription can be performed using various types of primers, e.g., primers comprising sequence-specific oligonucleotides that hybridize to target RNA to be detected, oligodT primers that hybridize to polyA tails of mRNA, or primers comprising random hexamers. In some embodiments cDNA produced by reverse transcription of mRNA may be labeled, e.g., with a fluorophore, to facilitate its detection.

In some embodiments described herein are assays and nucleic acid reagents (e.g., probes and/or primers) suitable for measuring expression of any one or more HSF-1 regulated genes and/or one or more cancer-stroma normalization genes described herein. One of ordinary skill in the art will appreciate that a set of reagents suitable for use in a given assay may depend, in general, on the particular type of assay. For assays that comprise detecting mRNA encoded by a given gene, the reagents typically comprise at least one probe or primer that hybridizes specifically to such mRNA or its complement (e.g., cDNA or cRNA). For example, microarrays typically comprise sequence-specific probes that hybridize to complementary DNA reverse transcribed from mRNA to be detected. One of ordinary skill in the art appreciates that microarrays encompass arrays in which different probes are disposed as discrete features on a substantially planar support (whereby different features are addressable based on location) or arrays in which different probes are attached to beads (bead arrays). The beads may be impregnated with different concentrations and/or combinations of fluorescent dyes to render them distinguishable or may be distinguishable by oligonucleotide barcodes attached thereto. Bead array technologies known in the include those available from Luminex and Illumina (Life Technologies). Such technologies may be used to measure expression of one or more HSF1-regulated genes and, in some embodiments, one or more cancer-stroma normalization genes. PCR assays typically use a pair of primers that hybridize to sequences at the 3′ ends of a region of DNA to be amplified (i.e., one primer is complementary to each strand). As known in the art, RNA to be detected would typically be reverse transcribed to cDNA prior to microarray hybridization or prior to the first round of PCR. Quantitative PCR methods use a reporter to measure the amount of amplified product in real time. Reporters can be non-sequence specific DNA dyes that intercalate into double-stranded DNA, such as SYBR Green and EvaGreen or sequence-specific fluorophore-labeled oligonucleotides, which may serve as primers for the PCR amplification or as probes that hybridize to the PCR product. Many sequence-specific PCR reporters use fluorescence quenching to ensure that fluorescence is detected only when amplification product from the target of interest is present. The PCR primer or target-specific oligonucleotide probe is labeled with a fluorophore whose fluorescence is quenched when the specific target DNA sequence is not present. Quenching may be accomplished by attaching a quencher molecule to the DNA primer or probe in combination with some process by which the reporter and quencher are separated when the primer or probe hybridizes to its specific target sequence. For example, hydrolysis-based assays (often called TaqMan or 5′ nuclease assays) use sequence-specific PCR primers and a sequence-specific, oligonucleotide probe labeled with a fluorescent reporter at the 5′ end and a quencher at the 3′ end. When the probe is intact, the fluorescence of the reporter is quenched due to its proximity to the quencher. The amplification reaction includes a combined annealing/extension step during which the probe hybridizes to the target, and the dsDNA-specific 5′→3′ exonuclease activity of Taq or Tth DNA polymerase cleaves off the reporter, separating it from the quencher, resulting in a fluorescence signal proportional to the amount of amplified product in the sample. One of ordinary skill in the art appreciates that a variety of other methodologies and probe/primer configurations may be used to allow specific detection of an amplification product.

NanoString assays for measuring gene expression typically use two sequence-specific probes for each gene of interest. In a typical implementation the first probe, a capture probe, contains an approximately 35- to 50-base sequence complementary to a particular target mRNA and a short common sequence coupled to an affinity tag such as biotin. The second probe, the reporter probe, contains a second approximately 35- to 50-base sequence complementary to the target mRNA, which sequence is coupled to a color-coded tag that provides the detection signal. The color-coded tag consists of a single-stranded nucleic acid molecule annealed to a series of complementary in vitro transcribed RNA segments each labeled with a specific fluorophore. The linear order of these differently colored RNA segments creates a unique code for each gene of interest. Probes for detecting multiple different RNAs are mixed together with total RNA in a hybridization reaction that proceeds in solution. Hybridization forms a structure comprising target RNA, capture probe, and reporter probe. After removal of unhybridized reporter and capture probes the remaining complexes are captured on a surface coated with an appropriate capture reagent (e.g., streptavidin). An applied electric field extends and orients each complex and the complexes are then immobilized in an elongated state and imaged. Each target molecule of interest is identified by the color code generated by the ordered fluorescent segments present on the reporter probe for that molecule. The level of expression is measured by counting the number of codes for each mRNA. (See, e.g., US Pat. Pub. No. 20100261026).

Reverse transcriptase multiplex ligation-dependent probe amplification (RT-MLPA) is a variation of the multiplex polymerase chain reaction that permits multiple targets to be amplified with a single primer pair (Schouten J P, et al. (2002). Relative quantification of 40 nucleic acid sequences by multiplex ligation-dependent probe amplification, Nucleic Acids Res. 30 (12): e57). Each probe consists of two oligonucleotides which hybridize to adjacent target sites on the target DNA (e.g., cDNA reverse transcribed from mRNA). One probe oligonucleotide contains the sequence recognized by the forward primer; the other probe contains the sequence recognized by the reverse primer. Only when both probe oligonucleotides are hybridized to their respective target sequences can they be ligated into a complete probe. Due to splitting the probe into two parts, only the sequence composed of the ligated oligonucleotides (and its complement) is amplified. Each complete ligated probe has a unique length, so that its resulting amplicons can be separated and identified by, e.g., electrophoresis, e.g., capillary electrophoresis. At least one of the primers (e.g., the forward primer) used for probe amplification is labeled, e.g., fluorescently labeled. Each amplicon generates a fluorescent peak which can be detected by, e.g., a capillary sequencer, allowing the quantity or relative quantity of each amplicon to be determined. Many sequences (e.g., up to 50) can be amplified and quantified using a single primer pair. Probes suitable for use in RT-MLPA assays can be designed according to principles known in the art. Software such as MLPA® Designer (PREMIER Biosoft, Palo Alto, Calif.) may be used.

Thus in some embodiments, reagents for measuring expression of a particular gene may comprise one or more sequence-specific probes that hybridize to mRNA encoded by the gene or to cDNA complementary to the mRNA, a pair of sequence-specific primers, or one or more sequence-specific primers (e.g., a primer pair) and a sequence-specific probe. In some embodiments, probes and/or primers for measuring expression of different genes may be located in different vessels (e.g., tubes, wells). For example, primer pairs for quantifying different mRNAs may be located in individual wells of a multiwell plate. In some embodiments, probes and/or primers for measuring expression of different genes are contained in the same vessel so that expression of multiple genes can be measured in the same reaction (multiplex assays). In some embodiments, different probes and/or primers may be physically associated with distinct detectable labels, allowing them to be distinguished.

It will be appreciated that any of the methods described herein for measuring mRNA may be used to measure the level of expression of one or more genes that are regulated by HSF1 in tumor-associated stromal cells and/or to measure the level of expression of one or more genes that are regulated by HSF1 in cancer cells and/or to measure the level of expression of one or more cancer-stroma normalization genes.

In some embodiments, the same type of method (e.g., IHC, microarray analysis, Nanostring analysis, RT-PCR, RT-MLPA, RNA-Seq) is used to measure HSF1 expression and/or activation (e.g., HSF1 activity) both in tumor-associated stromal cells and in cancer cells of a tumor. In some embodiments the measurements are performed on the same tumor sample. In some embodiments the measurements are performed on different samples from the same tumor. In some embodiments, HSF1 expression and/or activation is measured in tumor-associated stromal cells and in cancer cells of a tumor using different methods. For example, in some embodiments HSF1 protein level and/or HSF1 nuclear localization are measured using IHC in tumor-associated fibroblasts, and expression of one or more genes that are regulated by HSF1 is measured in cancer cells using a hybridization-based assay (e.g., RT-PCR or a Nanostring assay).

In some embodiments, the level of HSF1 activation is determined by measuring binding of HSF1 to the promoter of one or more HSF1-regulated genes, wherein binding of HSF1 to the promoter of an HSF1-regulated gene is indicative of HSF1 activation. In some embodiments, an HSF1-regulated gene is a gene whose expression level (e.g., as assessed based on mRNA or protein levels) is increased or decreased by at least a factor of 1.2 as a result of HSF1 activation. In some embodiments, an HSF1-regulated gene is among the 1,000 genes in the human genome whose expression is most strongly affected (increased or inhibited) by HSF1. In some embodiments, an HSF1-regulated gene is among the 1,000 genes in the human genome whose promoter is most strongly bound by HSF1 under conditions in which HSF1 is activated. Methods for measuring binding of a protein (e.g., HSF1) to DNA (e.g., genomic DNA) include, e.g., chromatin immunoprecipitation using an antibody to the protein followed by microarray hybridization to identify bound sequences, commonly referred to as ChIP-on-chip (see, e.g., U.S. Pat. Nos. 6,410,243; 7,470,507; 7,575,869); ChIP-Sequencing, which uses chromatin immunoprecipitation followed by high throughput sequencing to identify the bound DNA; and DamID (DNA adenine methyltransferase identification; see, e.g., Vogel M J, et al (2007). “Detection of in vivo protein-DNA interactions using DamID in mammalian cells”. Nat Protoc 2 (6): 1467-78).

In some embodiments, an assay to detect HSF1 expression or activation makes use of fluorescence resonance energy transfer (FRET).

In some embodiments, the level of an HSF1 gene product or the level of HSF1 activation is determined to be “increased” or “not increased” by comparison with a suitable control level or reference level. The terms “reference level” and “control level” may be used interchangeably herein. A suitable control level can be a level that represents a normal level of HSF1 gene product or HSF1 activation, e.g., a level of HSF1 gene product or HSF1 activation existing in cells or tissue in a non-diseased condition and in the substantial absence of stresses that activate the heat shock response. Thus any method that includes a step of (a) assessing (determining, measuring) the level of HSF1 expression and/or activation can comprise a step of (b) comparing the level of HSF1 expression and/or activation with a control level of HSF1 expression and/or activation, wherein if the level determined in (a) is greater than the control level, then the level determined in (a) is considered to be “increased” (or, if the level determined in (a) is not greater than the control level, i.e., is about the same as or lower than the control level, then the level determined in (a) is considered to be “not increased”. For example, if tumor-associated stromal cells of a tumor have an increased level of HSF1 expression and/or activation as compared to a control level, the tumor is classified as having a high risk of poor outcome, while if the tumor-associated stromal cells do not have a significantly increased level of HSF1 relative to a control level, the tumor is classified as having a low risk of poor outcome. Likewise it would be understood that the level of a gene product of an HSF1-regulated gene can be determined to be “increased” or “not increased” by comparison with a suitable control level or reference level and that any method that includes a step of (a) assessing (determining, measuring) the level of expression of an HSF1-regulated gene may comprise a step of (b) comparing the level of expression of the gene with a control level of expression wherein if the level determined in (a) is greater than the control level, then the level determined in (a) is considered to be “increased” (or, if the level determined in (a) is not greater than the control level, i.e., is about the same as or lower than the control level, then the level determined in (a) is considered to be “not increased” or, if the level determined in (a) is lower than the control level, then the level determined in (a) may be considered to be “decreased”. As described herein, assessing (determining, measuring) the level of expression of an HSF1-regulated gene may comprise measuring the level of a gene product of the gene, e.g., mRNA transcribed from the gene. Thus, determining whether expression of a gene is increased (or decreased) may comprise comparing the level of a gene product of a gene with a control level, wherein if the level of the gene product is greater than the control level, expression of the gene is considered to be “increased”, while if the level of the gene product is about the same as or lower than the control level, then the level may be considered to be “not increased” or, if the level of the gene product is lower than the control level, then the level of expression of the gene may be considered to be “decreased”. In some embodiments, an “increase”, “increased”, or like terms refers to an increase by a factor of about or at least 1.1, 1.2, 1.3, 1.4, 1.5, 1.6, 1.7, 1.8, 1.9, 2, 3, 4, 5, 6, 7, 8, 9, 10, or more, relative to a control level. In some embodiments, a “decrease”, “decreased”, or like terms refers to a decrease by a factor of about or at least 1.1, 1.2, 1.3, 1.4, 1.5, 1.6, 1.7, 1.8, 1.9, 2, 3, 4, 5, 6, 7, 8, 9, 10, or more, relative to a control level.

A control level may be determined in a variety of ways. In some embodiments a control level is an absolute level. In some embodiments a control level is a relative level. A comparison can be performed in various ways. For example, in some embodiments one or more samples comprising tumor-associated stromal cells are obtained from a tumor, and one or more samples are obtained from normal (non-tumor) tissue from the same patient. The relative level of HSF1 gene product or HSF1 activation in the sample(s) comprising tumor-associated stromal cells versus the non-tumor sample(s) is determined. In some embodiments, if the relative level (ratio) of HSF1 gene product in the samples comprising tumor-associated stromal cells versus the non-tumor sample(s) is greater than a predetermined value (indicating that tumor-associated stromal cells have increased HSF1), the tumor is classified as high risk. In some embodiments the predetermined value is, e.g., at least 1.5, 2, 2.5, 3, 5, 10, 20, or more. In some embodiments the predetermined value is between about 1.5 and about 10. A control level may be a historical measurement. For example, the data provided herein provide examples of levels of HSF1 expression and/or activation in various normal and tumor-associated stromal cells. In some embodiments a value may be semi-quantitative, qualitative or approximate. For example, visual inspection (e.g., using light microscopy) of a stained IHC sample can provide an assessment of the level of HSF1 expression or HSF1 activation without necessarily counting cells or nuclei or precisely quantifying the intensity of staining.

In some embodiments, e.g., in a method for classifying tumors, providing prognostic information, assessing tumor aggressiveness, predicting cancer outcome, providing treatment-specific predictive information, or selecting a treatment, a control level of expression of an HSF-regulated gene may be a level representative of tumors that have a good prognosis, low aggressiveness, or low propensity to metastasize or recur. In some embodiments an average expression level of two or more HSF1-regulated genes is obtained and compared with a control level. The two or more HSF1-regulated genes may be any set or subset of HSF1-regulated genes described herein.

In some embodiments of any of the methods, the expression level of a gene, e.g., an HSF1-regulated gene, may be normalized, e.g., using the expression level of a gene whose expression is not expected to change significantly in cancer cells versus non-transformed cells. In some embodiments actin or HPRT1 is used for normalization. In some embodiments the expression level of a gene in stromal cells (tumor-associated stromal cells or non-tumor-associated stromal cells) is normalized using the expression level of one or more members of the Stroma High, Cancer Low gene set. In some embodiments the expression level of a gene in cancer cells or non-cancer cells is normalized using the expression level of one or more members of the Cancer High, Stroma Low gene set. It would be understood that when comparing the level of expression of a gene in

Various risk categories may be defined. For example, tumors may be classified as at low, intermediate, or high risk of poor outcome. A variety of statistical methods may be used to correlate the risk of poor outcome with the relative or absolute level of HSF1 expression or HSF1 activation.

For purposes of description herein it is assumed that the control or reference level for comparison with a level present in tumor-associated stromal cells represents normal levels of HSF1 expression or HSF1 activation present in normal stromal cells and/or tissues not associated with a tumor and not exposed to heat shock or other stresses that would be expected to increase HSF1 expression and/or activation. Likewise, it is assumed that the control or reference level for comparison with a level present in cancer cells represents normal levels of HSF1 expression or HSF1 activation present in non-neoplastic cells, e.g., normal cells of the type from which the tumor arose or normal tissue in the organ in which the tumor arose or is present, and not exposed to heat shock or other stresses that would be expected to increase HSF1 expression and/or activation. However, it will be understood that a level of HSF1 expression or HSF1 activation characteristic of tumor-associated stromal cells could be used as a reference or control level for comparison with a level present in tumor-associated stromal cells from a tumor of interest and/or a level of HSF1 expression or HSF1 activation characteristic of cancer cells could be used as a reference or control level for comparison with a level present in cancer cells from a tumor of interest. In that case, the presence of HSF1 expression or HSF1 activation at a level comparable to, e.g., approximately the same, as or greater than the control level would be indicative of the presence of cancer, poor cancer prognosis, aggressive cancer phenotype, or to identify a subject who is a suitable candidate for treatment with an HSF1 inhibitor, while a decreased level of HSF1 expression or HSF1 activation as compared with the control level would be predictive of good cancer prognosis, less aggressive cancer phenotype, etc.

In some embodiments a method comprises classifying a tumor by comparing the level of expression and/or activation of HSF1 in tumor-associated stromal cells with the level of expression and/or activation of HSF1 in tumor-associated stromal cells in a representative cohort of tumors that have known outcomes. In some embodiments a method comprises classifying a tumor by comparing the level of expression and/or activation of HSF1 in tumor-associated stromal cells and in cancer cells with the level of expression and/or activation of HSF1 in tumor-associated stromal cells and cancer cells in a representative cohort of tumors that have known outcomes. In some embodiments, tumors classified among the upper 10%, 15%, 20%, or 25% of tumors by level of HSF1 expression and/or activation are determined to have a worse prognosis, be more aggressive, and/or be more likely to need intensive treatment than tumors classified in the lower 75% (or any lower percentile, such as the lower 60%, 50%, 40%, 30%, etc.). In some embodiments, tumors classified among the upper 30% or 35% of tumors by level of HSF1 expression and/or activation are determined to have a worse prognosis, be more aggressive, and/or be more likely to need intensive treatment than tumors classified in the lower 65% (or any lower percentile, such as the lower 60%, 50%, 40%, 30%, etc.) In some embodiments, tumors classified among the upper 50% of tumors by level of HSF1 expression and/or activation are determined to have a worse prognosis, be more aggressive, and/or be more likely to need intensive treatment than tumors classified in the lower 50% (or any lower percentile, such as the lower 40%, 30%, etc.) In some embodiments, tumors classified among the upper 75% of tumors by level of HSF1 expression and/or activation are determined to have a worse prognosis, be more aggressive, and/or be more likely to need intensive treatment than tumors classified in the lower 25% (or the lower 20%, 10%, etc.). In some embodiments, tumors classified among the lower 10%, 15%, 20%, or 25% of tumors by level of HSF1 expression and/or activation are determined to have a better prognosis, be less aggressive, and/or be less likely to need intensive treatment than tumors classified in the upper 75% (or the upper 60%, 50%, 40%, 30%, etc.). In some embodiments, tumors classified among the lower 33% of tumors by level of HSF1 expression and/or activation are determined to have a better prognosis, be less aggressive, and/or be less likely to need intensive treatment than tumors classified in the lower 67% (or the lower 60%, 50%, 40%, 30%, etc.). In some embodiments, any of the afore-mentioned comparisons or determinations may be based on the level of HSF1 expression and/or activation in tumor-associated stromal cells or may be based on the level of HSF1 expression and/or activation both in tumor-associated stromal cells and in cancer cells.

In some embodiments, any of the afore-mentioned comparisons or determinations may be based on the level of HSF1 activity as determined by measuring expression of one or more HSF1-regulated genes in tumor-associated stromal cells or cancer cells. For example, in some embodiments, tumors classified among the upper 10%, 15%, 20%, or 25% of tumors based on the level of HSF1 activity as determined by measuring expression of one or more HSF1-regulated genes are determined to have a worse prognosis, be more aggressive, and/or be more likely to need intensive treatment than tumors classified in the lower 75% (or any lower percentile, such as the lower 60%, 50%, 40%, 30%, etc.). In some embodiments, tumors classified among the upper 30% or 35% of tumors based on the level of HSF1 activity as determined by measuring expression of one or more HSF1-regulated genes are determined to have a worse prognosis, be more aggressive, and/or be more likely to need intensive treatment than tumors classified in the lower 65% (or any lower percentile, such as the lower 60%, 50%, 40%, 30%, etc.) In some embodiments, tumors classified among the upper 50% of tumors based on the level of HSF1 activity as determined by measuring expression of one or more HSF1-regulated genes are determined to have a worse prognosis, be more aggressive, and/or be more likely to need intensive treatment than tumors classified in the lower 50% (or any lower percentile, such as the lower 40%, 30%, etc.) In some embodiments, tumors classified among the upper 75% of tumors based on the level of HSF1 activity as determined by measuring expression of one or more HSF1-regulated genes are determined to have a worse prognosis, be more aggressive, and/or be more likely to need intensive treatment than tumors classified in the lower 25% (or the lower 20%, 10%, etc.). In some embodiments, tumors classified among the lower 10%, 15%, 20%, or 25% of tumors based on the level of HSF1 activity as determined by measuring expression of one or more HSF1-regulated genes are determined to have a better prognosis, be less aggressive, and/or be less likely to need intensive treatment than tumors classified in the upper 75% (or the upper 60%, 50%, 40%, 30%, etc.). In some embodiments, tumors classified among the lower 33% of tumors based on the level of HSF1 activity as determined by measuring expression of one or more HSF1-regulated genes are determined to have a better prognosis, be less aggressive, and/or be less likely to need intensive treatment than tumors classified in the lower 67% (or the lower 60%, 50%, 40%, 30%, etc.). In some embodiments, any of the afore-mentioned comparisons or determinations may be based on the level of HSF1 activity as determined by measuring expression of one or more HSF1-regulated genes in tumor-associated stromal cells or may be based on the level of HSF1 activity as determined by measuring expression of one or more HSF1-regulated genes both in tumor-associated stromal cells and in cancer cells.

In some embodiments clustering based on gene expression levels may be used to position a tumor with respect to tumors having known outcomes and/or known response to a particular treatment. If a tumor of interest clusters with tumors from subjects that had a poor outcome the tumor may be classified as having a poor prognosis. If a tumor of interest clusters with tumors from subjects that had a good outcome the tumor may be classified as having a good prognosis. If a tumor of interest clusters with tumors that responded well to a particular treatment, the tumor may be classified as likely to respond well to that treatment. If a tumor of interest clusters with tumors that did not respond well to a particular treatment, the tumor may be classified as not likely to respond well to that treatment.

Wherever relevant herein, a difference between two or more values (e.g., measurements) or groups, or a relationship between two or more variables, may be statistically significant. As used herein, “statistically significant” may refer to a p-value of less than 0.05 using an appropriate statistical test. One of ordinary skill in the art will be aware of appropriate statistical tests and models for assessing statistical significance, e.g., of differences in measurements, relationships between variables, etc., in a given context. Exemplary tests and models include, e.g., t-test, ANOVA, chi-square test, Wilcoxon rank sum test, log-rank test, Cox proportional hazards model, etc. In some embodiments multiple regression analysis may be used. In some embodiments, a p-value may be less than 0.025. In some embodiments, a p-value may be less than 0.01. In some embodiments a two-sided statistical test is used. In some embodiments, a result or outcome or difference between two or more values is “statistically significant” if it has less than a 5%, less than a 2.5%, or less than a 1% probability of occurring by chance.

Methods are sometimes stated herein mainly in terms of conclusions or predictions that can be made if increased HSF1 expression and/or activation is present. Methods could equally well be stated in terms of conclusions or predictions that can be made if increased HSF1 expression or increased HSF1 activation is not present. For example, if HSF1 expression and/or activation is low or absent in tumor-associated stromal cells of a sample, the sample would not be classified as cancer based on the assay. If HSF1 expression and/or activation is absent or low in a sample from an invasive tumor, the tumor would not be classified as having a poor prognosis based on the assay.

Any of the methods of the invention may, in certain embodiments, comprise assigning a score to a sample (or to a tumor from which a sample was obtained) based on the level of HSF1 expression and/or activation measured in the sample, e.g., based on the level of an HSF1 gene product and/or the level of HSF1 activation in tumor-associated stromal cells. In some embodiments any such method may further comprise assigning a score based on the level of HSF1 expression and/or activation in cancer cells. In some embodiments a combined score based on the level of HSF1 expression and/or activation in tumor-associated stromal cells and the level of HSF1 expression and/or activation in cancer cells is assigned.

In some embodiments a score is assigned based on assessing both HSF1 polypeptide level and HSF1 activation level. For example, a score can be assigned based on the number (e.g., percentage) of nuclei that are positive for HSF1 and the intensity of the staining in the positive nuclei. For example, a first score can be assigned based on the percentage positive nuclei, and a second score assigned based on staining intensity in the nuclei. In some embodiments, the two scores are added or multiplied to obtain a composite score. The range can be divided into multiple (e.g., 2 to 5) smaller ranges, and samples or tumors are assigned an overall HSF1 expression/activation score based on which subrange the composite score falls into. A higher score indicates, for example, increased aggressiveness, increased likelihood of poor outcome, poor prognosis. Thus in some aspects, the invention provides a method of assigning a score to a sample comprising cells, the method comprising steps of: (a) assigning a first score to the sample based on the number or percentage of cell nuclei that are positive for HSF1 protein; (b) assigning a second score to the sample based on the level of HSF1 protein in cell nuclei; and (c) obtaining a composite score by combining the scores obtained in step (a) and step (b). In some embodiments, combining the scores comprises adding the scores. In some embodiments combining the scores comprises multiplying the scores. In some embodiments the method further comprises assigning the sample to an HSF1 expression/activation category based on the composite score. It will be understood that if the sample is a tissue sample that comprises areas of tumor tissue and areas of normal tissue (e.g., as identified using standard histopathological criteria), the score(s) can be assigned based on assessing tumor tissue. Normal tissue may be used as a control. A portion of normal tissue well separated from the tumor tissue may be used.

In some embodiments, a score is assigned using a scale of 0 to 3, where 0 indicates no detectable HSF1 polypeptide in tumor-associated stromal cell nuclei, 1 indicates low, 2 indicates intermediate, and 3 indicates high levels of HSF1 in tumor-associated stromal cell nuclei. A higher score indicates a less favorable prognosis than a lower score, e.g., more likely occurrence of metastasis, shorter disease free survival, or shorter overall survival. A score can be obtained by evaluating one field or multiple fields in a cell or tissue sample. Multiple samples from a tumor may be evaluated in some embodiments. It will be understood that “no detectable HSF1” could mean that the level detected, if any, is not noticeably or not significantly different to background levels. It will be appreciated that a score can be represented using numbers or using any suitable set of symbols or words instead of, or in combination with numbers. For example, scores can be represented as 0, 1, 2; negative, positive; negative, low, high; −, +, ++, +++; 1+, 2+, 3+, etc.

In some embodiments, at least 20, 50, 100, 200, 300, 400, 500, 1000 cells, or more (e.g., tumor-associated stromal cells) are assessed to evaluate HSF1 expression and/or activation in cells of a sample or tumor, e.g., to assign a score to a sample or tumor. In some embodiments, samples or tumors that have low or absent HSF1 polypeptide in tumor-associated stromal cell nuclei, e.g., as assessed using IHC, may be considered negative for tumor-associated stromal cell HSF1.

The number of categories in a useful scoring or classification system can be at least 2, e.g., between 2 and 10, although the number of categories may be greater than 10 in some embodiments. The scoring or classification system often is effective to divide a population of tumors or subjects into groups that differ in terms of an outcome such as local progression, local recurrence, discovery or progression of regional or distant metastasis, death from any cause, or death directly attributable to cancer. An outcome may be assessed over a given time period, e.g., 2 years, 5 years, 10 years, 15 years, or 20 years from a relevant date. The relevant date may be, e.g., the date of diagnosis or approximate date of diagnosis (e.g., within about 1 month of diagnosis) or a date after diagnosis, e.g., a date of initiating treatment. Methods and criteria for evaluating progression, response to treatment, existence of metastases, and other outcomes are known in the art and may include objective measurements (e.g., anatomical tumor burden) and criteria, clinical evaluation of symptoms), or combinations thereof. For example, 1, 2, or 3-dimensional imaging (e.g., using X-ray, CT scan, or MRI scan, etc.) and/or functional imaging (e.g., PET scan) may be used to detect or assess lesions (local or metastatic), e.g., to measure anatomical tumor burden, detect new lesions, etc. In some embodiments, a difference between groups is statistically significant as determined using an appropriate statistical test or analysis method, which can be selected by one of ordinary skill in the art. In many embodiments, a difference between groups would be considered clinically meaningful or clinically significant by one of ordinary skill in the art.

Kits and Systems

In some aspects, the invention provides kits comprising reagents suitable for performing an assay to assess HSF1 expression or HSF1 activation, e.g., for use in a method of the invention. Such kits may contain, e.g., (i) a probe or primer (optionally labeled and/or attached to a support) for detecting, reverse transcribing, and/or amplifying an HSF1 RNA, (e.g., HSF1 mRNA); (ii) a probe or primer for detecting, reverse transcribing, and/or amplifying an RNA (e.g., mRNA) transcribed from a gene regulated by HSF1 in tumor stromal cells or regulated in cancer cells in a manner dependent on HSF expression and/or activation in tumor-associated stromal cells; (iii) an antibody that binds to an HSF1 polypeptide (e.g., for use in IHC); (iv) one or more control reagents; (v) a detection reagent such as a detectably labeled secondary antibody or a substrate; (vi) one or more control or reference samples that can be used for comparison purposes or to verify that a procedure for detecting HSF1 expression and/or activation is performed appropriately or is giving accurate results. A control reagent can be used for negative or positive control purposes. A control reagent may be, for example, a probe or primer that does not detect or amplify HSF1 mRNA or an antibody that does not detect HSF1 polypeptide or a purified HSF1 polypeptide or portion thereof (e.g., an HSF1 peptide). A probe, primer, antibody, or other reagent may be attached to a support, e.g., a bead, slide, chip, etc.

It will be understood that a kit may contain reagents (e.g., probes, primers, antibodies) suitable for measuring expression of any one or more HSF1-regulated genes described herein, e.g., any one or more HSF-G1 genes, any one or more HSF1-G2 genes, any one or more HSF1-G3 genes, any one or more HSF1-G4 genes, any one or more HSF1-SSS genes, any one or more Refined HSF1-SSS genes (i.e., any one or more genes listed in Table D) any one or more HSF1-CSS genes (e.g., any one or more genes listed in Table B or any one or more genes listed in Table C), any one or more cancer-stroma normalization genes, or combinations thereof. Exemplary combinations are described herein. For example, in some embodiments of particular interest, a kit comprises probes and/or primers suitable for measuring expression of between 5 and 50 HSF1-CSS genes and between 5 and 42 Refined HSF1-SSS genes, e.g., between 5 and 10, between 10 and 20, between 20 and 30, between 30 and 40, or between 40 and 50 HSF1-CSS genes and between 5 and 10, between 10 and 20, between 20 and 30, or between 30 and 42 Refined HSF1-SSS genes. In some embodiments a kit comprises probes and/or primers suitable for measuring expression of between 5 and 92 HSF1-CCSS genes, e.g., between 10, 20, 30, 40, 50, 60, 70, 80, and 92 HSF1-CCSS genes, e.g., between 10 and 80 HSF1-CCSS genes. In some embodiments a kit further comprises probes and/or primers suitable for measuring expression of one or more cancer-stroma normalization genes.

One of ordinary skill in the art would appreciate that the particular probes and/or primers for use in a given assay would, in general, depend on the particular type of assay. For example, an assay in which mRNA is detected using nanostring technology may utilize at least two probes that hybridize to mRNA of each assay target gene. An assay in which mRNA of a gene of interest is detected using PCR may utilize a pair of primers specific for an assay target gene and, in some embodiments, a reporter probe that hybridizes to a region of DNA that is amplified using the two primers.

In some embodiments a kit may comprise one or more enzymes for use in an assay implemented using the kit. For example, an assay that includes a step of reverse transcribing mRNA may comprise a reverse transcriptase. An assay that includes a nucleic acid amplification step may contain a polymerase, e.g., a DNA polymerase. For example, a kit for performing, e.g., a PCR assay, may include a thermostable DNA polymerase such as Taq polymerase or Pfu DNA polymerase. In some embodiments a kit may comprise dNTPs for reverse transcribing RNA and/or for amplifying DNA, rNTPs for transcribing RNA, oligodT primers for reverse transcribing mRNA, random hexamer primers for reverse transcribing RNA. In some embodiments a kit may comprise a buffer solution for extracting RNA from a biological sample comprising cells, an agent for stabilizing RNA prior to or after its extraction from cells, an agent for degrading or removing genomic DNA, or a combination thereof. A set of primers or probes may comprise one or more primers and/or probes included for control purposes, e.g., to confirm that appropriate kit components (e.g., enzymes) are active and present in an assay reaction.

Individual kit components may be packaged in separate containers (e.g., tubes, bottles, etc.) The individual component containers may be packaged together in a larger container such as a box for commercial supply. Optionally the kit comprises written material, e.g., instructions, e.g., in a paper or electronic format (e.g., on a computer-readable medium). Instructions may comprise directions for performing the assay and/or for interpreting results, e.g., in regard to tumor classification, diagnosis, prognosis, or treatment-specific prediction. Such material could be provided online. In some embodiments instructions include information regarding appropriate adjustment of reagent amounts or processes to detect HSF1 expression and/or activation specifically in tumor-associated stromal cells as distinct from cancer cells and/or in cancer cells as distinct from tumor-associated stromal cells. For example, a kit may comprise instructions as to appropriate dilution of primary antibody to provide an appropriate level of detection of HSF1 in tumor-associated stromal cells. In some embodiments a kit may comprise one or more reagents useful for distinguishing between tumor-associated stromal cells and cancer cells. For example, a kit may comprise an antibody that binds to a marker of tumor-associated stromal cells, e.g., SMA.

In some embodiments, the invention provides a system which is adapted or programmed to assess HSF1 expression or HSF1 activation, e.g., for use in a method of the invention. In some embodiments the system may include one or more instruments (e.g., a PCR machine), an automated cell or tissue staining apparatus, an imaging device (i.e., a device that produces an image), and/or one or more computer processors. The system may be programmed with parameters that have been selected or optimized for detection and/or quantification of an HSF1 gene product, e.g., in samples comprising tumor-associated stromal cells. The system may be adapted to perform the assay on multiple samples in parallel and/or may have appropriate software to analyze samples (e.g., using computer-based image analysis software) and/or provide an interpretation of the result. The system can comprise appropriate input and output devices, e.g., a keyboard, display, etc. In some embodiments the system is programmed to analyze samples both for cancer cell and for tumor-associated stromal cell HSF1 expression and/or activation. In some embodiments individual results, a composite result, or both, from such analyses may be reported.

In some embodiments, an assay is performed at one or more central testing facilities, which may be specially qualified or accredited (e.g., by a national or international organization which, in some embodiments, is a government agency or organization or a medical or laboratory professional organization) to perform the assay and, optionally, provide a result. A sample can be sent to the laboratory, and a result of the assay, optionally together with an interpretation, is provided to a requesting individual or entity. In some embodiments, determining the level of HSF1 expression or the level of HSF1 activation in a sample comprising tumor-associated stromal cells obtained from a tumor comprises providing a tumor sample to a testing facility. In some aspects, the invention provides a method comprising: providing to a testing facility (a) a sample obtained from a subject; and (b) instructions to perform an assay to assess the level of HSF1 expression or HSF1 activation (and, optionally, instructions to perform one or more additional assays, e.g., one or more additional assays described herein). In some embodiments the order specifies that the level of HSF1 expression and/or activation in tumor-associated stromal cells or tumor stroma is to be determined. In some embodiments a method comprises entering an order for an assay of HSF1 expression and/or HSF1 activation into an electronic ordering system, e.g., of a health care facility. In some aspects, the invention provides a method comprising: (a) providing to a testing facility a sample obtained from a subject; and (b) receiving results of an assay of HSF1 expression and/or HSF1 activation in tumor-associated stromal cells. In some aspects, the invention further provides a method comprising providing, e.g., electronically, a result of such an assay, to a requestor. In some embodiments a result is provided at least in part by entering the result into a computer, e.g., into a database, electronic medical record, laboratory information system (sometimes termed laboratory information management system), etc., wherein it may be accessed by or under direction of a requestor. In some embodiments a result may be provided via phone, voicemail, fax, text message, or email. In some embodiments a result is provided at least in part over a network, e.g., the Internet. In some aspects, the invention further provides a method comprising receiving, e.g., electronically, a sample and a request for an assay of HSF1 expression or HSF1 activation, performing such assay, and reporting the result of such assay to a requestor. A result can comprise one or more measurements, scores and/or a narrative description. In some embodiments, a result provided comprises a measurement, score, or image of the sample, with associated diagnostic, prognostic, or treatment-specific predictive information. In some embodiments, a result provided comprises a measurement, score, or image of the sample, without associated diagnostic, prognostic, or treatment-specific predictive information. The invention contemplates that an assay may be performed at a testing facility which is remote from the site where the sample is obtained from a subject (e.g., at least 1 kilometer away) although of course an assay may be performed at the site where the sample is obtained or any other site in various embodiments. It is contemplated that samples and/or results may be transmitted to one or more different entities, which may carry out one or more steps of an assay or a method of the invention or transmit or receive results thereof. All such activities are within the scope of various embodiments of the invention.

In some embodiments a method described herein is computer-assisted. “Computer-assisted” as used herein encompasses methods in which a computer is used to gather, process, manipulate, display, visualize, receive, transmit, store, or in any way handle or analyze information (e.g., data, results, images, etc.). A computer may be used, for example, in sample processing, automated sample staining, automated image analysis, sample tracking, transmitting a request for an assay, transmitting a result of an assay, storing a result, etc. A method may comprise causing the processor of a computer to execute instructions to gather, process, manipulate, display, receive, transmit, or store data or other information. The instructions may be embodied in a computer program product comprising a computer-readable medium. A computer-readable medium may be any tangible medium (e.g., a non-transitory storage medium) having computer usable program instructions embodied in the medium. Any combination of one or more computer usable or computer readable medium(s) may be utilized in various embodiments. A computer-usable or computer-readable medium may be or may be part of, for example but not limited to, an electronic, magnetic, optical, electromagnetic, infrared, or semiconductor system, apparatus, or device. Examples of a computer-readable medium include, e.g., a hard disk, a random access memory (RAM), a read-only memory (ROM), an erasable programmable read-only memory (e.g., EPROM or Flash memory), a portable compact disc read-only memory (CDROM), a floppy disk, an optical storage device, or a magnetic storage device. In some embodiments a method comprises transmitting or receiving data or other information over a communication network. Data or information may be generated at or stored on a first computer-readable medium at a first location, transmitted over the communication network, and received at a second location, where it may be stored on a second computer-readable medium. A communication network may, for example, comprise one or more intranets or the Internet. In some embodiments results of an assay are stored in a database, which may be stored on a computer-readable medium. In some embodiments result(s) are stored in association with a sample identifier. In some embodiments result(s) are stored in association with a subject identifier. In some embodiments results of an assay are stored in a subject's electronic health record. Additional information regarding a tumor may be stored as well. Such information may comprise, for example, an assessment of tumor grade, tumor stage, tumor type (e.g., cell type or tissue of origin) and/or results of assessing expression of one or more genes of interest. In some embodiments a result is provided in a report.

### EXEMPLIFICATION

### Example 1

### HSF1 is Activated in Cancer-Associated Fibroblasts within Human Tumors

Normal fibroblasts usually constitute a tumor-restrictive environment (Bissell and Hines, 2011). In mouse models, tumor suppressors such as p53 and PTEN can act in the stroma to limit tumor growth (Lujambio et al., 2013; Moskovits et al., 2006; Trimboli et al., 2009). In CAFs, p53 inhibits SDF1 secretion and activates an NFκB-mediated pro-inflammatory response that impairs tumor growth. (In other contexts, however, NFκB activation in CAFs can support malignancy, consistent with the pro-cancerous effects described for chronic inflammation and wound-healing responses (Coussens et al., 2013; Erez et al., 2010)). The tumor suppressor PTEN can also act in CAFs to suppress tumor growth through regulation of the ETS2 transcription factor. The inactivation of PTEN therefore helps to transform the stroma into a pro-tumorigenic microenvironment.

We speculated that there may be factors that actively support or enable malignancy in both cancer cells and the stroma and wondered whether tumors might hijack normal physiological pathways and programs in the stroma, subverting them to enable neoplastic growth and metastatic dissemination. In the present disclosure we demonstrate the existence of such a mechanism by describing the stromal function(s) of Heat Shock Factor 1 (HSF1) in tumor biology.

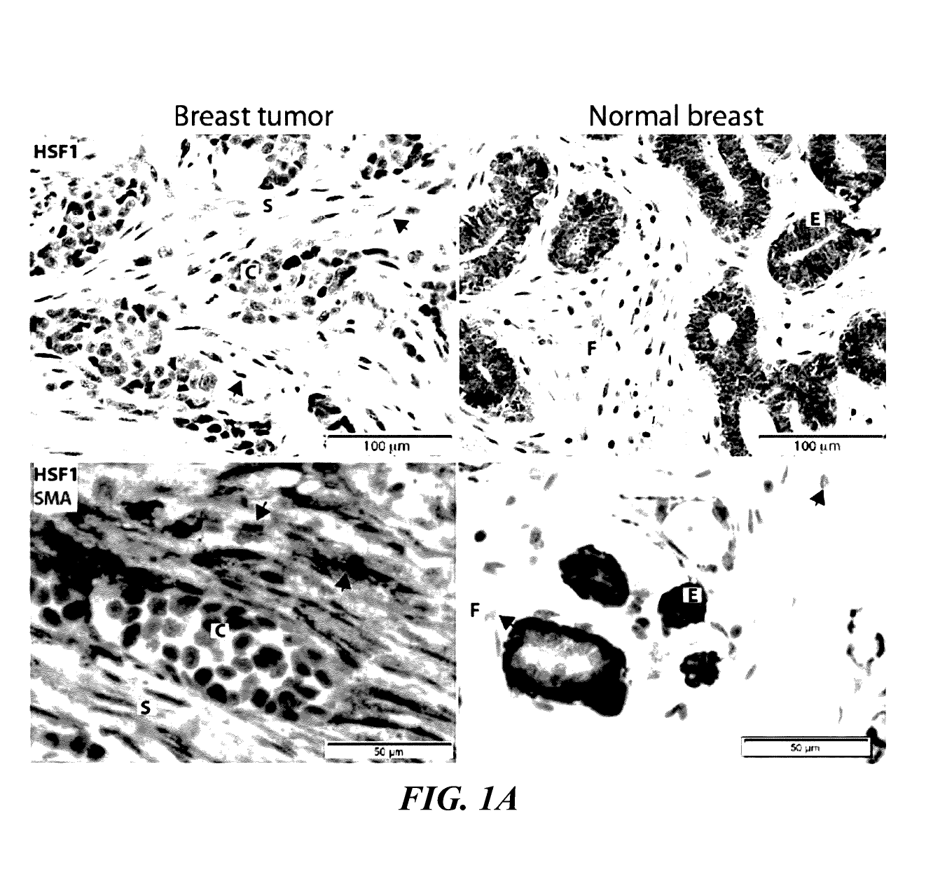

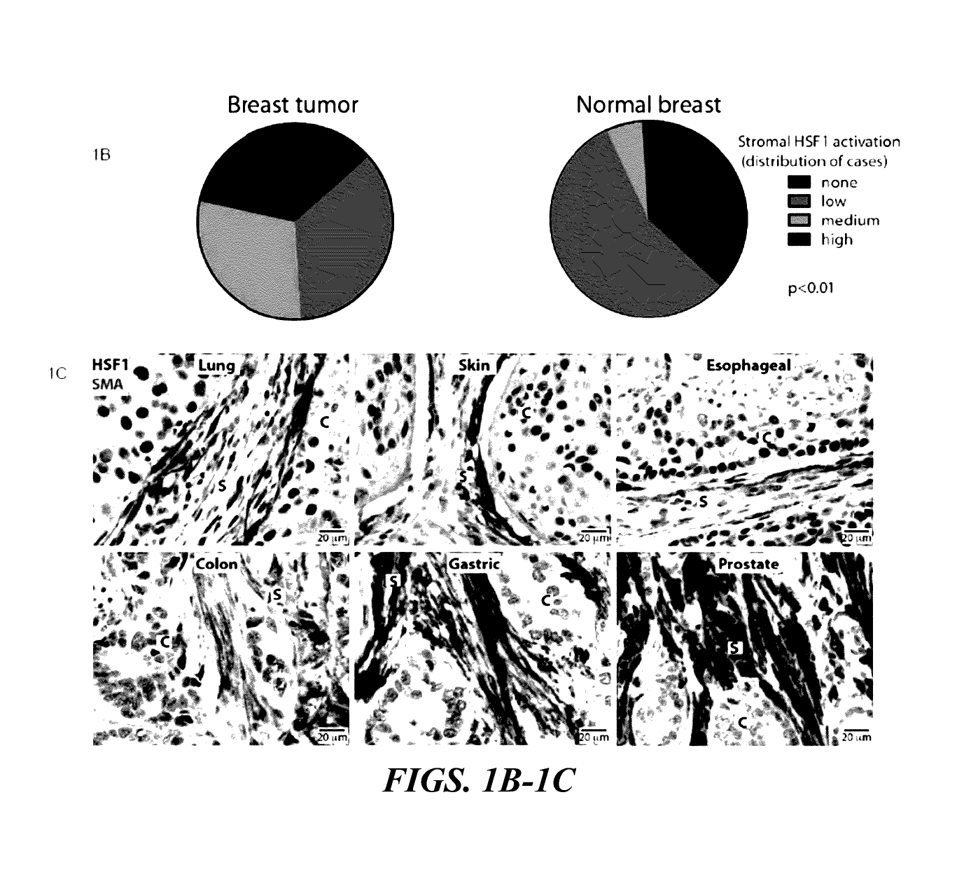

Under basal conditions in normal cells, HSF1 resides primarily in the cytoplasm. Upon activation, whether by physical stressors or malignant transformation, it accumulates in the nucleus (Morimoto, 2008; Santagata et al., 2011). To determine whether HSF1 is activated in cells of the tumor microenvironment we scored the staining intensity of this transcription factor in the nuclei of tumor-associated stroma within patient-derived breast cancer samples. Stromal cells residing in the lobules of neighboring, normal breast tissue in the same patient sections were used for comparison. These normal cells were almost invariably low or negative for nuclear HSF1. However, strong nuclear HSF1 staining was frequently present in the stromal cells situated in close proximity to the malignant cells (FIG. 1A upper panel, see 1B for quantification).

The morphology of the HSF1-positive stromal cells suggested that they were cancer-associated fibroblasts (CAFs). To confirm this, we co-stained tumor sections for HSF1 and smooth muscle actin (SMA). SMA stains normal myoepithelial cells comprising the basal layer that surrounds the inner luminal breast epithelial cells (FIG. 1A, lower right panel). It is not present in normal fibroblasts however, and is often used as a marker for stromal CAFs (Kalluri and Zeisberg, 2006; Quante et al., 2011). We also investigated markers of two other stromal components, leukocytes (LCA) and endothelial cells (CD31). Most of the HSF1-positive stromal cells in the tumors co-stained with SMA, suggesting that these are indeed CAFs (FIG. 1A, lower left panel, and Figure S1).

To test the generality of HSF1 activation in CAFs across different tumor types, we co-stained tumor sections from lung, skin, esophageal, colon, gastric and prostate carcinomas with antibodies for HSF1 and SMA (FIG. 1C). In all these tumor types, most SMA-positive CAFs also had strong nuclear HSF1 staining.

### Example 2

### Loss of Hsf1 in Fibroblasts Reduces Xenograft Tumor Growth

To explore the functional importance of HSF1 activation in a tractable model system, we analyzed xenografts of human MCF7 breast cancer cells injected subcutaneously into immunocompromised (NOD-scid) mice. As expected, the xenografts recruited endogenous stromal cells from their mouse hosts to support tumor formation. These CAFs, which were positive for the SMA marker, exhibited strong nuclear staining for HSF1 (Figure S2A).

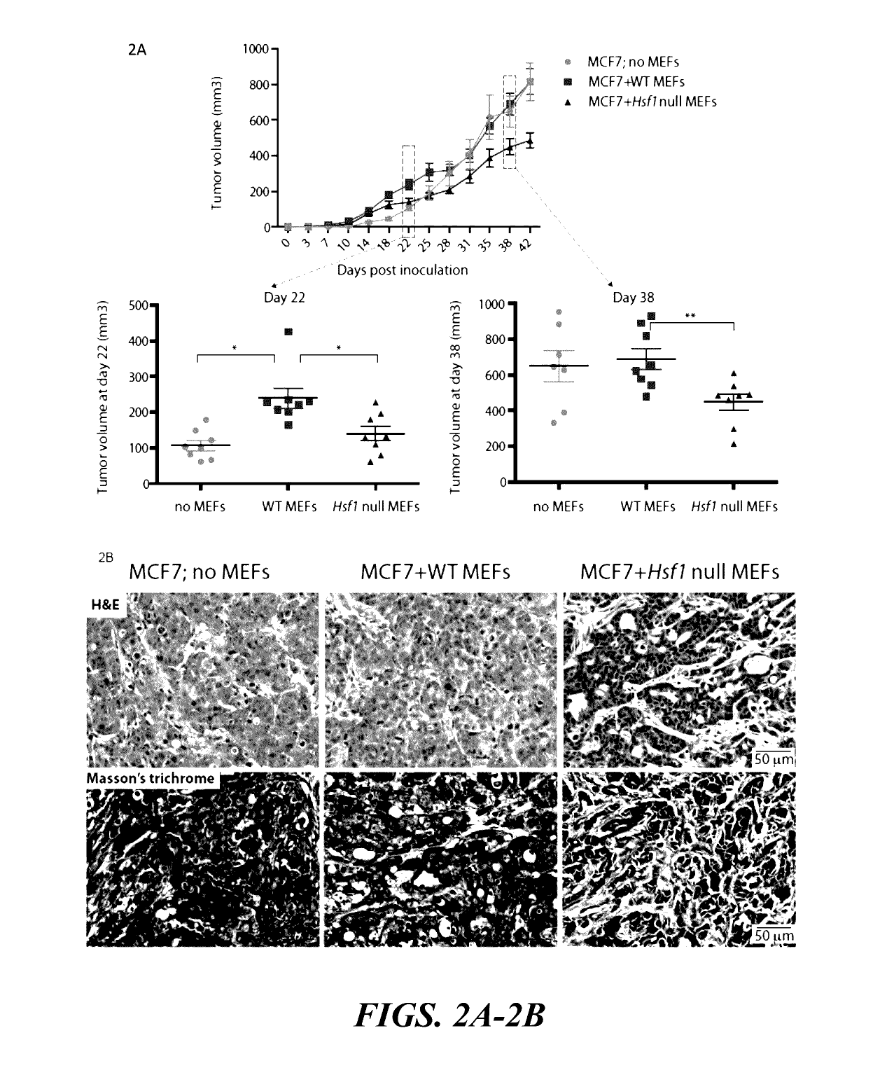

To test whether HSF1 activation in stromal cells plays a role in supporting the malignant tumor cells, we mixed primary mouse embryonic fibroblasts (MEFs)—either wild-type (WT) or Hsf1 null—with the MCF7 cancer cells and co-injected them subcutaneously into NOD-scid mice (FIG. 2A). Tumors arising from MCF7 cells co-injected with Hsf1 null MEFs (blue) grew significantly more slowly than those of mice co-injected with WT MEFs (red).

MCF7 cells injected without MEFs formed tumors more slowly than MCF7 cells co-injected with WT MEFs (FIG. 2A). With time, cells injected without MEF recruited WT host stroma and the tumors grew to the same size as those formed by co-injection with WT MEFs (FIG. 2A). However, throughout the experiment, tumors formed by MCF7 cells co-injected with Hsf1 null MEFs remained significantly smaller (FIG. 2A, blue).

To better understand this result, we excised tumors at the end of the experiment and examined their histology (FIG. 2B, upper panels). Tumors from mice injected with MCF7 cells only, and tumors from mice co-injected with WT MEFs shared a poorly differentiated and sheet-like morphology typical of high-grade tumors. In contrast, co-injection of MCF7 cells and Hsf1 null MEFs produced tumors with a more differentiated, stromal-rich architecture, indicative of a less malignant phenotype. Notably, some of the stromal fibroblasts were HSF1-positive, indicating that in addition to the injected stroma, the tumors had recruited some host stroma (Figure S2B). (This might explain why, even though slower, these tumors still grew.) Masson's trichrome staining indicated that the stroma-rich regions are mostly comprised of fibrous tissue and deposits of collagen (FIG. 2B, lower panels). These results suggest that, in response to cancer cells, HSF1 is activated in stromal CAFs to support tumor growth. Moreover, in the absence of this HSF1-driven response, fibroblasts actually exert an inhibitory effect on tumor expansion.

### Example 3

### Stromal HSF1 Regulates Cancer Cell Growth In Vitro

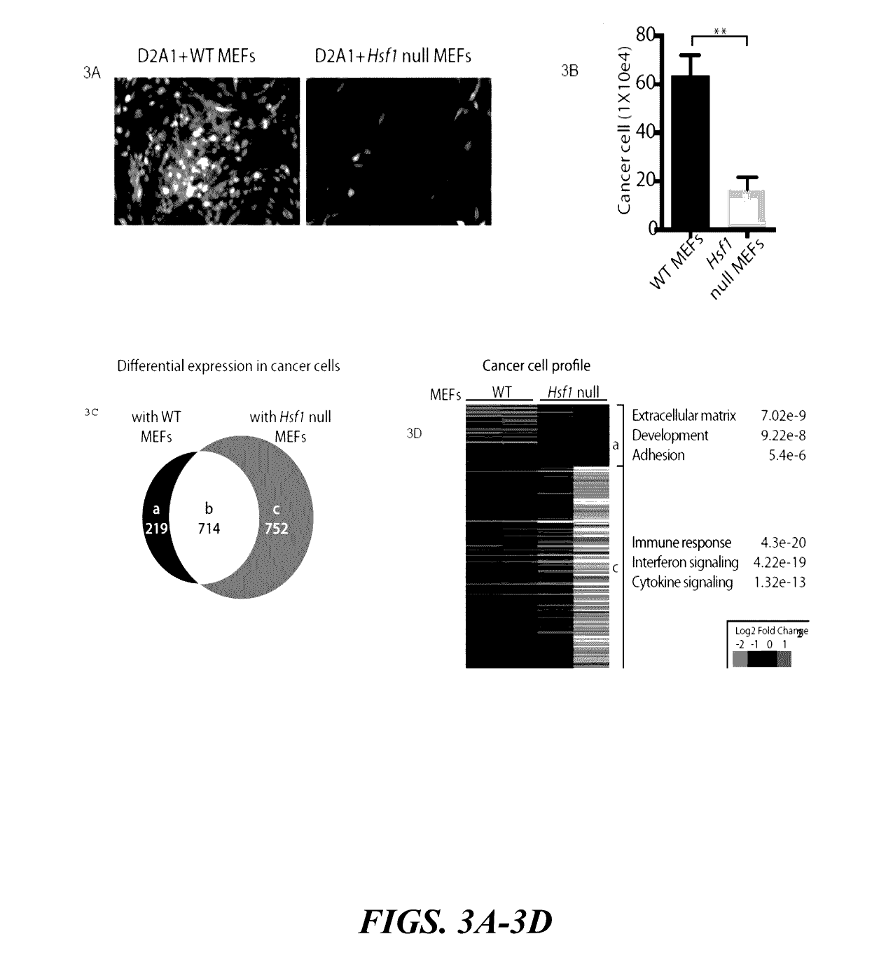

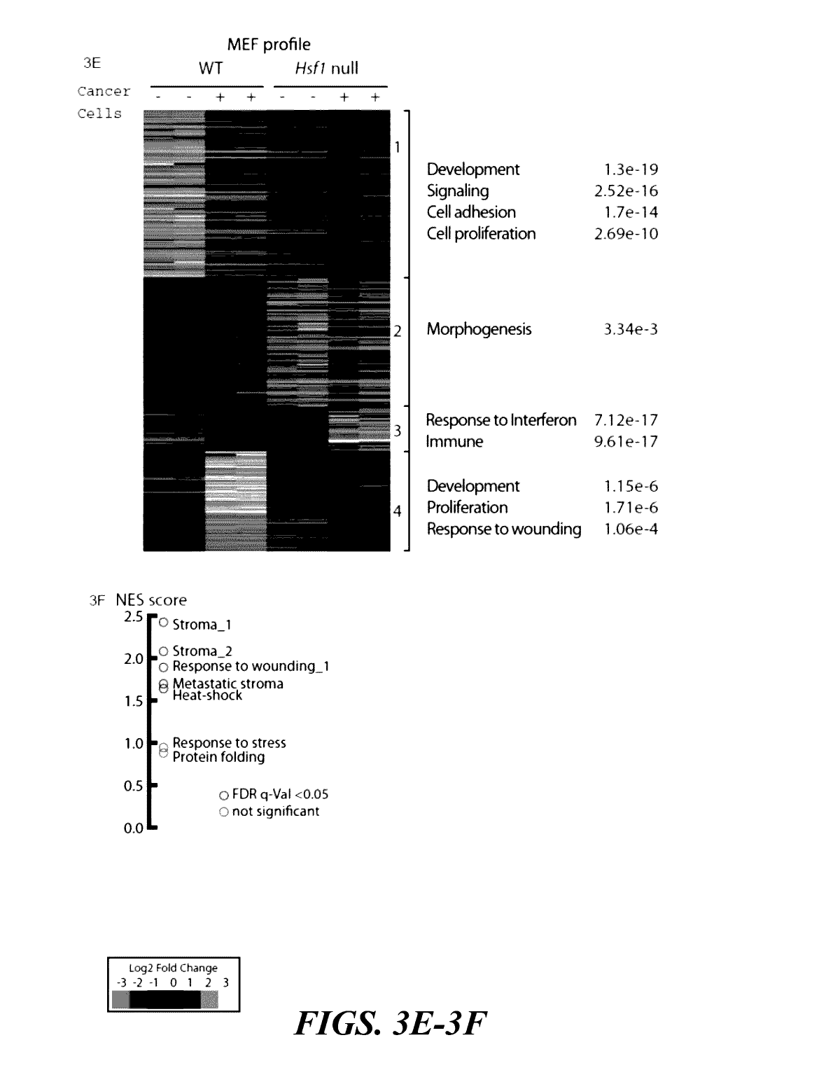

To investigate how the activation of HSF1 in stromal fibroblasts supports cancer cells, we plated fluorescently-labeled breast cancer cells onto feeder layers of either WT or Hsf1 null MEFs (FIG. 3A; phase images of feeder layers visualized in Figure S3A). We found a higher number of cancer cells in co-cultures with WT MEFs than with Hsf1 null MEFs. This held true for several different mammary cancer cell lines (mouse D2A1, FIGS. 3A and B; human MCF7, human HCC38 and mouse 4T7; Figure S3B-C).

To confirm that this effect is HSF1-dependent (and not simply a cell-line artifact) we employed MEFs that were deleted for WT Hsf1 but carried a tetracycline-repressible Hsf1 transgene (Bi-TetO-Hsf1). We repressed Hsf1 expression for 5 days and then co-cultured the MEFs with D2A1 cancer cells. This repression of Hsf1 resulted in decreased accumulation of cancer cells (Figure S3D). Thus, even short-term loss of Hsf1 impairs the ability of fibroblasts to support the growth of co-cultured cancer cells.

### Example 4

### Stromal HSF1 Drives a Transcriptional Program in Cancer Cells that Promotes Malignant Phenotypes

To test the effects of co-culture on gene expression, we separated cancer cells from fibroblasts by FACS sorting, extracted RNA and hybridized it to gene expression arrays. As a point of comparison, each cell type was grown alone (without co-culture), FACS sorted, and RNAs were isolated and analyzed in a similar manner.

In D2A1 cancer cells, regardless of the Hsf1 status of the co-cultured MEFs, the expression of ˜700 genes was altered by at least 2-fold following co-culture (FIG. 3C, group b, and Table S1). Of these, ˜400 genes were upregulated and -300 genes were downregulated. The upregulated set was enriched for genes involved in cellular differentiation, migration and extracellular matrix organization (Table S1). No significant functional enrichment was found in the downregulated set.

With specific regard to the Hsf1 status of the MEFs, approximately 200 genes were upregulated in cancer cells co-cultured with WT, but not with Hsf1 null MEFs (FIG. 3C, group a). This set was enriched for genes involved in extracellular matrix organization, development and adhesion (such as Dmp1, Dkk3, Thy1, Grem1, Sparc, Mmp2, and Mmp3, FIG. 3D and Table S1). In cancer cells co-cultured with Hsf1 null MEFs, ˜750 genes were uniquely upregulated (FIG. 3C, group c). Pro-inflammatory cytokines (such as Ccl5 and Ccl8) and immune responses (such as the response to type I interferon) were most significantly enriched in this group (FIG. 3D and Table S1). Thus, the activation of HSF1 in the stroma helps to reprogram cancer cells in at least two important ways. In a non-cell-autonomous manner it upregulates genes in cancer cells that enhance their malignant potential and downregulates genes that would trigger host immune defense responses.

### Example 5

### Stromal HSF1 Drives a Transcriptional Program in Fibroblasts that Supports Malignant Cells

Next, we examined a complementary question: how does co-culture with cancer cells affect HSF1-dependent gene expression in stromal fibroblasts? Profiling of the FACS-sorted MEFs showed that even in the absence of cancer cells, HSF1 regulated many genes involved in development, cell adhesion and proliferation (such as Fgf, Igf Col, Lama, Snail, and Sdf1; FIG. 3E, group 1; Tables S2 and S3). This suggests that HSF1 alters the basal phenotype of MEFs in culture and these alterations enhance the growth of cancer cells. In an HSF1-dependent manner, co-culture with cancer cells induced an additional cluster of genes involved in development, proliferation and response to wounding (such as Tgfβ1, Cxcl1, Cxcl3, and Vcam1; FIG. 3E, group 4 Tables S2 and S3). Also in an HSF1-dependent manner, cancer cells induced in MEFs a striking downregulation of genes involved in cellular immune responses (such as Cxcl10, Bst2, and C3; FIG. 3E, group 3; Tables S2 and S3). Thus, WT MEFs respond to cancer cells in a manner that would support tumor growth, whereas Hsf1 null MEFs respond in a manner likely to impede the process.

To further characterize the HSF1 stromal signature, we performed additional analyses of the genes that are differentially upregulated in WT vs Hsf1 null MEFs co-cultured with cancer cells (groups 1 and 4). We compared this list to publicly available gene-sets of stroma from human cancer patients, fibroblast wound healing responses, and the heat-shock response. Although some heat-shock-related genes were enriched, this was not the most prominent response. Rather, the HSF1 stromal signature was most highly enriched for genes previously characterized by their up-regulation in fibroblasts in response to wounding and in stromal cells isolated from human tumors (Beck et al., 2008; Dvorak, 1986; Karnoub et al., 2007) (FIG. 3F). We also compared this list to the HSF1-dependent gene expression signature in cancer cells (Mendillo et al., 2012) and found that these signatures were, if anything, anti-correlated. Thus, in fibroblasts HSF1 activates a transcriptional program that would support cancer and is profoundly different from the response activated by HSF1 in the cancer cells themselves, or in cells exposed to heat.

### Example 6

### The Effects of Stromal HSF1 Activation on Cancer Cells are Mediated by TGFβ and SDF1 Signaling

Unbiased analysis of gene set enrichment established that TGFβ signaling was one of the top categories regulated by HSF1 in MEFs co-cultured with cancer cells (group 4 in FIG. 3E). Because TGFβ, along with SDF1, has been previously found to promote CAF phenotypes (Kojima et al., 2010), we further interrogated both signaling pathways. We extracted RNA from both immortalized and 3 separate sets of primary WT or Hsf1 null MEFs (each derived from a different mating pair) and performed qPCR with primers targeting Tgfβ1, Tgfβ2, Tgfβ3 and Sdf1. This confirmed that the expression levels of Sdf1, Tgfβ1 and Tgfβ2 were significantly lower in Hsf1 null MEFs than in WT MEFs, even without co-culture with tumor cells (FIG. 4A and Figure S4A).

Next, we asked if TGFβ and SDF1 mediate HSF1's stromal support of cancer cells. To do this, we added these factors as purified recombinant proteins to co-cultures of D2A1 cancer cells with Hsf1 null MEFs. Combined addition of TGFβ1 and SDF1 restored cancer cell growth to the levels seen in cells co-cultured with WT MEFs (FIGS. 4B and C). Partial effects, that did not reach statistical significance, were achieved by addition of either factor alone (Figure S4B).

As a further test of its function, we repressed TGFβ signaling in the co-cultures by adding a TGFβ receptor type I/II (TβRI/II) dual inhibitor, LY2109761 to the media (Dituri et al., 2013). To control for direct effects of the inhibitor on the cancer cells themselves, we treated cancer cells with the inhibitor in the absence of MEFs. Treatment with LY2109761 did not affect cancer cells grown alone (Figure S4C). It did, however, significantly reduce their growth in co-culture with WT MEFs (FIG. 4D; p=0.008). A smaller effect, that did not reach statistical significance, was seen in co-culture with Hsf1 null MEFs (FIG. 4D; p=0.1). Taken together with the increased expression of Tgfβ and Sdf1 in WT MEFs compared to Hsf1 null MEFs (FIG. 4A), these results suggest that TGFβ and SDF1 are produced and secreted by fibroblasts in an HSF1-dependent manner.

Once secreted, TGFβ and SDF1 could be activating either the fibroblasts themselves, the cancer cells, or both. To investigate, we knocked down the expression of several signaling molecules downstream of TGFβ and SDF1 in either the cancer cells or the MEFs (see Experimental Procedures). Knock-down of Smad2, a key downstream mediator of TGFβ signaling, in WT MEFs, impaired the growth of co-cultured D2A1 cancer cells (FIG. 4E; Figure S4E). This growth defect could not be rescued by addition of recombinant TGFβ1 and SDF1 (FIG. 4E). Notably, Smad2 knockdown was only effective in the MEFs. Knockdown of the same gene in the D2A1 cells themselves had no effect on cell number (Figure S4D; Figure S4E). From this we conclude that HSF1 supports an autocrine TGFβ signaling loop in MEFs. As for SDF1, although we cannot discriminate whether it signals to the cancer cells or to the stroma, SDF1 expression is clearly upregulated by HSF1 in fibroblasts. Taken together, our data indicate that TGFβ and SDF1 are key mediators of the tumor-promoting activity of stromal HSF1.

### Example 7

### HSF1 Directly Binds HSEs of the Sdf1 Gene in Stromal Cells

Next we asked whether TGFβ and SDF1 are direct transcriptional targets of HSF1. HSF1 regulates transcription by binding to heat shock elements (HSEs) in target genes. A bioinformatic search for HSEs in genes of the TGFβ and SDF1 signaling pathways confirmed that the Tgfβ2 and Sdf1 genes themselves contain canonical HSEs (See Extended Experimental Procedures for details). No HSEs were found in Tgfβ1 or in any of the downstream signaling molecules mentioned above. To determine whether HSF1 directly regulates Tgfβ2 and Sdf1 expression in CAFs, we performed chromatin immunoprecipitation (ChIP) using anti-HSF1 antibodies and extracts prepared from MCF7 tumor xenografts. To focus specifically on the supporting mouse stromal cells, and not the human tumor cells, we performed quantitative PCR (qPCR) using primers flanking potential HSF1 binding sites that were specific to the mouse genes (Figure S4F). Primers for an intergenic region served as a negative control. Sdf1 was significantly amplified from stromal (mouse) DNA bound by HSF1 (FIG. 4F). No significant amplification was detected for Tgfβ2 (FIG. 4F). Together with the effects of HSF1 on the expression of these genes, these results suggest that the regulation of Tgfβ by HSF1 may be indirect. However, HSF1 directly binds and activates Sdf1.

### Example 8

### HSF1 Activation in Breast Cancer Stroma is Associated with Patient Outcome

Our findings in mouse xenografts and cell co-culture models indicate that stromal HSF1 contributes to tumor progression. To evaluate whether stromal HSF1 contributes to disease progression in human cancers, we first asked whether Hsf1 mRNA levels in the stroma correlate with disease outcome. We looked for this association in a publicly available mRNA dataset from 53 pure tumor stroma samples obtained from patients with primary breast tumors (stromal cells collected and separated from cancer cells by laser microdissection; (Finak et al., 2008)).

In this dataset, high Hsf1 levels significantly correlated with increased tumor grade (FIG. 5A) and poorer patient outcome (FIG. 5B). We further asked whether high stromal Hsf1 expression is associated with a specific breast cancer subtype. No significant association was found with estrogen receptor (ER, Figure S5A) or progesterone receptor (PR, Figure S5B) expression. (The number of triple negative tumors in this cohort was too small to determine a possible association with HSF1 expression). Hsf1 expression was, however, significantly higher in HER2-positive tumors as compared to HER2-negative tumors (FIG. 5C).

HSF1 is often activated post-transcriptionally without a change in its mRNA levels. To provide an independent assessment of the importance of its activation in breast cancer stroma, we assembled a new breast cancer cohort to evaluate HSF1 activation at the protein level by immunohistochemistry (IHC). (The assembly of this cohort was motivated by the fact that most available human tumor tissue arrays are not prepared with large enough cores for adequate assessment of the stroma.) We examined a total of 46 samples from patients with early stage breast cancer (Table S6), for whom we had both appropriate tissue sections as well as a minimum of 8 years of continuous clinical followup data. Tumor sections were scored in a blinded manner for nuclear HSF1 staining intensity in the cancer cells and in the stromal cells.

We found markedly reduced disease-free survival, as well as overall survival, in patients whose tumors had high stromal HSF1 activation (FIG. 5D and Figure S5C). In this cohort, HSF1 activation in the stromal cells was correlated with HSF1 activation in the cancer cells (p=0.01, Chi-square test). Indeed, high HSF1 activation in the cancer cells also showed a strong correlation with lower overall survival, and a trend towards association with poor disease-free survival, consistent with our previous findings (Mendillo et al., 2012; Santagata et al., 2011). However, these associations with patient outcome were weaker in cancer cells than in the stroma (Figure S5D and E). Moreover, in a multivariate model considering the independent contributions of HSF1 activation in the cancer cells and in the stroma to overall survival, only stromal HSF1 (and not cancer-cell HSF1), was a significant predictor of survival (Table S7). Stromal HSF1 was also an independent, significant predictive factor in a multivariate model considering various clinicopathologic factors (Table S7). The significant association of stromal HSF1 activation with poor patient outcome as demonstrated in two independent cohorts using very different methodologies indicates that stromal HSF1 could be a useful, independent prognostic indicator in breast cancer.

TABLE S6
Means and frequencies of participants' characteristics by HSF1 status
in the stroma from the breast cancer cohort. Related to FIG. 5.

| | Stromal HSF1 | | |
|---|---|---|---|
| Characteristic | High | Intermediate | Low |
| n (%) | 6 (13) | 11 (24) | 29 (63) |
| Age at diagnosis, mean of n, y | 58 | 55 | 52 |
| Sex | | | |
| Female, n (%) | 5 (83) | 11 (100) | 28 (96) |
| Male, n (%) | 1 (16.6) | 0 (0) | 1 (3.5) |
| Tumor grade, n¹* | 3 | 6 | 21 |
| Grade 1, n² (% of n) | 0 (0) | 0 | 1 (3.5) |
| Grade 2, 3, n² (% of n) | 3 (50) | 6 (54) | 20 (69) |
| Tumor subtype | | | |
| Luminal A^{†}, n (%) | 4 (66.6) | 5 (45) | 15 (52) |
| Luminal B^{‡}, n (%) | 0 | 2 (18) | 3 (10) |
| Her2+ enriched, n (%) | 2 (33.3) | 2 (18) | 2 (7) |
| TN^{§}, n (%) | 0 | 2 (18) | 9 (31) |
| Lymph node (LN) involvement | | | |
| Positive, n (%) | 1 (17)\hat{ } | 3 (27) | 11 (38) |
| Negative, n (%) | 4 (67)\hat{ } | 8 (73) | 18 (62) |

*n¹ = samples for which grading information was available
^{†}Luminal A tumors are defined as Estrogen receptor (ER⁺), Progesterone receptor (PR⁺), Her2⁻, Ki67 low
^{‡}Luminal B tumors are defined as ER⁺, PR⁺, Her2⁻, Ki67 intermediate/high
^{§}Triple negative (TN) tumors are defined as ER⁻, PR⁻, Her2⁻
\hat{ }LN data was not available for one patient

TABLE S7
Multivariate Cox proportional hazards regression analyses of breast
cancer-related survival by HSF1 activation (staining intensity and
nuclear localization) and clinicopathologic factors. Related to FIG. 5.

| | | |
|---|---|---|
| Characteristic | HR (95% CI) | P |

Stromal HSF1 activation (n = 46)

| | | | |
|---|---|---|---|
| Stromal HSF1 | 4.5 | (1.9-10.5) | 0.0006 |

Cancer-cell HSF1 activation (n = 46)

| | | | |
|---|---|---|---|
| Epithelial HSF1 | 2.8 | (1.3-5.7) | 0.007 |

Stromal and cancer-cell HSF1 activation (n = 46)

| | | | |
|---|---|---|---|
| Stromal HSF1 | 3.5 | (1.3-9) | 0.01 |
| Cancer-cell HSF1 | 1.7 | (0.7-18) | 0.24 |

Stromal HSF1 activation and other clinicopathologic
factors (n = 45)

| | | | |
|---|---|---|---|
| Stromal HSF1 | 82.4 | (3.89-1741) | 0.005 |
| Age | 1.02 | (0.9-1.12) | 0.7 |
| ER status | 0.6 | (0.18-1.83) | 0.35 |
| PR status | 0.16 | (0.02-1.37) | 0.1 |
| HER2 status | 0.4 | (0.14-1.25) | 0.1 |
| Ki67 status | 3.5e−06 | (3.9e−14-312) | 0.2 |
| LN | 63 | (1.63-2422) | 0.026 |

### Example 9

### HSF1 Activation in Early-Stage Lung Cancer Stroma is Associated with Poor Outcome

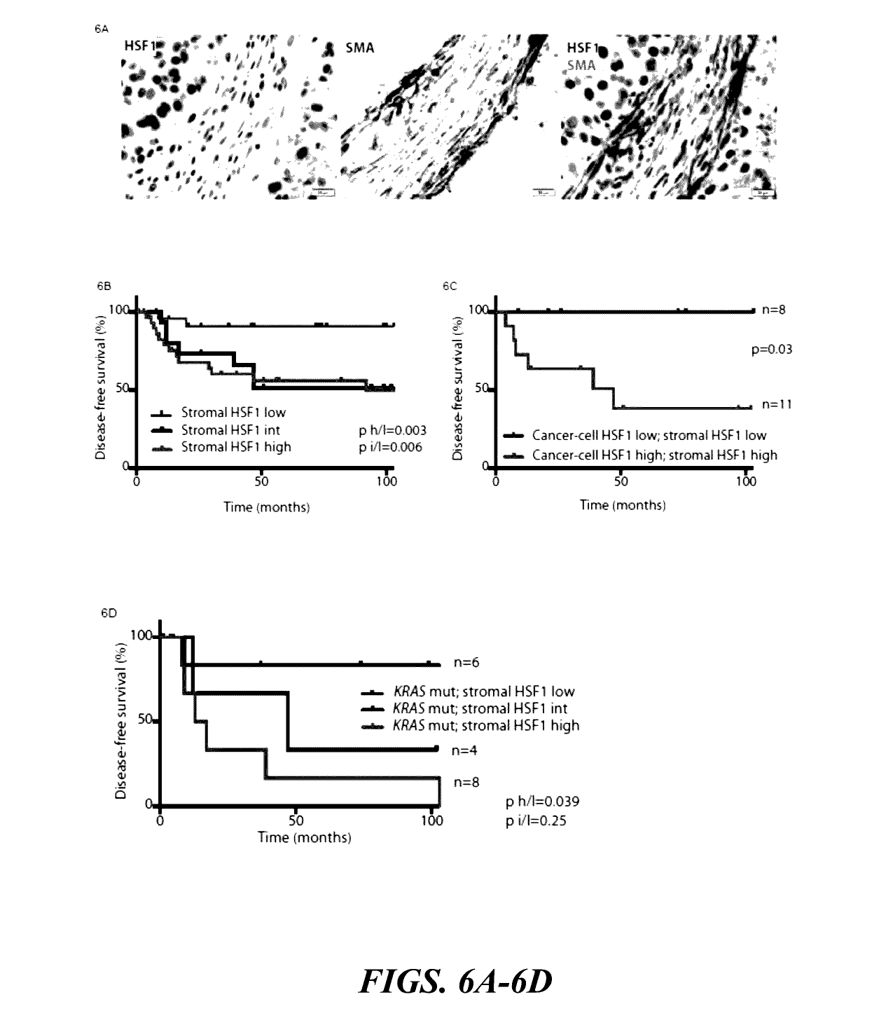

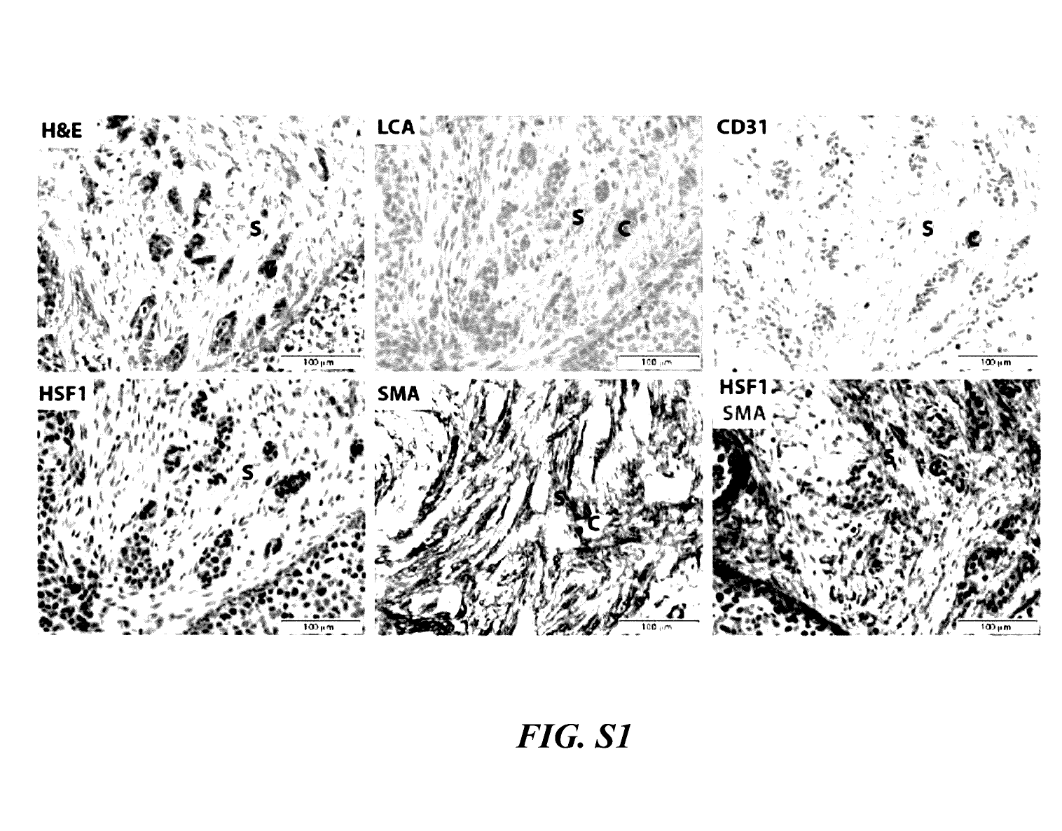

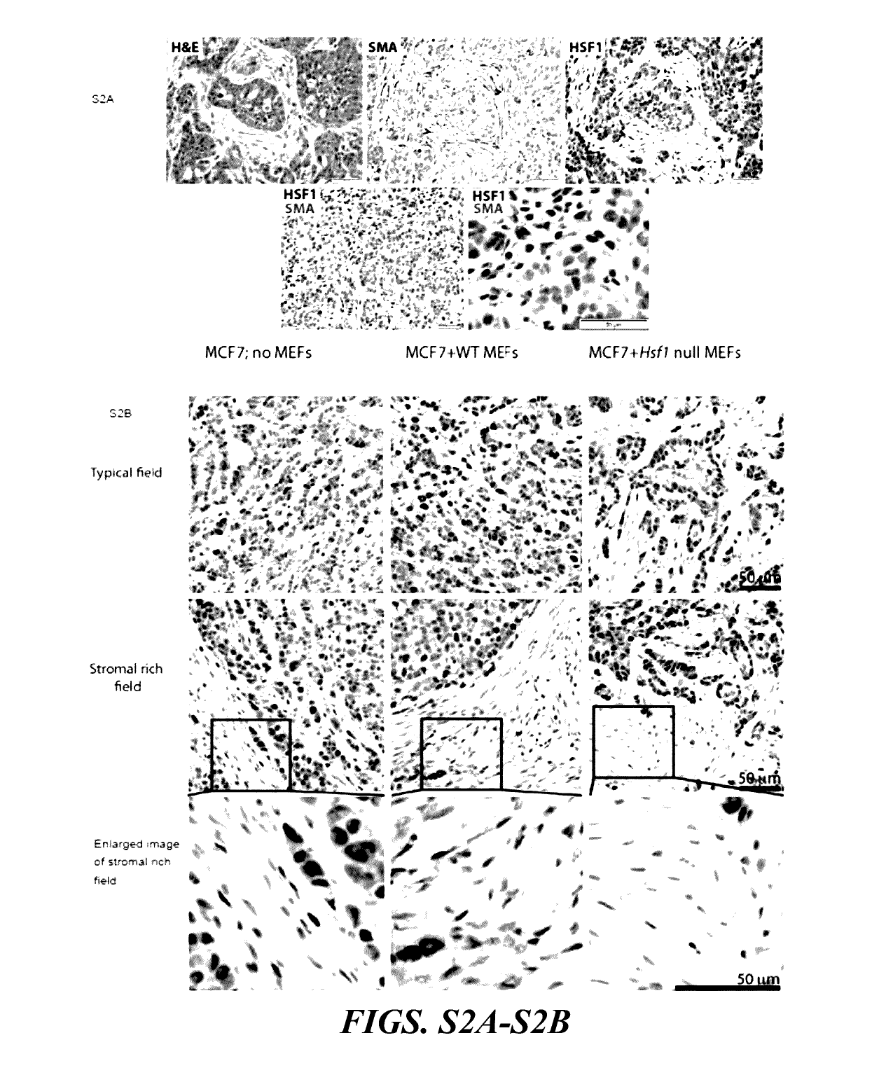

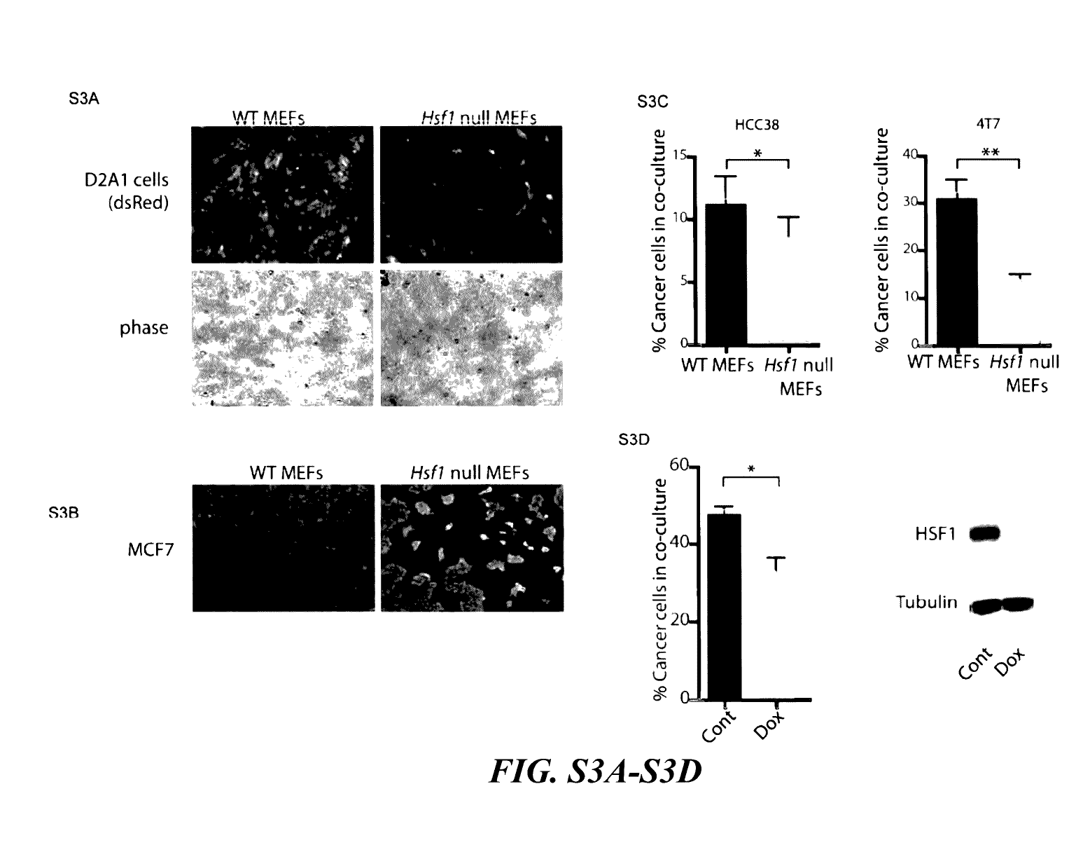

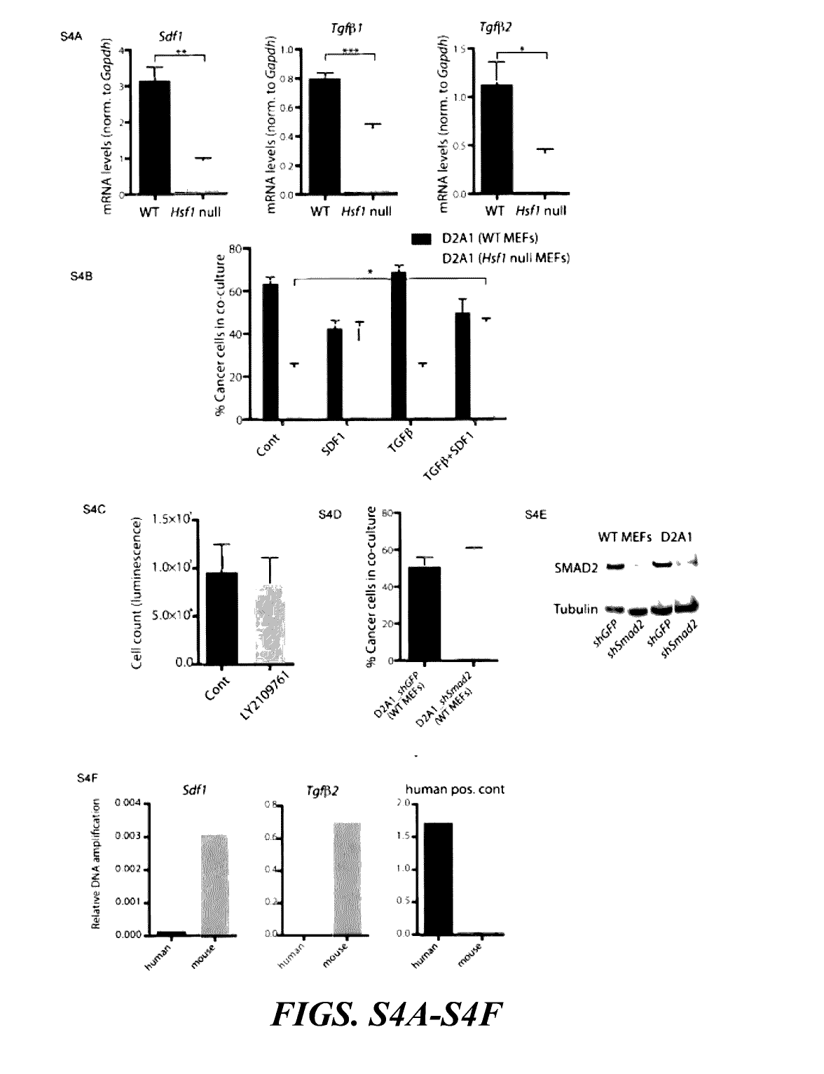

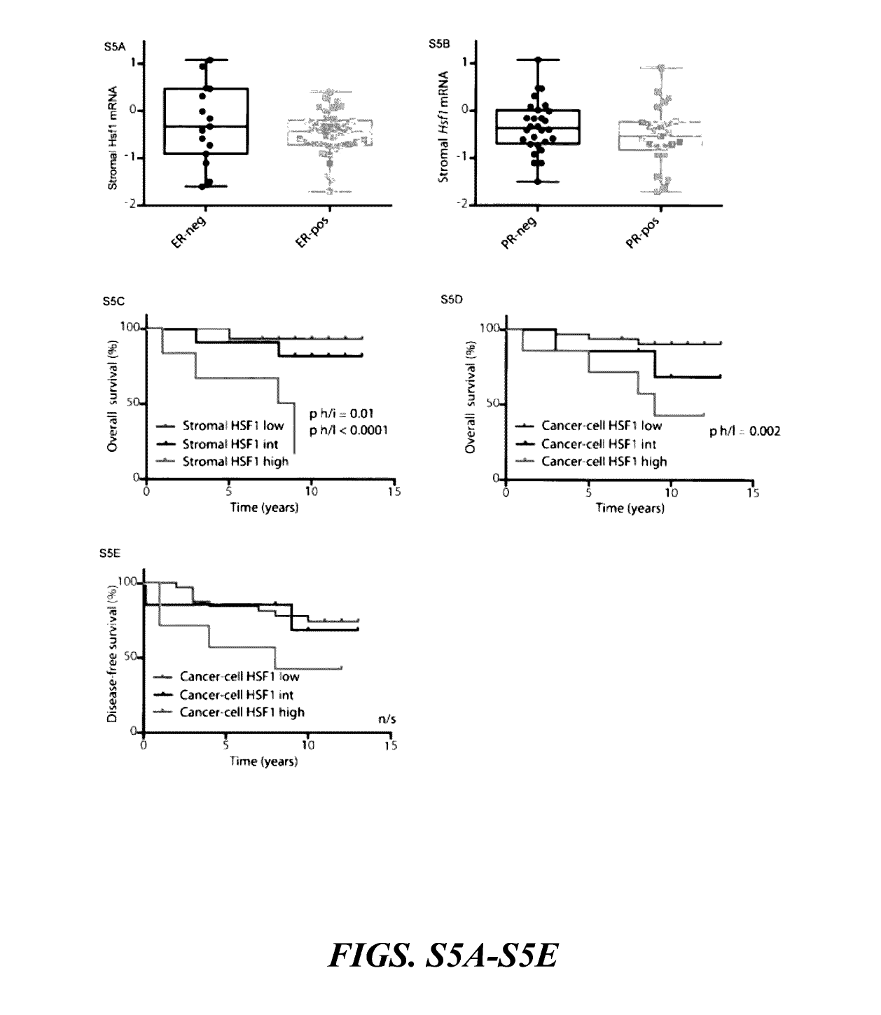

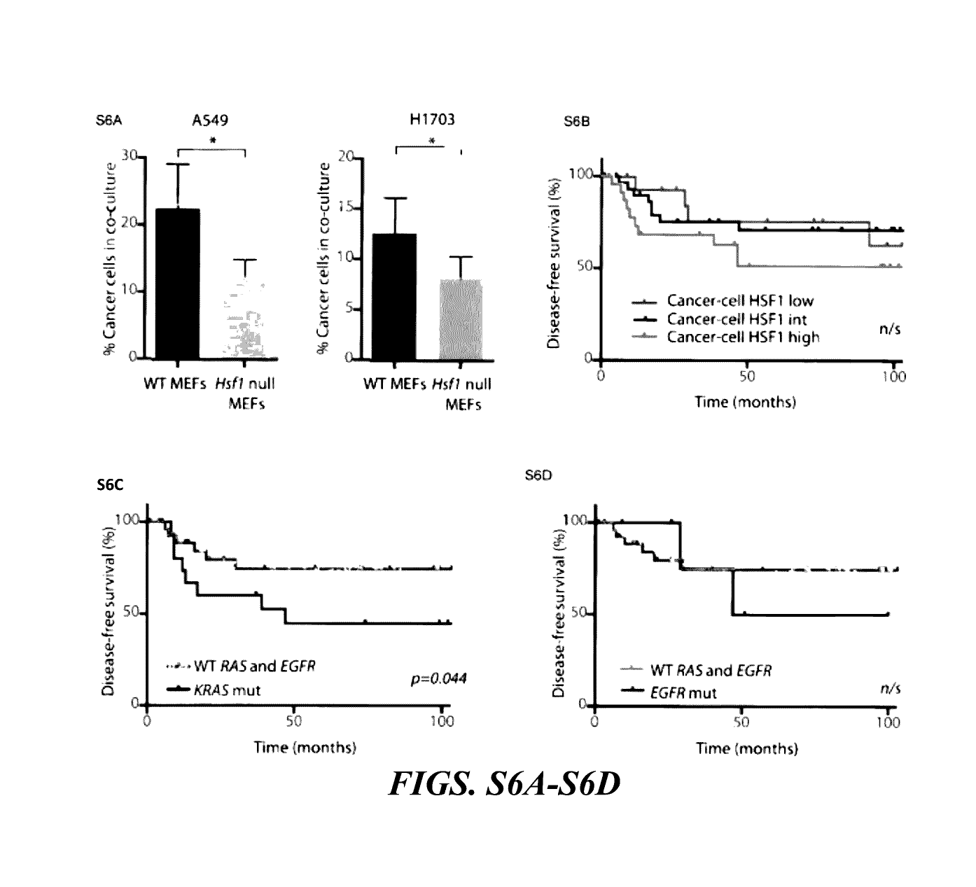

Our initial survey of human cancers showed that HSF1 is not only activated in breast cancer CAFs, but also in the CAFs of many other tumor types, including lung, colon, skin, esophageal, gastric and prostate (FIG. 1C and FIG. 6A). Of these tumor types, we had access to a cohort of lung cancer patients with appropriate tissue samples for stromal assessment of HSF1, together with clinical follow-up data. Pilot testing of human non-small cell lung cancer (NSCLC) cell lines (A549 and H1703), showed that these lines grew more robustly when co-cultured with WT MEFs than when co-cultured with Hsf1 null MEFs (Figure S6A).

A total of 72 samples from patients with stage 1 non-small cell lung adenocarcinoma (Table S8) (Sholl et al., 2010) were scored in a blinded manner for HSF1 activation (nuclear staining intensity) in cancer cells and stromal cells. Patients with stage I NSCLC have a 5-year survival of 60-70% (Goldstraw et al., 2007). Stromal HSF1 activation did not correlate with demographic factors such as age, sex or smoking status (Table S8). It did, however, show a significant correlation with patient outcome.

Disease-free survival was significantly shorter in lung cancer patients whose tumors expressed either high or intermediate HSF1 activity in the stroma (FIG. 6B). A similar trend was found for the survival of patients with high HSF1 in the cancer cells (Figure S6B). In this cohort, HSF1 activation in the cancer cells did not correlate with HSF1 activation in the stroma (p=0.28, Chi-square test). We therefore asked if evaluation of HSF1 activation in both cell types could improve our ability to predict patient outcome. Although the number of patients is small, it is striking that there was not a single recurrence in any of the patients that had low HSF1 activity in both the cancer cells and in the stroma over the course of followup (FIG. 6C).

To assess the independent contributions to outcome of increased HSF1 activation in cancer cells versus stromal cells, we fitted a multivariate Cox proportional hazards regression model to recurrence-free survival, considering stromal HSF1 activation separately from cancer-cell activation. Cancer-cell HSF1 was not independent from stroma in its association with disease progression. However, as in our breast cancer cohort, stromal HSF1 activation was significantly and independently associated with disease-free survival (Table S9).

To further refine our analysis, we genotyped the collection of tumor samples for the most commonly mutated oncogenes in lung adenocarcinoma, KRAS and EGFR (Pillai and Ramalingam, 2014), and tested the association of HSF1 activation and disease outcome with different mutations. In the 52 samples from our cohort that were successfully genotyped, KRAS mutations, but not EGFR mutations, correlated with poor disease-free survival (Figure S6C & S6D). We found no correlation between HSF1 activation (in either cancer cells or stroma) and KRAS or EGFR mutations per se. We did, however, find a significant association between high activity of HSF1 in the stroma and poor disease-free survival in patients with KRAS mutant tumors (FIG. 6D). Moreover, stromal HSF1 (but not cancer-cell HSF1) was an independent predictor of progression-free survival in several multivariate models considering KRAS and EGFR mutational status as well as clinicopathologic factors (Table S9). These clinical association data indicate that HSF1 status could serve as a useful independent prognostic marker in lung cancer.

TABLE S8
Means and frequencies of participants' characteristics by HSF1 status
in the stroma from the lung cancer cohort. Related to FIG. 6.

| | Stromal HSF1 | | |
|---|---|---|---|
| Characteristic | High | Intermediate | Low |
| n (%) | 33 (46) | 15 (21) | 24 (33) |
| Age at diagnosis, mean | 64 | 66 | 65 |
| of n, y | | | |
| Sex | | | |
| Female, n (%) | 18 (55) | 8 (53) | 17 (71) |
| Male, n (%) | 15 (45) | 7 (47) | 7 (29) |
| HSF1 in cancer cells | | | |
| High | 11 (33) | 3 (20) | 5 (21) |
| Intermediate | 15 (45) | 10 (66.6) | 11 (46) |
| low | 7 (21) | 2 (13.3) | 8 (33) |
| Tumor size, n (%) | | | |
| ≦3 cm | 22 (67) | 14 (93) | 22 (92) |
| >3 cm | 11 (33) | 1 (7) | 2 (8) |
| Stage, n (%) | | | |
| Ia | 18 (55) | 11 (73) | 14 (58)* |
| Ib | 15 (46) | 4 (27) | 9 (38)* |
| Predominant histologic subtype, | | | |
| n (%) | | | |
| Acinar | 14 (42) | 7 (47) | 9 (38) |
| Lepidic | 3 (9) | 3 (20) | 7 (29) |
| Mucinous Adeno | 1 (3) | 0 | 0 |
| Papillary | 6 (18) | 2 (13) | 6 (25) |
| Solid | 9 (27) | 3 (20) | 2 (8) |
| Smoking status, n (%) | | | |
| None | 3 (9) | 1 (7) | 4 (17) |
| Current | 17 (52) | 8 (53) | 12 (50) |
| Former | 9 (27) | 6 (40) | 8 (33) |
| No data | 4 (12) | 0 (0) | 0 (0) |
| Mutant KRAS, n (%)^{§} | 8 (35) | 4 (36) | 6 (35) |
| Mutant EGFR, n (%)\hat{ } | 1 (3.5) | 2 (15) | 3 (14) |
| WT EGFR and KRAS^{†} | 15 | 5 | 8 |

*Staging data was not available for one patient
^{§}Percent of mutants out of cases successfully genotyped for KRAS
\hat{ }Percent of mutants out of cases successfully genotyped for EGFR
^{†}Only samples that were successfully genotyped as WT for both KRAS and EGFR are listed

TABLE S9
Multivariate Cox proportional hazards regression analyses of lung
cancer progression by HSF1 activation (staining intensity and nuclear
localization), EGFR and KRAS mutational status and clinicopathologic
factors. Related to FIG. 6.

| | | |
|---|---|---|
| Characteristic | HR (95% CI) | P |

Stromal HSF1 activation (n = 72)

| | | | |
|---|---|---|---|
| Stromal HSF1 | 2.2 | (1.3-3.8) | 0.005 |

Cancer-cell HSF1 activation (n = 72)

| | | | |
|---|---|---|---|
| Cancer-cell HSF1 | 1.6 | (0.88-2.8) | 0.13 |

Stromal and cancer-cell HSF1 activation (n = 72)

| | | | |
|---|---|---|---|
| Stromal HSF1 | 1.5 | (0.84-2.6) | 0.006 |
| Cancer-cell HSF1 | 2.17 | (1.25-3.8) | 0.17 |

Stromal HSF1 activation, EGFR and KRAS status, other
clinicopathologic factors (n = 51)

| | | | |
|---|---|---|---|
| Stromal HSF1 | 2.38 | (1.15-4.9) | 0.02 |
| EGFR WT | 3.36 | (0.5-23) | 0.2 |
| Size | 0.6 | (0.33-1) | 0.07 |
| KRAS WT | 0.2 | (0.07-0.74) | 0.014 |
| Stage | 1.5 | (1-2.23) | 0.055 |
| Age | 1 | (0.94-1.08) | 0.9 |
| Sex | 0.18 | (0.05-0.7) | 0.015 |
| Smoking status | 2.4 | (0.7-8.08) | 0.15 |

Experimental Procedures Used in the Examples

Cell Culture:

D2A1 and 4T7 cells stably expressing dsRed, MCF7 cells stably expressing GFP and MEFs were cultured in Dulbecco's modified Eagle's medium supplemented with 10% fetal bovine serum (FBS). HCC38, A549 and H1703 cells stably expressing GFP were cultured in RPMI medium supplemented with 10% FBS. For co-culture experiments, immortalized MEFs were plated at near confluency, allowed to adhere for 24 h and then treated, where indicated, with 10 μg/ml mitomycin C (Sigma) for 2 h. Following 3 washes with PBS, cancer cells were seeded on top of the MEFs (1:5 ratio of cancer cells:fibroblasts), and allowed to grow for 72 h-96 h. Where indicated, MEFs were incubated with LY2109761 (I μM, Selleck chemicals) for 30 minutes before seeding of cancer cells. The same concentration of inhibitor was then added daily, throughout the experiment. Recombinant TGFβ (R&D systems, 240-B-002) and SDF1 (R&D systems, 460-SD-010) were added to co-cultures at the indicated concentrations once, when co-culture was started.

Bi-Tet-Hsf1 MEFs:

A construct encoding Hsf1 under the regulation of the rtTA/tet-O promoter (Bi-Tet-Hsf1) was targeted to the ROSA26S locus of C57BL/6×129 SvJae mice. These mice were then crossed with Hsf1 null mice to create a transgenic line in which the sole source of Hsf1 expression is Bi-Tet-Hsf1. Primary MEFs were isolated and immortalized by overexpression of HPV E6/E7. Where indicated, cells were treated with 2 μg/ml doxycycline to inhibit Hsf1 expression.

Flow Cytometry:

For expression profiling, co-cultures were sorted using a FACS-Aria (BD-biosciences) instrument. To avoid contamination of one cell type by the other, and to allow optimal sorting of each cell type, co-culture was repeated twice, in duplicate. In one set of replicates, only the D2A1 cells were collected after co-culture. In the other set of replicates, only the MEFs were collected. In this set, the MEFs were not treated with mitomycin C before co-culture. Sorting was repeated 3 times. Twice, as a pilot to confirm the purity of each cell type and the extraction protocol, the cells were collected into media and then half of each cell type was replated and half was used for RNA extraction. In the third repeat, the cells were collected into RNA extraction buffer and processed as detailed below. For all other experiments, a Guava EasyCyte (Millipore) cytometer was used, 10000 cells/sample were analyzed and the fraction of cancer cells was calculated using FlowJo 8.8.7 software.

Gene Expression Analysis:

RNA from duplicate samples was extracted and purified with RNAeasy mini kit (Qiagen, 74104), reverse transcribed with high capacity reverse transcription kit (Applied Biosciences, 4368813) and hybridized to duplicate SurePrint Agilent microarrays (Agilent, G4852A). Data were analyzed using Cluster, GOrilla, and MSigDB, and visualized using Java TreeView (See details in Extended Experimental Procedures). All microarray raw data have been deposited in a public database (Gene Expression Omnibus (GEO) accession GSE56252).

shRNA Knockdown of Genes in the TGFβ and SDF1 Signaling Networks:

The following genes were successfully knocked down using pLKO lentiviral vectors from the RNAi consortium shRNA library (see details in Extended Experimental Procedures), in D2A1 cells and in MEFs (>50% reduction in mRNA or protein expression was achieved): Smad2, Smad3, Smad4, TgfβR2. In addition, knockdown of Cxcr4 was attempted, but was not successful. Stable knockdown was achieved by selection with 1 g/ml puromycin. Knockdown levels were confirmed by qPCR (primers specified in the Extended Experimental Procedures). Knockdown of Smad2 in D2A1 cells was confirmed by western blot with anti-SMAD2 antibody (BD Biosciences, BDB610842).

ChIP-qPCR:

Flash-frozen tumor xenografts (0.5 cm³ each) were processed using a tissue pulverizer, fixed in formalin and homogenized with a Dounce homogenizer, and then processed as described previously (Lee et al., 2006; Mendillo et al., 2012). A cocktail of rat monoclonal HSF1 antibodies (Thermoscientific. RT-629-PABX) was used to IP HSF1, and normal rat IgG (Jackson ImmunoResearch Laboratories, 012-000-003) was used as control. For qPCR, RT2 SYBR Green qPCR Mastermix (SABiosciences) was used with the primers listed in the extended experimental procedures on a 7700 ABI Cycler/Detection System.

Xenografts:

MCF7 cells (1×10⁶) in PBS were inoculated subcutaneously in the right inguinal region of each mouse. Where indicated, 1×10⁶ MCF7 cells were mixed with 3×10⁶ wt or Hsf1 null primary MEFs prior to injection in a similar manner. Tumor growth was monitored by serial caliper measurements twice weekly. Mice were sacrificed and tumors were excised when volume reached 1.5 cm³ or overlying skin became ulcerated. Half the resected tissue was flash frozen for ChIP and half fixed in 10% formalin, processed using standard methods, cut into 5 mm sections and immunostained as described below.

Immunohistochemistry of Tissues, Scoring and Patient Outcome Analysis:

Paraffin blocks of human breast tumor and normal tissue from 12 patients were obtained from the Brigham and Women's Hospital (BWH, Boston, Mass.) Department of Pathology archive in accordance with regulations for excess tissue use stipulated by the BWH institutional review board. The following tissue microarrays were purchased from Pantomics (Richmond, Calif.): multi tumor array (MTU-951), colon cancer (COC1503), and prostate cancer (PRC-961). Whole sections of 72 lung tumors were retrieved from the archives of BWH. Whole sections of 46 breast tumors were retrieved from the archives of Rabin Medical Center (Israel). All samples were processed, stained and scored as described in Extended Experimental Procedures. Outcome analysis was performed on 46 breast cancer patients, and 72 lung cancer patients. Time to progression of disease and overall survival were estimated by the Kaplan-Meier (KM) method using GraphPad Prism 6 (GraphPad Software, Inc., La Jolla, Calif.). Unless indicated otherwise the log-rank test was used to assess statistical significance. All statistical tests were two-sided, and p<0.05 was considered statistically significant. To assess the independent contributions of epithelial and stromal HSF1 activation, as well as clinicopathologic factors, to multivariate prognostic models of outcome in breast and lung cancer, we performed multivariate Cox proportional hazards regression analysis and determined the Hazard Ratio (HR), 95% confidence interval around the HR, and coefficient P-value for HSF1 and the clinicopathologic parameters. The analyses were implemented using the coxph function in the survival package in R (http://www.r-project.org/).

EGFR and KRAS Genotyping Methods:

Total nucleic acid was extracted from formalin-fixed, paraffin-embedded surgical specimens of the lung cohort described above using a modified FormaPure System (Agencourt Bioscience Corporation, Beverly, Mass.). SNaPshot mutational analysis of a panel of cancer genes that included EGFR and KRAS was performed using primers listed in the Extended Experimental Procedures as previously described (Dias-Santagata et al., 2010).

Stromal Hsf1 mRNA Profiling and Patient Outcome Analysis:

Stromal gene expression profiling data for 53 breast cancers were obtained from GEO (GSE9014) for the Finak et al. study (Finak et al., 2008). The clinical data were obtained from the Supplementary Information Section of this publication. Arrays were performed in duplicate, replicate samples were averaged prior to analysis. The median stromal HSF1 expression level was used to categorize patients as showing low- and high-stromal expression of HSF1. The survival distributions in the stromal HSF1-low and stromal HSF1-high patients were compared by performing Kaplan-Meier analysis. The associations between Hsf1 expression, tumor grade and molecular subtype are presented by box & whisker plots. The bottom and top of the box represent the first and third quartiles, the band inside the box is the median and the ends of the whiskers represent the minimum and maximum of the data. Statistical significance was assessed with the log-rank test using GraphPad Prism 6. All statistical tests were two-sided, and p<0.05 was considered significant.

Extended Experimental Procedures

Gene Expression Analysis:

Genes differentially expressed in cancer cells upon co-culture (FIG. 3C) were determined as follows: For each gene in the array, the values were normalized to the average expression in cancer cells without MEFs. Genes for which the absolute value of the log₂(ratio) of cancer cells grown with MEFs vs without MEFs or the log₂(ratio) of cancer cells grown with WT vs Hsf1 null MEFs was greater than 1 were selected (1,381 and 1,275 genes, respectively, 1,997 total). Of these genes, 312 were excluded due to significant variation between biological replicates, yielding a final list of 1,685 differentially expressed genes (Table S1). Genes differentially expressed between cancer cells co-cultured with WT versus Hsf1 null MEFs were clustered by k-means clustering.

The gene expression analysis of the MEFs was performed as follows: out of 36,413 genes on the array, only those genes whose expression varied by at least one standard deviation from the mean across all conditions were included. In addition, genes with significant variation between duplicates (p<0.05 in a student's t-test) were excluded. This left 1434 genes, which were clustered by k-means clustering (Table S3). Selective gene set enrichment analysis (FIG. 3F) was performed by comparing the genes in groups 1 and 4 of FIG. 3E with “stromal” gene-sets from MSigDB (Table S4), and the DTF core stroma gene-set from (Beck et al., 2008). The following three additional gene-sets based on GEO (GSE45851) from (Mendillo et al., 2012) were also included: HEATSHOCK-UP includes genes upregulated in cancer cells in response to heat shock. CANCER_SIHSF1_UP and CANCER_SIHSF1_down include genes upregulated or downregulated in response to Hsf1 knockdown in cancer cells.

TABLE S3
G1 (Name: WT_UP; Information: The full list of genes enriched in
FIG. 3E, group 1)

0610040B09Rik, 0610040J01Rik, 1110021L09Rik, 1600021P15Rik, 1700030C10Rik,
1700080F18Rik, 2010002N04Rik, 2210403K04Rik, 2810432L12Rik, 2900062L11Rik,
2900076A13Rik, 3632451O06Rik, 3830612M24, 4833427G06Rik, 4930426L09Rik,
4930573O21Rik, 4930593A02Rik, 6030451C04Rik, 6530401D17Rik, 6720432D03Rik,
9030425E11Rik, 9330179D12Rik, 9430020K01Rik, A_55_P2138235, A030001D16Rik,
A630033H20Rik, Abca9, Abi3bp, Acan, Actg2, Adad2, Adam19, Adam23, Adamts1, Adh7,
Adm, Agtr1a, Agtr2, Ak5, Aldh1l2, Alx1, Apba1, Aplp1, Arhgap20, Arhgef19, Armcx6,
Arsi, Asphd2, Aspn, Atf5, Atp1b1, B3gnt9-ps, Bai2, BC022960, BC028528, Bcat1, Bel11b,
Begain, Bgn, Bhlhe22, Bmper, Btbd11, Bves, C030027H14Rik, C230029M16, C230060E24,
C230098O21Rik, C530028O21Rik, C79468, Cacna1d, Cacna1g, Cacna2d3, Cap2, Car6,
Car9, Cav3, Cbln1, Ccdc37, Ccl24, Ccnd2, Cd248, Cdh11, Cdk11, Cdo1, Cdsn, Ces1, Chac1,
Chodl, chr10: 8606683-8665783_R, chr15: 32167516-32174417_R, chr15: 32167516-32174427_R,
chr18: 38477934-38478445_F, chr2: 50152329-50289487_F, chr5: 149765314-149771786_R,
chr5: 67854954-67869163_F, chr5: 67865049-67868521_F, chr5: 91480533-91502237_R,
chr5: 92556322-92558910_F, chr8: 89996710-90049453_F, chr8: 90048940-90049461 _F,
chr9: 114412295-114418384_R, chr9: 114416254-114416844_R, Chrna10,
chrX: 165792277-165807952_F, Chst10, Chst2, Chst8, Clec11a, Clec3b, Cnn1, Cnnm1,
Col11a1, Col12a1, Col5a2, Col5a3, Col6a1, Col6a2, Col8a1, Col8a2, Cox6a2, Cpa2, Cpxm1,
Crabp1, Creb3l1, Crip1, Crispld2, Crlf1, Crlf2, Cspg4, Ctgf, Ctla2a, Ctnnd2, Ctsc, Ctsh,
Ctxn3, Cxcl12, Cxcl15, Cxcl5, Cygb, Cyp26b1, Cyp7b1, Cys1, D430040L24Rik,
D630002G06Rik, Daam2, Dact1, Dact3, Dchs1, Dclk3, Ddit41, Ddx4, Dgkg, Dhrs3, Dio3,
Dio3os, Dkk2, Dkk3, Dlk1, Dlx5, Dpt, Dpysl3, Dsp, Ebf1, Ece1, Edil3, Edn1, Efs, Ehd3,
Eln, Emid2, Emx2, Eng, ENSMUST00000030185, ENSMUST00000037953,
ENSMUST00000058139, ENSMUST00000061184, ENSMUST00000066153,
ENSMUST00000066194, ENSMUST00000070646, ENSMUST00000094140,
ENSMUST00000096145, ENSMUST00000097736, ENSMUST00000100820,
ENSMUST00000101487, ENSMUST00000105413, ENSMUST00000105988,
ENSMUST00000107676, ENSMUST00000108435, ENSMUST00000110346,
ENSMUST00000110529, ENSMUST00000111103, ENSMUST00000113367,
ENSMUST00000113576, Epb4.1l4a, Epha3, Epha4, Evi2a, Eya2, Fam105a, Fam129a,
Fam131b, Fam198b, Fam20a, Fam59b, Fat4, Fbln2, Fbln5, Fbn1, Fbn2, Fbxl7, Fbxw18,
Fbxw25, Fgf12, Fgf18, Fgf7, Fgfr3, Fhl2, Flt1, Fndc1, Foxc2, Foxs1, Fst, Fxyd5, Fyn, Fzd8,
Galnt13, Galnt14, Galnt9, Galntl1, Gap43, Gas2, Gas6, Gata6, Gdap111, Gdf10, Gdf6, Ggt7,
Gimap9, Gli1, Glis1, Glrx, Gm12824, Gm13272, Gm13275, Gm14066, Gm15226,
Gm15470, Gm15645, Gm2496, Gm3604, Gm3932, Gm5144, Gm7278, Gm8272, Gm9885,
Gnao1, Gng8, Gpc3, Gpm6b, Gpr124, Gpr153, Gpr176, Gpr85, Gpx7, Grem1, Gria3, Gsc,
Gsdmd, Gstt1, Gucy1a3, H19, Has2, Has2as, Hmga2, Hoxb9, Hoxc13, Hoxc6, Hoxc9, Hunk,
Igdcc4, Igf1, Igtbp2, Igfbp3, Iglon5, Igsf10, Il18, Il1rl1, Il1rn, Il20ra, Impact, Inhba, Insc,
Islr, Ism1, Isoc2b, Itga11, Itga8, Itga9, Itln1, Itm2a, Jag1, Jdp2, Kcnf1, Kcnip3, Kcnk2,
Kcnk6, Kdelr3, Kif26b, Klhl29, Krt15, Lama2, Lama4, Layn, Lgr5, Lhfpl4, Lims2,
LOC100044727, LOC100045652, LOC100046471, LOC100047308, LOC100047419,
LOC625164, LOC636126, LOC640605, LOC677253, Lox, Loxl3, Lpin3, Lpl, Lrp2, Lrp4,
Lrrc17, Lrrc32, Lrrc33, Lrrtm3, Lsp1, Ltbp1, Lypd1, Mab21l1, Mab21l2, Maf, Maob,
Mapk13, Marcks, Mdfi, Megf6, Meis1, Mest, Mfap2, Mfap4, Mfap5, Mgll, Mid1, Mmp19,
Mmp2, Mn1, Mpped2, Msmp, Mtap1a, Mxra7, Mylk, Myo1b, Myocd, Ncam1, Ndn, Ndst3,
Ndufa4l2, Nell2, Nfatc4, Nid1, Nipal2, Nkd1, Nkx2-5, Nlrp5, Nnmt, Notch3, Npr3, Nrg1,
Nsg1, Ntrk3, Nts, Nuak1, Olfml1, Omd, Optc, Osr1, Otud7a, Panx1, Panx3, Pappa, Pax3,
Pcdh10, Pcdh19, Pcdhb16, Pdgfd, Pdgfrb, Pdp1, Peli2, Penk, Pex5l, Phf19, Pi16, Pir, Pitx1,
Plac9, Plcd1, Plcl1, Plcl2, Pld5, Pltp, Plxna2, Pmepa1, Podn, Podnl1, Podxl2, Postn, Ppap2b,
Ppbp, Prdm16, Prr5, Prrx1, Prss12, Prss23, Prss35, Ptgir, Ptgr1, Pth1r, Ptn, Ptx3, Ramp2,
Rarres1, Rasgrp2, Ras112, Rassf6, Rbpms, Rftn1, Rftn2, Rgs10, Rgs17, Rgs4, Rhoj, Rnase4,
Rnd3, Rp139l, Rsph9, Rspo1, Rspo2, Rtn4rl1, Runx1t1, S1pr3, Sa1l1, Sec16b, Serpina3f,
Serpina3i, Serpinb1a, Serpine1, Serpinf1, Setbp1, Sfrp1, Sfrp2, Sh3gl3, Shisa2, Siglecg,
Sirpa, Skor1, Slc12a2, Slc16a3, Slc1a6, Slc22a17, Slc2a10, Slc43a1, Slc4a11, Slco1a5,
Slco4a1, Slit2, Slit3, Smoc2, Snap91, Sncaip, Sned1, Snord123, Sntg1, Soat2, Srpx2, Srrm4,
Stac, Sulf1, Susd2, Svep1, Syt8, Sytl2, Tagln, Tbkbp1, Tbx4, Tbxa2r, Tcfl5, Tex13a, Tg,
Tgfb1i1, Thbd, Thbs1, Thbs2, Thy1, Timp3, Tlr1, Tlr6, Tmeff2, Tmem178, Tmem45a,
Tmem47, Tmem59l, Tnfsf11, Tnik, Tppp, Tprg, Trib3, Trpv2, Tshz3, Tslp, Tspan11,
Tspan17, Tspan2, Tspan6, Ttc9, Ttll11, Tubb2b, Tubb3, Unc5c, Usp13, Vat11, Vax2os1,
Vax2os2, Vill, Wif1, Wisp1, Wisp2, Wnt10b, Wnt6, Wscd2, XIST, Xist, Xpnpep2, Zcchc5,
Zdhhc14, Zfp105, Zfp783.

TABLE S3
G2 (Name: WT_DOWN; Information: The full list of genes enriched in
FIG. 3E, group 2)

1600016N20Rik, 1700003F12Rik, 1700008P20Rik, 1700016C15Rik, 1700019G17Rik,
1700020O03Rik, 1700048O20Rik, 1810014F10Rik, 1810019J16Rik, 1810037I17Rik,
2010107G23Rik, 2310015B20Rik, 2310042D19Rik, 2610528A11Rik, 4732419C18Rik,
4930506M07Rik, 5730469M10Rik, 6330509M05Rik, 8430408G22Rik, 8430427H17Rik,
9930023K05Rik, AA388235, Aard, Abat, Abca8b, Abcc3, Abi3, Acox2, Acrbp, Acss1,
Actn3, Acyp2, Adcy5, Adhfe1, Adora1, Afap1l2, AI428936, AI448005, AI596198, Aim2,
Aldh1b1, Aldoc, Aloxe3, Ano1, Ap1s3, Aqp3, Arhgef6, Atf3, Atp1b2, AU022751,
AW011956, B4galnt4, Batf3, BC021767, BC057022, BC066028, Bdh2, Bend4, Bex1, Bex4,
Bfsp2, Bmf, Bmp7, Btn2a2, Bzrap1, C230037E05Rik, C77370, Cadm4, Calm14, Capn6,
Car13, Casz1, Cbs, Ccdc160, Ccl27a, Cd274, Cd74, Cd83, Cd96, Cdkn1c, Celsr1, Cfi, Cgn,
Chi3l1, Chmp4c, Chn2, chr1: 138579972-138587381_F, chr1: 6199946-6200603_R,
chr10: 62267317-62269871_R, chr10: 69669025-69686275_F, chr10: 69679644-69682458_F,
chr11: 53596919-53672731_R, chr14: 43929100-43945100_F, chr2: 94046243-94070067_R,
chr2: 94082835-94101939_R, chr3: 137742666-137744470_F, chr5: 120269342-120279542_R,
chr6: 57583950-57624216_F, chr7: 111526943-111545093_R, chr7: 67111261-67120697_R,
chr8: 48480224-48480698_R, chr9: 4260788-4261404_F, Chrdl2, Cilp, Cilp2,
Cited1, Clic5, Cmbl, Cmpk2, Cmtm8, Col11a2, Crabp2, Creld1, Csf2ra, Cxcl16, Cyp4f14,
D230034L24Rik, D330045A20Rik, D630039A03Rik, D8Ertd82e, Dapl1, Defa-rs4, Defa-
rs7, Defb1, Depdc7, Dnaic1, Dpcr1, Dpyd, Dusp27, Dusp28, E130102H24Rik,
E530001K10Rik, Egln1, Ell3, Emb, En2, Enpep, ENSMUSG00000068790,
ENSMUST00000023458, ENSMUST00000070702, ENSMUST00000090647,
ENSMUST00000099340, ENSMUST00000100388, ENSMUST00000102549,
ENSMUST00000105041, ENSMUST00000109491, ENSMUST00000109709,
ENSMUST00000112728, ENSMUST00000113743, ENSMUST00000117486,
ENSMUST00000118651, Epha1, Epha7, Ercc4, Exd1, F5, Fand2a, Fam117a, Fam174b,
Fam176a, Fam180a, Fam187b, Fam55b, Fam73a, Fbln7, Fbp2, Fbxo32, Fev, Egf5, Fibin,
Foxq1, Fras1, Frat1, Frmd3, Galnt12, Gata3, Gca, Gm10406, Gm10439, Gm12216,
Gm12250, Gm13138, Gm13308, Gm14446, Gm15085, Gm15144, Gm16525, Gm1973,
Gm2897, Gm3020, Gm3099, Gm3115, Gm3187, Gm3252, Gm3411, Gm3667, Gm3696,
Gm4841, Gm4951, Gm5215, Gm5458, Gm5480, Gm567, Gm5797, Gm765, Gm9132, Gmpr,
Gpr120, Gpr160, Grhl3, Gstm6, Gstm7, Gyltl1b, H2-Ab1, H28, Hapln1, He, Hck, Hdac11,
Hook1, Hoxa9, Hoxd10, Hoxd11, Hoxd13, Hoxd4, Hoxd8, Hoxd9, Hrc, Icosl, Ifi44, Ifih1,
Il17re, Il28ra, Il3ra, Inadl, Inpp5j, Insl6, Iqgap2, Itga10, Itga3, Jag2, Jmjd1c, Jsrp1, Kenh2,
Kcnk1, Kcnk5, Klf15, Klf8, Klhl13, Klhl30, Klk8, Krt19, Kynu, Lhpp, Lhx9, Lingo4, Lipi,
LOC100039927, LOC100044290, LOC100044365, LOC100044641, LOC100044755,
LOC100044854, LOC100045787, LOC100047292, LOC100048441, LOC635918, Lrrc14b,
Lsr, Luzp4, Macc1, Macrod1, Map3k15, Matn4, Mbnl3, Meox1, Mfsd6, Micalcl, Mlana,
Mll3, Mpa2l, Mpzl2, Msx2, Mug1, Mybpc2, Myc, Myh14, Nags, Nap1l3, Ndrg2, Negr1,
Neu1, Neurl3, Nipal3, Nup210, Ociad2, Olfr1384, Parm1, Pcsk4, Pctp, Pde2a, Pde3b, Pde9a,
Pdlim3, Pgam2, Pknox2, Pla2g4e, Plcd4, Plekha7, Plin4, Plscr4, Poc1b, Pparg, Ppfibp2,
Ppm1e, Ppp1r14d, Ppp1r16b, Ppp1r9a, Prickle1, Prickle3, Prlr, Prodh, Prosapip1, Psmb8,
Psmb9, Psmd9, Ptchd1, Ptgds, Ptpn18, Ptprq, Rab3b, Raet1c, Rapgef5, Rasgef1a, Rasl11a,
Rbm47, Rhbdl3, Rhov, Rhpn2, Ripk4, Rnf128, Rnf207, Robo4, Rps6ka1, Rragb, Rsad2,
Rundc3b, Scly, Scrg1, Scx, Sdr39u1, Selenbp1, Sema4d, Shroom2, Slc16a4, Slc25a43,
Slc26a4, Slc29a2, Slc35f2, Slc38a1, Slc9a2, Slfn2, Sln, Smpdl3a, Snrpn, Spib, Spint2,
Srcin1, Srd5a1, Srpk3, St3ga16, Steap4, Stxbp3b, Susd4, Syt1, Tacstd2, Tbx5, Tcam1,
Tceal5, Tfpi, Tha1, Tjp2, Tmem125, Tmem144, Tmem180, Tmem20, Tmem22, Tmem51,
Tmem56, Tmigd1, Tmprss6, Tnfsf10, Tnnc1, Tnnc2, Tnnt1, Tnnt3, Tpd52l1, Trim30, Trim6,
Trim63, Trim79, Trpv4, Tspan12, Tspan13, Ttc22, Ubd, Ubtd1, Upb1, Uqcc, Vgll2,
Wbscr17, Ybx2, Ydjc, Ypel1, Zbtb16, Zdhhc23, Zfp583, Zfp612, Zfp820.

TABLE S3
G3 (Name: UPtumor_KOhigh; Information: The full list of genes
enriched in FIG. 3E, group 3)

1190003J15Rik, 1810011O10Rik, 2200002D01Rik, 2310033P09Rik, 6030429G01Rik,
Ampd3, Aox1, Apol9a, Apol9b, Artn, Aspg, BC006779, Bcl3, Bmyc, Bst2, C3, Casp4, Ccl5,
Cfb, chr1: 175705225-E75717250_R, chr12: 10581841-10582233_F, chr236112819-36114110_F,
chr8: 19759348-19759959_R, Cldn9, Cst6, Cxcl10, Cyb561, D14Ertd668e,
Dhdpsl, Dhx58, Dll1, Dnahc9, Dusp9, Dyrk3, Echdc3, ENSMUST00000077662,
ENSMUST00000098144, ENSMUST00000111210, ENSMUST00000113874,
ENSMUST00000116010, ENSMUST00000118006, Eps8l2, F11r, F830014O18Rik, Fggy,
Fosb, Gata4, Gbe1, Gbp1, Gbp11, Gbp2, Gbp3, Gbp4, Gbp6, Gbp9, Gdpd1, Gdpd2,
Gm1966, Gm4229, Gm4902, Gm7035, Gm9640, Gm9706, Gng2, Gprc5c, Gvin1, H2-D1,
H2-K1, H2-Q2, H2-Q5, H2-Q6, H2-Q7, H2-Q8, H2-T23, I830012O16Rik, Ica1, Ica1l,
Ifi203, Ifi27l2a, Ifit1, Ifit3, Igtp, Iigp1, Inmt, Irf7, Irf9, Irgm2, Kank3, Lbp, Ldhd, Lgals3bp,
LOC100047388, LOC235882, LOC547349, LOC630285, LOC631406, LOC675328,
LOC676689, LOC677149, LOC677644, Mapt, Mmp13, Mnda1, Msrb2, Mt2, Mx1, Mx2,
Nr1h3, Oas1a, Oas1f, Oas2, Oas3, Oasl1, Oasl2, Olfr1223, Parp14, Perp, Phfl1, Pik3r5,
Plscr1, Ppl, Ptgs1, Pthlh, Ptpn6, Rhbdf2, Rnfl82, Rorc, Rtp4, Scube3, Slc47a2, Slpi, Stap2,
Syngr1, Tgfbi, Tgm2, Tgm5, Tgtp2, Tinagl1, Tnfrsf21, Trim54, Uba7, Usp18, Xafl, Zbp1.

TABLE S3-G4
(Name: UPtumor_WThigh; Information: The full list of genes
enriched in FIG. 3E, group 4)

1700112E06Rik, 1810020O05Rik, 2310002L13Rik, 2410076I21Rik, 2900040C04Rik,
4632428N05Rik, 4930431P03Rik, 4930583H14Rik, 4931408A02Rik, 5730559C18Rik,
6430601O08Rik, 9330188P03Rik, A_55_P2106815, A_55_P2389893, A230065H16Rik,
A430089I19Rik, AA467197, AA684185, Abcg1, Acot1, Acsbg1, Adamts7, Adssl1,
AF067061, AI467606, AI661453, AI662270, Akr1c18, Aldh3a1, Angptl4, Angptl7, Aqp5,
Areg, Arhgef3, Asgr1, Aspa, Atp9a, BC016201, BC030476, BC061237, Blnk, Btc, C4b,
Cadm1, Cd14, Chi3l3, chr12: 119825125-119850750_R, chr14: 105988692-105994145_F,
chr17: 15226181-15250333_F, chr18: 70400082-70404236_R, chr2: 181663381-181664097_F,
chr4: 88583425-88584070_R, chr5: 54053882-54054174_F, Chst1, Cp,
Csnk1g3, Cxcl3, D13Ertd608e, Dbndd1, Dhh, Dio2, Dmp1, Dnajc22, Dpep3,
ENSMUST00000033688, ENSMUST00000101281, ENSMUST00000110056,
ENSMUST00000119870, Etv1, Fam110c, Fam49a, Fcna, Fes, Flywch2, Gabrd, Galntl2,
Gjb4, Glod5, Glrp1, Glud2, Gm10375, Gm10408, Gm10845, Gm11427, Gm13032, Gm3265,
Gm3652, Gm3761, Gm3923, Gm4072, Gm4326, Gm4340, Gm4492, Gm4638, Gm4653,
Gm4728, Gm4907, Gm5604, Gm5860, Gm7455, Gpr157, Gpr97, Hamp2, Hao1, Hoxa2,
Hoxa5, Hpse, Igfbp4, Igfbp6, Il34, Il6ra, Il7, Isyna1, Itgb7, Krt18, Krt8, Lcn2,
LOC100038937, LOC100042220, LOC100046186, LOC100046361, LOC100046544,
LOC100046616, LOC100047159, LOC433068, LOC638189, LOC639063, LOC639910,
LOC674800, LOC677262, LOC677430, Lrrc19, Mc3r, Mgst2, Mmp10, Mmp3, Msln, Naip2,
Napsa, Ngef, Nin1, Nppb, Nt5e, Olfr1221, Olfr1466, Padi2, Padi3, Pcbd1, Pgf, Pgm5, Phlda1,
Pip, Pkd1l2, Pkp1, Pkp3, Pla2g7, Pllp, Podx1, Prelid2, Prss46, Psg23, Ramp3, Rapgef3, Rarb,
Rbbp9, Rbp1, Rg9mtd2, Rgs16, Rnf183, S100a8, Sema4b, Sema6a, Sh3tc2, Sigirr, Slc16a2,
Slc22a4, Slc2a5, Slc47a1, Slc6a2, Slc7a7, Slco2a1, Sox2, Sox2ot, Spp1, Stab1, Stac2, Tafl2,
Tapbpl, Tas2r109, Tas2rl13, Tes, Thrb, Tm4sfl, Tmeml71, Tnfrsfl8, Tnnt2, Trem2,
Trp53il1, Upp1, Vipr1, Wfdc2, XM_001476722, Zscan4a, Zscan4e, Zscan4f,
2810405K02Rik, 3300005D01Rik, 5430435G22Rik, 5830444B04Rik, 6720407P12Rik,
Apob48r, Apol10b, Aqp1, Arhgap22, Arhgap23, Atp6v0e2, AU018778, Bcl6, Cav1,
Ccdc109b, Ccnd1, Cdyl2, Cela1, Chd7, chr18: 13107755-13107844_F, chr18: 3003075-3016150_R,
chr18: 5162836-5165729_R, chr8: 119090566-119097735_R, chr8: 119092623-119094219_R,
Clca1, Clca2, Col18a1, Col4a5, Csgalnact1, Ctsw, Cx3cl1, Cxcl1, Cxcr7,
Cyp1b1, D6Mm5e, Defb48, Dlk2, Dusp4, Dusp6, Eml2, ENSMUST00000052354,
ENSMUST00000100997, Ereg, Etv4, Etv5, Fblim1, Fosl1, Gfra2, Glipr1, Gm10635,
Gm13043, Gm2022, Gm6684, Gm6742, Grpr, Gstk1, Guca1a, H60a, H60b, Hectd2, Hey1,
Hsd11b1, Hsf4, Htatip2, Id4, Il11, Il18rap, Itih2, Kcnn4, Khdrbs3, Kif21b, Kirrel3, Lhfpl2,
LOC100048500, Lonrf3, Ltbp2, Megf10, Mtus2, Mustn1, Ndrg4, Nipal1, Nkd2, Nlrp2,
Nr2fl, Nrp1, Oplah, Osmr, P2rx7, P2ry2, Pabpc1l, Prkg2, Pyroxd2, Rap1gap, Rgl1, Rhox5,
Runx1, Scara5, Sema5a, Serpina3g, Sgk1, Siae, Slc25a45, Snai1, Sod3, Sox9, Sphk1, Spry1,
St3gal1, Tbx3, Tgfb1, Tmem200a, Tnfaip2, Tnfrsfl1b, Tnn, Trib2, Ugt1a6b, Vcam1, Wt1,
Zfp503, Zfp608.

TABLE S4
Related to FIG. 3F

| NAME | SIZE | ES | NES | NOM p-val | FDR q-val | FWER p-val |
|---|---|---|---|---|---|---|
| BECK_2008_DTF_{—} | 57 | 0.80714685 | 2.4290578 | 0 | 0 | 0 |
| CORE_STROMA | | | | | | |
| CANCER_SIHSF1_UP | 134 | 0.6359371 | 2.135734 | 0 | 0 | 0 |
| DURAND_STROMA_{—} | 269 | 0.5750911 | 2.128967 | 0 | 0 | 0 |
| MAX_UP_M2581 | | | | | | |
| ROY_WOUND_BLOOD_{—} | 47 | 0.6686722 | 1.9466351 | 0 | 0 | 0 |
| VESSEL_UP_M7337 | | | | | | |
| SUNG_METASTASIS_{—} | 88 | 0.5333181 | 1.7007246 | 0 | 0.003381106 | 0.014 |
| STROMA_UP_M9483 | | | | | | |
| HEATSHOCK_UP | 100 | 0.50786966 | 1.6446015 | 0.001779359 | 0.004592915 | 0.022 |
| ROY_WOUND_BLOOD_{—} | 16 | 0.60460496 | 1.3814436 | 0.09864604 | 0.071436726 | 0.309 |
| VESSEL_DN_M2176 | | | | | | |
| RESPONSE_TO_{—} | 154 | 0.3764523 | 1.3050678 | 0.055655297 | 0.12162316 | 0.526 |
| WOUNDING_M5634 | | | | | | |
| CHANG_CORE_SERUM_{—} | 172 | 0.30692938 | 1.0748878 | 0.29858658 | 0.52121156 | 0.983 |
| RESPONSE_DN_M5793 | | | | | | |
| CHANG_CORE_SERUM_{—} | 177 | 0.27332214 | 0.964867 | 0.5273973 | 0.79661185 | 1 |
| RESPONSE_UP_M5042 | | | | | | |
| RESPONSE_TO_{—} | 431 | 0.25125414 | 0.96408314 | 0.57165605 | 0.7262072 | 1 |
| STRESS_M14874 | | | | | | |
| THEILGAARD_NEUTROPHIL_{—} | 66 | 0.3083899 | 0.9483893 | 0.5750916 | 0.70759326 | 1 |
| AT_SKIN_WOUND_UP_M2117 | | | | | | |
| SUNG_METASTASIS_{—} | 44 | 0.32428306 | 0.925436 | 0.56396395 | 0.71491784 | 1 |
| STROMA_DN_M2904 | | | | | | |
| PROTEIN_FOLDING_M2393 | 53 | 0.30171806 | 0.8842106 | 0.6660714 | 0.76645523 | 1 |
| THEILGAARD_NEUTROPHIL_{—} | 203 | 0.2301372 | 0.82677877 | 0.8949153 | 0.83326566 | 1 |
| AT_SKIN_WOUND_DN_M11234 | | | | | | |
| CANCER_SIHSF1_DOWN | 127 | −0.4099834 | −1.4268515 | 0.004716981 | 0.13569026 | 0.213 |

Immunohistochemistry of Tissues, Scoring and Patient Outcome Analysis:

Formalin-fixed, paraffin-embedded (FFPE) sections were deparaffinized, blocked with 3% H₂O₂ and antigen retrieval was performed by using a pressure cooker with Dako citrate buffer (pH 6.0) at 120° C.±2° C., 15±5 PSI. Slides were blocked with 3% normal rabbit serum, and the antibodies and dilutions listed in the table below were used. Visualization was achieved with 3,30-diaminobenzidine (DAB) as a chromogen (Dako Envision+ System) for single staining, and DAB in combination with alkaline phosphatase (ImmRRESS-AP) for double staining. Counterstaining was performed with Mayer-hematoxylin. Immunostained breast sections were scored by a pathologist (SS), using light microscopy. The BWH breast sections were also scanned and scored by the Scanscope digital slide system (Aperio). Lung sections were scored independently by two pathologists (SS and LMS). A 0 to 3 scale was used for scoring, with 0-1 for no/low-level nuclear staining, 2 for intermediate and 3 for high nuclear staining. Pathologists were blinded to the survival outcomes of the participants and to the scores given by the other pathologist. An average combined score was calculated for each case. Discrepant cases (3 out of 72, in which the difference between scores was equal to or larger than 2) were re-reviewed and a consensus score was established.

TABLE S2
shRNA clones used in this study

| Gene | TRC number |
|---|---|
| Smad2 | TRCN0000327370 |
| Smad2 | TRCN0000327446 |
| TgfβR2 | TRCN0000294600 |
| TgfβR2 | TRCN0000022627 |
| Smad3 | TRCN0000089026 |
| Smad3 | TRCN0000314114 |
| Smad4 | TRCN0000025881 |
| Smad4 | TRCN0000362661 |
| Cxcr4 | TRCN0000028704 (did not work) |
| Cxcr4 | TRCN0000028724 |

TABLE 55
Oligonucleotides used in this study
(SEQ ID NOS: 1-28 respectively)

| NAME | SEQUENCE |
|---|---|
| GAPDH-qPCR-F | TTGATGGCAACAATCTCCAC |
| GAPDH-qPCR-R | CGTCCCGTAGACAAAATGGT |
| Tgfβ1-qPCR-F | CAACCCAGGTCCTTCCTAAA |
| Tgfβ1-qPCR-R | GGAGAGCCCTGGATACCAAC |
| Tgfβ2-qPCR-F | TTGTTGAGACATCAAAGCGG |
| Tgfβ2-qPCR-R | ATAAAATCGACATGCCGTCC |
| Tgfβ3-qPCR-F | TCTCCACTGAGGACACATTGA |
| Tgfβ3-qPCR-R | ATTCGACATGATCCAGGGAC |
| Sdf1-qPCR-17 | TTTCAGATGCTTGACGTTGG |
| Sdf1-qPCR-R | GCGCTCTGCATCAGTGAC |
| Smad2-qPCR-F | TTTGCTGTACTCAGTCCCCA |
| Smad2-qPCR-R | TGAGCTTGAGAAAGCCATCA |
| Smad3-qPCR-F | ACAGGCGGCAGTAGATAACG |
| Smad3-q PCR-R | AACGTGAACACCAAGTGCAT |
| Smad4-qPCR-F | GGCTGTCCTTCAAAGTCGTG |
| Smad4-qPCR-R | GGTTGTCTCACCTGGAATTGA |
| TgfPR2-qPCR-F | CTGGCCATGACATCACTGTT |
| TgfPR2-qPCR-R | GTCGGATGTGGAAATGGAAG |
| Cxer4-qPCR-F | ACTCACACTGATCGGTTCCA |
| Cxer4-qPCR-R | AGGTGCAGGTAGCAGTGACC |
| Sdf1 HSE-qPCR-F | CTTAGGCTGCTTCTGGCACT |
| Sdf1 HSE-qPCR-R | TTTGAGCTTCTGGCCAAGTT |
| Tgfβ2 HSE-qPCR-F | GACCCCACATCTCCTGCTAA |
| Tgfβ2 HSE-qPCR-R | GGATCCATTTCCATCCAAG |
| DHER1-qPCR-F | ACCTGGTCGGCTGCACCT |
| DHER1-qPCR-R | TTGCCCTGCCATGTCTCG |
| Mouse intergenic-qPCR-F | ATGTCAGGCCCATGAACGAT |
| Mouse intergenic-qPCR-R | GCATTCATGGAGTCCAGGCTTT |

The primers listed below were used for targeted mutation analysis of EGFR and KRAS and for detecting recurrent insertions and deletions in EGFR exons 19 and 20.

PCR primers (SEQ ID NOS: 29-46, respectively):
KRAS exon 2,
5′-ACGTTGGATGTCATTATTTTTATTATAAGGCCTGCT
G-3′ (forward)
and
5′-ACGTTGGATGAGAATGGTCCTGCACCAGTAA-3′ (reverse),
KRAS exon 3,
5′-ACGTTGGATGGTTTCTCCCTTCTCAGGATTC-3′ (forward)
and
5′-ACGTTGGATGCCCACCTATAATGGTGAATATCTT
C-3′ (reverse),
KRAS exon 4,
5′-ACGTTGGATGAACAGGCTCAGGACTTAGCAA-3′ (forward)
and
5′-ACGTTGGATGTTATTTCAGTGTTACTTACCTGTCTT
G-3′ (reverse),
EGFR exon 7,
5′-ACGTTGGATGCTACAACCCCACCACGTACC-3′ (forward)
and
5′-ACGTTGGATGCAGTTAGAGGGCCCACAGAG-3′ (reverse),
EGFR exon 15,
5′-ACGTTGGATGCCAGTGTGCCCACTACATTG-3′ (forward)
and
5′-ACGTTGGATGCTTCCAGACCAGGGTGTTGT-3′ (reverse),
EGFR exon 18,
5′-ACGTTGGATGCCAACCAAGCTCTCTTGAGG-3′ (forward)
and
5′-ACGTTGGATGCCTTATACACCGTGCCGAAC-3′ (reverse),
EGFR exon 19,
5′-ACGTTGGATGTCGAGGATTTCCTTGTTGGC-3′ (forward)
and
5′-ACGTTGGATGGATCCCAGAAGGTGAGAAAG-3′ (reverse),
EGFR exon 20,
5′-ACGTTGGATGTGTTCCCGGACATAGTCCAG-3′ (forward)
and
5′-ACGTTGGATGATCTGCCTCACCTCCACCGT-3′ (reverse),
EGFR exon 21,
5′-ACGTTGGATGCCTCCTTCTGCATGGTATTC-3′ (forward)
and
5′-ACGTTGGATGGCAGCATGTCAAGATCACAG-3′ (reverse).
Extension primers:
KRAS.34 extR
(SEQ ID NO: 47)
5′-GACTGACTGCTCTTGCCTACGCCAC-3′,
KRAS.35 extF
(SEQ ID NO: 48)
5′-CTGACtCTTGTGGTAGTTGGAGCTG-3′,
KRAS.37 extF
(SEQ ID NO: 49)
5′-TGACTGACtGATGGTAGTTGGAGCTGGT-3′,
KRAS.38 extF
(SEQ ID NO: 50)
5′-GACTGACTGACGGTAGTTGGAGCTGGTG-3′,
KRAS.181 extF
(SEQ ID NO: 51)
5′-ATTCTCGACACAGCAGGT-3′,
KRAS.182 extF
(SEQ ID NO: 52)
5′-ACTGACTGACTGACTGACTGACTGACTGACTGACTGACTGACTG
ATCTCGACACAGCAGGTC-3′,
KRAS.183 extR
(SEQ ID NO: 53)
5′-TGACTGACTGACTGACTGACTGACTGACTGACTGCTCATTGCAC
TGTACTCCTC-3′,
KRAS.436 extR
(SEQ ID NO: 54)
5′-GACTGACTGACTGACTGACTGACTGACTGACTGACTGACTGGACT
GACTGACTGACTGGTTACTTACCTGTCTTGTCTTTG-3′,
KRAS.437 extF
(SEQ ID NO: 55)
5′-TGACTGACTGACTGACTGACTGACTGACTGACTGGACTGACTGAC
TGACTGACTGGACTGACTGAGAATTCCTTTTATTGAAACATCAG-3′,
EGFR.866 extR
(SEQ ID NO: 56)
5′-TGACTGACTGACTGACTGACTGACTGACTGACTGCTTCTTCACGC
AGGTG-3′,
EGER.793 extR
(SEQ ID NO: 57)
5′-GACTGACTGACTGACTGACTGACTGACTGACTGACTGACTGACTG
ACTGGTTGTTTTCTCCCATGACT-3′,
EGFR.2155 extF
(SEQ ID NO: 58)
5′-GACTGACTGACTGACTGACTGACTGTCAAAAAGATCAAAGTGCT
G-3′,
EGFR.2156 extF
(SEQ ID NO: 59)
5′-ACTGACTGACTGACTGACTGACTGACTGACTGACTGACTGACCAAA
AAGATCAAAGTGCTGG-3′,
EGFR.2235 49 del xtF
(SEQ ID NO: 60)
5′-CTGACTGACTGACTGACTGTTCCCGTCGCTATCAA-3′,
EGFR.2236 50 del extF
(SEQ ID NO: 61)
5′-CTGACTGACTGACTGACTGTCCCGTCGCTATCAAG-3′,
EGFR.2369 extR
(SEQ ID NO: 62
5′-CTGACTGACTGACTGACTGACTGACTGACTAAGGGCATGAGCTG
C-3′,
EGFR.2573 extF
(SEQ ID NO: 63)
5′-GACTGACTGACTGACTGACTGACTGACAGATCACAGATTTTGGG
C-3′.
Sizing assay primers (in/del):
NED-EGFR_Ex19_F,
(forward; SEQ ID NO: 64)
5′-GCACCATCTCACAATTGCCAGTTA-3′
and
EGFR-Ex19-REV1,
(reverse; SEQ ID NO: 65)
5′-AAAAGGTGGGCCTGAGGTTCA-3′,
VIC-EGFR_Ex20_F2:
(forward; SEQ ID NO: 66)
5′-CGAAGCCACACTGACGTG-3
and
EGFR_Ex20_R_2:
(reverse; SEQ ID NO: 67)
5′-CCGTATCTCCCTTCCCTGAT-3′

Location of HSEs:

Tgfβ2—mm10 chr1:186,698,878-186,698,892
SDF1—mm10 chr6:117,173,089-17,173,102

Antibodies and dilutions used in this study

| | | | Primary | | |
|---|---|---|---|---|---|
| Antibody | Company | Catalog# | dilution | Secondary | Stain System |
| LCA | Dako | M071 | 600 | polymer-M | Envision |
| CD31 | Dako | M0823 | 30 | polymer-M | Envision |
| SMA (single/double | Vector | VP-S281 | 50/40-AP | M.O.M | ImmPress/AP |
| stain) | | | | | |
| HSF1 Ab4 | Thermo | RT-629 | 1:500 stroma | rabbit-anti- | ABC |
| | | | 1:1000 tumor | Rat | |

### Example 10

### Identification of HSF1 Stromal Gene Signature Subset

A combination of experimental and bioinformatic approaches was used to define a set of HSF1-regulated genes of particular use for assessing the level of HSF1 activity in tumor-associated stromal cells for purposes of cancer classification, diagnosis, prognosis, treatment-specific prediction, and treatment selection. The pipeline started with the Group 1 and Group 4 gene lists described above. As described above, these 871 genes have increased expression in Hsf1+/+ versus Hsf1−/−MEFs. These 871 genes map to 562 genes in the Finak, G. et al. dataset discussed above (GEO (GSE9014)). Rohinitib (RHT) is a member of the rocaglamide class of compounds that was identified as a potent inhibitor of HSF1 activity in a recent chemical screening effort (Santataga, S., et al., 2013). Its structure is depicted below.

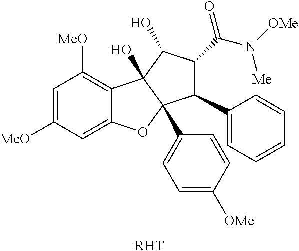 [SMILES: COc1ccc([C@@]23Oc4cc(OC)cc(OC)c4[C@]2(O)[C@H](O)[C@H](C(=O)N(C)OC)[C@H]3c2ccccc2)cc1 ; "RHT"]

The 562 genes that mapped to genes in the Finak dataset were tested to determine the effect of RHT on their expression in Hsf1+/+MEFs versus Hsf1−/−95 MEFs. It was found that expression of 95 of these genes was suppressed by RHT in an HSF1-dependent fashion (expression was reduced in RHT treated Hsf1+/+MEFs but not in Hsf1−/−MSFs). A filter was applied to these genes to identify those that were differentially expressed in tumors in the Finak dataset with poor versus good outcomes. In particular, genes that were associated with poor outcome and displayed greater expression in tumors with poor versus good outcome (<0.05 by t-test AND >0.5 (Poor—Good log 2 expression) were identified. This resulted in the following set of 15 high-priority genes that are in Group 1 or Group 4 AND show a greater reduction in expression upon exposure to RHT in HSF1 WT MEFs versus HSF1 null MEFS AND are expressed at higher levels in stroma from patients with poor outcome than in patients with a good outcome: CDSN, COL4A5, MDFI, MFAP5, MXRA7, NID1, OLFML1, SLC16A3, SLC22A17, SLC2A10, SLC4A11, SLC6A2, TES, TUBB3, XPNPEP2 (the “15 gene set”). This high priority set was expanded to include 25 additional genes (the “25 gene set”) from the 562 gene set that met the following criteria: they were associated with poor outcome in the Finak dataset AND displayed greater expression in tumors with poor versus good outcome (<0.05 by t-test AND >0.77 (Poor—Good log 2 expression): ADM, AK5, ANGPTL4, CHD7, COL11A1, FGFR3, GNAO1, 1D4, ITLN1, KRT15, LCN2, LOXL3, OPLAH, OSMR, PANX3, PCBD1, RAP1GAP, S100A8, SLC2A5, SPHK1, SPP1, STAC2, THRB, UNC5C, WNT6. TGFB1 and CXCL12 were added to the combined 15 gene set plus 25 gene set of HSF1-regulated genes because of their important role in HSF1 biology, resulting in a set of 42 genes that constitute a refined HSF1 stromal gene signature (Table D).

Table D: Refined HSF1 Tumor Stromal Signature Set (Refined HSF1-SSS Signature Set)

CDSN, COL4A5, MDFI, MFAP5, MXRA7, NID1, OLFML1, SLC16A3, SLC22A17, SLC2A10, SLC4A11, SLC6A2, TES, TUBB3, XPNPEP2, ADM, AK5, ANGPTL4, CHD7, COL11A1, FGFR3, GNAO1, ID4, ITLN1, KRT15, LCN2, LOXL3, OPLAH, OSMR, PANX3, PCBD1, RAP1GAP, S100A8, SLC2A5, SPHK1, SPP1, STAC2, THRB, UNC5C, WNT6, TGFB1, CXCL12.

### Example 11

### Identification of Cancer-Tumor Stroma Normalization Genes

We sought to identify a panel of genes whose expression level could be used to determine the proportion of cancer cells and stromal cells in a tumor sample, thereby allowing the expression level of the HSF1 cancer cell signature genes by cancer cells and the expression level of the HSF1 stromal cell signature genes by stromal cells to be derived from gene expression measurements made on a mixed population of cancer cells and stromal cells. Studies by others have measured gene expression profiles in breast cancer cells and tumor-associated stromal cells isolated from breast tumor samples using laser capture microdissection (Ma, X., et al., Breast Cancer Research 2009, 11:R7 (doi:10.1186/bcr2222), Winslow, S., et al. Breast Cancer Research (2015) 17:23). We analyzed the datasets described in Ma, et al. 2009, and Winslow, et al. 2015, to identify genes that were both (i) more highly expressed in cancer cells than in stromal cells (cancer:stroma expression level ratio >1) and (ii) exhibited low variance in cancer cell expression level within the set of tumors considered (variance <1). This analysis identified the following set of 8 genes, referred to as the Cancer High, Stroma Low set: DSG2, DSP, ELF3, IRF6, MYO5B, MYO6, PTPLB, TRPS1. In addition, we analyzed these datasets to identify genes that were both (i) more highly expressed in stromal cells than in cancer cells (stroma:cancer expression level ratio >2.48) and (ii) exhibited low variance in stromal expression level within the set of tumors considered (variance <1). This analysis identified the following set of 8 genes, referred to as the Stroma High, Cancer Low set: BGN, CFH, LTBP2, MRC1, PECAM1, SLCO2B1, TCF4, WIPF1.

Table E: Combined Cancer-Stroma Normalization Set

DSG2, DSP, ELF3, IRF6, MYO5B, MYO6, PTPLB, TRPS1, BGN, CFH, LTBP2, MRC1, PECAM1, SLCO2B1, TCF4, WIPF1

The Cancer High, Stroma Low and Stroma High, Cancer Low gene sets can be used to determine the proportion of cancer cells and tumor-associated stromal cells in a given tumor sample. For example, a set of samples can be prepared by mixing cancer cells and tumor-associated stromal cells in known proportions (e.g., ranging from 0% cancer cells, 100% tumor-associated stromal cells to 100% cancer cells, 0% tumor-associated stromal cells. The set may include about 5 to 50 samples, or more. Such cells may be obtained using laser capture microdissection, for example. The expression of the Cancer High, Stroma Low and Stroma High, Cancer Low gene sets in these samples is measured. The cancer:stroma ratios of the samples may be evenly distributed across the range in increments of, e.g., 2%, 5%, 7.5%, 10%, etc. (e.g., 5% cancer cells: 95% stromal cells, 10% cancer cells: 90% stromal cells, 15% cancer cells: 85% stromal cells, etc.) or may be unevenly distributed across the range. The precise distribution is not crucial so long as a sufficient number of samples with sufficiently varied ratios of cancer cells to tumor-associated stromal cells is analyzed, so that the relationship between the measured gene expression levels and the proportion of tumor-associated stromal cells and cancer cells can be determined. Once this relationship is ascertained, it can be applied to gene expression levels measured in a biological sample from a tumor to determine the proportion of tumor-associated stromal cells and cancer cells in that sample and/or to deconvolute a gene expression level of one or more genes of interest (e.g., one or more HSF1-regulated genes) into a component attributable to tumor stromal cells and a component attributable to cancer stromal cells and/or to normalize a gene expression level of one or more genes of interest (e.g., one or more HSF1-regulated genes) to indicate the expression level that would have been measured had the sample been composed entirely or cancer cells or entirely of tumor-associated stromal cells. One of ordinary skill in the art will appreciate that the analysis may be performed in a variety of ways. For example, clustering analysis, regression analysis, or other techniques known in the art may be applied.

### REFERENCES

Akerfelt, M., Morimoto, R. I., and Sistonen, L. (2010). Heat shock factors: integrators of cell stress, development and lifespan. Nat Rev Mol Cell Biol 11, 545-555.
Beck, A. H., Espinosa, I., Gilks, C. B., van de Rijn, M., and West, R. B. (2008). The fibromatosis signature defines a robust stromal response in breast carcinoma. Lab Invest 88, 591-601.
Bissell, M. J., and Hines, W. C. (2011). Why don't we get more cancer? A proposed role of the microenvironment in restraining cancer progression. Nat Med 17, 320-329.
Chang, H. Y., Sneddon, J. B., Alizadeh, A. A., Sood, R., West, R. B., Montgomery, K., Chi, J. T., van de Rijn, M., Botstein, D., and Brown, P. O. (2004). Gene expression signature of fibroblast serum response predicts human cancer progression: similarities between tumors and wounds. PLoS Biol 2, E7.
Chou, S. D., Prince, T., Gong, J., and Calderwood, S. K. (2012). mTOR is essential for the proteotoxic stress response, HSF1 activation and heat shock protein synthesis. Plos One 7, e39679.
Coussens, L. M., Zitvogel, L., and Palucka, A. K. (2013). Neutralizing tumor-promoting chronic inflammation: a magic bullet? Science 339, 286-291.
Dai, C., Santagata, S., Tang, Z., Shi, J., Cao, J., Kwon, H., Bronson, R. T., Whitesell, L., and Lindquist, S. (2012). Loss of tumor suppressor NFl activates HSF1 to promote carcinogenesis. J Clin Invest 122, 3742-3754.
Dai, C., Whitesell, L., Rogers, A. B., and Lindquist, S. (2007). Heat shock factor 1 is a powerful multifaceted modifier of carcinogenesis. Cell 130, 1005-1018.
Dias-Santagata, D., Akhavanfard, S., David, S. S., Vernovsky, K., Kuhlmann, G., Boisvert, S. L., Stubbs, H., McDermott, U., Settleman, J., Kwak, E. L., et al. (2010). Rapid targeted mutational analysis of human tumours: a clinical platform to guide personalized cancer medicine. Embo Mol Med 2, 146-158.
Dituri, F., Mazzocca, A., Peidro, F. J., Papappicco, P., Fabregat, I., De Santis, F., Paradiso, A., Sabba, C., and Giannelli, G. (2013). Differential Inhibition of the TGF-beta Signaling Pathway in HCC Cells Using the Small Molecule Inhibitor LY2157299 and the D10 Monoclonal Antibody against TGF-beta Receptor Type II. Plos One 8, e67109.
Dvorak, H. F. (1986). Tumors: wounds that do not heal. Similarities between tumor stroma generation and wound healing. N Engl J Med 315, 1650-1659.
Erez, N., Truitt, M., Olson, P., Arron, S. T., and Hanahan, D. (2010). Cancer-Associated Fibroblasts Are Activated in Incipient Neoplasia to Orchestrate Tumor-Promoting Inflammation in an NF-kappaB-Dependent Manner. Cancer Cell 17, 135-147.
Fang, F., Chang, R., and Yang, L. (2012). Heat shock factor 1 promotes invasion and metastasis of hepatocellular carcinoma in vitro and in vivo. Cancer 118, 1782-1794.
Finak, G., Bertos, N., Pepin, F., Sadekova, S., Souleimanova, M., Zhao, H., Chen, H., Omeroglu, G., Meterissian, S., Omeroglu, A., et al. (2008). Stromal gene expression predicts clinical outcome in breast cancer. Nat Med 14, 518-527.
Goldstraw, P., Crowley, J., Chansky, K., Giroux, D. J., Groome, P. A., Rami-Porta, R., Postmus, P. E., Rusch, V., and Sobin, L. (2007). The IASLC Lung Cancer Staging Project: proposals for the revision of the TNM stage groupings in the forthcoming (seventh) edition of the TNM Classification of malignant tumours. J Thorac Oncol 2, 706-714.
Hanahan, D., and Coussens, L. M. (2012). Accessories to the crime: functions of cells recruited to the tumor microenvironment. Cancer Cell 21, 309-322.
Hanahan, D., and Weinberg, R. A. (2011). Hallmarks of cancer: the next generation. Cell 144, 646-674.
Henderson, B., and Kaiser, F. (2013). Do reciprocal interactions between cell stress proteins and cytokines create a new intra-/extra-cellular signalling nexus? Cell Stress Chaperones.
Jin, X., Moskophidis, D., and Mivechi, N. F. (2011). Heat shock transcription factor 1 is a key determinant of HCC development by regulating hepatic steatosis and metabolic syndrome. Cell Metab 14, 91-103.
Kalluri, R., and Zeisberg, M. (2006). Fibroblasts in cancer. Nat Rev Cancer 6, 392-401.
Karnoub, A. E., Dash, A. B., Vo, A. P., Sullivan, A., Brooks, M. W., Bell, G. W., Richardson, A. L., Polyak, K., Tubo, R., and Weinberg, R. A. (2007). Mesenchymal stem cells within tumour stroma promote breast cancer metastasis. Nature 449, 557-563.
Kojima, Y., Acar, A., Eaton, E. N., Mellody, K. T., Scheel, C., Ben-Porath, I., Onder, T. T., Wang, Z. C., Richardson, A. L., Weinberg, R. A., et al. (2010). Autocrine TGF-beta and stromal cell-derived factor-1 (SDF-1) signaling drives the evolution of tumor-promoting mammary stromal myofibroblasts. Proc Natl Acad Sci USA 107, 20009-20014.
Lee, T. I., Johnstone, S. E., and Young, R. A. (2006). Chromatin immunoprecipitation and microarray-based analysis of protein location. Nat Protoc 1, 729-748.
Lu, P., Weaver, V. M., and Werb, Z. (2012). The extracellular matrix: a dynamic niche in cancer progression. J Cell Biol 196, 395-406.
Lujambio, A., Akkari, L., Simon, J., Grace, D., Tschaharganeh, D. F., Bolden, J. E., Zhao, Z., Thapar, V., Joyce, J. A., Krizhanovsky, V., et al. (2013). Non-cell-autonomous tumor suppression by p53. Cell 153, 449-460.
Luo, J., Solimini, N. L., and Elledge, S. J. (2009). Principles of cancer therapy: oncogene and non-oncogene addiction. Cell 136, 823-837.
Maity, T. K., Henry, M. M., Tulapurkar, M. E., Shah, N. G., Hasday, J. D., and Singh, I. S. (2011). Distinct, gene-specific effect of heat shock on heat shock factor-1 recruitment and gene expression of CXC chemokine genes. Cytokine 54, 61-67.
Mendillo, M. L., Santagata, S., Koeva, M., Bell, G. W., Hu, R., Tamimi, R. M., Fraenkel, E., Ince, T. A., Whitesell, L., and Lindquist, S. (2012). HSF1 drives a transcriptional program distinct from heat shock to support highly malignant human cancers. Cell 150, 549-562.
Meng, L., Gabai, V. L., and Sherman, M. Y. (2010). Heat-shock transcription factor HSF1 has a critical role in human epidermal growth factor receptor-2-induced cellular transformation and tumorigenesis. Oncogene 29, 5204-5213.
Min, J. N., Huang, L., Zimonjic, D. B., Moskophidis, D., and Mivechi, N. F. (2007). Selective suppression of lymphomas by functional loss of Hsf1 in a p53-deficient mouse model for spontaneous tumors. Oncogene 26, 5086-5097.
Morimoto, R. I. (2008). Proteotoxic stress and inducible chaperone networks in neurodegenerative disease and aging. Genes Dev 22, 1427-1438.
Moskovits, N., Kalinkovich, A., Bar, J., Lapidot, T., and Oren, M. (2006). p53 Attenuates cancer cell migration and invasion through repression of SDF-1/CXCL12 expression in stromal fibroblasts. Cancer Res 66, 10671-10676.
Newman, A. C., Nakatsu, M. N., Chou, W., Gershon, P. D., and Hughes, C. C. (2011). The requirement for fibroblasts in angiogenesis: fibroblast-derived matrix proteins are essential for endothelial cell lumen formation. Mol Biol Cell 22, 3791-3800.
Olumi, A. F., Grossfeld, G. D., Hayward, S. W., Carroll, P. R., Tlsty, T. D., and Cunha, G. R. (1999). Carcinoma-associated fibroblasts direct tumor progression of initiated human prostatic epithelium. Cancer Res 59, 5002-5011.
Orimo, A., Gupta, P. B., Sgroi, D. C., Arenzana-Seisdedos, F., Delaunay, T., Naeem, R., Carey, V. J., Richardson, A. L., and Weinberg, R. A. (2005). Stromal fibroblasts present in invasive human breast carcinomas promote tumor growth and angiogenesis through elevated SDF-1/CXCL12 secretion. Cell 121, 335-348.
Pelham, H. R. (1982). A regulatory upstream promoter element in the Drosophila hsp 70 heat-shock gene. Cell 30, 517-528.
Pickup, M., Novitskiy, S., and Moses, H. L. (2013). The roles of TGFbeta in the tumour microenvironment. Nat Rev Cancer 13, 788-799.
Pillai, R. N., and Ramalingam, S. S. (2014). Advances in the diagnosis and treatment of non-small cell lung cancer. Mol Cancer Ther 13, 557-564.
Place, A. E., Jin Huh, S., and Polyak, K. (2011). The microenvironment in breast cancer progression: biology and implications for treatment. Breast Cancer Res 13, 227.
Prahlad, V., Cornelius, T., and Morimoto, R. I. (2008). Regulation of the cellular heat shock response in Caenorhabditis elegans by thermosensory neurons. Science 320, 811-814.
Qiu, W., Hu, M., Sridhar, A., Opeskin, K., Fox, S., Shipitsin, M., Trivett, M., Thompson, E. R., Ramakrishna, M., Gorringe, K. L., et al. (2008). No evidence of clonal somatic genetic alterations in cancer-associated fibroblasts from human breast and ovarian carcinomas. Nat Genet 40, 650-655.
Quante, M., Tu, S. P., Tomita, H., Gonda, T., Wang, S. S., Takashi, S., Baik, G. H., Shibata, W., Diprete, B., Betz, K. S., et al. (2011). Bone marrow-derived myofibroblasts contribute to the mesenchymal stem cell niche and promote tumor growth. Cancer Cell 19, 257-272.
Raychaudhuri, S., Loew, C., Korner, R., Pinkert, S., Theis, M., Hayer-Hartl, M., Buchholz, F., and Hartl, F. U. (2014). Interplay of Acetyltransferase EP300 and the Proteasome System in Regulating Heat Shock Transcription Factor 1. Cell 156, 975-985.
Sakurai, H., and Enoki, Y. (2010). Novel aspects of heat shock factors: DNA recognition, chromatin modulation and gene expression. FEBS J 277, 4140-4149.
Santagata, S., Hu, R., Lin, N. U., Mendillo, M. L., Collins, L. C., Hankinson, S. E., Schnitt, S. J., Whitesell, L., Tamimi, R. M., Lindquist, S., et al. (2011). High levels of nuclear heat-shock factor 1 (HSF1) are associated with poor prognosis in breast cancer. Proc Natl Acad Sci USA 108, 18378-18383.
Santagata, S., Mendillo, M. L., Tang, Y. C., Subramanian, A., Perley, C. C., Roche, S. P., Wong, B., Narayan, R., Kwon, H., Koeva, M., et al. (2013). Tight coordination of protein translation and HSF1 activation supports the anabolic malignant state. Science 341, 1238303.
Santagata, S., Xu, Y. M., Wijeratne, E. M., Kontnik, R., Rooney, C., Perley, C. C., Kwon, H., Clardy, J., Kesari, S., Whitesell, L., et al. (2012). Using the heat-shock response to discover anticancer compounds that target protein homeostasis. Acs Chem Biol 7, 340-349.
Sasaki, H., Sato, T., Yamauchi, N., Okamoto, T., Kobayashi, D., Iyama, S., Kato, J., Matsunaga, T., Takimoto, R., Takayama, T., et al. (2002). Induction of heat shock protein 47 synthesis by TGF-beta and IL-1 beta via enhancement of the heat shock element binding activity of heat shock transcription factor 1. J Immunol 168, 5178-5183.
Saturno, G., Valenti, M., De Haven Brandon, A., Thomas, G. V., Eccles, S., Clarke, P. A., and Workman, P. (2013). Combining Trail with PI3 Kinase or HSP90 inhibitors enhances apoptosis in colorectal cancer cells via suppression of survival signaling. Oncotarget 4, 1185-1198.
Scott, K. L., Nogueira, C., Heffernan, T. P., van Doorm, R., Dhakal, S., Hanna, J. A., Min, C., Jaskelioff, M., Xiao, Y., Wu, C. J., et al. (2011). Proinvasion metastasis drivers in early-stage melanoma are oncogenes. Cancer Cell 20, 92-103.
Shamovsky, I., and Nudler, E. (2008). New insights into the mechanism of heat shock response activation. Cell Mol Life Sci 65, 855-861.
Sholl, L. M., Barletta, J. A., Yeap, B. Y., Chirieac, L. R., and Hornick, J. L. (2010). Sox2 protein expression is an independent poor prognostic indicator in stage I lung adenocarcinoma. Am J Surg Pathol 34, 1193-1198.
Siegel, P. M., and Massague, J. (2003). Cytostatic and apoptotic actions of TGF-beta in homeostasis and cancer. Nat Rev Cancer 3, 807-821.
Solimini, N. L., Luo, J., and Elledge, S. J. (2007). Non-oncogene addiction and the stress phenotype of cancer cells. Cell 130, 986-988.
Spaeth, E. L., Dembinski, J. L., Sasser, A. K., Watson, K., Klopp, A., Hall, B., Andreeff, M., and Marini, F. (2009). Mesenchymal stem cell transition to tumor-associated fibroblasts contributes to fibrovascular network expansion and tumor progression. Plos One 4, e4992.
Straussman, R., Morikawa, T., Shee, K., Barzily-Rokni, M., Qian, Z. R., Du, J., Davis, A., Mongare, M. M., Gould, J., Frederick, D. T., el al. (2012). Tumour micro-environment elicits innate resistance to RAF inhibitors through HGF secretion. Nature 487, 500-504.
Tomasek, J. J., Gabbiani, G., Hinz, B., Chaponnier, C., and Brown, R. A. (2002). Myofibroblasts and mechano-regulation of connective tissue remodelling. Nat Rev Mol Cell Biol 3, 349-363.
Trimboli, A. J., Cantemir-Stone, C. Z., Li, F., Wallace, J. A., Merchant, A., Creasap, N., Thompson, J. C., Caserta, E., Wang, H., Chong, J. L., el al. (2009). Pten in stromal fibroblasts suppresses mammary epithelial tumours. Nature 461, 1084-1091.
Wang, X., Khaleque, M. A., Zhao, M. J., Zhong, R., Gaestel, M., and Calderwood, S. K. (2006). Phosphorylation of HSF1 by MAPK-activated protein kinase 2 on serine 121, inhibits transcriptional activity and promotes HSP90 binding. J Biol Chem 281, 782-791.
Whitesell, L., and Lindquist, S. (2009). Inhibiting the transcription factor HSF1 as an anticancer strategy. Expert Opin Ther Targets 13, 469-478.
Wilson, T. R., Fridlyand, J., Yan, Y., Penuel, E., Burton, L., Chan, E., Peng, J., Lin, E., Wang, Y., Sosman, J., et al. (2012). Widespread potential for growth-factor-driven resistance to anticancer kinase inhibitors. Nature 487, 505-509.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more element(s), feature(s), or limitation(s) found in any other claim, e.g., any other claim that is dependent on the same base claim. Any one or more claims can be modified to explicitly exclude any one or more embodiment(s), element(s), feature(s), etc. For example, any particular type of tumor, tumor characteristic, or therapeutic agent, can be excluded from any one or more claims. Any one or more genes or combinations of genes can be excluded from any set of genes used in any of the methods described herein. Any one or more reagents for measuring expression of any one or more genes or combinations of genes can be excluded from any composition or kit described herein.

It should be understood that (i) any method of classification, assessment, diagnosis, prognosis, treatment-specific prediction, treatment selection, treatment, etc., can include a step of providing a sample, e.g., a sample obtained from a subject in need of classification, assessment, diagnosis, prognosis, treatment-specific prediction, treatment selection, or treatment for cancer, e.g., a tumor sample obtained from the subject; (ii) any method of classification, assessment, diagnosis, prognosis, treatment-specific prediction, treatment selection, treatment, etc., can include a step of providing a subject in need of classification, assessment, diagnosis, prognosis, treatment-specific prediction, treatment selection, or treatment for cancer.

Where the claims recite a method, certain aspects of the invention provide a product, e.g., a kit, agent, or composition, suitable for performing the method.

Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact. In some embodiments a request is fulfilled, e.g., a method or step is performed, in exchange for a fee or other consideration and/or pursuant to an agreement between a requestor and an individual or entity performing the method or step. It should also be understood that unless otherwise indicated or evident from the context, any product or composition described herein may be considered “isolated”. It should also be understood that, where applicable, unless otherwise indicated or evident from the context, any method or step of a method that may be amenable to being performed mentally or as a mental step or using a writing implement such as a pen or pencil, and a surface suitable for writing on, such as paper, may be expressly indicated as being performed at least in part, substantially, or entirely, by a machine, e.g., a computer, device (apparatus), or system, which may, in some embodiments, be specially adapted or designed to be capable of performing such method or step or a portion thereof.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. It will be understood that any description or exemplification of a term anywhere herein may be applied wherever such term appears herein (e.g., in any aspect or embodiment in which such term is relevant) unless indicated or clearly evident otherwise.

TABLE S1
List of genes differentially expressed in cancer cells upon coculture with MEFs (FIG. 3C-D)

| GeneName | T_wWTF_A | T_wWTF_B | T_wKOF_A | T_wKOF_B | VENN GROUP |
|---|---|---|---|---|---|
| Ccl3 | −6.0717335 | −5.656696 | −6.166608 | −5.8526799 | overlap (group b) |
| ENSMUST00000121047 | −4.1599208 | −4.1675228 | −3.801389 | −4.3897531 | overlap (group b) |
| Olfr286 | −3.8147001 | −3.8618275 | −3.698263 | −3.6900611 | overlap (group b) |
| ENSMUST00000108691 | −3.7098672 | −3.4700757 | −3.751978 | −3.436985 | overlap (group b) |
| ENSMUST00000098926 | −2.834851 | −2.8120283 | −2.893539 | −2.9600832 | overlap (group b) |
| A730082K24Rik | −2.8399778 | −2.7484296 | −2.68299 | −2.8944941 | overlap (group b) |
| Six3 | −2.5184897 | −2.5530725 | −2.627577 | −2.6234243 | overlap (group b) |
| 1700058M13Rik | −2.3382683 | −2.5329158 | −2.236141 | −2.5418489 | overlap (group b) |
| Pex11b | −2.4000229 | −2.3888193 | −2.249536 | −2.4523519 | overlap (group b) |
| Gm3235 | −2.435208 | −1.9817064 | −2.233508 | −2.1958312 | overlap (group b) |
| Pcdha9 | −2.0141867 | −2.0703006 | −2.598782 | −2.1350553 | overlap (group b) |
| chr7: 39351858-46480344_R | −2.3465575 | −2.3003069 | −2.033649 | −2.0703013 | overlap (group b) |
| Vmn2r11 | −1.9436648 | −2.4168512 | −2.179906 | −2.1628945 | overlap (group b) |
| ENSMUST00000079376 | −1.8675754 | −2.2839875 | −2.109333 | −2.2580906 | overlap (group b) |
| Stxbp5l | −1.8848028 | −1.9626358 | −1.744271 | −1.8441381 | overlap (group b) |
| Macc1 | −2.0697244 | −1.8658962 | −1.946507 | −1.5500066 | overlap (group b) |
| Hoxd1 | −1.8744268 | −1.9004555 | −1.686395 | −1.7475146 | overlap (group b) |
| ENSMUST00000106901 | −1.743679 | −1.7011743 | −2.092619 | −1.5412813 | overlap (group b) |
| Edn1 | −1.9983584 | −1.6315394 | −1.656715 | −1.7765086 | overlap (group b) |
| Crot | −2.0735513 | −1.9414064 | −1.354497 | −1.5341684 | overlap (group b) |
| 4930570N19Rik | −1.7537292 | −1.6878852 | −1.676938 | −1.6583622 | overlap (group b) |
| ENSMUST00000054973 | −1.747442 | −1.7131314 | −1.708054 | −1.5912783 | overlap (group b) |
| Cldn3 | −1.5946683 | −1.3491817 | −1.468511 | −2.2578043 | overlap (group b) |
| Rogdi | −1.5056174 | −1.5672938 | −1.624413 | −1.8675698 | overlap (group b) |
| Kctd8 | −1.6220208 | −1.4788112 | −1.716528 | −1.7401209 | overlap (group b) |
| chr13: 24833082-24838462_F | −1.5263164 | −1.5210026 | −1.680264 | −1.8011105 | overlap (group b) |
| ENSMUST00000098691 | −1.6667871 | −1.6241655 | −1.672645 | −1.5457448 | overlap (group b) |
| chr15: 101088974-101089576_I | −1.5674321 | −1.678277 | −1.369831 | −1.8847651 | overlap (group b) |
| Lyve1 | −1.484621 | −1.5430934 | −1.756446 | −1.6714991 | overlap (group b) |
| chr12: 12768528-12849976_R | −1.6993862 | −1.6879858 | −1.478367 | −1.5614905 | overlap (group b) |
| Gm2586 | −1.7082847 | −1.4649833 | −1.466467 | −1.6736029 | overlap (group b) |
| Defb12 | −1.62949 | −1.6051255 | −1.536052 | −1.4576162 | overlap (group b) |
| 2900064F13Rik | −1.4866545 | −1.3383418 | −1.751797 | −1.6372931 | overlap (group b) |
| ENSMUST00000106832 | −1.2135581 | −1.3412571 | −1.697641 | −1.9460496 | overlap (group b) |
| Sall4 | −1.6295137 | −1.4370608 | −1.562365 | −1.5585003 | overlap (group b) |
| C78660 | −1.5672273 | −1.5278189 | −1.607742 | −1.4210066 | overlap (group b) |
| BC005764 | −1.4646861 | −1.3952904 | −1.620897 | −1.6139322 | overlap (group b) |
| Mfsd2a | −1.7901028 | −1.5041742 | −1.153229 | −1.6272954 | overlap (group b) |
| Gjc3 | −1.5389364 | −1.2136627 | −1.641808 | −1.6188675 | overlap (group b) |
| ENSMUST00000035860 | −1.3593614 | −1.7323822 | −1.435608 | −1.4575224 | overlap (group b) |
| AI852580 | −1.3926812 | −1.5413558 | −1.408553 | −1.6344069 | overlap (group b) |
| F2rl1 | −1.5454702 | −1.6887363 | −1.225515 | −1.4501033 | overlap (group b) |
| Ankrd1 | −2.0736153 | −1.8244138 | −0.986262 | −1.012353 | overlap (group b) |
| Hs3st3a1 | −1.571418 | −1.3762012 | −1.25399 | −1.6836229 | overlap (group b) |
| Rarb | −1.6423822 | −1.7138054 | −1.176119 | −1.3446893 | overlap (group b) |
| 4930577H14Rik | −1.2591121 | −1.3713579 | −1.75346 | −1.4773931 | overlap (group b) |
| chr5: 35975265-35982561_F | −0.437938 | −1.939671 | −1.362307 | −2.0854339 | overlap (group b) |
| 4930543E12Rik | −1.2628506 | −1.4492147 | −1.55141 | −1.4203919 | overlap (group b) |
| B9d2 | −1.474971 | −1.4035009 | −1.407761 | −1.3975757 | overlap (group b) |
| ENSMUST00000112310 | −1.315314 | −1.2531923 | −1.494829 | −1.617896 | overlap (group b) |
| ENSMUST00000101487 | −1.874656 | −1.3649237 | −1.055948 | −1.3706992 | overlap (group b) |
| Nr2e1 | −1.3652453 | −1.6030208 | −1.361503 | −1.301115 | overlap (group b) |
| Clca4 | −1.5670633 | −1.1405251 | −1.212915 | −1.6896961 | overlap (group b) |
| 4921509O07Rik | −1.5110173 | −1.3923728 | −1.416737 | −1.2875546 | overlap (group b) |
| ENSMUST00000090806 | −1.5719877 | −1.1200468 | −1.307634 | −1.5749613 | overlap (group b) |
| Olfr1318 | −1.5547926 | −1.7706273 | −0.512691 | −1.6800247 | overlap (group b) |
| LOC633965 | −1.1708187 | −0.9799562 | −1.647848 | −1.6973799 | overlap (group b) |
| Olfr1484 | −1.485099 | −0.9507626 | −1.399263 | −1.6001157 | overlap (group b) |
| 4921510H08Rik | −1.4388609 | −1.13019 | −1.360765 | −1.4933773 | overlap (group b) |
| Nanos2 | −1.2424777 | −1.4781976 | −1.340998 | −1.3562976 | overlap (group b) |
| Ttf1 | −1.4125874 | −1.4039329 | −1.118354 | −1.4584471 | overlap (group b) |
| ENSMUST00000024228 | −1.3428005 | −1.2454226 | −1.344923 | −1.4512256 | overlap (group b) |
| Snora74a | −0.9505969 | −1.0467793 | −1.312939 | −2.0669965 | overlap (group b) |
| AA414993 | −1.3103556 | −1.28606 | −1.407217 | −1.3602098 | overlap (group b) |
| LOC100046350 | −1.2252124 | −1.4687695 | −1.22677 | −1.428793 | overlap (group b) |
| LOC635967 | −1.4986595 | −1.369846 | −1.500304 | −0.9718149 | overlap (group b) |
| ENSMUST00000085433 | −1.229421 | −1.2066164 | −1.471782 | −1.4153214 | overlap (group b) |
| 4833427F10Rik | −1.1486616 | −1.5999113 | −1.372702 | −1.1916447 | overlap (group b) |
| Gm3840 | −1.0762234 | −1.5321332 | −1.425613 | −1.2486693 | overlap (group b) |
| chr8: 26866152-26872502_F | −1.6130588 | −1.5215106 | −1.029881 | −1.1169716 | overlap (group b) |
| Amica1 | −1.1754767 | −1.3124339 | −0.942478 | −1.8416745 | overlap (group b) |
| LOC100048662 | −0.5489915 | −1.3551546 | −1.636653 | −1.7219003 | overlap (group b) |
| chr8: 122807653-122823000_R | −1.3061177 | −1.4245776 | −1.269356 | −1.2536327 | overlap (group b) |
| Trav10d | −1.4150347 | −1.3234865 | −1.133983 | −1.3763259 | overlap (group b) |
| Serpine1 | −1.6887658 | −1.5643021 | −1.055555 | −0.9338809 | overlap (group b) |
| LOC100047979 | −1.2143393 | −1.4826414 | −1.379548 | −1.1626937 | overlap (group b) |
| Tbxas1 | −1.599031 | −1.4932233 | −1.221613 | −0.9174922 | overlap (group b) |
| Gpx5 | −1.3377939 | −1.0172148 | −1.262277 | −1.6136945 | overlap (group b) |
| ENSMUST00000051440 | −1.2782588 | −1.3253862 | −1.325386 | −1.2854098 | overlap (group b) |
| ENSMUST00000093246 | −0.9069555 | −1.1599416 | −1.425597 | −1.6867809 | overlap (group b) |
| Vmn1r89 | −1.2076526 | −1.2197623 | −1.372994 | −1.3736091 | overlap (group b) |
| 1700037F03Rik | −1.1397497 | −1.2180946 | −1.264371 | −1.5514532 | overlap (group b) |
| ENSMUST00000114137 | −1.4647313 | −1.0042754 | −1.436825 | −1.2335102 | overlap (group b) |
| Poteg | −1.3388199 | −0.6411636 | −1.478461 | −1.6708476 | overlap (group b) |
| Gm11826 | −1.3277431 | −1.2493165 | −1.263379 | −1.2814778 | overlap (group b) |
| ENSMUST00000120588 | −1.2417071 | −1.1317259 | −1.447736 | −1.2995346 | overlap (group b) |
| chr12: 116850878-116855932_I | −1.3679422 | −1.2290089 | −1.269237 | −1.2538261 | overlap (group b) |
| LOC100045853 | −1.404782 | −1.478486 | −1.141513 | −1.0817273 | overlap (group b) |
| Pilra | −0.8654935 | −0.9625753 | −1.810981 | −1.4610712 | overlap (group b) |
| Olfr167 | −1.1053092 | −1.2528875 | −1.339208 | −1.4003275 | overlap (group b) |
| Opn3 | −1.7603665 | −0.6085984 | −1.459792 | −1.2228996 | overlap (group b) |
| Pcnxl2 | −1.199235 | −1.199235 | −1.488742 | −1.1547525 | overlap (group b) |
| Hist1h2ac | −0.2227083 | −1.972218 | −1.42395 | −1.4004914 | overlap (group b) |
| BC019943 | −1.2791628 | −1.2355203 | −1.145528 | −1.353311 | overlap (group b) |
| chr12: 16447157-16447657_F | −1.235963 | −1.2512439 | −1.161954 | −1.3569462 | overlap (group b) |
| Kcnmb1 | −1.2667708 | −1.332903 | −0.97514 | −1.4281257 | overlap (group b) |
| 0610040F04Rik | −1.1664727 | −1.3674427 | −1.191593 | −1.2768401 | overlap (group b) |
| ENSMUST00000030185 | −1.1222597 | −1.220224 | −1.319553 | −1.3396818 | overlap (group b) |
| Olfr473 | −1.3965106 | −1.6581973 | −0.468207 | −1.4690926 | overlap (group b) |
| Cpeb2 | −1.5860512 | −1.2009729 | −0.855496 | −1.32981 | overlap (group b) |
| ENSMUST00000018719 | −1.2201364 | −1.4685923 | −0.956867 | −1.3027643 | overlap (group b) |
| ENSMUST00000054846 | −1.1417762 | −1.4007254 | −0.980299 | −1.4108395 | overlap (group b) |
| Triml2 | −1.5320711 | −1.4597367 | −0.505819 | −1.4326363 | overlap (group b) |
| XM_001474671 | −0.7947665 | −1.5216901 | −1.245512 | −1.3663551 | overlap (group b) |
| Bhlha15 | −1.3329798 | −0.9797409 | −1.230743 | −1.3795009 | overlap (group b) |
| 1810019N24Rik | −0.2730294 | −1.2425191 | −1.414646 | −1.9439547 | overlap (group b) |
| chr1: 122655019-122660142_F | −1.7381308 | −1.641531 | −0.594319 | −0.8957987 | overlap (group b) |
| LOC673748 | −1.2844846 | −0.9248897 | −1.331612 | −1.3242607 | overlap (group b) |
| ENSMUST00000055830 | −1.083469 | −1.2020852 | −1.564348 | −1.0101121 | overlap (group b) |
| 1700003G18Rik | −0.8361826 | −1.1607608 | −1.238722 | −1.623235 | overlap (group b) |
| Gsdma2 | −1.208089 | −1.2408255 | −1.321435 | −1.088076 | overlap (group b) |
| Vmn2r15 | −1.0823536 | −1.3257311 | −1.215004 | −1.2264695 | overlap (group b) |
| Slc47a1 | −0.9995982 | −0.7400395 | −1.474895 | −1.6234486 | overlap (group b) |
| Chrnb1 | −1.3085684 | −1.1029976 | −1.007421 | −1.3900042 | overlap (group b) |
| ENSMUST00000110063 | −1.1000209 | −0.9901092 | −1.151131 | −1.5540191 | overlap (group b) |
| AI845619 | −1.282485 | −0.9350975 | −1.383427 | −1.1872679 | overlap (group b) |
| Ddah1 | −1.37723 | −1.3078422 | −0.945459 | −1.1564374 | overlap (group b) |
| ENSMUST00000103658 | −1.0090346 | −1.1259118 | −1.293433 | −1.3585726 | overlap (group b) |
| 1700121C10Rik | −0.9599393 | −0.7014939 | −1.558305 | −1.5466634 | overlap (group b) |
| Nipal1 | −1.2223548 | −0.9979967 | −1.156319 | −1.3794524 | overlap (group b) |
| S100a7a | −1.6063921 | −1.2921144 | −0.494065 | −1.3432392 | overlap (group b) |
| Cacna1f | −1.2109969 | −1.0554627 | −1.145231 | −1.3199313 | overlap (group b) |
| Olfr1065 | −1.4530663 | −1.5816807 | −0.366548 | −1.3284273 | overlap (group b) |
| Gm3693 | −1.2501224 | −1.2019389 | −1.186229 | −1.0727561 | overlap (group b) |
| P2ry12 | −1.6767571 | −1.3097306 | −0.595744 | −1.1211503 | overlap (group b) |
| Cd300lb | −1.5146814 | −1.2776098 | −0.539104 | −1.35313 | overlap (group b) |
| LOC100045340 | −1.0195974 | −1.2869448 | −1.073217 | −1.2970589 | overlap (group b) |
| Gm5793 | −1.142144 | −1.2675549 | −1.172827 | −1.0801386 | overlap (group b) |
| Reg3a | −1.2526391 | −1.1932062 | −1.256014 | −0.9593072 | overlap (group b) |
| Lpin3 | −0.9139308 | −1.0982323 | −1.256227 | −1.3881321 | overlap (group b) |
| Tmem171 | −1.1921936 | −0.8778818 | −0.851758 | −1.7213212 | overlap (group b) |
| E030011O05Rik | −1.3592753 | −1.3271098 | −1.039653 | −0.9148915 | overlap (group b) |
| Pcdhb11 | −1.1244819 | −1.2600695 | −1.142164 | −1.1133414 | overlap (group b) |
| Gm4614 | −1.0869817 | −1.1671995 | −1.301372 | −1.0655592 | overlap (group b) |
| Gm3925 | −1.1675933 | −1.3016318 | −1.207005 | −0.9441759 | overlap (group b) |
| Fam55b | −0.8675515 | −1.0875414 | −1.501779 | −1.1626795 | overlap (group b) |
| ENSMUST00000097608 | −1.0539473 | −1.1415736 | −1.306296 | −1.0856852 | overlap (group b) |
| Cst8 | −1.2440279 | −0.8829435 | −1.137471 | −1.321585 | overlap (group b) |
| ENSMUST00000126399 | −1.0867099 | −1.2222975 | −1.185903 | −1.0755694 | overlap (group b) |
| 5031415H12Rik | −0.8251915 | −1.4692002 | −0.82615 | −1.4432925 | overlap (group b) |
| Gm6282 | −1.2476722 | −0.8944334 | −1.221747 | −1.1988062 | overlap (group b) |
| Psg19 | −1.3410604 | −0.9268523 | −1.132474 | −1.1585386 | overlap (group b) |
| ENSMUST00000103607 | −1.0373957 | −1.0379777 | −1.174695 | −1.3036817 | overlap (group b) |
| LOC629952 | −1.0394995 | −1.1533147 | −0.879586 | −1.4811404 | overlap (group b) |
| Nup210 | −1.3725021 | −1.3710521 | −0.971811 | −0.8289611 | overlap (group b) |
| Hist1h3a | −0.6147697 | −1.306523 | −1.046818 | −1.5742203 | overlap (group b) |
| Gm6890 | −1.2361667 | −1.0166965 | −1.086823 | −1.2018393 | overlap (group b) |
| Sorbs2 | −1.3122026 | −0.7615607 | −1.154366 | −1.312862 | overlap (group b) |
| chr3: 57414750-57427825_F | −1.1636633 | −1.2220462 | −1.263053 | −0.887407 | overlap (group b) |
| Olfr275 | −0.9866628 | −1.1529892 | −1.136905 | −1.2551844 | overlap (group b) |
| Lamc3 | −1.451009 | −1.478697 | −0.916882 | −0.682654 | overlap (group b) |
| Mir17hg | −0.8986977 | −0.5654764 | −1.193088 | −1.8651765 | overlap (group b) |
| Acoxl | −1.1241449 | −0.8239996 | −1.217381 | −1.3522982 | overlap (group b) |
| ENSMUST00000103553 | −1.0197558 | −1.2474454 | −1.039655 | −1.2048424 | overlap (group b) |
| Tas2r143 | −0.9090407 | −1.3220467 | −1.133694 | −1.144822 | overlap (group b) |
| ENSMUST00000115382 | −1.1312288 | −1.0292531 | −1.314245 | −1.0161852 | overlap (group b) |
| Dusp9 | −1.2913617 | −1.1401364 | −0.811845 | −1.2449983 | overlap (group b) |
| Srgap3 | −1.5460769 | −1.3725883 | −0.6429 | −0.9088261 | overlap (group b) |
| Gm3624 | −1.0923715 | −1.0166374 | −1.097165 | −1.2529065 | overlap (group b) |
| F3 | −1.5747206 | −1.2905882 | −0.881385 | −0.7121615 | overlap (group b) |
| Tfrc | −1.1259389 | −0.9999217 | −1.025275 | −1.2833679 | overlap (group b) |
| Lyz2 | −1.017388 | −0.7213481 | −1.290976 | −1.4046538 | overlap (group b) |
| Pfpl | −1.3159322 | −1.1301156 | −0.981043 | −0.9991604 | overlap (group b) |
| Zfp735 | −0.9463158 | −1.3368436 | −0.91135 | −1.2222164 | overlap (group b) |
| LOC100044651 | −0.8288045 | −1.0137387 | −1.032314 | −1.529945 | overlap (group b) |
| Atp4b | −1.1579163 | −1.0154098 | −1.066238 | −1.1537617 | overlap (group b) |
| ENSMUST00000107887 | −1.5329263 | −1.3583676 | −0.408333 | −1.0849319 | overlap (group b) |
| Phox2b | −1.1163442 | −0.9207377 | −1.106496 | −1.2357794 | overlap (group b) |
| ENSMUST00000110869 | −1.2423342 | −1.0053794 | −0.9993 | −1.126247 | overlap (group b) |
| Atp1b1 | −1.318955 | −1.472151 | −0.883207 | −0.6956761 | overlap (group b) |
| Gm8347 | −1.133242 | −1.0410922 | −0.963999 | −1.2251241 | overlap (group b) |
| Dnajc27 | −1.1113567 | −0.7533444 | −1.119929 | −1.3756025 | overlap (group b) |
| chr1: 43240750-43252000_R | −0.9576329 | −1.0398514 | −1.083629 | −1.2773893 | overlap (group b) |
| ENSMUST00000063311 | −1.1026066 | −1.3088655 | −0.688818 | −1.2427334 | overlap (group b) |
| Vmn1r101 | −0.9762546 | −1.1315896 | −1.158242 | −1.0757011 | overlap (group b) |
| 6230415J03Rik | −0.3846959 | −0.8988717 | −1.029243 | −2.0272284 | overlap (group b) |
| LOC100046661 | −1.1754748 | −1.135487 | −1.254786 | −0.750336 | overlap (group b) |
| Gyk | −1.0947491 | −0.9877543 | −0.948584 | −1.2749274 | overlap (group b) |
| Gm6818 | −1.167532 | −1.1447092 | −1.038718 | −0.9498419 | overlap (group b) |
| Cnga3 | −1.1965359 | −1.1278974 | −0.987212 | −0.9803191 | overlap (group b) |
| Gm11527 | −0.6003292 | −1.206748 | −1.251406 | −1.231839 | overlap (group b) |
| Pdxk-ps | −1.0408276 | −0.8978945 | −1.017256 | −1.3305665 | overlap (group b) |
| 4930528F23Rik | −1.16896 | −1.0759332 | −1.131477 | −0.901749 | overlap (group b) |
| Mycl1 | −1.0029085 | −0.8235168 | −0.9122 | −1.5373051 | overlap (group b) |
| Sufu | −1.092396 | −1.0683859 | −0.994682 | −1.0928595 | overlap (group b) |
| ENSMUST00000109290 | −1.0659827 | −0.8349301 | −1.157251 | −1.1809993 | overlap (group b) |
| Ace3 | −1.180152 | −0.9818983 | −1.122076 | −0.9449717 | overlap (group b) |
| LOC100046290 | −1.1206806 | −0.8885846 | −1.084939 | −1.132794 | overlap (group b) |
| Gm13111 | −1.1191613 | −1.020957 | −1.161515 | −0.9242496 | overlap (group b) |
| Mrgpra7 | −0.9697322 | −1.1358211 | −0.953648 | −1.1651526 | overlap (group b) |
| Clec14a | −1.1247062 | −1.0087993 | −0.933209 | −1.1473479 | overlap (group b) |
| Pnmal1 | −1.0034837 | −1.0318933 | −0.953147 | −1.2176421 | overlap (group b) |
| Olfr1155 | −1.0255443 | −1.2532117 | −1.046761 | −0.8788162 | overlap (group b) |
| Rap2c | −1.0515631 | −0.8891809 | −1.228667 | −1.0325914 | overlap (group b) |
| TC1677549 | −1.0504463 | −1.1362825 | −1.050446 | −0.9645161 | overlap (group b) |
| Gm9638 | −1.1532167 | −0.9256254 | −1.034938 | −1.0853925 | overlap (group b) |
| chr10: 81111250-81130625_R | −0.6024109 | −1.2283538 | −1.089092 | −1.27756 | overlap (group b) |
| LOC630837 | −1.2289511 | −0.9745184 | −1.055008 | −0.9382322 | overlap (group b) |
| ENSMUST00000026614 | −1.1272736 | −0.8455635 | −1.067324 | −1.1560691 | overlap (group b) |
| ENSMUST00000112339 | −1.1631516 | −0.9359269 | −1.124443 | −0.9682791 | overlap (group b) |
| Gm3510 | −0.8503028 | −0.9615259 | −1.30475 | −1.0634055 | overlap (group b) |
| Cyp2c44 | −1.3696104 | −1.381963 | −0.908995 | −0.5172789 | overlap (group b) |
| Triml1 | −1.1250223 | −1.2752432 | −0.968035 | −0.8063077 | overlap (group b) |
| chr12: 4554378-4560302_F | −1.2019897 | −0.8698213 | −0.994354 | −1.1082628 | overlap (group b) |
| Gm8970 | −1.0371948 | −1.0972819 | −0.817759 | −1.2152949 | overlap (group b) |
| 4930550C14Rik | −1.0158334 | −1.0444922 | −1.248718 | −0.8562336 | overlap (group b) |
| Ccl6 | −1.5732118 | −1.0849352 | −0.670254 | −0.8292743 | overlap (group b) |
| C430017C20Rik | −0.9399238 | −0.9848788 | −1.287195 | −0.9447169 | overlap (group b) |
| Cyp19a1 | −0.9227975 | −0.9733689 | −1.134905 | −1.1146782 | overlap (group b) |
| ENSMUST00000088356 | −1.0517483 | −1.4793991 | −0.472438 | −1.1393013 | overlap (group b) |
| Olfr1087 | −1.1171818 | −1.1715562 | −0.864348 | −0.9826266 | overlap (group b) |
| Serpinb6e | −1.0102254 | −1.1756032 | −0.835249 | −1.1110629 | overlap (group b) |
| Vmn2r13 | −1.2600688 | −0.9356193 | −0.66046 | −1.2738039 | overlap (group b) |
| Olfr1137 | −1.1603344 | −0.9331097 | −1.082238 | −0.9468862 | overlap (group b) |
| chr14: 115443612-115445950_I | −0.8324982 | −0.5009698 | −1.064665 | −1.7231829 | overlap (group b) |
| Fam115c | −0.9651235 | −1.1338053 | −1.063644 | −0.9566203 | overlap (group b) |
| Mlph | −1.0679715 | −0.842248 | −1.241402 | −0.9658443 | overlap (group b) |
| Vmn1r15 | −0.9567724 | −1.1624861 | −0.886611 | −1.1064696 | overlap (group b) |
| ENSMUST00000097454 | −0.7964109 | −0.9067274 | −1.106036 | −1.3011036 | overlap (group b) |
| ENSMUST00000111753 | −1.0863394 | −1.0309696 | −0.942661 | −1.0373744 | overlap (group b) |
| Usp44 | −1.0764535 | −0.7681573 | −1.147521 | −1.093895 | overlap (group b) |
| ENSMUST00000118036 | −1.2591577 | −1.1142547 | −0.819882 | −0.8913153 | overlap (group b) |
| chr18: 35973241-35973632_F | −1.1706961 | −0.8690231 | −0.880428 | −1.1521185 | overlap (group b) |
| 8430422H06Rik | −1.1687966 | −1.0700785 | −0.933616 | −0.8925403 | overlap (group b) |
| 1700008K24Rik | −1.1376368 | −1.1156105 | −0.803626 | −1.0061993 | overlap (group b) |
| 4832428D23Rik | −0.9598856 | −1.0848585 | −1.09831 | −0.9190305 | overlap (group b) |
| Olfr583 | −0.9934975 | −0.8958689 | −1.193512 | −0.9521674 | overlap (group b) |
| ENSMUST00000097665 | −0.7311556 | −0.9136507 | −1.229076 | −1.1536531 | overlap (group b) |
| ENSMUST00000103740 | −1.3542044 | −1.0429307 | −0.769564 | −0.8574488 | overlap (group b) |
| Fcer1a | −1.1814237 | −0.86495 | −0.983345 | −0.986512 | overlap (group b) |
| ENSMUST00000087544 | −0.4270065 | −0.8785735 | −1.434127 | −1.2737505 | overlap (group b) |
| LOC100045379 | −0.9708069 | −0.9021684 | −1.090684 | −1.0496084 | overlap (group b) |
| Fam132b | −1.1155929 | −0.6230214 | −1.013823 | −1.2607059 | overlap (group b) |
| Hsbp1l1 | −1.1591144 | −1.4669148 | −0.196785 | −1.1836598 | overlap (group b) |
| Polr3g | −0.7043692 | −0.6618381 | −1.040809 | −1.5948457 | overlap (group b) |
| Insrr | 0.45840492 | 0.61497745 | 1.6337516 | 1.2973589 | overlap (group b) |
| Peli3 | 1.09469174 | 1.08570019 | 0.8605584 | 0.9736708 | overlap (group b) |
| Pam | 0.76021444 | 1.46127337 | 0.5218787 | 1.2728347 | overlap (group b) |
| Col5a3 | 0.96635217 | 0.91882323 | 0.816882 | 1.3149856 | overlap (group b) |
| Gm5077 | 1.2866095 | 0.97940601 | 0.7148248 | 1.041914 | overlap (group b) |
| Il17re | 0.99095428 | 0.82421349 | 0.5678546 | 1.6410465 | overlap (group b) |
| Vsig8 | 1.40116435 | 1.19681167 | 0.6952617 | 0.7314711 | overlap (group b) |
| Gzmm | 0.53631797 | 1.15265396 | 0.9693544 | 1.370363 | overlap (group b) |
| Ndrg4 | 1.42034552 | 1.14177543 | 0.9965914 | 0.4740929 | overlap (group b) |
| Tm6sf1 | 1.1976404 | 0.69977947 | 0.9089887 | 1.22778 | overlap (group b) |
| Lman1l | 0.95039213 | 0.69213266 | 0.8853631 | 1.5151484 | overlap (group b) |
| Sult2b1 | 1.0442924 | 0.5419409 | 1.139499 | 1.3254611 | overlap (group b) |
| Tceal1 | 0.34877171 | 0.88450885 | 0.8854108 | 1.9527914 | overlap (group b) |
| Ust | 0.57883828 | 0.59497181 | 1.4726909 | 1.4255866 | overlap (group b) |
| Slurp1 | 1.22356651 | 0.66906641 | 1.1011048 | 1.0821839 | overlap (group b) |
| Mfap5 | 1.13018253 | 0.97816337 | 0.7902082 | 1.177732 | overlap (group b) |
| Apoc1 | 1.41618434 | 1.10331948 | 0.6444799 | 0.921822 | overlap (group b) |
| Kifc2 | 1.18389572 | 0.93079269 | 0.9641225 | 1.0087886 | overlap (group b) |
| chr8: 108120629-108123201_F | 0.71762187 | 0.82286696 | 0.9550723 | 1.5956066 | overlap (group b) |
| Slc23a3 | 0.9362645 | 0.90873856 | 0.8619917 | 1.3874246 | overlap (group b) |
| Pbxip1 | 1.00092645 | 0.85393173 | 0.8316521 | 1.4165556 | overlap (group b) |
| ENSMUST00000066077 | 1.05720095 | 1.61666091 | 0.6582372 | 0.7754514 | overlap (group b) |
| Exd1 | 0.48562776 | 0.64832505 | 1.1541769 | 1.820612 | overlap (group b) |
| Rbm43 | 0.76792781 | 0.59079109 | 1.2271331 | 1.5248877 | overlap (group b) |
| Klf10 | 0.98473651 | 0.96019825 | 0.9152594 | 1.2516558 | overlap (group b) |
| Id4 | 1.24279193 | 1.41922904 | 0.7925316 | 0.6592434 | overlap (group b) |
| Gpr146 | 0.86893304 | 0.63193793 | 1.142913 | 1.4724801 | overlap (group b) |
| Ch25h | 0.58763262 | 0.83934326 | 0.8402452 | 1.8648309 | overlap (group b) |
| Klra2 | 0.70232781 | 0.77429472 | 1.1774581 | 1.4779939 | overlap (group b) |
| 2410066E13Rik | 0.85167197 | 0.88373832 | 1.3625274 | 1.0472195 | overlap (group b) |
| Pgbd5 | 1.24445346 | 1.25021601 | 0.382969 | 1.2746552 | overlap (group b) |
| Fst | 0.64652629 | 0.64704937 | 0.9043246 | 1.9573513 | overlap (group b) |
| Hbp1 | 1.15025437 | 0.91361231 | 0.9851537 | 1.1106493 | overlap (group b) |
| Ap3m2 | 0.65426185 | 0.77639504 | 1.2266564 | 1.5042312 | overlap (group b) |
| D2Ertd295e | 0.57569639 | 0.9775317 | 1.381103 | 1.227242 | overlap (group b) |
| chr2: 115900355-115900904_F | 0.89075262 | 0.68729875 | 1.0610421 | 1.5235297 | overlap (group b) |
| Pros1 | 1.09881047 | 1.44530505 | 0.4839539 | 1.1346662 | overlap (group b) |
| Gm12824 | 1.15496254 | 1.23050294 | 0.4219248 | 1.3696695 | overlap (group b) |
| BC022960 | 1.27439447 | 1.41583584 | 0.7154654 | 0.7955506 | overlap (group b) |
| Chn1 | 0.59952412 | 0.93361225 | 1.1239967 | 1.546539 | overlap (group b) |
| Hsd3b7 | 1.19430336 | 0.95778963 | 1.0418335 | 1.0130327 | overlap (group b) |
| chr10: 120058696-120076196_I | 1.49781181 | 1.13275191 | 0.7007824 | 0.8796669 | overlap (group b) |
| Fam71e1 | 1.10693942 | 1.34187929 | 0.6848963 | 1.078307 | overlap (group b) |
| Magee1 | 0.50486369 | 0.69008371 | 1.0380331 | 1.9925137 | overlap (group b) |
| 4933407E14Rik | 0.70249133 | 0.82275639 | 1.1203971 | 1.5849922 | overlap (group b) |
| C030039L03Rik | 1.41584145 | 1.1804506 | 0.7331152 | 0.9021133 | overlap (group b) |
| Morn5 | 1.02356093 | 0.83880259 | 0.9589967 | 1.413492 | overlap (group b) |
| ENSMUST00000051089 | 0.69343979 | 1.0983513 | 0.9586168 | 1.485324 | overlap (group b) |
| 5730469M10Rik | 0.8778742 | 0.73821069 | 1.0102011 | 1.6156014 | overlap (group b) |
| Gpr126 | 0.65397089 | 0.99939286 | 1.1734743 | 1.4168944 | overlap (group b) |
| LOC100044968 | 0.68692086 | 0.86148353 | 1.0686045 | 1.641552 | overlap (group b) |
| Pde4a | 1.07144082 | 1.11574631 | 0.7416597 | 1.3642777 | overlap (group b) |
| Al131754 | 1.07814983 | 0.57516463 | 1.6839923 | 0.9636418 | overlap (group b) |
| Efhc1 | 0.89886523 | 1.02013282 | 0.8553601 | 1.5301599 | overlap (group b) |
| Fam124a | 0.63234411 | 0.87483774 | 0.8144851 | 1.9852208 | overlap (group b) |
| ENSMUST00000113367 | 1.32236177 | 1.62657541 | 0.5515467 | 0.813305 | overlap (group b) |
| Mmp23 | 0.80711284 | 0.84020962 | 0.9129683 | 1.7621473 | overlap (group b) |
| Tgfbr3 | 0.84868721 | 1.21453109 | 0.8846411 | 1.3865308 | overlap (group b) |
| Galntl2 | 1.16745018 | 0.87337041 | 1.0127548 | 1.283987 | overlap (group b) |
| Plce1 | 0.72897511 | 0.83126317 | 1.4252136 | 1.3724933 | overlap (group b) |
| Prickle2 | 0.99081939 | 1.26381327 | 0.9634347 | 1.1424655 | overlap (group b) |
| Serping1 | 0.71290035 | 0.48964665 | 1.1905275 | 1.9781633 | overlap (group b) |
| Chd5 | 0.6932118 | 0.98652584 | 1.0879644 | 1.6071517 | overlap (group b) |
| Spp1 | 1.10517414 | 1.43150254 | 1.0043469 | 0.8342445 | overlap (group b) |
| 4930412F09Rik | 0.75549526 | 0.61465464 | 1.5923384 | 1.4132328 | overlap (group b) |
| Csrp2 | 1.35586084 | 1.4997071 | 0.6854649 | 0.8523938 | overlap (group b) |
| Arhgef19 | 1.52922504 | 1.60289933 | 0.4925373 | 0.7700436 | overlap (group b) |
| ENSMUST00000108284 | 0.85030425 | 0.99044212 | 0.6902818 | 1.864734 | overlap (group b) |
| BC031353 | 0.9699233 | 0.92350277 | 1.1268885 | 1.3838032 | overlap (group b) |
| Smarca1 | 0.8944522 | 1.27893302 | 0.7539322 | 1.4777181 | overlap (group b) |
| Plxna2 | 1.44909722 | 1.16792404 | 0.4911691 | 1.3000276 | overlap (group b) |
| Frat1 | 0.65643673 | 0.77597842 | 1.0189609 | 1.9587202 | overlap (group b) |
| Synm | 0.8128426 | 0.92636401 | 0.7773649 | 1.9009903 | overlap (group b) |
| Il11 | 0.9692363 | 0.74314992 | 1.4019452 | 1.3041255 | overlap (group b) |
| Mgst1 | 1.22168107 | 1.28811628 | 0.8368703 | 1.0731225 | overlap (group b) |
| Aspa | 1.26328808 | 1.37038775 | 0.8973559 | 0.8899199 | overlap (group b) |
| Calcoco1 | 1.25302646 | 0.99895391 | 1.020649 | 1.1563888 | overlap (group b) |
| Naaa | 0.72992139 | 0.93128768 | 0.9530689 | 1.8191726 | overlap (group b) |
| Zfp286 | 0.81448806 | 0.8742671 | 1.0481792 | 1.7007844 | overlap (group b) |
| Sh3rf3 | 1.00784394 | 1.0905992 | 0.6348994 | 1.7058464 | overlap (group b) |
| Unc93b1 | 0.84513578 | 1.02256142 | 1.2103014 | 1.3640713 | overlap (group b) |
| Psd3 | 1.10657342 | 1.20966379 | 0.8449312 | 1.2854986 | overlap (group b) |
| Slc7a4 | 1.75081236 | 0.1171237 | 0.972063 | 1.6221128 | overlap (group b) |
| N4bp2l1 | 0.79079762 | 1.17511062 | 1.0243351 | 1.4744406 | overlap (group b) |
| Klra22 | 0.71505665 | 0.75461522 | 1.237667 | 1.7701503 | overlap (group b) |
| Galnt3 | 1.24670604 | 0.09344761 | 1.248117 | 1.8897715 | overlap (group b) |
| Car11 | 1.34791285 | 1.02407341 | 0.7795413 | 1.3367275 | overlap (group b) |
| Crybb3 | 0.95054627 | 0.9443986 | 0.6921454 | 1.9033739 | overlap (group b) |
| ENSMUST00000034373 | 1.3686181 | 0.89810516 | 1.1864148 | 1.0423337 | overlap (group b) |
| Aspg | 1.0622077 | 0.74823574 | 0.9928449 | 1.6969946 | overlap (group b) |
| Krt15 | 1.38215756 | 1.31523272 | 0.6422432 | 1.1646175 | overlap (group b) |
| Alox5ap | 1.46310382 | 1.18515942 | 0.797029 | 1.0622183 | overlap (group b) |
| Gm347 | 0.64747188 | 0.98460463 | 1.1026718 | 1.7774188 | overlap (group b) |
| Diras2 | 0.64699496 | 0.67286182 | 1.0942017 | 2.1009831 | overlap (group b) |
| Prph | 1.47717067 | 1.52464275 | 0.5171371 | 0.9973328 | overlap (group b) |
| C1rb | 0.63223684 | 0.6854053 | 1.3719825 | 1.8337856 | overlap (group b) |
| Basp1 | 0.68083413 | 0.66926042 | 1.1407942 | 2.0565429 | overlap (group b) |
| Itgbl1 | 1.3966494 | 1.51007999 | 0.8028408 | 0.842338 | overlap (group b) |
| 5830444B04Rik | 1.34174204 | 1.4439007 | 0.7474178 | 1.0191695 | overlap (group b) |
| Flrt1 | 1.32834337 | 1.31365347 | 0.6240945 | 1.293701 | overlap (group b) |
| chr11: 93994795-94017045_F | 0.87308339 | 1.06457793 | 1.0659294 | 1.5668299 | overlap (group b) |
| Galm | 0.90520818 | 0.86968098 | 1.3187251 | 1.4768457 | overlap (group b) |
| Clec2d | 0.93774588 | 0.41429547 | 1.6098212 | 1.6106701 | overlap (group b) |
| Meig1 | 0.78821322 | 0.75167242 | 1.4383985 | 1.6062606 | overlap (group b) |
| C1ql3 | 0.98872161 | 1.07515095 | 0.7747724 | 1.753149 | overlap (group b) |
| 1110021L09Rik | 1.38090177 | 1.71104369 | 0.4776105 | 1.0280491 | overlap (group b) |
| Hic1 | 1.26012552 | 1.083354 | 0.8367862 | 1.4240021 | overlap (group b) |
| Dbp | 1.17354496 | 1.05084289 | 1.2164131 | 1.180322 | overlap (group b) |
| Ell3 | 0.79988642 | 1.02250248 | 1.3245031 | 1.4743824 | overlap (group b) |
| Pcdhb20 | 1.44314188 | 1.8310974 | 0.3703326 | 0.9958647 | overlap (group b) |
| Fbxo36 | 1.21670646 | 1.41095786 | 0.8949512 | 1.1313462 | overlap (group b) |
| Lrrn4cl | 0.98924786 | 0.88764178 | 1.0568396 | 1.7258759 | overlap (group b) |
| Rundc3a | 2.07018305 | 0.63386951 | 0.8603676 | 1.0967978 | overlap (group b) |
| Tgfb3 | 1.56068728 | 1.36216777 | 0.5241485 | 1.2190323 | overlap (group b) |
| Crispld2 | 1.602351 | 0.98739449 | 0.9664957 | 1.1125799 | overlap (group b) |
| Gm5141 | 1.16659087 | 1.36760379 | 0.9392888 | 1.1979256 | overlap (group b) |
| Hsd17b11 | 0.79127112 | 0.77603558 | 1.1240724 | 1.9821909 | overlap (group b) |
| Cd99 | 0.72843281 | 0.96063878 | 1.0118428 | 1.9787588 | overlap (group b) |
| Ltbp2 | 1.39397067 | 1.63015794 | 0.4769902 | 1.1798251 | overlap (group b) |
| ENSMUST00000100423 | 1.0816964 | 1.15289159 | 0.8616443 | 1.5853048 | overlap (group b) |
| Podxl2 | 1.65240031 | 1.66325585 | 0.2985212 | 1.0755871 | overlap (group b) |
| Ngef | 1.06850267 | 0.86446187 | 1.6229563 | 1.1508859 | overlap (group b) |
| Stard10 | 1.03541321 | 1.06129737 | 1.0918174 | 1.5285481 | overlap (group b) |
| Angpt2 | 0.95025711 | 0.9381101 | 1.2068477 | 1.6327357 | overlap (group b) |
| Dennd2d | 0.76003108 | 0.8343607 | 1.3600466 | 1.7754737 | overlap (group b) |
| Gstm7 | 1.55462328 | 1.5353406 | 0.6921231 | 0.9523851 | overlap (group b) |
| ENSMUST00000119870 | 1.39171406 | 1.33629036 | 1.0531135 | 0.9670096 | overlap (group b) |
| Rnasel | 0.48644104 | 0.9083799 | 1.5494042 | 1.8091236 | overlap (group b) |
| Gm6556 | 0.74057994 | 0.72792834 | 1.2059539 | 2.0792061 | overlap (group b) |
| Fas | 0.81899743 | 0.71484526 | 1.1389026 | 2.0822002 | overlap (group b) |
| Dll1 | 0.65334479 | 1.36052482 | 1.376755 | 1.3909893 | overlap (group b) |
| Galnt14 | 1.33149805 | 1.39046578 | 0.912479 | 1.1539807 | overlap (group b) |
| Itln1 | 0.95171298 | 0.89743611 | 0.9579655 | 1.9896736 | overlap (group b) |
| Mdga1 | 1.52024718 | 1.47521782 | 0.5358668 | 1.267452 | overlap (group b) |
| Gpc2 | 0.61679631 | 1.05051885 | 1.2242535 | 1.9192198 | overlap (group b) |
| Hr | 1.32294327 | 1.4674731 | 0.8780459 | 1.1509652 | overlap (group b) |
| Scx | 0.95268666 | 0.97346059 | 0.854568 | 2.0441945 | overlap (group b) |
| B2m | 0.78960615 | 0.84904492 | 1.3267067 | 1.8651753 | overlap (group b) |
| Glod5 | 1.26651333 | 1.25379331 | 1.178528 | 1.1331324 | overlap (group b) |
| chr3: 95502970-95503399_R | 1.23711274 | 1.19929939 | 0.947866 | 1.4582741 | overlap (group b) |
| Arhgap8 | 1.16160681 | 1.01564348 | 1.5692106 | 1.1078337 | overlap (group b) |
| Nox4 | 0.68357439 | 0.76102952 | 1.3133148 | 2.1114251 | overlap (group b) |
| LOC100041708 | 0.67780746 | 0.94669251 | 1.54688 | 1.6985325 | overlap (group b) |
| Gbp8 | 0.83570806 | 1.39251254 | 0.9754655 | 1.6764002 | overlap (group b) |
| Onecut2 | 0.95975326 | 0.92647618 | 0.9053537 | 2.1015588 | overlap (group b) |
| Orm3 | 0.83122966 | 0.90357192 | 0.9937697 | 2.1708848 | overlap (group b) |
| ENSMUST00000102549 | 1.12533805 | 1.20540244 | 1.2497879 | 1.3203748 | overlap (group b) |
| Gm3256 | 1.36339644 | 1.04304401 | 1.0912244 | 1.4183891 | overlap (group b) |
| Tubb2b | 1.70369867 | 1.04741389 | 0.9797204 | 1.1922743 | overlap (group b) |
| Adamts4 | 1.08019585 | 1.15868664 | 1.0268301 | 1.6606431 | overlap (group b) |
| Ppp1r14a | 1.10158094 | 1.4994543 | 0.9949215 | 1.334882 | overlap (group b) |
| LOC641136 | 0.82049131 | 0.73095419 | 1.4524319 | 1.9275113 | overlap (group b) |
| Casp12 | 0.56556055 | 0.95936995 | 1.1159785 | 2.2955431 | overlap (group b) |
| C030027H14Rik | 1.31472353 | 1.72935888 | 0.5095115 | 1.3884024 | overlap (group b) |
| Mrgprf | 1.04737666 | 0.87346395 | 1.4396941 | 1.5819379 | overlap (group b) |
| Oplah | 1.39465413 | 1.19986819 | 1.0991677 | 1.2520315 | overlap (group b) |
| Ralgds | 1.54273991 | 1.05715998 | 1.2161902 | 1.136627 | overlap (group b) |
| Cxxc5 | 1.7608482 | 0.71799272 | 0.8881629 | 1.5861439 | overlap (group b) |
| Sipa1l2 | 1.07421297 | 1.01185773 | 1.0454394 | 1.8216912 | overlap (group b) |
| Blnk | 0.99836544 | 0.4932181 | 1.6137744 | 1.8559224 | overlap (group b) |
| Arrdc3 | 1.34192848 | 1.42565308 | 1.1060115 | 1.0950964 | overlap (group b) |
| Hspa12b | 0.75003546 | 1.03869731 | 1.1632138 | 2.0223811 | overlap (group b) |
| Hist1h1c | 1.1848074 | 1.16063892 | 1.0684285 | 1.5691004 | overlap (group b) |
| Dhrs3 | 1.32070375 | 1.20464659 | 0.8664178 | 1.5980909 | overlap (group b) |
| Gng11 | 0.94304984 | 0.56835746 | 1.193236 | 2.2926626 | overlap (group b) |
| Rgs2 | 1.7326911 | 1.43141811 | 0.856517 | 0.9826083 | overlap (group b) |
| Col27a1 | 1.18848919 | 1.20706621 | 0.7613383 | 1.8509462 | overlap (group b) |
| ENSMUST00000118006 | 1.47203085 | 0.38254963 | 1.427029 | 1.7262652 | overlap (group b) |
| Tlr2 | 0.84787278 | 0.68530343 | 1.3988783 | 2.0776487 | overlap (group b) |
| Olfml3 | 1.39343303 | 1.32722335 | 0.7236684 | 1.5826447 | overlap (group b) |
| C1ql1 | 0.89148518 | 1.09144165 | 1.056045 | 1.9930544 | overlap (group b) |
| Tesc | 1.44385542 | 1.34717547 | 0.6354104 | 1.6103864 | overlap (group b) |
| ENSMUST00000100820 | 1.71386717 | 1.73290455 | 0.617795 | 0.9989827 | overlap (group b) |
| Rorc | 1.29072127 | 1.33234623 | 0.9838678 | 1.5161325 | overlap (group b) |
| Gm3806 | 0.90323405 | 0.77066421 | 1.5879005 | 1.8690019 | overlap (group b) |
| chr1: 87348082-87409907_F | 0.71787093 | 1.02793624 | 1.6606598 | 1.7290921 | overlap (group b) |
| Itgb4 | 1.55087193 | 1.40305154 | 0.8961057 | 1.2894743 | overlap (group b) |
| Atp6v0a4 | 1.86199151 | 1.65700359 | 0.816129 | 0.8094266 | overlap (group b) |
| Ppap2b | 1.57067365 | 1.87562104 | 0.5873498 | 1.1476989 | overlap (group b) |
| Egr1 | 1.72677111 | 0.83463033 | 1.1232316 | 1.5101638 | overlap (group b) |
| LOC677636 | 1.37804201 | 1.3135796 | 1.3152392 | 1.1963577 | overlap (group b) |
| Rprm | 1.51163156 | 1.50849186 | 0.7006654 | 1.4869587 | overlap (group b) |
| ENSMUST00000063354 | 1.76295074 | −0.0444042 | 1.6533883 | 1.8526424 | overlap (group b) |
| Lrpap1 | 1.3385725 | 1.47702552 | 1.0897925 | 1.3433166 | overlap (group b) |
| ENSMUST00000039987 | 1.37580931 | 1.48634847 | 0.6037733 | 1.7841885 | overlap (group b) |
| Xdh | 0.85104933 | 0.81857189 | 1.6313063 | 1.9510016 | overlap (group b) |
| Cyb561 | 1.63337032 | 1.5093131 | 0.9834213 | 1.1396108 | overlap (group b) |
| Sema3f | 1.36741351 | 1.46164474 | 0.8717202 | 1.5789815 | overlap (group b) |
| Adamtsl4 | 1.37372047 | 1.52459678 | 0.8779101 | 1.5067051 | overlap (group b) |
| Ppapdc3 | 1.51704033 | 1.54292015 | 0.8197268 | 1.4146944 | overlap (group b) |
| Fcgrt | 1.47489102 | 1.49497628 | 0.928339 | 1.4059671 | overlap (group b) |
| Egr2 | 1.72674401 | 1.20912733 | 1.0208947 | 1.3474637 | overlap (group b) |
| Rpl38 | 1.33079529 | 1.25342504 | 1.4307886 | 1.2921825 | overlap (group b) |
| Fbxo32 | 1.42867681 | 1.57233622 | 0.9536629 | 1.3588242 | overlap (group b) |
| ENSMUST00000057750 | 1.2682015 | 1.64100085 | 0.8209246 | 1.583605 | overlap (group b) |
| Raet1e | 1.20086953 | 0.98482389 | 1.0337286 | 2.0968871 | overlap (group b) |
| C1ra | 1.00261762 | 0.95023594 | 1.4544544 | 1.9145545 | overlap (group b) |
| Vill | 1.59946374 | 1.5705398 | 0.7894083 | 1.3716831 | overlap (group b) |
| Gpc4 | 0.8030959 | 0.93150971 | 1.5068309 | 2.0900843 | overlap (group b) |
| Apol7a | 0.98739161 | 0.89873485 | 1.5386882 | 1.9213521 | overlap (group b) |
| Disp2 | 1.30106792 | 1.66418166 | 1.2675433 | 1.1179393 | overlap (group b) |
| Pink1 | 1.19009651 | 1.19382519 | 1.3279473 | 1.645956 | overlap (group b) |
| Flot2 | 1.46163878 | 1.57067364 | 0.7737379 | 1.5598976 | overlap (group b) |
| Aldh1l1 | 1.36459399 | 1.29955347 | 1.1468982 | 1.5628967 | overlap (group b) |
| Fap | 1.14450884 | 1.35348912 | 0.8827287 | 1.9950352 | overlap (group b) |
| Ccno | 1.52018647 | 1.24676445 | 1.4080794 | 1.2185927 | overlap (group b) |
| Pion | 0.96680047 | 1.17587341 | 1.4312248 | 1.8204616 | overlap (group b) |
| Gm11818 | 1.26536147 | 0.73331685 | 1.1689352 | 2.2364023 | overlap (group b) |
| chr17: 26911397-26917047_F | 1.05309315 | 1.20606427 | 1.3626757 | 1.787023 | overlap (group b) |
| Egr4 | 1.15820091 | 1.22942294 | 0.9246748 | 2.1122255 | overlap (group b) |
| P2ry6 | 1.17968644 | 1.28063002 | 1.462563 | 1.5080318 | overlap (group b) |
| Fgd2 | 1.75361555 | 1.46842992 | 0.9377364 | 1.2805459 | overlap (group b) |
| Vwa5a | 1.28545929 | 1.46618734 | 1.1838568 | 1.5082546 | overlap (group b) |
| Layn | 1.25299073 | 1.10606822 | 1.1688028 | 1.9164909 | overlap (group b) |
| Col2a1 | 1.69056253 | 1.79427592 | 0.9155716 | 1.0506233 | overlap (group b) |
| Rtn4r | 1.11280191 | 1.11584528 | 1.2673508 | 1.9595365 | overlap (group b) |
| P4ha3 | 1.2247789 | 1.23714362 | 1.1635444 | 1.8307898 | overlap (group b) |
| Styk1 | 1.07796643 | 1.25556436 | 1.0608389 | 2.0694281 | overlap (group b) |
| Mrc2 | 1.06090863 | 1.3830658 | 0.9063661 | 2.120871 | overlap (group b) |
| Trnp1 | 1.01212334 | 1.18232698 | 1.1617033 | 2.121569 | overlap (group b) |
| Ephx1 | 1.30605626 | 1.06059927 | 1.1844361 | 1.9507014 | overlap (group b) |
| Ccdc88b | 0.99701901 | 1.12659157 | 1.563047 | 1.8279378 | overlap (group b) |
| Sned1 | 1.31231027 | 1.23111331 | 1.2706848 | 1.7154921 | overlap (group b) |
| Gm10134 | 1.49500321 | 1.6834483 | 0.7013539 | 1.65639 | overlap (group b) |
| Adamts5 | 1.03350716 | 0.83447697 | 1.3912394 | 2.2832371 | overlap (group b) |
| Igfbp6 | 1.32837177 | 1.37350846 | 1.4464748 | 1.3941077 | overlap (group b) |
| Gpx7 | 1.59105209 | 1.60510442 | 0.7491613 | 1.6080148 | overlap (group b) |
| Il1rl1 | 1.27944285 | 1.23050193 | 1.5854625 | 1.461275 | overlap (group b) |
| Hoxa13 | 0.94664408 | 0.86123606 | 1.5443674 | 2.2094362 | overlap (group b) |
| chr4: 149445201-149445902_F | 1.46973151 | 1.4508195 | 1.0636918 | 1.5896544 | overlap (group b) |
| Amy1 | 1.39894985 | 1.11125339 | 1.4186101 | 1.6553708 | overlap (group b) |
| D0H4S114 | 1.70200062 | 1.94316771 | 0.5047419 | 1.4521341 | overlap (group b) |
| Ctnnd2 | 1.19755457 | 1.57760086 | 1.1516281 | 1.680592 | overlap (group b) |
| Mylip | 1.58226191 | 1.55418349 | 1.1089826 | 1.3918539 | overlap (group b) |
| Thumpd1 | 0.89977559 | 1.0242267 | 1.5977499 | 2.1273469 | overlap (group b) |
| Prdm6 | 1.80345628 | 2.00506969 | 0.484113 | 1.3635891 | overlap (group b) |
| Il17d | 1.10435694 | 1.10823778 | 1.2983206 | 2.1496572 | overlap (group b) |
| Hopx | 1.27086173 | 1.34239483 | 1.0378901 | 2.0511161 | overlap (group b) |
| Npas4 | 1.0906461 | 1.12650345 | 1.2148561 | 2.2728774 | overlap (group b) |
| Dbndd1 | 1.78970504 | 1.79405178 | 0.9285271 | 1.1954524 | overlap (group b) |
| Sp140 | 1.16209803 | 1.04159527 | 1.6898115 | 1.8288611 | overlap (group b) |
| ENSMUST00000106341 | 1.93045695 | 1.74495471 | 0.6679673 | 1.3791503 | overlap (group b) |
| Vcam1 | 1.50045528 | 1.52895159 | 1.1913925 | 1.5181867 | overlap (group b) |
| Tcp11l2 | 1.12503034 | 1.18353087 | 1.4604572 | 1.9713405 | overlap (group b) |
| Cdsn | 1.14908671 | 1.05305113 | 1.51546 | 2.0302432 | overlap (group b) |
| Apod | 1.08559522 | 1.17948654 | 1.4892319 | 1.9942488 | overlap (group b) |
| Gm8221 | 0.95454919 | 1.00190541 | 1.7577605 | 2.0516954 | overlap (group b) |
| Zik1 | 0.91873225 | 1.16172802 | 1.2989394 | 2.3917201 | overlap (group b) |
| Hfe | 1.08794422 | 1.09093073 | 1.5542704 | 2.063827 | overlap (group b) |
| Cbr2 | 1.59206718 | 1.37219113 | 0.9507069 | 1.8972626 | overlap (group b) |
| Usp2 | 1.38645839 | 1.50635396 | 1.1306533 | 1.7949698 | overlap (group b) |
| Sema3d | 0.76728265 | 1.46851727 | 1.104779 | 2.4834255 | overlap (group b) |
| 1190002H23Rik | 1.23440522 | 1.53173009 | 1.5111267 | 1.5576598 | overlap (group b) |
| Alcam | 1.32480967 | 1.5338926 | 1.4469548 | 1.5588824 | overlap (group b) |
| 4833427G06Rik | 1.53990987 | 1.81501286 | 1.0334322 | 1.4774728 | overlap (group b) |
| Ypel3 | 1.51964671 | 1.5201921 | 1.2385958 | 1.6004931 | overlap (group b) |
| Tagln | 1.25704233 | 1.15044764 | 1.1182131 | 2.3591989 | overlap (group b) |
| Fam131a | 1.55243249 | 1.78731313 | 0.6485853 | 1.8978213 | overlap (group b) |
| Fgfr2 | 1.13004102 | 1.24709141 | 1.1937303 | 2.3298995 | overlap (group b) |
| Lrrc56 | 1.55976641 | 1.64747387 | 1.2828727 | 1.4319742 | overlap (group b) |
| Plat | 1.55845859 | 1.84179231 | 0.9033876 | 1.622271 | overlap (group b) |
| Xpnpep2 | 1.52354454 | 1.31843999 | 1.5142612 | 1.5860895 | overlap (group b) |
| Pid1 | 1.76608238 | 1.58702993 | 1.2047585 | 1.4097725 | overlap (group b) |
| Kcnb1 | 1.05994625 | 1.27711855 | 1.2447122 | 2.3931606 | overlap (group b) |
| ENSMUST00000110529 | 1.16424186 | 1.46738811 | 1.3089287 | 2.0415949 | overlap (group b) |
| Tnfrsf25 | 2.06609469 | 1.82017204 | 0.7447182 | 1.3533014 | overlap (group b) |
| Akr1b7 | 1.26616227 | 1.08979212 | 1.6952618 | 1.9408456 | overlap (group b) |
| Matn2 | 1.75034189 | 1.75370266 | 0.9006345 | 1.5972772 | overlap (group b) |
| Ebf1 | 2.04041358 | 1.67943827 | 0.795816 | 1.4871277 | overlap (group b) |
| Scg5 | 1.90793487 | 1.69349592 | 1.0342631 | 1.3759743 | overlap (group b) |
| Kctd12 | 1.71636724 | 1.89443063 | 0.662092 | 1.7474299 | overlap (group b) |
| Wnt4 | 1.73647289 | 1.45986981 | 0.9461225 | 1.8898278 | overlap (group b) |
| Cyp4v3 | 1.06517393 | 1.2852466 | 1.5025168 | 2.1887928 | overlap (group b) |
| Acy3 | 1.43413718 | 1.31267798 | 1.4470745 | 1.8583217 | overlap (group b) |
| S100a14 | 1.46792033 | 1.68678867 | 1.1222727 | 1.8161098 | overlap (group b) |
| C1s | 1.49386351 | 1.63890484 | 1.2960115 | 1.6835529 | overlap (group b) |
| Col10a1 | 1.39960248 | 1.41825557 | 1.4310987 | 1.9003591 | overlap (group b) |
| 6030419C18Rik | 1.21908596 | 1.32866063 | 1.3203451 | 2.2934753 | overlap (group b) |
| Fgf7 | 1.64791506 | 1.58053085 | 0.9002577 | 2.037103 | overlap (group b) |
| ENSMUST00000118651 | 1.15835679 | 1.38777915 | 1.5204648 | 2.1092108 | overlap (group b) |
| Slc22a18 | 1.94897171 | 1.566477 | 1.0700893 | 1.6023241 | overlap (group b) |
| Cobl | 1.41025503 | 0.72531273 | 1.6200213 | 2.4364811 | overlap (group b) |
| ENSMUST00000105413 | 1.45145769 | 1.75674978 | 0.8266078 | 2.1923234 | overlap (group b) |
| Fgf10 | 1.90170606 | 2.07330204 | 0.544043 | 1.7368951 | overlap (group b) |
| Plb1 | 1.32685416 | 0.97577964 | 1.7223089 | 2.2418469 | overlap (group b) |
| Lgals7 | 1.62977419 | 1.97351653 | 1.2623743 | 1.4082048 | overlap (group b) |
| Notch3 | 1.61156729 | 1.70084823 | 1.0382825 | 1.9307059 | overlap (group b) |
| A430110N23Rik | 1.78805245 | 1.72859652 | 0.9867655 | 1.7902664 | overlap (group b) |
| Plxdc1 | 1.85686559 | 1.9484861 | 0.8570276 | 1.6435958 | overlap (group b) |
| Fibin | 1.16720727 | 1.34087467 | 1.1118224 | 2.6881601 | overlap (group b) |
| Mid1 | 1.60862179 | 1.70792581 | 1.2168267 | 1.802822 | overlap (group b) |
| Tnn | 1.73958856 | 1.48271054 | 1.1539676 | 1.9688436 | overlap (group b) |
| Neto2 | 1.13547671 | 1.3697946 | 1.4593582 | 2.4131524 | overlap (group b) |
| Scn1b | 1.72640013 | 1.83294644 | 1.0732546 | 1.7650523 | overlap (group b) |
| Plac9 | 1.93527716 | 1.76112483 | 1.0939783 | 1.6199362 | overlap (group b) |
| S100a16 | 1.98020911 | 1.65864058 | 1.0446839 | 1.7374579 | overlap (group b) |
| Caps2 | 1.79779535 | 1.71152988 | 1.4149726 | 1.5269365 | overlap (group b) |
| Flt1 | 1.47630487 | 1.36845114 | 1.3261751 | 2.2898184 | overlap (group b) |
| Tgm5 | 2.25349969 | 1.84417758 | 0.9242109 | 1.4512018 | overlap (group b) |
| Crip2 | 2.17904739 | 2.00395393 | 0.8346535 | 1.4580506 | overlap (group b) |
| Tmprss6 | 1.55994914 | 1.33146233 | 1.2313737 | 2.3636112 | overlap (group b) |
| Gm7676 | 1.46376646 | 1.25614518 | 1.5285579 | 2.2528797 | overlap (group b) |
| Podn | 1.78272898 | 1.86408469 | 1.0176581 | 1.8530815 | overlap (group b) |
| Gm6366 | 1.43924024 | 1.86941411 | 1.0430007 | 2.1775177 | overlap (group b) |
| Hoxc13 | 1.75304993 | 1.8484695 | 1.0802224 | 1.8851334 | overlap (group b) |
| Mxd4 | 1.83617764 | 1.79201762 | 1.0824116 | 1.8756562 | overlap (group b) |
| Clu | 1.99053854 | 1.65848003 | 1.0964436 | 1.8699482 | overlap (group b) |
| Hey1 | 2.01924414 | 1.88901677 | 1.3291366 | 1.3828781 | overlap (group b) |
| Vdr | 1.99219442 | 2.07238247 | 0.9412021 | 1.6153737 | overlap (group b) |
| Setbp1 | 2.00177729 | 1.96938182 | 0.7603184 | 1.9019147 | overlap (group b) |
| Cpeb1 | 1.77947118 | 2.09756221 | 0.8798491 | 1.8828879 | overlap (group b) |
| Npy1r | 1.50714587 | 1.20707043 | 1.411046 | 2.5324189 | overlap (group b) |
| Igsf10 | 2.03131558 | 2.04067971 | 0.7495124 | 1.8451736 | overlap (group b) |
| Atp10a | 1.3511435 | 1.43358111 | 1.3980928 | 2.4992529 | overlap (group b) |
| Lrrc8e | 1.46589601 | 1.41635771 | 1.6256043 | 2.2866143 | overlap (group b) |
| Prkg1 | 1.05105841 | 1.41347278 | 1.7245687 | 2.6708642 | overlap (group b) |
| Gpr56 | 2.17481269 | 2.01514285 | 1.1561078 | 1.5198691 | overlap (group b) |
| Zfp521 | 1.59519478 | 1.57199827 | 1.6026995 | 2.1622629 | overlap (group b) |
| Slc1a3 | 1.52013066 | 1.49794304 | 1.3262868 | 2.5892213 | overlap (group b) |
| ENSMUST00000074855 | 1.35358979 | 1.23544484 | 1.6392319 | 2.7071967 | overlap (group b) |
| ENSMUST00000107402 | 1.4427963 | 1.59509141 | 1.5886267 | 2.3117035 | overlap (group b) |
| Tbx18 | 1.57062461 | 1.66313504 | 1.758265 | 1.968173 | overlap (group b) |
| Aldh3b1 | 1.57830493 | 1.42167637 | 1.7771811 | 2.2058398 | overlap (group b) |
| Figf | 1.50380351 | 1.4613847 | 1.6203587 | 2.4048663 | overlap (group b) |
| A930005I04Rik | 1.34933943 | 1.36985078 | 1.6563952 | 2.6212189 | overlap (group b) |
| Grik5 | 1.29905922 | 1.48993795 | 1.7884417 | 2.4230824 | overlap (group b) |
| Unc5c | 1.33329445 | 1.3425115 | 1.7134801 | 2.6335429 | overlap (group b) |
| Ccdc129 | 1.36477247 | 1.47857554 | 1.9500737 | 2.2377809 | overlap (group b) |
| Tmem119 | 2.30904523 | 2.11029569 | 0.9035296 | 1.716174 | overlap (group b) |
| Ifitm1 | 1.64611182 | 1.44334645 | 1.6017543 | 2.3525693 | overlap (group b) |
| Ednra | 1.98256599 | 1.96298385 | 1.2060412 | 1.9303522 | overlap (group b) |
| Cys1 | 1.56498003 | 1.86183707 | 1.3386923 | 2.3260922 | overlap (group b) |
| Ppl | 2.0392353 | 2.07593149 | 1.1053755 | 1.9605684 | overlap (group b) |
| Wt1 | 1.54540296 | 1.82472394 | 1.4398737 | 2.3808106 | overlap (group b) |
| Sgcd | 1.31898764 | 1.40412733 | 1.7884884 | 2.6887854 | overlap (group b) |
| Trem2 | 1.69528329 | 1.5453418 | 2.1364383 | 1.8313111 | overlap (group b) |
| Tmem53 | 1.76675025 | 1.69099297 | 1.4772724 | 2.2911084 | overlap (group b) |
| Uchl1 | 1.57477061 | 1.83524881 | 1.5162789 | 2.3077579 | overlap (group b) |
| Smad9 | 1.3609157 | 1.27193633 | 1.7513519 | 2.8550981 | overlap (group b) |
| C1qtnf1 | 2.08645291 | 1.92800972 | 1.2407217 | 1.9929143 | overlap (group b) |
| Peg13 | 1.63245516 | 1.71525258 | 1.3949762 | 2.6015267 | overlap (group b) |
| Cstad | 1.96020129 | 2.21018847 | 1.3969153 | 1.8041176 | overlap (group b) |
| Wipf1 | 1.29905881 | 1.4691645 | 1.8856264 | 2.7251845 | overlap (group b) |
| Lxn | 1.90133863 | 1.75477102 | 1.7534725 | 2.0004767 | overlap (group b) |
| Ecm1 | 1.61418071 | 1.72144924 | 1.7448954 | 2.3591065 | overlap (group b) |
| Tcn2 | 1.95336097 | 1.65986942 | 1.6702556 | 2.1733598 | overlap (group b) |
| Nfatc4 | 2.15642804 | 2.02668892 | 1.3110549 | 1.9973563 | overlap (group b) |
| Nbl1 | 1.86025779 | 1.60994694 | 1.2908554 | 2.7327644 | overlap (group b) |
| Hist1h2bc | 1.61147742 | 1.30045119 | 2.0096607 | 2.59831 | overlap (group b) |
| Smad6 | 2.27123783 | 1.92791703 | 1.362838 | 2.019116 | overlap (group b) |
| Meis2 | 1.60225002 | 1.58150955 | 1.6250018 | 2.7784016 | overlap (group b) |
| Il18 | 1.89900822 | 2.13176854 | 1.8115754 | 1.8288109 | overlap (group b) |
| Lpar4 | 1.68922964 | 1.97120628 | 1.4914533 | 2.5483955 | overlap (group b) |
| Aox1 | 2.08431114 | 1.7072738 | 1.6242685 | 2.2999473 | overlap (group b) |
| Gpc3 | 2.17564498 | 2.39600466 | 1.2705307 | 1.8738112 | overlap (group b) |
| Bmp2 | 1.54055008 | 1.61717136 | 1.3973677 | 3.2065688 | overlap (group b) |
| Slc43a2 | 2.33650189 | 2.20574008 | 1.4360477 | 1.7992065 | overlap (group b) |
| Gdpd2 | 1.79430784 | 1.48399821 | 1.6397028 | 2.8777941 | overlap (group b) |
| Slpi | 1.99841862 | 1.510522 | 1.8326217 | 2.4922914 | overlap (group b) |
| Gm5067 | 1.62266328 | 1.58203204 | 1.8772873 | 2.7691163 | overlap (group b) |
| Cxx1c | 1.84075409 | 1.89969269 | 1.7928167 | 2.3234124 | overlap (group b) |
| Aldh3a1 | 1.97439874 | 1.60144704 | 1.9498636 | 2.366989 | overlap (group b) |
| ENSMUST00000097840 | 2.31521819 | 2.38078236 | 1.1555898 | 2.0517244 | overlap (group b) |
| Ppp1r3d | 1.49443878 | 1.85625172 | 1.8419639 | 2.8081064 | overlap (group b) |
| Plekha4 | 1.88552068 | 1.64462174 | 2.0568523 | 2.484248 | overlap (group b) |
| Tcfap2b | 1.35992867 | 1.85472577 | 1.9326157 | 2.9924075 | overlap (group b) |
| Rab3il1 | 2.00040493 | 1.70968075 | 1.7581372 | 2.6889511 | overlap (group b) |
| Fam198b | 1.65670305 | 1.47923004 | 2.2516685 | 2.7860956 | overlap (group b) |
| Wnt5b | 1.77577706 | 1.76841086 | 1.8153574 | 2.8151924 | overlap (group b) |
| Btc | 2.14193211 | 2.48388711 | 1.6938131 | 1.891401 | overlap (group b) |
| Rarres1 | 2.38216483 | 2.64283177 | 1.013571 | 2.1978921 | overlap (group b) |
| 1110036O03Rik | 1.75381726 | 1.74055633 | 1.7923492 | 2.9781149 | overlap (group b) |
| chr10: 83185606-83225506_F | 1.8206047 | 2.05601338 | 1.6399263 | 2.7556794 | overlap (group b) |
| Dio2 | 2.60588133 | 2.34083885 | 1.6540896 | 1.7172907 | overlap (group b) |
| Pik3ip1 | 2.04069006 | 1.83895227 | 1.9933156 | 2.452755 | overlap (group b) |
| Trp53inp1 | 2.03286659 | 1.93716097 | 1.8326381 | 2.5643191 | overlap (group b) |
| Gm6949 | 1.65294063 | 1.81797737 | 1.9600138 | 2.9508152 | overlap (group b) |
| 6330407I18Rik | 1.44678977 | 1.7505791 | 1.8594536 | 3.3330691 | overlap (group b) |
| Ckb | 1.48770695 | 2.0076031 | 1.8224184 | 3.0796429 | overlap (group b) |
| Ugt1a6b | 2.25673518 | 1.93379312 | 2.192379 | 2.1666311 | overlap (group b) |
| Col6a2 | 2.33850974 | 2.15132819 | 1.252953 | 2.8352576 | overlap (group b) |
| Mmp9 | 2.57009741 | 2.73692977 | 1.3059304 | 2.0099071 | overlap (group b) |
| Trpm6 | 2.37535288 | 2.25376379 | 1.767263 | 2.2852665 | overlap (group b) |
| Sobp | 2.24778089 | 2.20368976 | 1.3040076 | 2.9493228 | overlap (group b) |
| Tox | 2.29241073 | 2.3214469 | 1.3888662 | 2.7244324 | overlap (group b) |
| Cd302 | 2.31465462 | 1.88769574 | 1.7090198 | 2.8276229 | overlap (group b) |
| Mxra8 | 2.09129728 | 1.96085596 | 1.8331382 | 2.8797307 | overlap (group b) |
| Scara5 | 2.60746085 | 2.4937484 | 1.8480601 | 1.8396955 | overlap (group b) |
| Atoh8 | 1.94782608 | 1.80923217 | 1.8474394 | 3.2026154 | overlap (group b) |
| chr10: 83181374-83249774_F | 1.84503721 | 1.97425812 | 2.0530894 | 3.0340857 | overlap (group b) |
| Lama2 | 2.40887533 | 2.51174659 | 1.357705 | 2.6719641 | overlap (group b) |
| Cacna2d1 | 2.21696169 | 2.36993337 | 1.4504159 | 2.9386949 | overlap (group b) |
| Calhm2 | 2.43047306 | 2.18503966 | 1.6044224 | 2.7730162 | overlap (group b) |
| Plagl1 | 1.99106771 | 2.15794134 | 2.1698297 | 2.7452817 | overlap (group b) |
| Id1 | 2.67963548 | 2.49323033 | 1.8994381 | 1.9936408 | overlap (group b) |
| Ptger1 | 2.71679769 | 2.60547723 | 1.4476467 | 2.3262861 | overlap (group b) |
| BC016201 | 2.82811417 | 2.49561032 | 1.8175469 | 1.9557919 | overlap (group b) |
| Cdo1 | 2.3671275 | 2.90266249 | 1.3641944 | 2.4646596 | overlap (group b) |
| Tspan7 | 2.21355881 | 2.16540696 | 1.7621316 | 2.9734457 | overlap (group b) |
| Fzd8 | 1.89298158 | 2.33588316 | 2.0044052 | 3.0224581 | overlap (group b) |
| Ehd3 | 2.67212246 | 2.5757674 | 1.42881 | 2.6254034 | overlap (group b) |
| Fbln7 | 1.8768057 | 1.80562459 | 2.1050737 | 3.5278418 | overlap (group b) |
| A530047J11Rik | 2.00257602 | 2.21826175 | 1.8370079 | 3.2813577 | overlap (group b) |
| ENSMUST00000072014 | 1.61561414 | 2.1207784 | 2.1015619 | 3.5910487 | overlap (group b) |
| Eid2 | 1.86238929 | 1.94293616 | 2.3080063 | 3.3318957 | overlap (group b) |
| Phactr1 | 2.42286717 | 2.51059916 | 2.2368319 | 2.3507641 | overlap (group b) |
| AU018778 | 2.74232232 | 2.28142926 | 2.2688788 | 2.3292311 | overlap (group b) |
| Etl4 | 1.83934083 | 2.21240326 | 2.3058328 | 3.5100394 | overlap (group b) |
| Colec12 | 2.6828868 | 2.3640758 | 2.2105583 | 2.6579692 | overlap (group b) |
| Rbp4 | 2.70081629 | 2.61549719 | 1.4233605 | 3.2038327 | overlap (group b) |
| Plcd1 | 2.60941942 | 2.54611697 | 2.0152922 | 2.83536 | overlap (group b) |
| Nlrp2 | 2.190605 | 2.24824322 | 2.5102986 | 3.0697747 | overlap (group b) |
| Bdh2 | 2.3475274 | 2.13909296 | 2.1600257 | 3.4294202 | overlap (group b) |
| Fzd1 | 2.09367476 | 2.31258901 | 2.235443 | 3.4593662 | overlap (group b) |
| ENSMUST00000026672 | 2.20436431 | 1.98366076 | 2.3790545 | 3.5676383 | overlap (group b) |
| Sema3c | 2.38154033 | 2.31491678 | 2.2687948 | 3.2515569 | overlap (group b) |
| Rora | 1.78239422 | 2.33756589 | 2.3932168 | 3.7054242 | overlap (group b) |
| Fbn1 | 2.44377818 | 2.4315971 | 2.0586282 | 3.3219897 | overlap (group b) |
| Gpr64 | 2.04495654 | 2.37701808 | 2.3612067 | 3.5287745 | overlap (group b) |
| Il13ra2 | 2.20086156 | 2.01770583 | 2.7587251 | 3.3971636 | overlap (group b) |
| Grb14 | 2.28014092 | 2.01697829 | 2.6127351 | 3.569327 | overlap (group b) |
| Wnt5a | 2.25039687 | 2.28244558 | 2.3926735 | 3.6309844 | overlap (group b) |
| LOC677253 | 2.41739092 | 2.37736554 | 2.2514891 | 3.7073276 | overlap (group b) |
| Dpysl3 | 2.97124751 | 2.75084939 | 2.247444 | 3.0079505 | overlap (group b) |
| Myl9 | 2.53179404 | 2.41161404 | 2.4483789 | 3.8048354 | overlap (group b) |
| Smc1b | 2.9471489 | 2.8536715 | 2.2168521 | 3.1937784 | overlap (group b) |
| Sash1 | 2.88049199 | 2.12057965 | 2.793032 | 3.5016057 | overlap (group b) |
| Tshz3 | 2.80900656 | 2.64396512 | 2.8516189 | 3.0068133 | overlap (group b) |
| H2-DMb1 | 2.58209901 | 2.60415105 | 2.4082765 | 3.7189204 | overlap (group b) |
| Hmga2 | 2.86861764 | 2.75458073 | 2.4576902 | 3.3505276 | overlap (group b) |
| Col5a2 | 2.87631915 | 2.921333 | 2.3749214 | 3.3686624 | overlap (group b) |
| Murc | 2.65075616 | 2.64709576 | 2.3771947 | 4.0164542 | overlap (group b) |
| Col6a1 | 3.1694039 | 3.08786466 | 2.2108233 | 3.3146408 | overlap (group b) |
| Nod1 | 2.54306573 | 2.88980834 | 2.7346203 | 3.6492158 | overlap (group b) |
| Cables1 | 3.21419849 | 3.30613962 | 2.1095395 | 3.3493237 | overlap (group b) |
| Lama4 | 3.29338336 | 3.41029843 | 1.9296496 | 3.3512638 | overlap (group b) |
| Lrrc32 | 3.31814728 | 3.37931549 | 2.0780384 | 3.2471112 | overlap (group b) |
| Id3 | 3.18837233 | 2.8534198 | 2.6552291 | 3.4420943 | overlap (group b) |
| Pdgfrl | 2.71494226 | 2.68700197 | 2.6262884 | 4.1673383 | overlap (group b) |
| Ass1 | 2.88743532 | 2.67823929 | 3.0885849 | 3.8657744 | overlap (group b) |
| Tmem45a | 3.54989535 | 3.588317 | 2.101631 | 3.302621 | overlap (group b) |
| Hdc | 2.78265171 | 2.97416924 | 3.2788036 | 3.5935354 | overlap (group b) |
| Gm12892 | 2.66028192 | 2.72451086 | 3.1062981 | 4.1606 | overlap (group b) |
| Nov | 2.91142171 | 3.18364801 | 2.7357526 | 4.0911866 | overlap (group b) |
| Svep1 | 3.77253453 | 3.7532829 | 2.2367674 | 3.487316 | overlap (group b) |
| Ccdc80 | 3.29651962 | 3.2754286 | 2.8708743 | 4.2847545 | overlap (group b) |
| Serpine2 | 3.49262312 | 3.29576021 | 2.8463309 | 4.2457937 | overlap (group b) |
| Zrsr1 | 2.76063304 | 3.19004968 | 3.2158497 | 4.7178562 | overlap (group b) |
| Itih2 | 3.2387779 | 3.10371948 | 3.2548753 | 4.4009055 | overlap (group b) |
| Dcn | 3.99538419 | 3.52048512 | 2.689037 | 3.8295549 | overlap (group b) |
| Bmp4 | 3.54590507 | 3.52254819 | 3.1853507 | 3.854744 | overlap (group b) |
| Cdh2 | 3.52744975 | 3.27614698 | 3.2928243 | 4.2626011 | overlap (group b) |
| Sulf1 | 4.01086335 | 4.25755678 | 2.69803 | 3.9371458 | overlap (group b) |
| Lox | 3.90719508 | 3.96495832 | 2.8236298 | 4.2865876 | overlap (group b) |
| Pcsk6 | 3.47758286 | 3.26889103 | 3.6688598 | 4.758052 | overlap (group b) |
| Osmr | 3.66053781 | 3.49729784 | 3.4852289 | 4.9040572 | overlap (group b) |
| Limch1 | 3.84980927 | 3.88006197 | 3.8274111 | 4.9609447 | overlap (group b) |
| Vmn2r-ps134 | 4.27121904 | 4.0161521 | 3.525605 | 5.0339968 | overlap (group b) |
| Tgfb1i1 | 3.92708287 | 3.89216363 | 4.1630312 | 5.1706237 | overlap (group b) |
| Clip3 | 4.44918067 | 4.48759706 | 4.1940322 | 5.379874 | overlap (group b) |
| Ogn | 4.92903396 | 4.73733326 | 3.7387346 | 5.2581375 | overlap (group b) |
| Slc1a6 | 5.07321788 | 5.22484578 | 3.7311896 | 4.9253398 | overlap (group b) |
| Thbd | 4.68690894 | 4.67593361 | 4.3121416 | 5.5643725 | overlap (group b) |
| Nid2 | 4.43382333 | 4.65537063 | 4.8610542 | 5.8546048 | overlap (group b) |
| Cxcl14 | 4.90705875 | 4.88203769 | 4.2325695 | 5.8134388 | overlap (group b) |
| Sepp1 | 4.69712074 | 4.31144933 | 4.7245623 | 6.1778634 | overlap (group b) |
| Vegfc | 4.65988157 | 4.52007338 | 4.5980453 | 6.1515146 | overlap (group b) |
| Cdh13 | 4.88034533 | 4.90276816 | 4.7282258 | 5.9650563 | overlap (group b) |
| Serpina3g | 5.06123287 | 4.6227579 | 4.730218 | 6.3324458 | overlap (group b) |
| Prss23 | 5.33990463 | 5.1630208 | 4.718427 | 5.9052831 | overlap (group b) |
| Gypc | 5.39965408 | 5.20280412 | 4.7123038 | 6.0995578 | overlap (group b) |
| ENSMUST00000099633 | 5.19735806 | 5.45285539 | 4.8131862 | 6.1386179 | overlap (group b) |
| Nsg1 | 5.94008847 | 5.87456139 | 5.2339677 | 6.4852349 | overlap (group b) |
| Col1a2 | 5.73674201 | 5.558587 | 5.3334615 | 6.9837633 | overlap (group b) |
| Rcn3 | 6.61194186 | 6.52510076 | 5.9781685 | 7.0967531 | overlap (group b) |
| Plxdc2 | 6.94875971 | 6.83100533 | 5.4629864 | 7.081763 | overlap (group b) |
| Ppic | 6.86812387 | 6.73813108 | 6.3990333 | 7.7768092 | overlap (group b) |
| Bicc1 | 7.26971871 | 7.24760502 | 7.3152521 | 8.5363965 | overlap (group b) |
| Col3a1 | 7.22158631 | 7.49755672 | 6.8840065 | 8.8263908 | overlap (group b) |
| Penk | 9.36919214 | 9.43757866 | 1.1077095 | 2.1262162 | WT Fib up (group a) |
| Thy1 | 5.84099473 | 5.66672941 | 0.1937312 | −0.2892767 | WT Fib up (group a) |
| Dkk3 | 5.6434941 | 5.68412447 | −0.220431 | 0.2991557 | WT Fib up (group a) |
| Sfrp2 | 4.26893236 | 4.57287398 | −1.923777 | −0.381873 | WT Fib up (group a) |
| Igfbp2 | 4.77425744 | 4.81920132 | 0.0628123 | 0.9704432 | WT Fib up (group a) |
| Cxcl15 | 4.03283924 | 4.09665277 | 0.145893 | 0.152786 | WT Fib up (group a) |
| Rftn1 | 4.96780954 | 4.86720402 | 0.7489641 | 1.3789964 | WT Fib up (group a) |
| Grem1 | 4.61431733 | 4.01470866 | 0.1502766 | 0.8617583 | WT Fib up (group a) |
| Vat1l | 3.99793925 | 3.72162602 | 0.3533977 | 0.2650582 | WT Fib up (group a) |
| Dlk1 | 3.27828457 | 3.13523698 | 0.2136892 | 0.2986274 | WT Fib up (group a) |
| 1500015O10Rik | 3.2422618 | 3.36086824 | −0.370974 | 1.0753994 | WT Fib up (group a) |
| Gas6 | 6.8578766 | 6.77649724 | 3.2314421 | 4.516854 | WT Fib up (group a) |
| Omd | 3.98419377 | 3.49509369 | 0.5152866 | 1.1574345 | WT Fib up (group a) |
| Crlf1 | 4.41666203 | 4.31840473 | 1.1148681 | 1.8502252 | WT Fib up (group a) |
| Smoc2 | 5.33665466 | 5.1286442 | 1.5612682 | 3.1341601 | WT Fib up (group a) |
| Msmp | 2.76992706 | 2.90694654 | −0.185036 | 0.1460977 | WT Fib up (group a) |
| Megf10 | 2.22779805 | 2.32259383 | −0.675999 | −0.3907124 | WT Fib up (group a) |
| Itga11 | 5.33288428 | 5.08083867 | 1.8046581 | 3.1423114 | WT Fib up (group a) |
| Alx1 | 2.4850183 | 2.68749344 | −0.144394 | −0.058558 | WT Fib up (group a) |
| Lrrc17 | 4.2252172 | 4.56050456 | 1.1832726 | 2.2384241 | WT Fib up (group a) |
| Chst8 | 3.44128071 | 3.43823054 | 0.2616353 | 1.3226228 | WT Fib up (group a) |
| Itga8 | 3.30305601 | 3.70580622 | 0.7545345 | 0.99707 | WT Fib up (group a) |
| Postn | 4.49266325 | 4.49989878 | 1.0864048 | 2.7437382 | WT Fib up (group a) |
| Mmp3 | 3.19341095 | 3.09945667 | 0.5712533 | 0.5894047 | WT Fib up (group a) |
| Fbn2 | 2.14584286 | 2.2877652 | −0.395678 | −0.28517 | WT Fib up (group a) |
| Fbxl7 | 2.87192374 | 3.17726028 | 0.455219 | 0.4921118 | WT Fib up (group a) |
| Mmp2 | 4.45318566 | 4.20432417 | 1.4227596 | 2.1371227 | WT Fib up (group a) |
| ENSMUST00000066153 | 3.81230603 | 4.11681983 | 0.4844596 | 2.5112947 | WT Fib up (group a) |
| 0610040J01Rik | 2.40349572 | 2.47648244 | −0.161604 | 0.1364558 | WT Fib up (group a) |
| Slit3 | 2.35834264 | 2.47415961 | −0.071792 | 0.0080883 | WT Fib up (group a) |
| Tspan6 | 3.90084335 | 3.646318 | 0.9388463 | 1.8219154 | WT Fib up (group a) |
| Mgp | 4.93868041 | 4.74377651 | 1.802046 | 3.167167 | WT Fib up (group a) |
| Tbx4 | 2.38185869 | 2.94386913 | 0.526912 | 0.1149447 | WT Fib up (group a) |
| Dmp1 | 2.26175452 | 2.63709041 | −0.253348 | 0.5677056 | WT Fib up (group a) |
| Cav3 | 2.46661621 | 2.51305768 | −0.077946 | 0.6897737 | WT Fib up (group a) |
| Zfp105 | 2.00095155 | 1.99967326 | 0.0332405 | −0.314178 | WT Fib up (group a) |
| Edil3 | 1.98622553 | 2.10645325 | −0.138036 | −0.0211097 | WT Fib up (group a) |
| H60a | −0.0143679 | −0.0025662 | −1.848974 | −2.3934802 | WT Fib up (group a) |
| Itm2a | 2.07038887 | 2.50858986 | 0.2072341 | 0.1495711 | WT Fib up (group a) |
| Ctsh | 1.88818537 | 2.11755037 | −0.044016 | −0.1379771 | WT Fib up (group a) |
| Thbs2 | 2.4864318 | 2.47439452 | 0.2593308 | 0.5267604 | WT Fib up (group a) |
| Meis1 | 1.81034245 | 1.80851699 | −0.299011 | −0.1405821 | WT Fib up (group a) |
| Rspo1 | 2.30584398 | 2.46315449 | 0.2329567 | 0.4799227 | WT Fib up (group a) |
| Serpinf1 | 5.05080456 | 4.99857548 | 2.2340766 | 3.7593139 | WT Fib up (group a) |
| Dsp | 1.99039042 | 2.12121517 | 0.1369454 | −0.0738326 | WT Fib up (group a) |
| Pdgfrb | 1.60911205 | 1.85620008 | −0.461974 | −0.0853782 | WT Fib up (group a) |
| H60b | 0.04773058 | −0.2765958 | −1.819626 | −2.3605903 | WT Fib up (group a) |
| Sparcl1 | 2.7630564 | 2.73752871 | 0.2029739 | 1.4450636 | WT Fib up (group a) |
| Ptn | 2.19517674 | 2.13452171 | −0.087652 | 0.5695857 | WT Fib up (group a) |
| Mest | 1.11447269 | 1.50471676 | −0.516442 | −0.7056553 | WT Fib up (group a) |
| Ctsw | 0.13193668 | −0.040164 | −1.771572 | −1.9649539 | WT Fib up (group a) |
| A_55_P2069530 | −0.1122117 | −0.8787775 | −1.8733 | −2.9331553 | WT Fib up (group a) |
| Iglon5 | 3.82350383 | 3.75302659 | 1.3723125 | 2.404734 | WT Fib up (group a) |
| Slc6a2 | 2.57237478 | 2.2799043 | 0.1001858 | 0.9572135 | WT Fib up (group a) |
| Islr | 3.08415778 | 2.81883878 | 0.4087651 | 1.7865142 | WT Fib up (group a) |
| Pcdh10 | 1.7103271 | 1.89024157 | −0.064752 | −0.0136075 | WT Fib up (group a) |
| Sncaip | 2.06998329 | 2.09385873 | 0.3245304 | 0.1681176 | WT Fib up (group a) |
| C530028O21Rik | 2.16903385 | 2.38862108 | 0.5048866 | 0.4784282 | WT Fib up (group a) |
| Eya2 | 2.32388342 | 2.52359355 | 0.5015643 | 0.7755579 | WT Fib up (group a) |
| 1700030C10Rik | 1.61911487 | 1.67214647 | −0.158869 | 0.0465638 | WT Fib up (group a) |
| Nlrp5 | 1.4197028 | 1.72024284 | −0.027866 | −0.1988194 | WT Fib up (group a) |
| Nts | 2.05375757 | 2.32970014 | 0.5216482 | 0.4980292 | WT Fib up (group a) |
| Ces1 | 0.5202375 | 0.51377879 | −1.175879 | −1.1319567 | WT Fib up (group a) |
| 2810026P18Rik | 0.47307364 | 0.40765246 | −0.911437 | −1.5416931 | WT Fib up (group a) |
| Adm | 2.47538001 | 2.32320198 | 0.3168382 | 1.1588062 | WT Fib up (group a) |
| Gstt1 | 3.27562792 | 2.90732042 | 1.1991872 | 1.6686777 | WT Fib up (group a) |
| Mfap2 | 1.5552917 | 1.47114323 | −0.239617 | −0.0258337 | WT Fib up (group a) |
| Rtn4rl1 | 2.0173718 | 2.05027028 | 0.2049988 | 0.5918001 | WT Fib up (group a) |
| Lpl | 2.18912999 | 2.0705864 | 0.391325 | 0.6000773 | WT Fib up (group a) |
| Gpr97 | 1.58087853 | 1.42573082 | −0.162899 | −0.0891081 | WT Fib up (group a) |
| Vamp1 | 1.70405096 | 1.65069971 | −0.019514 | 0.1298542 | WT Fib up (group a) |
| Mn1 | 1.66894213 | 2.03006493 | 0.0468602 | 0.4269693 | WT Fib up (group a) |
| Galntl1 | 1.41147103 | 1.3089013 | −0.259589 | −0.2298693 | WT Fib up (group a) |
| Fgf18 | 2.44818015 | 2.23700601 | 0.4306671 | 1.0923875 | WT Fib up (group a) |
| H19 | 1.28160441 | 1.49173713 | −0.175895 | −0.1972656 | WT Fib up (group a) |
| Hunk | 1.92871873 | 1.81045116 | 0.226672 | 0.3795073 | WT Fib up (group a) |
| Cacna1g | 2.14821874 | 2.04931981 | 0.3390521 | 0.7511989 | WT Fib up (group a) |
| Rnase4 | 5.33633326 | 5.07666163 | 3.2942939 | 4.0228421 | WT Fib up (group a) |
| Adad2 | 1.01530652 | 1.14306206 | −0.398008 | −0.5376942 | WT Fib up (group a) |
| Ramp2 | 1.19342557 | 1.27493796 | −0.176268 | −0.3982908 | WT Fib up (group a) |
| Cspg4 | 2.37909653 | 2.47047631 | 0.6790328 | 1.1410782 | WT Fib up (group a) |
| Pax3 | 1.70157921 | 1.45452522 | 0.198057 | −0.0528181 | WT Fib up (group a) |
| Abcb9 | 1.64286243 | 1.51032215 | −0.005386 | 0.1913305 | WT Fib up (group a) |
| Col11a1 | 2.12143179 | 2.17248255 | 0.2774326 | 1.0564789 | WT Fib up (group a) |
| Foxc2 | 2.63237435 | 2.7536103 | 0.7986118 | 1.6323743 | WT Fib up (group a) |
| Hoxb9 | 1.86037721 | 1.9073478 | 0.2387966 | 0.5745504 | WT Fib up (group a) |
| ENSMUST00000110056 | −0.0604007 | −0.2397433 | −1.816924 | −1.4356983 | WT Fib up (group a) |
| ENSMUST00000055842 | −0.1615818 | 0.28785196 | −1.324067 | −1.4884913 | WT Fib up (group a) |
| Car9 | 2.36230605 | 2.35476732 | 0.7129467 | 1.0750358 | WT Fib up (group a) |
| Gng8 | 1.82781733 | 1.44913657 | −0.004934 | 0.3585622 | WT Fib up (group a) |
| Abi3bp | 3.65778601 | 4.36576747 | 1.9382316 | 3.1797766 | WT Fib up (group a) |
| 9030425E11Rik | 1.39453071 | 0.85881412 | −0.549952 | −0.0602582 | WT Fib up (group a) |
| chr18: 13107755-13107844_F | 1.43671875 | 1.6300495 | 0.1665796 | 0.0542977 | WT Fib up (group a) |
| Ctla2a | 1.16833508 | 1.55141476 | −0.120372 | −0.0015899 | WT Fib up (group a) |
| Olfr658 | −0.8199143 | −0.1039155 | −1.947783 | −1.8151321 | WT Fib up (group a) |
| Runx1t1 | 3.21972409 | 3.45107008 | 1.2759625 | 2.5583799 | WT Fib up (group a) |
| Fam20a | 1.57175778 | 1.4327504 | 0.12644 | 0.0496535 | WT Fib up (group a) |
| Aspn | 3.18164916 | 3.90857551 | 1.1046444 | 3.1634743 | WT Fib up (group a) |
| Hoxc9 | 1.39581576 | 1.66383296 | 0.155356 | 0.0828015 | WT Fib up (group a) |
| Pdgfd | 1.29127765 | 1.78919542 | −0.138048 | 0.4127256 | WT Fib up (group a) |
| Tlr1 | 1.26494589 | 1.48001695 | −0.144454 | 0.0987275 | WT Fib up (group a) |
| Tmem178 | 1.50826496 | 1.04950334 | −0.109229 | −0.1010269 | WT Fib up (group a) |
| Ifi27l1 | 1.417792 | 1.02152764 | −0.158724 | −0.1615199 | WT Fib up (group a) |
| 6530401D17Rik | 0.77582444 | 0.18608217 | −1.04164 | −0.7167247 | WT Fib up (group a) |
| A230065H16Rik | 2.50637944 | 2.57149916 | 0.9128083 | 1.4535526 | WT Fib up (group a) |
| 4930471M09Rik | 0.76799258 | 0.43719208 | −0.64689 | −0.8530303 | WT Fib up (group a) |
| Zfp783 | 1.50109984 | 1.58009296 | 0.1661691 | 0.2371237 | WT Fib up (group a) |
| Nid1 | 1.42351611 | 1.4497685 | −0.174893 | 0.3801714 | WT Fib up (group a) |
| Wisp2 | 5.13339548 | 4.68821255 | 2.8027455 | 4.3673329 | WT Fib up (group a) |
| Ank | 1.27484357 | 1.45539464 | 0.1413468 | −0.0587642 | WT Fib up (group a) |
| Chrdl1 | 1.48231551 | 1.36516637 | −0.051573 | 0.2542844 | WT Fib up (group a) |
| Qpct | 1.95808316 | 2.06085442 | 0.2590962 | 1.1249595 | WT Fib up (group a) |
| Rpl39l | 1.19732231 | 1.15004121 | −0.073114 | −0.2113305 | WT Fib up (group a) |
| ENSMUST00000050650 | 0.18423277 | −0.4978036 | −1.339169 | −1.56062 | WT Fib up (group a) |
| Pla2g7 | 2.70344527 | 2.66622239 | 1.4201374 | 1.3867502 | WT Fib up (group a) |
| Maob | 0.96848767 | 1.46605555 | −0.256295 | 0.1394811 | WT Fib up (group a) |
| Cyp7b1 | 1.10539203 | 1.36940581 | 0.0782665 | −0.1432705 | WT Fib up (group a) |
| Pip | −0.4309722 | −0.3215035 | −1.306618 | −1.9809034 | WT Fib up (group a) |
| Klhl29 | 1.3466251 | 1.61577344 | 0.1837595 | 0.2557513 | WT Fib up (group a) |
| Crip1 | 1.5548998 | 1.29333283 | 0.1748084 | 0.1716618 | WT Fib up (group a) |
| Lrrc15 | 1.50244935 | 1.23159544 | 0.0338208 | 0.2056002 | WT Fib up (group a) |
| Igdcc4 | 1.20889187 | 1.34746935 | −0.096765 | 0.1711301 | WT Fib up (group a) |
| Cd248 | 1.30243721 | 1.38364396 | −0.201701 | 0.4085856 | WT Fib up (group a) |
| Cxcr6 | 1.50607728 | 1.14775329 | −0.084494 | 0.2629599 | WT Fib up (group a) |
| Wif1 | 1.35923311 | 1.88291696 | 0.4006848 | 0.3709601 | WT Fib up (group a) |
| Mxra7 | 1.34105895 | 1.34544905 | −0.057659 | 0.2777016 | WT Fib up (group a) |
| Slc22a17 | 1.14396054 | 0.88832142 | 0.0438731 | −0.475628 | WT Fib up (group a) |
| Dkk2 | 1.03238557 | 1.09260992 | −0.190972 | −0.1444293 | WT Fib up (group a) |
| Cpb2 | 0.22112005 | −0.1969794 | −0.965469 | −1.4586521 | WT Fib up (group a) |
| Tnfrsf18 | 1.1740711 | 1.17904335 | 0.1010303 | −0.19596 | WT Fib up (group a) |
| Kazald1 | 2.38884463 | 2.22987889 | 1.0464645 | 1.1369271 | WT Fib up (group a) |
| Il1rn | 1.10013384 | 1.38429775 | −0.09522 | 0.1640782 | WT Fib up (group a) |
| Clec11a | 1.17359819 | 1.1411439 | −0.116354 | 0.01697 | WT Fib up (group a) |
| Gm8013 | 0.22143476 | −1.2290558 | −1.817524 | −1.6041865 | WT Fib up (group a) |
| Tmeff2 | 1.55703811 | 1.82692718 | 0.1658172 | 0.8040594 | WT Fib up (group a) |
| Dact1 | 0.96553893 | 1.2221974 | −0.092546 | −0.1307798 | WT Fib up (group a) |
| Cacna1a | 0.25449172 | 0.41749039 | −0.64395 | −1.0939299 | WT Fib up (group a) |
| Inmt | 2.45027005 | 1.73705344 | 0.7187161 | 1.0627494 | WT Fib up (group a) |
| AK020212 | 0.40125888 | 0.57983008 | −0.87181 | −0.5479679 | WT Fib up (group a) |
| Pmepa1 | 0.89902688 | 0.75515189 | −0.62379 | −0.1097916 | WT Fib up (group a) |
| AW742560 | 0.32908041 | 0.21828314 | −1.047233 | −0.7883817 | WT Fib up (group a) |
| Gpr153 | 2.3918109 | 2.55477407 | 0.8364234 | 1.7281946 | WT Fib up (group a) |
| Bgn | 2.09113683 | 1.68040518 | 0.6026872 | 0.7914488 | WT Fib up (group a) |
| 3632451O06Rik | 0.88962008 | 1.45826602 | −0.122109 | 0.096071 | WT Fib up (group a) |
| Gdf6 | 0.91546246 | 1.0401381 | −0.119688 | −0.2677429 | WT Fib up (group a) |
| ENSMUST00000112842 | −0.1292775 | −0.0966918 | −1.265002 | −1.2999707 | WT Fib up (group a) |
| 5730407M17Rik | 0.27924331 | −0.2922855 | −1.148823 | −1.2018176 | WT Fib up (group a) |
| Ddit4l | 0.97721759 | 1.36913448 | 0.0538916 | −0.031217 | WT Fib up (group a) |
| Gpm6b | 0.81871007 | 1.13789265 | −0.271322 | −0.094957 | WT Fib up (group a) |
| Pcdhb6 | 1.53647763 | 1.39740423 | 0.2533047 | 0.3586677 | WT Fib up (group a) |
| Pi16 | 2.64880024 | 2.6325479 | 1.0619178 | 1.9086333 | WT Fib up (group a) |
| LOC630474 | 0.22624529 | −0.4692882 | −1.185046 | −1.366238 | WT Fib up (group a) |
| Mapk13 | 1.48868291 | 1.34101478 | 0.139158 | 0.3946229 | WT Fib up (group a) |
| Megf6 | 2.09003339 | 1.99866784 | 1.0559032 | 0.7479288 | WT Fib up (group a) |
| Cxcl12 | 0.93005238 | 0.8463728 | −0.539764 | 0.0321985 | WT Fib up (group a) |
| Agtr1a | 1.41913632 | 1.32728047 | 0.1770754 | 0.2874094 | WT Fib up (group a) |
| Spon2 | 3.19757414 | 2.96441491 | 1.780191 | 2.1067215 | WT Fib up (group a) |
| Asgr1 | 2.66465171 | 2.70463286 | 1.3455394 | 1.7520766 | WT Fib up (group a) |
| AI450241 | 0.80996361 | 0.7585307 | −0.519222 | −0.1711943 | WT Fib up (group a) |
| chr1: 138592780-138622719_F | 3.01484386 | 0.50673752 | 0.4140797 | 0.8506343 | WT Fib up (group a) |
| chr1: 88384134-88384902_F | 0.06022653 | −0.2793732 | −1.078434 | −1.3891115 | WT Fib up (group a) |
| Mgll | 1.35344577 | 1.18344855 | 0.0672457 | 0.2240838 | WT Fib up (group a) |
| ENSMUST00000112175 | 0.40530067 | 0.43123519 | −0.53805 | −0.8673497 | WT Fib up (group a) |
| chr16: 34524589-34573439_R | 0.38895677 | 0.85981836 | −0.728222 | −0.2597022 | WT Fib up (group a) |
| Fbln5 | 3.15797253 | 3.11497806 | 1.7210525 | 2.3179133 | WT Fib up (group a) |
| 2310007J06Rik | 0.95624831 | 0.91655161 | −0.250382 | −0.1071195 | WT Fib up (group a) |
| chr15: 102026122-102033597_I | 0.55491514 | 0.25950182 | −0.743494 | −0.6696436 | WT Fib up (group a) |
| Cnnm1 | 1.02578423 | 1.41638429 | 0.0081424 | 0.2093903 | WT Fib up (group a) |
| Col8a2 | 0.96667749 | 1.14724623 | −0.320248 | 0.2131614 | WT Fib up (group a) |
| Crygs | 1.18233177 | 1.07885137 | −0.15345 | 0.2075956 | WT Fib up (group a) |
| Pcdhb14 | 1.3374421 | 1.81405182 | 0.3149255 | 0.6335761 | WT Fib up (group a) |
| Pllp | 1.55326844 | 1.58862535 | 0.54206 | 0.4030885 | WT Fib up (group a) |
| LOC100045975 | 0.48012729 | 0.37647719 | −0.542125 | −0.7949519 | WT Fib up (group a) |
| Lphn3 | 1.30743716 | 1.79056269 | 0.2992756 | 0.6225126 | WT Fib up (group a) |
| Sgca | 1.34665691 | 1.27155122 | −0.147197 | 0.5906136 | WT Fib up (group a) |
| chr13: 108653722-108654406_I | −1.1271831 | −0.6123423 | −2.337821 | −1.5761738 | WT Fib up (group a) |
| Lef1 | 1.64070005 | 1.53550979 | 0.0379648 | 0.9643457 | WT Fib up (group a) |
| Loxl3 | 2.06427957 | 2.05154724 | 0.6835175 | 1.2584962 | WT Fib up (group a) |
| Spn | 1.45135696 | 1.47929872 | 0.501825 | 0.2602075 | WT Fib up (group a) |
| D18Ertd653e | 0.53415146 | 0.69814419 | −0.489111 | −0.4342732 | WT Fib up (group a) |
| Gm4146 | −0.0174682 | −0.1339814 | −0.908839 | −1.3944706 | WT Fib up (group a) |
| chr5: 91480533-91502237_R | 0.34799041 | 0.76937797 | −0.55159 | −0.4743477 | WT Fib up (group a) |
| Gm6372 | 1.505192 | 1.9666815 | 0.4617959 | 0.8704444 | WT Fib up (group a) |
| Mgst3 | 1.45181799 | 1.74738614 | 0.340244 | 0.7300746 | WT Fib up (group a) |
| Csgalnact1 | 1.02092864 | 1.32242547 | 0.2074173 | 0.0073862 | WT Fib up (group a) |
| chr6: 4841700-4847400_R | 0.68469383 | 0.64972282 | −0.404802 | −0.3869695 | WT Fib up (group a) |
| Ptchd3 | 0.34480776 | −0.1792325 | −0.863871 | −1.0921194 | WT Fib up (group a) |
| C81189 | 0.22010349 | −0.2183895 | −0.867388 | −1.2478837 | WT Fib up (group a) |
| Cd1d1 | 0.93375449 | 0.79881889 | −0.133586 | −0.2457394 | WT Fib up (group a) |
| chr1: 162965309-162970704_F | −0.3362106 | −0.7390523 | −1.304642 | −1.8805772 | WT Fib up (group a) |
| Slc9a5 | 0.192707 | 0.0963058 | −0.604945 | −1.2157873 | WT Fib up (group a) |
| chr18: 61809278-61825191_R | 0.61283772 | 0.29937658 | −0.410065 | −0.784078 | WT Fib up (group a) |
| Pcdhb21 | 1.22141287 | 1.41365205 | 0.043149 | 0.4871819 | WT Fib up (group a) |
| Dmrt1 | 0.31087519 | −0.1453174 | −0.793438 | −1.1447629 | WT Fib up (group a) |
| Cacng5 | −0.9487877 | −0.2055632 | −1.878332 | −1.3786769 | WT Fib up (group a) |
| chr15: 85534202-85537090_F | −0.1185329 | −0.0340584 | −1.024663 | −1.2280841 | WT Fib up (group a) |
| Efnb1 | 1.06788502 | 0.90359637 | −0.270817 | 0.1460147 | WT Fib up (group a) |
| D630002G06Rik | −0.3190713 | 0.00223436 | −1.192072 | −1.2095902 | WT Fib up (group a) |
| chrX: 54636915-54638388_R | 0.67832609 | 1.12738272 | −0.240054 | −0.0260406 | WT Fib up (group a) |
| Angpt1 | 2.30935924 | 2.3242842 | 0.7281617 | 1.8369296 | WT Fib up (group a) |
| Tle2 | 1.52051857 | 1.53699624 | 0.3785216 | 0.6131587 | WT Fib up (group a) |
| Ankle1 | 0.29658794 | −0.0948977 | −0.792398 | −1.0640959 | WT Fib up (group a) |
| A030001D16Rik | 0.4826133 | 0.57430022 | −0.657534 | −0.3433907 | WT Fib up (group a) |
| Cetn4 | 0.84139785 | 1.00902838 | −0.218443 | 0.0122558 | WT Fib up (group a) |
| Prdm16 | 1.00501533 | 1.340456 | 0.404191 | −0.1083124 | WT Fib up (group a) |
| chr9: 43926204-43928496_F | 0.2438333 | 0.48981185 | −0.607211 | −0.7086786 | WT Fib up (group a) |
| 5830403F22Rik | 0.17017163 | 0.01619655 | −0.897919 | −0.9638997 | WT Fib up (group a) |
| B3gnt9-ps | 0.61498109 | 0.56698631 | −0.532341 | −0.3298783 | WT Fib up (group a) |
| Efs | 1.12880984 | 1.2016358 | 0.0940018 | 0.1938559 | WT Fib up (group a) |
| Camk1g | 0.69249984 | 0.35706133 | −0.398083 | −0.5818516 | WT Fib up (group a) |
| chr19: 5834117-5835940_R | 0.32742829 | 0.10154646 | −0.524568 | −1.0725142 | WT Fib up (group a) |
| chr9: 43921700-43939600_F | 0.45511825 | 0.52274624 | −0.540316 | −0.5074748 | WT Fib up (group a) |
| Hmgn2l6 | 0.37088738 | 0.40794441 | −0.698515 | −0.5469915 | WT Fib up (group a) |
| Gdf10 | 1.09467928 | 0.98300475 | −0.002441 | 0.0578279 | WT Fib up (group a) |
| Idh1 | 0.13960472 | 0.55803559 | −0.569292 | −0.754071 | WT Fib up (group a) |
| Actg2 | −0.9477811 | −1.1905546 | −2.208283 | −1.9421226 | WT Fib up (group a) |
| Pcdhb16 | 1.06846142 | 1.21861241 | −0.087908 | 0.363086 | WT Fib up (group a) |
| Hmg1l1 | 0.28879723 | 0.59735456 | −0.299997 | −0.8195987 | WT Fib up (group a) |
| Kif26b | 1.87851135 | 2.1176503 | 0.7250965 | 1.2701125 | WT Fib up (group a) |
| ENSMUST00000110158 | −0.4117018 | −0.136051 | 0.4555294 | 0.9977732 | KO Fib up (group c) |
| Robo3 | −0.0209769 | −0.1325497 | 0.6748052 | 1.1736968 | KO Fib up (group c) |
| C4b | 3.79730084 | 3.53079645 | 3.934478 | 5.3992484 | KO Fib up (group c) |
| Cxx1b | 5.12491824 | 5.03236013 | 5.4038818 | 6.7596229 | KO Fib up (group c) |
| 1700024P16Rik | −1.4888346 | −1.5806689 | −0.492807 | −0.5692161 | KO Fib up (group c) |
| ENSMUST00000114877 | 0.03817983 | 0.19074814 | 1.2157894 | 1.0222634 | KO Fib up (group c) |
| Myoz2 | −0.2725226 | −0.1479629 | 0.5166884 | 1.0751926 | KO Fib up (group c) |
| Fstl3 | −1.1294343 | −0.8321314 | −0.11562 | 0.1666453 | KO Fib up (group c) |
| 0610010O12Rik | 0.0444931 | −0.2222391 | 0.6421557 | 1.1951709 | KO Fib up (group c) |
| Gm2881 | 0.35364434 | 0.47165819 | 1.2849226 | 1.5559902 | KO Fib up (group c) |
| Gm13109 | −0.1030105 | 0.04066775 | 1.1665117 | 0.7884132 | KO Fib up (group c) |
| Klra15 | 0.33918216 | 0.18983984 | 1.2737525 | 1.2758457 | KO Fib up (group c) |
| Cd200r2 | 0.09108576 | 0.01108012 | 0.8594155 | 1.2666048 | KO Fib up (group c) |
| Car12 | −0.338639 | −0.3921344 | 0.6838319 | 0.6109993 | KO Fib up (group c) |
| Kif5c | 1.65711747 | 1.79343613 | 2.2256444 | 3.2560686 | KO Fib up (group c) |
| Nupr1 | 0.05306155 | −0.0381744 | 0.7137489 | 1.3330203 | KO Fib up (group c) |
| 9630033F20Rik | −0.2675891 | −0.4266722 | 0.5184051 | 0.82403 | KO Fib up (group c) |
| 9930111J21Rik1 | −0.0037155 | −0.0632797 | 0.876542 | 1.0982127 | KO Fib up (group c) |
| Zc3hav1 | 0.15502652 | 0.11746376 | 1.0446742 | 1.2766125 | KO Fib up (group c) |
| Hap1 | 0.18517982 | −0.0824637 | 1.0362797 | 1.1153846 | KO Fib up (group c) |
| Cyp26a1 | 0.22112465 | 0.24837182 | 1.2079997 | 1.3110932 | KO Fib up (group c) |
| chr6: 57560819-57569928_F | −0.0599478 | −0.1440047 | 0.9483203 | 0.9011424 | KO Fib up (group c) |
| 2700008G24Rik | 0.746497 | 0.79213966 | 1.1847898 | 2.4073995 | KO Fib up (group c) |
| Pomc | 0.56799264 | 1.08109292 | 1.3783488 | 2.3277013 | KO Fib up (group c) |
| Plin4 | 2.0944134 | 1.87703859 | 2.4083195 | 3.6224573 | KO Fib up (group c) |
| Tnfsf15 | 0.54768184 | 0.70335549 | 1.292511 | 2.0208377 | KO Fib up (group c) |
| Ptpn22 | −0.1071094 | −0.4402739 | 0.8842216 | 0.6394182 | KO Fib up (group c) |
| Cyp4f14 | 0.59883542 | 0.56092992 | 1.0379305 | 2.1935078 | KO Fib up (group c) |
| Gm2371 | 0.17045399 | −0.2509354 | 0.738648 | 1.2535092 | KO Fib up (group c) |
| Bnip3 | −0.4030337 | −0.4741753 | 0.616122 | 0.579532 | KO Fib up (group c) |
| Pvr | −1.4065728 | −1.4889027 | −0.455911 | −0.3654696 | KO Fib up (group c) |
| ENSMUST00000043406 | 0.70073767 | 0.8108145 | 1.2712006 | 2.315784 | KO Fib up (group c) |
| Npc2 | 0.77094091 | 0.73509062 | 1.7137464 | 1.8688026 | KO Fib up (group c) |
| Ifi202b | 1.19064605 | 1.27130106 | 2.0884726 | 2.4549446 | KO Fib up (group c) |
| Serpinb6b | −0.8535663 | −0.5431455 | 0.2738399 | 0.4151361 | KO Fib up (group c) |
| 9130017N09Rik | −0.5696097 | −0.2091461 | 0.5873033 | 0.7198969 | KO Fib up (group c) |
| Taf9b | 0.10118391 | 0.40314668 | 1.0750542 | 1.5184392 | KO Fib up (group c) |
| Lancl3 | −0.3315559 | −0.0187945 | 0.5769544 | 1.163069 | KO Fib up (group c) |
| Ncf4 | 1.08480687 | 1.05298381 | 1.7742355 | 2.4549438 | KO Fib up (group c) |
| Ninj2 | 0.02555934 | 0.07383051 | 1.0754463 | 1.1178326 | KO Fib up (group c) |
| Gnb4 | 0.13941511 | 0.28563837 | 1.0125036 | 1.5158678 | KO Fib up (group c) |
| Rnf31 | 0.17673403 | 0.00602687 | 1.0961588 | 1.191146 | KO Fib up (group c) |
| Arrdc4 | 1.21151624 | 1.4345377 | 2.0310231 | 2.7199863 | KO Fib up (group c) |
| Gm5133 | −0.0773974 | 0.00331128 | 1.0017406 | 1.0312955 | KO Fib up (group c) |
| Esr1 | 0.12987502 | 0.25690957 | 1.2908919 | 1.2062671 | KO Fib up (group c) |
| Larp6 | 1.50185558 | 1.10995014 | 1.967235 | 2.7569171 | KO Fib up (group c) |
| LOC674761 | 0.93470989 | 0.1449155 | 1.4688168 | 1.7278196 | KO Fib up (group c) |
| Gpnmb | 3.13359201 | 3.02783283 | 3.516525 | 4.7636059 | KO Fib up (group c) |
| Nckap5 | 0.76607409 | 0.90815615 | 1.1956667 | 2.6036645 | KO Fib up (group c) |
| Pnpt1 | −0.3469333 | −0.1373667 | 0.7610746 | 0.8822718 | KO Fib up (group c) |
| C920025E04Rik | 0.16295375 | 0.16295375 | 1.0595457 | 1.3948689 | KO Fib up (group c) |
| Gca | 0.53592581 | 0.52050951 | 0.9535168 | 2.2433277 | KO Fib up (group c) |
| Lrrc14b | −1.7992969 | −1.1982514 | −0.356433 | −0.5003246 | KO Fib up (group c) |
| Psmb10 | 0.17434043 | 0.23008332 | 1.1163616 | 1.4315013 | KO Fib up (group c) |
| Pde1b | −1.2614198 | −1.166033 | 0.1435316 | −0.4250656 | KO Fib up (group c) |
| Gm2015 | 0.37835307 | 0.09606237 | 0.9824639 | 1.640141 | KO Fib up (group c) |
| Adh1 | 0.29943604 | 0.57870594 | 1.4588874 | 1.5693186 | KO Fib up (group c) |
| Jam3 | 3.61928992 | 3.67315127 | 4.1365057 | 5.3080736 | KO Fib up (group c) |
| Zbtb16 | 0.65283464 | 0.96415633 | 1.1603263 | 2.6129473 | KO Fib up (group c) |
| Mmp13 | −1.0344474 | −0.3925953 | 0.5047379 | 0.2272405 | KO Fib up (group c) |
| Rbm46 | 0.57149277 | 0.71071595 | 1.2799986 | 2.1620525 | KO Fib up (group c) |
| Evc2 | 2.57589051 | 2.38264646 | 3.0595381 | 4.0592528 | KO Fib up (group c) |
| Nr1h4 | 0.3315848 | 0.30386405 | 1.401023 | 1.3974505 | KO Fib up (group c) |
| LOC640793 | 0.14603188 | 0.50741009 | 1.4786427 | 1.3471295 | KO Fib up (group c) |
| Hhex | 0.68899905 | 0.28934866 | 1.1708182 | 1.981163 | KO Fib up (group c) |
| Rab20 | −0.9496064 | −0.8271544 | −0.046503 | 0.4462479 | KO Fib up (group c) |
| Hspb2 | −0.7882204 | −0.4520219 | 0.3287617 | 0.6081383 | KO Fib up (group c) |
| Cxcl3 | −2.5571581 | −1.9521339 | −0.854202 | −1.4772137 | KO Fib up (group c) |
| Apln | −1.3862297 | −1.1658628 | −0.117815 | −0.2521063 | KO Fib up (group c) |
| Col1a1 | 5.52777377 | 5.32678793 | 5.7793493 | 7.2590731 | KO Fib up (group c) |
| Aoc3 | 1.06766391 | 1.44717949 | 1.524363 | 3.1792693 | KO Fib up (group c) |
| Hfm1 | 0.08201507 | 0.32498511 | 1.4210356 | 1.1778295 | KO Fib up (group c) |
| Slco3a1 | 2.12800395 | 1.89294947 | 2.5114946 | 3.7081846 | KO Fib up (group c) |
| Rnd1 | −1.2494773 | −1.114362 | −0.188698 | 0.02541 | KO Fib up (group c) |
| Chchd10 | −0.2461197 | −0.5142586 | 0.5905925 | 0.8522132 | KO Fib up (group c) |
| Zmynd12 | −0.1488062 | −0.1926605 | 0.9910514 | 0.8720228 | KO Fib up (group c) |
| Rspo3 | 3.41650158 | 3.55509083 | 3.7936008 | 5.3834235 | KO Fib up (group c) |
| Itih5 | 0.20063895 | 0.18813447 | 1.6139245 | 0.9871676 | KO Fib up (group c) |
| 1110002E22Rik | −1.9008705 | −1.6278481 | −0.736787 | −0.5790202 | KO Fib up (group c) |
| Iffo1 | 2.07850695 | 1.96564368 | 2.6400808 | 3.6212487 | KO Fib up (group c) |
| Daxx | 0.00924025 | 0.02873277 | 0.9150869 | 1.3426162 | KO Fib up (group c) |
| Cd34 | 0.63938582 | 0.47037363 | 1.571811 | 1.7591117 | KO Fib up (group c) |
| LOC790956 | −0.249682 | −0.3251043 | 0.9644673 | 0.6828745 | KO Fib up (group c) |
| Slc7a11 | −0.6156407 | −0.1043931 | 0.7617403 | 0.7447363 | KO Fib up (group c) |
| ENSMUST00000055994 | −0.2023916 | −0.2582448 | 0.6782523 | 1.0971168 | KO Fib up (group c) |
| Stx3 | −0.3352636 | −0.0350866 | 0.6782969 | 1.1878252 | KO Fib up (group c) |
| Tigit | −1.8621259 | −1.8823938 | −0.344973 | −1.1614257 | KO Fib up (group c) |
| Ascc3 | −0.1832788 | −0.0846456 | 0.9369383 | 1.0339618 | KO Fib up (group c) |
| Fli1 | 0.6191873 | 0.43220966 | 0.9387753 | 2.3523482 | KO Fib up (group c) |
| Col6a4 | −0.0852827 | 0.01251368 | 1.1437694 | 1.0267217 | KO Fib up (group c) |
| Trim35 | 3.02432229 | 3.07977266 | 3.7349332 | 4.6129142 | KO Fib up (group c) |
| Pml | 0.39190429 | 0.29574936 | 1.3060144 | 1.6262598 | KO Fib up (group c) |
| Sp110 | 0.39151851 | 0.41608971 | 1.5312379 | 1.5360575 | KO Fib up (group c) |
| Lrrc51 | 0.37389812 | 0.26131514 | 1.2370852 | 1.6583207 | KO Fib up (group c) |
| Emilin2 | 0.88615134 | 0.77079585 | 1.7421771 | 2.180893 | KO Fib up (group c) |
| Tff1 | 0.64344639 | 0.26039588 | 1.6523436 | 1.5255574 | KO Fib up (group c) |
| Gsta1 | −1.9227751 | −1.7724171 | −0.309399 | −1.1109255 | KO Fib up (group c) |
| Ccdc68 | 0.75838555 | 1.04013358 | 1.5217846 | 2.5523681 | KO Fib up (group c) |
| 5430410E06Rik | 0.3346622 | 0.32653568 | 1.4504259 | 1.4916633 | KO Fib up (group c) |
| LOC635676 | 1.625138 | −0.0186356 | 1.85946 | 2.0291144 | KO Fib up (group c) |
| Pla2g2e | 0.91437431 | 0.8834762 | 1.5582547 | 2.5221102 | KO Fib up (group c) |
| Olfr224 | 1.00727886 | 0.79552923 | 1.4139892 | 2.6743234 | KO Fib up (group c) |
| Gm4340 | 0.16429264 | −0.3255854 | 1.3027282 | 0.8220302 | KO Fib up (group c) |
| Cfh | 3.52500535 | 3.40276544 | 3.7433547 | 5.4715339 | KO Fib up (group c) |
| Pdlim4 | 1.48949674 | 1.41093716 | 2.0389369 | 3.1505469 | KO Fib up (group c) |
| 1810020O05Rik | −0.8903921 | −0.8748589 | 0.4445847 | 0.0800197 | KO Fib up (group c) |
| Slc9a7 | −0.2542075 | −0.0054632 | 0.6935084 | 1.3370776 | KO Fib up (group c) |
| AK044029 | 0.66824025 | 0.99570652 | 1.4709637 | 2.4900551 | KO Fib up (group c) |
| AW112010 | 0.86314382 | 1.06481387 | 1.7506466 | 2.4783123 | KO Fib up (group c) |
| Rasgrp3 | 0.52214699 | 0.56778965 | 1.5491999 | 1.8448992 | KO Fib up (group c) |
| Kcnab3 | −1.8242888 | −1.4900693 | −0.18623 | −0.8218799 | KO Fib up (group c) |
| Tmco4 | 0.49933929 | 0.37101264 | 1.3070704 | 1.8735412 | KO Fib up (group c) |
| Prr11 | 1.90239801 | 1.74940407 | 2.4595522 | 3.5035462 | KO Fib up (group c) |
| Oas1g | 0.07486205 | 0.35175219 | 1.2699516 | 1.4693745 | KO Fib up (group c) |
| 5830408B19Rik | 0.85878216 | 0.91358051 | 1.4918441 | 2.5988683 | KO Fib up (group c) |
| Loxl1 | 2.42565884 | 2.16792857 | 2.8919623 | 4.0257442 | KO Fib up (group c) |
| Kcnab1 | 0.80653113 | 1.17551282 | 1.9236506 | 2.3905092 | KO Fib up (group c) |
| Txnip | 1.22950459 | 1.29886734 | 2.3835385 | 2.4809735 | KO Fib up (group c) |
| Mvp | 0.1088668 | −0.0316648 | 1.1271173 | 1.286965 | KO Fib up (group c) |
| Il18bp | −0.2013526 | −0.2758765 | 0.5462431 | 1.3143772 | KO Fib up (group c) |
| Sh2d1b1 | −0.0875966 | −0.0402466 | 0.8105573 | 1.4036145 | KO Fib up (group c) |
| H2afy3 | 2.61364429 | 2.65498317 | 3.2554236 | 4.3569768 | KO Fib up (group c) |
| Fam46a | 0.60372274 | 0.65251431 | 1.6184456 | 1.9818922 | KO Fib up (group c) |
| Dub1a | −0.0773299 | −0.0519911 | 0.9690478 | 1.2468332 | KO Fib up (group c) |
| Lbp | 1.75642854 | 1.69914755 | 2.358937 | 3.4447318 | KO Fib up (group c) |
| Capn6 | 0.30337745 | 0.20560551 | 1.2677603 | 1.5920537 | KO Fib up (group c) |
| chr9: 78104935-78116974_F | −1.7243367 | −1.6560386 | −0.136064 | −0.8904847 | KO Fib up (group c) |
| Neu1 | 0.12736254 | 0.17974236 | 0.9669532 | 1.6945931 | KO Fib up (group c) |
| 9530008L14Rik | −0.1600539 | −0.1195424 | 0.6437731 | 1.4334656 | KO Fib up (group c) |
| Akr1c14 | 1.35949759 | 1.81785222 | 1.916086 | 3.6193647 | KO Fib up (group c) |
| Sell | 0.07177334 | 0.20705404 | 1.496166 | 1.1409988 | KO Fib up (group c) |
| AI646023 | 2.17385792 | 2.35541901 | 2.7212236 | 4.1674168 | KO Fib up (group c) |
| Gm2034 | 0.32794707 | 0.16026263 | 1.0954194 | 1.7538468 | KO Fib up (group c) |
| Fstl1 | 6.37591495 | 6.51327338 | 6.9568578 | 8.2955675 | KO Fib up (group c) |
| Scrg1 | −0.1472368 | −0.1488809 | 0.6930716 | 1.3761822 | KO Fib up (group c) |
| Ltb | 0.47853492 | 0.57813309 | 1.8118545 | 1.610574 | KO Fib up (group c) |
| Macrod1 | −0.0297071 | −0.1182359 | 0.8663935 | 1.3524152 | KO Fib up (group c) |
| H2-M11 | 0.40082306 | −0.0380979 | 1.4839048 | 1.2516633 | KO Fib up (group c) |
| Irak3 | 0.95210706 | 1.28595128 | 1.5697443 | 3.0459908 | KO Fib up (group c) |
| ENSMUST00000101339 | 0.02035461 | 0.03648815 | 1.2128437 | 1.2242656 | KO Fib up (group c) |
| Hoxa9 | 0.43280404 | 0.16746933 | 1.1410348 | 1.8406483 | KO Fib up (group c) |
| Igf2bp1 | −1.0589581 | −0.9906964 | 0.0753563 | 0.2615324 | KO Fib up (group c) |
| Tmem229b | 0.87140691 | 0.7980304 | 1.84096 | 2.2152802 | KO Fib up (group c) |
| Siglecg | −1.5133531 | −1.5266097 | −0.216656 | −0.4303585 | KO Fib up (group c) |
| Stxbp6 | 0.99258228 | 1.56343518 | 1.8922002 | 3.0630134 | KO Fib up (group c) |
| Vgf | −0.3293674 | −0.521323 | 0.8755589 | 0.6734692 | KO Fib up (group c) |
| Evc | 1.41330202 | 1.87282551 | 1.9956634 | 3.6908681 | KO Fib up (group c) |
| chr6: 57583950-57624216_R | 0.05001153 | 0.2451452 | 1.4231451 | 1.2741499 | KO Fib up (group c) |
| Fam26e | 1.35429103 | 1.88182242 | 2.0557595 | 3.5866434 | KO Fib up (group c) |
| BC034090 | 0.36923697 | 0.7826489 | 1.277571 | 2.2834739 | KO Fib up (group c) |
| 6330509M05Rik | −0.3126548 | −0.2462414 | 0.7526319 | 1.1003555 | KO Fib up (group c) |
| Ablim1 | 0.70935214 | 0.74967062 | 1.4423142 | 2.4318591 | KO Fib up (group c) |
| ENSMUST00000109958 | 0.08373236 | 0.00631949 | 0.9286592 | 1.5828567 | KO Fib up (group c) |
| LOC625360 | 0.16134245 | −0.2252135 | 1.2830414 | 1.0774751 | KO Fib up (group c) |
| chr5: 15146475-15170100_F | 0.40451099 | 0.70639721 | 0.873401 | 2.6658326 | KO Fib up (group c) |
| Mtus1 | 1.29839648 | 1.22540135 | 1.8369208 | 3.1210611 | KO Fib up (group c) |
| Casz1 | 0.04399134 | 0.08052537 | 1.1586501 | 1.4032104 | KO Fib up (group c) |
| Gem | 0.53909814 | 0.85478808 | 1.4964346 | 2.3355478 | KO Fib up (group c) |
| Tmem125 | −0.3559758 | −0.1847338 | 0.6617021 | 1.236215 | KO Fib up (group c) |
| BC023969 | 1.38946982 | 1.46557833 | 2.2633302 | 3.0356137 | KO Fib up (group c) |
| Arhgap29 | 2.22899019 | 2.31414914 | 2.9612796 | 4.0264273 | KO Fib up (group c) |
| Zscan4a | −0.1423506 | −0.1028197 | 1.3840794 | 0.8155244 | KO Fib up (group c) |
| Fkbp10 | 7.09714327 | 6.6805492 | 7.4961007 | 8.7291843 | KO Fib up (group c) |
| Mical1 | 4.53838314 | 4.43564485 | 5.0744586 | 6.3485765 | KO Fib up (group c) |
| Lce3b | −0.0251977 | 0.0129439 | 1.15025 | 1.3007385 | KO Fib up (group c) |
| Tpm2 | 0.75563251 | 0.70504543 | 1.3406329 | 2.5980849 | KO Fib up (group c) |
| D13Ertd608e | 0.1639789 | −0.0097826 | 1.0396508 | 1.5980473 | KO Fib up (group c) |
| Chac1 | −0.0104385 | −0.3291676 | 0.8505107 | 1.2965231 | KO Fib up (group c) |
| ENSMUST00000093501 | 0.67038467 | 0.73383654 | 1.672026 | 2.2218429 | KO Fib up (group c) |
| Inhbb | 2.43486957 | 2.26872229 | 3.1121349 | 4.0834098 | KO Fib up (group c) |
| C1qtnf5 | 1.5486098 | 1.523553 | 1.9860001 | 3.5794501 | KO Fib up (group c) |
| Flnc | −0.7338864 | −0.4818089 | 0.4478472 | 0.8335349 | KO Fib up (group c) |
| Ctgf | 2.54788231 | 2.48903321 | 3.2907894 | 4.2577396 | KO Fib up (group c) |
| Ptgds | 0.37694522 | 0.41009871 | 1.0757547 | 2.2319203 | KO Fib up (group c) |
| Cdc42ep5 | 1.32706838 | 1.31840841 | 2.1012589 | 3.0657892 | KO Fib up (group c) |
| Ifnb1 | 0.15227993 | 0.41759085 | 1.1757474 | 1.9180311 | KO Fib up (group c) |
| Gm6904 | 0.49951488 | 0.0436051 | 1.3202406 | 1.7683585 | KO Fib up (group c) |
| Mitd1 | 0.15942452 | 0.26028518 | 1.3807746 | 1.5891793 | KO Fib up (group c) |
| Ndrg1 | −1.0179291 | −1.0004454 | 0.3114382 | 0.2214233 | KO Fib up (group c) |
| Tmie | −0.0420933 | −0.1274307 | 1.2610692 | 1.1220658 | KO Fib up (group c) |
| Epb4.1l3 | 5.20392524 | 4.92888185 | 5.6855561 | 7.0033102 | KO Fib up (group c) |
| Ror2 | 3.51840351 | 3.44487095 | 4.1285685 | 5.3920148 | KO Fib up (group c) |
| 9530082P21Rik | 0.92518636 | 0.45475757 | 1.9326776 | 2.0062626 | KO Fib up (group c) |
| Cnksr1 | 0.14749142 | 0.38445317 | 1.6420866 | 1.4543491 | KO Fib up (group c) |
| Plekha7 | 0.37255684 | 0.43668717 | 1.1231783 | 2.2532492 | KO Fib up (group c) |
| Tap2 | 0.97619631 | 0.98524092 | 2.1219017 | 2.407461 | KO Fib up (group c) |
| H2-Q10 | 0.42313044 | 0.37317232 | 1.5372782 | 1.8282836 | KO Fib up (group c) |
| Stmn4 | −0.4774302 | −0.7416776 | 0.7165233 | 0.6337548 | KO Fib up (group c) |
| Gm8094 | −0.0390677 | 0.08017691 | 1.3464452 | 1.2647275 | KO Fib up (group c) |
| Ttc30a1 | 0.49650885 | 0.75139285 | 1.4644115 | 2.3634723 | KO Fib up (group c) |
| chr3: 146515333-146557069_F | 1.21021864 | 1.16987098 | 2.022751 | 2.9470173 | KO Fib up (group c) |
| Fabp4 | 2.62552115 | 2.5958597 | 3.0403834 | 4.7736063 | KO Fib up (group c) |
| Fads3 | −0.9690307 | −1.0310968 | −0.009185 | 0.6075686 | KO Fib up (group c) |
| Tspan13 | 1.07186051 | 0.94138698 | 1.7855723 | 2.8270074 | KO Fib up (group c) |
| Shisa5 | 0.26271737 | 0.33678726 | 1.6233317 | 1.5781161 | KO Fib up (group c) |
| Syt1 | −0.1897823 | −0.2872407 | 1.460214 | 0.6713969 | KO Fib up (group c) |
| Pigz | 0.01001676 | −0.0037364 | 1.4236848 | 1.2134349 | KO Fib up (group c) |
| Nlrc5 | 0.47455936 | 0.68297684 | 1.8022904 | 1.9882303 | KO Fib up (group c) |
| Il15ra | −0.0646507 | −0.0152484 | 1.209719 | 1.3434125 | KO Fib up (group c) |
| Adamts2 | 2.68744187 | 2.44500062 | 3.4572654 | 4.3089781 | KO Fib up (group c) |
| Gbp11 | 1.62986764 | 1.13577398 | 2.4471366 | 2.9556553 | KO Fib up (group c) |
| Parp11 | 0.05228327 | 0.13284544 | 1.2957888 | 1.5267011 | KO Fib up (group c) |
| Aldh1b1 | −0.1966264 | 0.04003767 | 1.2371987 | 1.2453209 | KO Fib up (group c) |
| F11r | −0.2430058 | 0.19029083 | 0.9605448 | 1.6261266 | KO Fib up (group c) |
| Prex2 | 0.06995524 | −0.0519665 | 1.3341583 | 1.3268387 | KO Fib up (group c) |
| Mettl7b | −0.4423785 | −0.54974 | 0.6660492 | 0.9853819 | KO Fib up (group c) |
| Isg20 | 0.17242733 | 0.07484815 | 1.345964 | 1.5508345 | KO Fib up (group c) |
| Tuba8 | −0.4580956 | −0.1384695 | 0.7500793 | 1.3085286 | KO Fib up (group c) |
| chr1: 175660642-175671878_R | 0.60507557 | 0.40629371 | 1.8320614 | 1.8376775 | KO Fib up (group c) |
| Msx1 | 1.51424315 | 1.48458298 | 2.2008713 | 3.4637093 | KO Fib up (group c) |
| Gulp1 | 1.36357677 | 1.46651679 | 2.0963595 | 3.3998073 | KO Fib up (group c) |
| Pcp4l1 | 1.6912887 | 2.04985477 | 2.4752455 | 3.9362717 | KO Fib up (group c) |
| Slc6a13 | 0.02946275 | 0.07747958 | 1.7052195 | 1.0751926 | KO Fib up (group c) |
| Enox2 | 1.49899457 | 1.65796821 | 2.3786854 | 3.4524688 | KO Fib up (group c) |
| ENSMUST00000105988 | 1.1375574 | 0.98505691 | 2.0229709 | 2.7800404 | KO Fib up (group c) |
| 8430427H17Rik | 1.00015652 | 0.99340888 | 1.8391206 | 2.8351345 | KO Fib up (group c) |
| Rasgef1b | 0.89631819 | 0.95114608 | 1.8799743 | 2.650572 | KO Fib up (group c) |
| Zfr2 | 1.26927925 | 1.30559829 | 2.2679371 | 2.9909151 | KO Fib up (group c) |
| ENSMUST00000111852 | 0.62122388 | 0.11855695 | 1.735021 | 1.6904584 | KO Fib up (group c) |
| Dub1 | 0.14241166 | −0.0054197 | 1.3045872 | 1.5197418 | KO Fib up (group c) |
| Six2 | 1.78224525 | 1.64613424 | 2.4474738 | 3.6732829 | KO Fib up (group c) |
| Fut1 | 0.03346643 | 0.03707166 | 1.1289052 | 1.6373557 | KO Fib up (group c) |
| Slfn10-ps | 0.31978999 | 0.06553476 | 1.4493086 | 1.6333982 | KO Fib up (group c) |
| Gadd45b | −1.076818 | −1.2971938 | 0.0249488 | 0.3035462 | KO Fib up (group c) |
| Sept4 | −0.3282572 | −0.5228723 | 0.7067097 | 1.1453077 | KO Fib up (group c) |
| A030001D20Rik | 0.73821978 | 0.81910653 | 2.1097521 | 2.1590739 | KO Fib up (group c) |
| Angptl7 | 1.6915503 | 1.39771924 | 2.679463 | 3.1362439 | KO Fib up (group c) |
| Thsd7a | −0.0019126 | 0.11241305 | 1.0565695 | 1.7811209 | KO Fib up (group c) |
| Pdk4 | 1.88930674 | 1.78825722 | 2.5327089 | 3.872088 | KO Fib up (group c) |
| Gm8909 | 0.13546129 | 0.01353954 | 1.5567692 | 1.3262785 | KO Fib up (group c) |
| Dock10 | 0.19721199 | 0.21794146 | 1.3296154 | 1.8223284 | KO Fib up (group c) |
| Samd5 | 1.2757395 | 1.09626718 | 1.8850843 | 3.2244817 | KO Fib up (group c) |
| Gm13101 | −0.199995 | −0.2409252 | 1.3014028 | 0.9973715 | KO Fib up (group c) |
| Cox6a2 | 0.24530509 | 0.3134122 | 1.7354796 | 1.5651807 | KO Fib up (group c) |
| Sorl1 | 0.48757847 | 1.07201779 | 1.6562679 | 2.6460946 | KO Fib up (group c) |
| 4732456N10Rik | 0.12900724 | 0.30194691 | 1.130604 | 2.0438641 | KO Fib up (group c) |
| Gm13138 | 0.11665186 | 0.02598227 | 1.0438857 | 1.8451094 | KO Fib up (group c) |
| Ddit3 | −0.0881086 | −0.4095479 | 0.8431671 | 1.406757 | KO Fib up (group c) |
| Baiap2l1 | −0.4091222 | −0.0679219 | 0.7386452 | 1.5330831 | KO Fib up (group c) |
| Lgals8 | 0.34053865 | 0.47463173 | 1.6756436 | 1.8922452 | KO Fib up (group c) |
| Trpv4 | 0.38816017 | 0.50309117 | 1.273543 | 2.3706917 | KO Fib up (group c) |
| chr5: 15033568-15033987_F | 0.51686166 | 0.70331165 | 1.288574 | 2.6948534 | KO Fib up (group c) |
| Abca8b | 0.35843433 | −0.0085817 | 1.1647319 | 1.969304 | KO Fib up (group c) |
| Gngt2 | 2.12567848 | 1.92932483 | 3.0346306 | 3.8059376 | KO Fib up (group c) |
| Trim69 | 0.24497349 | 0.05386729 | 1.1729569 | 1.9153747 | KO Fib up (group c) |
| Nsd1 | 1.04883315 | 1.21476553 | 2.0462825 | 3.0164931 | KO Fib up (group c) |
| Ifi204 | 0.69463311 | 0.96024137 | 2.0826049 | 2.380042 | KO Fib up (group c) |
| Gbp10 | 0.2519489 | −0.3814433 | 1.3020487 | 1.3780806 | KO Fib up (group c) |
| C2 | 0.20613241 | −0.1652469 | 1.3904634 | 1.4603416 | KO Fib up (group c) |
| C1qtnf4 | 0.82794283 | 1.132675 | 1.7412028 | 3.039184 | KO Fib up (group c) |
| Fsd1 | 0.5450951 | 0.59759788 | 1.2402696 | 2.7235763 | KO Fib up (group c) |
| Irf5 | 0.85484838 | 0.6327423 | 2.0863406 | 2.2232611 | KO Fib up (group c) |
| Aim2 | −0.0694246 | 0.42292991 | 1.4223127 | 1.7578808 | KO Fib up (group c) |
| Thsd1 | 1.39592493 | 1.68679451 | 2.2687816 | 3.6422248 | KO Fib up (group c) |
| ENSMUST00000113886 | 0.06378457 | −0.1999467 | 1.2442213 | 1.4493925 | KO Fib up (group c) |
| Ms4a4d | −0.0511515 | 0.02361625 | 0.9754602 | 1.8334672 | KO Fib up (group c) |
| Rgnef | 0.28696383 | 0.57477893 | 1.4898969 | 2.2111564 | KO Fib up (group c) |
| Cyp1b1 | 1.09214698 | 0.79769329 | 2.1496965 | 2.5808336 | KO Fib up (group c) |
| Lifr | −0.3924011 | −0.3156311 | 0.6818679 | 1.4527948 | KO Fib up (group c) |
| Tusc1 | 0.88909522 | 1.15845015 | 1.8827164 | 3.0093485 | KO Fib up (group c) |
| Wbscr17 | 0.19501367 | 0.32932576 | 1.3393307 | 2.0312272 | KO Fib up (group c) |
| Gm5458 | 0.23314547 | 0.15841659 | 1.3417629 | 1.8964312 | KO Fib up (group c) |
| Smoc1 | 1.88507359 | 1.70536757 | 2.5003517 | 3.941849 | KO Fib up (group c) |
| Cryab | −1.0622532 | −1.1845719 | 0.3454539 | 0.2637324 | KO Fib up (group c) |
| Sp100 | 0.24778968 | 0.18354509 | 1.642533 | 1.6449889 | KO Fib up (group c) |
| Rhbdl3 | 0.24023702 | 0.50054884 | 1.2168744 | 2.3802605 | KO Fib up (group c) |
| Zfp677 | 0.89315194 | 0.96830243 | 1.7574968 | 2.9610042 | KO Fib up (group c) |
| Emb | 0.26591469 | 0.39654795 | 0.9900448 | 2.5304923 | KO Fib up (group c) |
| Msl3l2 | 0.51131486 | 0.54626237 | 1.5031654 | 2.4134172 | KO Fib up (group c) |
| Klf8 | 0.37303846 | 0.11467253 | 1.2711924 | 2.0788635 | KO Fib up (group c) |
| Gm6270 | −2.099689 | −2.1088007 | 0.630144 | −1.9750414 | KO Fib up (group c) |
| Arhgdig | 0.25017121 | 0.37337295 | 1.4001424 | 2.0978614 | KO Fib up (group c) |
| Gli3 | 2.85594863 | 3.33883984 | 3.7781318 | 5.2981648 | KO Fib up (group c) |
| Cebpd | 0.41609556 | −0.1546346 | 1.2892445 | 1.8686178 | KO Fib up (group c) |
| Il15 | 0.64928366 | 0.80171473 | 2.3138907 | 2.0358277 | KO Fib up (group c) |
| Pla1a | −0.5901098 | −0.6771415 | 0.5705297 | 1.0650306 | KO Fib up (group c) |
| Inha | 0.73960185 | 0.69672048 | 1.6271271 | 2.7219762 | KO Fib up (group c) |
| 1700019B21Rik | −0.2461382 | 0.15591204 | 1.1155399 | 1.7081156 | KO Fib up (group c) |
| Dntt | −0.0199064 | −0.0092065 | 1.5381059 | 1.3483455 | KO Fib up (group c) |
| ENSMUST00000099340 | −1.9201665 | −1.8258484 | −0.310729 | −0.5184315 | KO Fib up (group c) |
| Tlr3 | 0.5604786 | 0.76945888 | 2.0152497 | 2.2329881 | KO Fib up (group c) |
| Znfx1 | 0.39832202 | 0.61418269 | 1.8625485 | 2.0733564 | KO Fib up (group c) |
| chr1: 175654606-175672031_R | 0.94076162 | 1.06469548 | 2.3930704 | 2.5372396 | KO Fib up (group c) |
| 1700007G11Rik | −0.812836 | −1.0771626 | 0.2134013 | 0.8228862 | KO Fib up (group c) |
| LOC100048309 | 0.85418043 | 0.8067621 | 2.2175577 | 2.3696757 | KO Fib up (group c) |
| Crct1 | −0.0221504 | −0.1033326 | 1.2109383 | 1.6012092 | KO Fib up (group c) |
| Aloxe3 | −0.1096672 | −0.2448416 | 1.2472959 | 1.3419482 | KO Fib up (group c) |
| Fign | 0.67976697 | 0.97507018 | 1.8012561 | 2.80019 | KO Fib up (group c) |
| Fam92a | 4.37089939 | 4.43512832 | 5.1723108 | 6.5950352 | KO Fib up (group c) |
| Hoxb6 | 0.8618405 | 0.77416112 | 3.0874736 | 1.5122716 | KO Fib up (group c) |
| Gda | 0.74704587 | 0.62999292 | 1.7562836 | 2.5866869 | KO Fib up (group c) |
| Prdm5 | 4.01079008 | 3.62180862 | 4.7034381 | 5.8985426 | KO Fib up (group c) |
| Abcb1a | −0.4898031 | −0.4690288 | 0.7825317 | 1.2294333 | KO Fib up (group c) |
| Aqp3 | 0.6801804 | 0.88968091 | 1.9068343 | 2.6352206 | KO Fib up (group c) |
| ENSMUST00000115995 | −0.1068012 | −0.1694739 | 0.9445382 | 1.7533219 | KO Fib up (group c) |
| AU022751 | 0.02079511 | −0.0156808 | 0.9947645 | 1.9940209 | KO Fib up (group c) |
| chr5: 14943275-14944205_F | 0.46284495 | 0.67901851 | 1.0725076 | 3.0556866 | KO Fib up (group c) |
| Gbp9 | 1.78514857 | 1.88193497 | 3.1641456 | 3.4929625 | KO Fib up (group c) |
| Pnpla7 | −0.0512663 | 0.33413053 | 1.2960381 | 1.9882324 | KO Fib up (group c) |
| Parp10 | 0.91293872 | 0.51899801 | 2.199698 | 2.2374234 | KO Fib up (group c) |
| Gm2022 | 0.88161352 | 1.21282329 | 2.2464502 | 2.8539207 | KO Fib up (group c) |
| Armcx2 | 3.82114273 | 3.71889745 | 4.6536438 | 5.9073258 | KO Fib up (group c) |
| LOC632263 | 1.26083645 | 1.03625177 | 2.3251012 | 2.9942024 | KO Fib up (group c) |
| Tmem140 | 2.82820248 | 2.54308019 | 3.8249351 | 4.5784067 | KO Fib up (group c) |
| Nudt16 | 0.96499497 | 1.07972968 | 2.0668609 | 3.0115806 | KO Fib up (group c) |
| Gm8232 | −0.1495913 | 0.1071305 | 1.0828596 | 1.9158601 | KO Fib up (group c) |
| Zmynd15 | 0.11322975 | 0.20867132 | 1.2981732 | 2.066475 | KO Fib up (group c) |
| Kng1 | 0.34809015 | 0.13489634 | 1.3079386 | 2.2268027 | KO Fib up (group c) |
| Tnc | −0.6343651 | −0.4867253 | 0.5888272 | 1.342273 | KO Fib up (group c) |
| LOC641235 | 0.40442059 | −0.0406695 | 1.8631564 | 1.5637053 | KO Fib up (group c) |
| Gucy1b3 | 0.53137375 | 0.57399539 | 1.4838342 | 2.6877253 | KO Fib up (group c) |
| chr9: 4260788-4261404_F | 0.581217 | 1.01105711 | 1.8043208 | 2.8561588 | KO Fib up (group c) |
| Acta2 | 0.69141662 | 0.61874815 | 1.5510911 | 2.8354223 | KO Fib up (group c) |
| ENSMUST00000111210 | 1.00721684 | 1.17164326 | 2.6685378 | 2.5907241 | KO Fib up (group c) |
| Ly6f | 0.14450818 | 0.15133482 | 1.5300211 | 1.8466138 | KO Fib up (group c) |
| Ttll7 | 0.78505284 | 0.72264304 | 1.7323582 | 2.857866 | KO Fib up (group c) |
| chr13: 60524145-60528559_R | 0.3464999 | 0.29611558 | 1.2721746 | 2.457119 | KO Fib up (group c) |
| Cdk15 | 0.78162123 | 0.73705148 | 1.838426 | 2.7701352 | KO Fib up (group c) |
| Abcb4 | 0.90400864 | 1.04386462 | 2.133032 | 2.9081124 | KO Fib up (group c) |
| Tmlhe | 0.62641477 | 1.04761033 | 1.7764966 | 2.9963562 | KO Fib up (group c) |
| E030037K03Rik | 0.16314482 | −0.1835057 | 1.0798585 | 1.9988989 | KO Fib up (group c) |
| ENSMUST00000074428 | 2.02333047 | 1.91362569 | 3.0126126 | 4.0329279 | KO Fib up (group c) |
| Dlx2 | 1.59819026 | 1.87669374 | 2.7166485 | 3.8693818 | KO Fib up (group c) |
| Prickle1 | −1.4629446 | −1.3334466 | −0.11669 | 0.4346232 | KO Fib up (group c) |
| Trex1 | 0.85997053 | 0.66974002 | 2.1593424 | 2.4860544 | KO Fib up (group c) |
| Egfr | 2.73967343 | 2.85859872 | 3.8070409 | 4.9094119 | KO Fib up (group c) |
| Ifi27l2b | −1.8292301 | −1.6020781 | −0.177508 | −0.1345804 | KO Fib up (group c) |
| Trim25 | 0.34906307 | 0.32244108 | 1.7178145 | 2.0800113 | KO Fib up (group c) |
| chr3: 146645022-146646977_F | 2.13019085 | 1.90761059 | 2.9735108 | 4.1991375 | KO Fib up (group c) |
| chr3: 137736327-137752677_F | −2.079809 | −1.9317308 | −0.885975 | 0.0152022 | KO Fib up (group c) |
| Gm3020 | 0.18623475 | 0.12371144 | 1.4127188 | 2.0415429 | KO Fib up (group c) |
| chr1: 175705225-175717250_R | 1.19994856 | 1.21022366 | 2.7334246 | 2.827546 | KO Fib up (group c) |
| Bst1 | 0.22913078 | 0.3195527 | 1.4479969 | 2.2522439 | KO Fib up (group c) |
| Cpxm2 | 0.3401482 | 0.89967384 | 1.5172375 | 2.8808885 | KO Fib up (group c) |
| chr3: 146629425-146649125_F | 1.74142592 | 1.71336016 | 2.7281694 | 3.8914328 | KO Fib up (group c) |
| Abcb1b | 2.70198411 | 2.77973153 | 3.8906705 | 4.761486 | KO Fib up (group c) |
| Rnf207 | 0.45686203 | 0.31732555 | 1.4453892 | 2.5096865 | KO Fib up (group c) |
| Frmd3 | 0.56009202 | 0.11876626 | 1.4027535 | 2.4613478 | KO Fib up (group c) |
| AU040829 | 1.2459653 | 1.36407882 | 2.3805331 | 3.4254581 | KO Fib up (group c) |
| Trim63 | 0.58392126 | 0.41364181 | 1.4132426 | 2.785839 | KO Fib up (group c) |
| Gm8212 | 0.04348331 | 0.11827232 | 1.2679773 | 2.1111411 | KO Fib up (group c) |
| Hoxd11 | 0.05224546 | 0.15734125 | 1.1697138 | 2.2589466 | KO Fib up (group c) |
| 1700048O20Rik | −0.2288361 | −0.200963 | 1.0556559 | 1.7380539 | KO Fib up (group c) |
| Creld1 | 0.07245597 | 0.17429213 | 1.4542821 | 2.0170645 | KO Fib up (group c) |
| Pde8b | −1.131398 | −0.7166649 | 0.7564695 | 0.626265 | KO Fib up (group c) |
| Gm8122 | 0.24297944 | 0.30443449 | 1.68699 | 2.1033489 | KO Fib up (group c) |
| chr11: 119254413-119296960_I | −0.2751667 | −0.0963629 | 1.5848821 | 1.2924206 | KO Fib up (group c) |
| Ifi203 | 1.22398775 | 1.27762092 | 2.8143014 | 2.9376931 | KO Fib up (group c) |
| Gm2737 | 1.32668342 | 1.22381096 | 2.1909557 | 3.6125725 | KO Fib up (group c) |
| Gm3187 | 0.51851301 | −0.0227965 | 1.6974662 | 2.0531951 | KO Fib up (group c) |
| LOC100044824 | 1.15842995 | 1.29822528 | 2.6642993 | 3.0501026 | KO Fib up (group c) |
| 2610528A11Rik | −0.2378629 | −0.3739244 | 1.1492791 | 1.4973127 | KO Fib up (group c) |
| Mlkl | −0.4849982 | −0.6072027 | 1.1551603 | 1.0163945 | KO Fib up (group c) |
| Obsl1 | 1.00885969 | 1.18687703 | 2.2422194 | 3.2246563 | KO Fib up (group c) |
| Acta1 | −0.8621419 | −1.2212395 | 0.3268494 | 0.8658607 | KO Fib up (group c) |
| Serpinb1a | 2.67528912 | 2.28322764 | 3.3496552 | 4.8869697 | KO Fib up (group c) |
| LOC100041034 | 0.43847029 | 2.02794714 | 2.7358841 | 3.0125404 | KO Fib up (group c) |
| ENSMUST00000111363 | 0.43490315 | 0.35095374 | 1.5366196 | 2.546886 | KO Fib up (group c) |
| Gdf15 | −0.3895817 | −0.2045545 | 1.2367273 | 1.4714447 | KO Fib up (group c) |
| Boc | 1.36983646 | 1.04730183 | 2.5457612 | 3.1773513 | KO Fib up (group c) |
| Akr1c18 | −0.1837415 | −0.0822297 | 1.3901602 | 1.6553601 | KO Fib up (group c) |
| AA467197 | 0.80602644 | 0.78663417 | 2.4367772 | 2.4690245 | KO Fib up (group c) |
| ENSMUST00000060283 | 0.09308592 | 0.3243071 | 3.4116993 | 0.3243071 | KO Fib up (group c) |
| Tapbp | 0.81875545 | 0.97175876 | 2.3062644 | 2.8033609 | KO Fib up (group c) |
| Lcn2 | 1.68887456 | 1.32277458 | 2.8176214 | 3.523276 | KO Fib up (group c) |
| Zfp820 | 0.07477098 | 0.35185995 | 1.4890613 | 2.2749235 | KO Fib up (group c) |
| Mme | 1.26478188 | 1.58857678 | 2.4090061 | 3.7940649 | KO Fib up (group c) |
| Cyba | 3.76581854 | 3.77805027 | 4.8014223 | 6.0935767 | KO Fib up (group c) |
| Gm6325 | −0.2701412 | −0.0841464 | 1.0027674 | 1.9983676 | KO Fib up (group c) |
| Veph1 | 0.42623245 | 0.46221918 | 1.8042172 | 2.4484679 | KO Fib up (group c) |
| Pgam2 | −0.4060695 | −0.65336 | 0.7395334 | 1.573854 | KO Fib up (group c) |
| Gm2397 | 1.18515875 | 0.66770093 | 2.3932855 | 2.8498922 | KO Fib up (group c) |
| Gm9640 | 2.36609347 | 2.22180931 | 4.0545022 | 3.9337618 | KO Fib up (group c) |
| Oas1b | 0.76786975 | 0.66215079 | 2.3405086 | 2.493738 | KO Fib up (group c) |
| 5830417l10Rik | 2.90880643 | 2.93722835 | 3.9948653 | 5.2560405 | KO Fib up (group c) |
| Gm12250 | 1.88613078 | −0.0045558 | 1.9978817 | 3.2926158 | KO Fib up (group c) |
| Hmgn3 | 3.69222244 | 3.61889959 | 4.7419016 | 5.9788818 | KO Fib up (group c) |
| Sema3e | 0.88893335 | 1.26268098 | 2.2703486 | 3.2968687 | KO Fib up (group c) |
| Slfn8 | 0.33301545 | 0.30316883 | 2.0083227 | 2.0499556 | KO Fib up (group c) |
| Fhl1 | 0.37969574 | 0.73333269 | 1.809604 | 2.7306467 | KO Fib up (group c) |
| Klhl30 | −2.6031652 | −2.2424508 | −0.917077 | −0.4943416 | KO Fib up (group c) |
| Ralgps2 | 0.51013474 | 0.56219155 | 1.9157602 | 2.5917696 | KO Fib up (group c) |
| LOC675328 | 0.58841862 | 0.1822136 | 1.9657935 | 2.2419188 | KO Fib up (group c) |
| Rasl11a | 1.79550258 | 2.32301642 | 2.9670522 | 4.5924066 | KO Fib up (group c) |
| Ripk3 | 4.9015105 | 4.57895009 | 5.889885 | 7.0342998 | KO Fib up (group c) |
| Cyb5r2 | −0.2506115 | −0.37921 | 1.3388384 | 1.4755271 | KO Fib up (group c) |
| Ptx3 | 5.80357407 | 5.76984466 | 6.9247037 | 8.1050334 | KO Fib up (group c) |
| Spats2l | 0.52089702 | 0.53668685 | 1.7423523 | 2.7737571 | KO Fib up (group c) |
| chr1: 84961943-84962737_F | 0.01467471 | 0.13920694 | 1.4469367 | 2.171611 | KO Fib up (group c) |
| Cnn1 | 2.66557525 | 2.70385079 | 3.7753045 | 5.0781098 | KO Fib up (group c) |
| chr11: 119250824-119286324_I | 0.01877502 | −0.0214042 | 1.3637174 | 2.1197374 | KO Fib up (group c) |
| S1pr1 | 0.9040608 | 0.68458095 | 2.0930264 | 2.9889011 | KO Fib up (group c) |
| Trim14 | 0.30486558 | 0.28836514 | 1.9599201 | 2.1300104 | KO Fib up (group c) |
| Plcxd2 | −1.1422619 | −1.1599406 | 0.3456191 | 0.8575118 | KO Fib up (group c) |
| Tgfbi | 1.76219579 | 1.60896547 | 2.8284527 | 4.0514056 | KO Fib up (group c) |
| Nmi | 1.02636238 | 0.88451952 | 2.7459051 | 2.6769055 | KO Fib up (group c) |
| Arl6ip1 | 2.08301173 | 1.92449985 | 3.315024 | 4.2063163 | KO Fib up (group c) |
| Anxa8 | 2.29698123 | 2.08529712 | 3.3885921 | 4.5123665 | KO Fib up (group c) |
| LOC100047292 | 0.33521401 | 0.58473361 | 1.6631827 | 2.7838818 | KO Fib up (group c) |
| Cdh17 | −1.1792316 | −0.5407685 | 0.738136 | 1.0765949 | KO Fib up (group c) |
| Tpd52l1 | −0.4208202 | −0.3465816 | 0.9942859 | 1.7776931 | KO Fib up (group c) |
| Batf2 | 0.42781684 | 0.34586053 | 2.0547892 | 2.2617627 | KO Fib up (group c) |
| Oit3 | −0.46332 | −0.4129789 | 0.8648742 | 1.8079879 | KO Fib up (group c) |
| 1700009N14Rik | −0.4903995 | −0.5320979 | 1.0571195 | 1.4695752 | KO Fib up (group c) |
| Rasgef1a | 1.11120422 | 0.96352091 | 2.4048849 | 3.2217468 | KO Fib up (group c) |
| Pmaip1 | 1.00621864 | 0.9732551 | 2.3507029 | 3.1845097 | KO Fib up (group c) |
| ENSMUST00000114885 | 0.41960509 | 0.74634721 | 2.1720192 | 2.549929 | KO Fib up (group c) |
| Ifitm6 | 0.93068613 | 0.61881949 | 2.062851 | 3.0448251 | KO Fib up (group c) |
| Stag3 | 4.24112263 | 4.23695508 | 5.5355073 | 6.5078466 | KO Fib up (group c) |
| H2-K1 | 0.5205011 | 0.59680781 | 2.2542616 | 2.4319221 | KO Fib up (group c) |
| Abi3 | 1.29580391 | 1.27643535 | 2.4732377 | 3.6731293 | KO Fib up (group c) |
| LOC235882 | 1.46329167 | 1.57567617 | 3.3815996 | 3.2316701 | KO Fib up (group c) |
| Tdrd7 | −0.0872884 | 0.04840731 | 1.7756404 | 1.7611013 | KO Fib up (group c) |
| ENSMUST00000082107 | −0.0748471 | −0.2194312 | 1.4511192 | 1.831024 | KO Fib up (group c) |
| Apobec1 | −0.9507938 | −1.2750915 | 0.7036154 | 0.647411 | KO Fib up (group c) |
| Adar | 0.23450925 | 0.18486086 | 1.8857357 | 2.1150706 | KO Fib up (group c) |
| F5 | −0.0613049 | 0.45532159 | 1.3995529 | 2.5782012 | KO Fib up (group c) |
| 1700016C15Rik | −0.4645791 | −0.1806101 | 0.9209935 | 2.0355028 | KO Fib up (group c) |
| Gm1673 | 4.87944689 | 4.46311144 | 5.921022 | 7.0270036 | KO Fib up (group c) |
| Amdhd2 | 3.23039694 | 3.13039452 | 4.3413287 | 5.6282566 | KO Fib up (group c) |
| Ifit2 | 0.07779823 | −0.0075558 | 1.5590438 | 2.1230167 | KO Fib up (group c) |
| Tbc1d9 | 0.14881156 | 0.03758852 | 1.4729747 | 2.3268794 | KO Fib up (group c) |
| Vps16 | 3.95067997 | 3.75412953 | 5.0908529 | 6.2282523 | KO Fib up (group c) |
| Rhov | 0.23597338 | 0.16374271 | 1.2825327 | 2.7382572 | KO Fib up (group c) |
| Syde2 | 0.7597385 | 0.97395018 | 2.1773455 | 3.1884618 | KO Fib up (group c) |
| ENSMUST00000036295 | 1.07844679 | 1.09959847 | 2.4727499 | 3.3460978 | KO Fib up (group c) |
| chr13: 98267049-98267604_F | −0.3160219 | −0.3652181 | 1.0679517 | 1.90498 | KO Fib up (group c) |
| Ebf3 | 2.37022026 | 2.71685093 | 3.7340656 | 5.0167668 | KO Fib up (group c) |
| Icos | 0.05290678 | −0.0346451 | 1.3321427 | 2.3612199 | KO Fib up (group c) |
| Lamc2 | −1.0818145 | −1.3815914 | 0.5173791 | 0.6951284 | KO Fib up (group c) |
| Twist2 | 3.04469164 | 2.80880878 | 4.033701 | 5.5072387 | KO Fib up (group c) |
| Spib | 0.16993152 | −0.0380937 | 1.3950716 | 2.4320381 | KO Fib up (group c) |
| Trim34 | 0.15357153 | 0.00017221 | 1.9676638 | 1.8881822 | KO Fib up (group c) |
| Cd93 | −1.4562077 | −1.6363249 | 0.6246661 | −0.0109372 | KO Fib up (group c) |
| Gm9782 | 0.30953278 | −0.4335737 | 1.8849937 | 1.697686 | KO Fib up (group c) |
| F830016B08Rik | 0.04704499 | −0.0364693 | 2.0076599 | 1.718742 | KO Fib up (group c) |
| Ifitm3 | 0.92180265 | 0.93169925 | 2.7543064 | 2.829344 | KO Fib up (group c) |
| LOC100044430 | 0.63850228 | 0.63956686 | 2.4436107 | 2.568314 | KO Fib up (group c) |
| Gtsf1 | −1.3791865 | −1.6844673 | 0.3711269 | 0.3293507 | KO Fib up (group c) |
| Soat2 | −0.0261928 | −0.1686891 | 1.8854791 | 1.6847537 | KO Fib up (group c) |
| Aif1 | 0.56020263 | 0.59859376 | 2.0897869 | 2.8394443 | KO Fib up (group c) |
| Fgd3 | −1.7464916 | −1.9192286 | 0.3505558 | −0.239166 | KO Fib up (group c) |
| H2-Q8 | 0.92067825 | 0.83490794 | 2.6068096 | 2.9380643 | KO Fib up (group c) |
| Gm7035 | 0.74103969 | 0.61557466 | 2.4375682 | 2.7160604 | KO Fib up (group c) |
| Krt42 | −0.0909461 | 0.00984102 | 1.6776026 | 2.0447971 | KO Fib up (group c) |
| Gm9132 | −0.1795913 | −1.1190136 | 0.9261935 | 1.5867238 | KO Fib up (group c) |
| LOC631406 | 0.46935132 | 0.63800357 | 2.2365191 | 2.6877869 | KO Fib up (group c) |
| Isl2 | 0.89992168 | 0.93027653 | 2.5763573 | 3.0779441 | KO Fib up (group c) |
| ENSMUST00000042610 | 0.27753632 | 0.08665841 | 1.9668248 | 2.2266216 | KO Fib up (group c) |
| H2-T9 | 0.59484277 | 0.68860072 | 2.3507066 | 2.7776561 | KO Fib up (group c) |
| Plac8 | −0.0041373 | 0.05082673 | 1.4810778 | 2.4126365 | KO Fib up (group c) |
| B3galnt1 | 0.86804796 | 0.98640581 | 2.3949977 | 3.3231904 | KO Fib up (group c) |
| Adora1 | 0.14907672 | 0.41806466 | 1.5045395 | 2.9312002 | KO Fib up (group c) |
| Ap3m1-ps | 0.70046467 | 0.65181381 | 2.430037 | 2.8049322 | KO Fib up (group c) |
| LOC100044874 | 0.29722967 | 0.22818529 | 2.1361961 | 2.272521 | KO Fib up (group c) |
| Gm10775 | 0.35454008 | 0.40168381 | 2.3505543 | 2.2939359 | KO Fib up (group c) |
| Slamf9 | 0.59652303 | 0.53392949 | 2.2920314 | 2.7325297 | KO Fib up (group c) |
| Selenbp1 | 1.57714316 | 1.00955191 | 2.7185312 | 3.7655637 | KO Fib up (group c) |
| BC066028 | 0.19041641 | 0.17617124 | 1.7838035 | 2.4936415 | KO Fib up (group c) |
| AF067061 | −0.3082184 | −0.3117804 | 1.7417837 | 1.5828012 | KO Fib up (group c) |
| Tmem130 | −0.4411045 | −0.675286 | 1.283553 | 1.5488485 | KO Fib up (group c) |
| Pparg | 0.31698463 | 0.24441445 | 1.7880696 | 2.7341586 | KO Fib up (group c) |
| Spint2 | 0.38133327 | 0.40428717 | 2.0151085 | 2.7365533 | KO Fib up (group c) |
| Trim21 | 1.57235159 | 1.41564901 | 3.3813834 | 3.5843843 | KO Fib up (group c) |
| AI747699 | 1.00184135 | 1.32229655 | 2.5754113 | 3.727538 | KO Fib up (group c) |
| Birc3 | 0.48135913 | 0.72948385 | 1.9904297 | 3.2034703 | KO Fib up (group c) |
| Psca | 0.1862987 | 0.1906798 | 1.822694 | 2.5557024 | KO Fib up (group c) |
| Got1 | 4.18447579 | 3.79321312 | 5.4599123 | 6.5311617 | KO Fib up (group c) |
| Parp12 | 0.58327714 | 0.81484235 | 2.7279633 | 2.6844377 | KO Fib up (group c) |
| 1700003F12Rik | 0.25362073 | 0.40866034 | 1.746253 | 2.936143 | KO Fib up (group c) |
| chr11: 119250824-119286324_I | −0.1414983 | −0.1594347 | 1.5552391 | 2.2046765 | KO Fib up (group c) |
| AW011956 | −1.4309237 | −1.2129772 | 0.0760088 | 1.3532044 | KO Fib up (group c) |
| Vopp1 | 5.53862285 | 5.42801038 | 7.0457136 | 8.0070473 | KO Fib up (group c) |
| Lrch2 | 1.25256027 | 0.84970539 | 2.6366533 | 3.5558764 | KO Fib up (group c) |
| Klra4 | 1.50112416 | 1.07936176 | 3.1941368 | 3.4932062 | KO Fib up (group c) |
| Gm3115 | 0.38947012 | 0.37530216 | 2.1602117 | 2.7127513 | KO Fib up (group c) |
| Cmtm8 | 0.14040759 | −0.2821692 | 1.6148973 | 2.3566099 | KO Fib up (group c) |
| Prr5l | 2.04960368 | 1.85296407 | 3.5042592 | 4.5206704 | KO Fib up (group c) |
| chr3: 137742666-137744470_F | −2.6785427 | −2.521596 | −0.724144 | −0.3489854 | KO Fib up (group c) |
| P2rx5 | 0.27639955 | 0.29788441 | 1.8350886 | 2.867759 | KO Fib up (group c) |
| Lypd6 | 0.04122027 | 0.18692918 | 1.7788514 | 2.5825261 | KO Fib up (group c) |
| Gm15056 | −0.1420408 | −0.2047364 | 1.7199481 | 2.0670468 | KO Fib up (group c) |
| Gm3696 | −0.361364 | −0.1959404 | 1.730059 | 1.8473603 | KO Fib up (group c) |
| Pak3 | 0.04334795 | 0.28553301 | 1.6142489 | 2.8527257 | KO Fib up (group c) |
| Slc16a3 | −0.4802491 | −0.7435451 | 1.8546998 | 1.0608914 | KO Fib up (group c) |
| LOC100045787 | 0.19173723 | 0.07049819 | 1.8314472 | 2.5927301 | KO Fib up (group c) |
| Herc5 | 0.14884901 | 0.30845979 | 2.2726488 | 2.3491506 | KO Fib up (group c) |
| Fam111a | 3.42160975 | 3.25342303 | 4.8578684 | 5.9986445 | KO Fib up (group c) |
| Stat1 | 0.45146829 | 0.55803996 | 2.3596407 | 2.8405106 | KO Fib up (group c) |
| Cxcl16 | 0.29978687 | 0.14448941 | 1.9026887 | 2.7421175 | KO Fib up (group c) |
| chr13: 58474388-58487288_R | 2.56524056 | 2.36233696 | 4.0743442 | 5.0550573 | KO Fib up (group c) |
| Mpeg1 | 0.04504358 | −0.0630432 | 1.6900439 | 2.5079897 | KO Fib up (group c) |
| Sgce | 4.09450662 | 4.11026403 | 5.4208446 | 7.0063009 | KO Fib up (group c) |
| Irf9 | 1.75991965 | 1.67855108 | 3.7324404 | 3.929385 | KO Fib up (group c) |
| chr16: 22718344-22725594_R | −0.036645 | −0.0118041 | 4.1171817 | 0.0702663 | KO Fib up (group c) |
| Cd274 | 0.15046144 | 0.52075102 | 2.2159995 | 2.6936072 | KO Fib up (group c) |
| H2-T10 | 0.32338492 | 0.44852668 | 2.3963136 | 2.6224875 | KO Fib up (group c) |
| Nrip3 | 0.86270212 | 0.3183816 | 2.2910743 | 3.1436584 | KO Fib up (group c) |
| Atf3 | −0.8108321 | −1.438071 | 0.6360008 | 1.3727573 | KO Fib up (group c) |
| ENSMUST00000111637 | 0.40678083 | 0.55216829 | 2.5383225 | 2.7035228 | KO Fib up (group c) |
| Hoxa11as | 4.22424709 | 4.23411938 | 5.634969 | 7.1130521 | KO Fib up (group c) |
| 9230105E10Rik | 0.55562893 | 0.50435358 | 2.4809898 | 2.8741535 | KO Fib up (group c) |
| H2-Q5 | 0.74297202 | 0.5177812 | 2.5763648 | 2.9820142 | KO Fib up (group c) |
| Eif2ak2 | 0.67292214 | 0.88038787 | 2.7124538 | 3.1448727 | KO Fib up (group c) |
| Irg1 | 0.26261595 | 0.26660939 | 2.4357439 | 2.3995361 | KO Fib up (group c) |
| Zfp185 | 0.7021672 | 0.96760559 | 2.5822848 | 3.4065575 | KO Fib up (group c) |
| AI428936 | 0.27375653 | 0.25805949 | 1.848147 | 3.0278496 | KO Fib up (group c) |
| Renbp | 1.62073069 | 1.69332199 | 3.2100207 | 4.4483837 | KO Fib up (group c) |
| Hoxa11 | 3.08263485 | 3.11776889 | 4.7935889 | 5.7567199 | KO Fib up (group c) |
| LOC677149 | 0.4559471 | 0.33056299 | 2.3506957 | 2.7996447 | KO Fib up (group c) |
| Ccl8 | −0.6277099 | −0.2634967 | 1.2515937 | 2.2243914 | KO Fib up (group c) |
| Gm8995 | −0.2587372 | −0.095572 | 1.5470606 | 2.4723804 | KO Fib up (group c) |
| H2-Q7 | 0.81662577 | 0.58609635 | 2.780875 | 2.995603 | KO Fib up (group c) |
| Dtx3l | 0.44447178 | 0.86377873 | 2.6476989 | 3.0470901 | KO Fib up (group c) |
| Hdgfrp3 | 3.02514203 | 3.03144121 | 4.694017 | 5.7586081 | KO Fib up (group c) |
| Nrn1 | 1.81361347 | 1.80983329 | 3.0998114 | 4.9197894 | KO Fib up (group c) |
| Apol9b | 1.59257912 | 1.58332728 | 3.929889 | 3.6435485 | KO Fib up (group c) |
| Lgals9 | 0.33395148 | 0.35637103 | 2.4168821 | 2.6904847 | KO Fib up (group c) |
| Grb10 | 1.18761779 | 1.06564617 | 2.7707584 | 3.901343 | KO Fib up (group c) |
| 9930023K05Rik | −0.085636 | 0.1540832 | 1.9817024 | 2.518931 | KO Fib up (group c) |
| chr6: 3333825-3349525_R | 0.93450948 | 1.39130684 | 3.4681062 | 3.2955877 | KO Fib up (group c) |
| C79246 | −0.0135232 | −0.0796554 | 1.8727749 | 2.478666 | KO Fib up (group c) |
| H2-T23 | 0.78033861 | 0.77053731 | 2.974745 | 3.0244196 | KO Fib up (group c) |
| Ddx58 | 1.31958075 | 1.39582885 | 3.4479969 | 3.7193036 | KO Fib up (group c) |
| 2410075B13Rik | 1.3465163 | 1.44829101 | 3.1299574 | 4.1174445 | KO Fib up (group c) |
| Gm9382 | 0.13788704 | −0.0754508 | 2.9537592 | 1.5719113 | KO Fib up (group c) |
| Gbp4 | 2.59974316 | 2.4776205 | 4.4044441 | 5.1382033 | KO Fib up (group c) |
| Morn4 | 1.73214545 | 1.67589938 | 3.6524547 | 4.2239406 | KO Fib up (group c) |
| Pamr1 | −0.5903543 | −0.9715454 | 0.884387 | 2.0254478 | KO Fib up (group c) |
| chr3: 87757295-87774970_F | 0.52565263 | 0.14730456 | 2.1015353 | 3.0526838 | KO Fib up (group c) |
| Csf2ra | 1.59525735 | 1.55089979 | 3.2744501 | 4.3610674 | KO Fib up (group c) |
| Gm13308 | 0.3100679 | 0.47339151 | 2.0417067 | 3.2330807 | KO Fib up (group c) |
| Calml4 | 3.08992147 | 3.0066897 | 4.6000188 | 5.9905931 | KO Fib up (group c) |
| Hoxd8 | 0.17233695 | 0.11822955 | 1.5827837 | 3.2130097 | KO Fib up (group c) |
| Zscan4f | −0.3365512 | 0.04340091 | 2.7008994 | 1.5140456 | KO Fib up (group c) |
| Tacstd2 | 0.3752735 | 0.29733897 | 2.1673394 | 3.0167293 | KO Fib up (group c) |
| Apol9a | 1.55951817 | 1.54391964 | 3.6335073 | 3.998048 | KO Fib up (group c) |
| Usp-ps | 0.0257126 | 0.05005757 | 2.1611953 | 2.4502907 | KO Fib up (group c) |
| Lass4 | 3.00100799 | 3.03699785 | 4.5814378 | 5.9941767 | KO Fib up (group c) |
| H2-D1 | 0.89381313 | 0.64816723 | 2.7337647 | 3.3534699 | KO Fib up (group c) |
| 2310043J07Rik | −0.3092101 | −0.4466147 | 1.7370691 | 2.0605904 | KO Fib up (group c) |
| BC021767 | −1.2850839 | −1.3522047 | 0.6333854 | 1.3038447 | KO Fib up (group c) |
| Hrct1 | 2.27593917 | 2.39813374 | 4.1716567 | 5.0801747 | KO Fib up (group c) |
| Pknox2 | 1.10737734 | 1.00845615 | 2.6208028 | 4.0737657 | KO Fib up (group c) |
| St3gal6 | 0.38246843 | 0.21468673 | 2.0349051 | 3.1461774 | KO Fib up (group c) |
| Ifi35 | 0.63471898 | 0.68077634 | 2.8612825 | 3.0456646 | KO Fib up (group c) |
| ENSMUST00000023246 | 0.43481556 | 0.17586641 | 2.5456575 | 2.6573799 | KO Fib up (group c) |
| LOC100044854 | −0.1014148 | −0.4194837 | 1.5852191 | 2.4923374 | KO Fib up (group c) |
| Slc43a3 | 1.5564905 | 1.39412586 | 3.2621254 | 4.2902691 | KO Fib up (group c) |
| Irgm1 | 1.31506982 | 1.43172841 | 3.475913 | 3.8787382 | KO Fib up (group c) |
| Ndrg2 | 0.07044003 | 0.51137449 | 2.056621 | 3.1348245 | KO Fib up (group c) |
| Samd9l | 1.81117112 | 2.04831409 | 3.8911118 | 4.5808519 | KO Fib up (group c) |
| ENSMUST00000113874 | 0.986629 | 0.72186997 | 3.0750134 | 3.2563526 | KO Fib up (group c) |
| H2-Q6 | 1.0101998 | 0.50274226 | 2.9494728 | 3.188772 | KO Fib up (group c) |
| Parp9 | 1.28737248 | 1.36379019 | 3.5416691 | 3.7441947 | KO Fib up (group c) |
| Tnfsf10 | −0.4211517 | −0.6810188 | 1.3020566 | 2.2391559 | KO Fib up (group c) |
| Ptprq | −0.2846764 | −0.0649604 | 1.5555339 | 2.757057 | KO Fib up (group c) |
| Ptgs2 | 1.60786559 | 1.36070173 | 3.3695951 | 4.2622661 | KO Fib up (group c) |
| Trim54 | −2.5582418 | −2.2078388 | −0.24432 | 0.1537242 | KO Fib up (group c) |
| Cgn | −1.3618833 | −1.4705914 | 0.5668765 | 1.2775609 | KO Fib up (group c) |
| Jag2 | 0.64830743 | 1.08990008 | 3.8136633 | 2.6325401 | KO Fib up (group c) |
| chr1: 175653557-175671940_R | 0.93374723 | 1.0178404 | 3.1280986 | 3.5330622 | KO Fib up (group c) |
| Pdpn | 0.89625739 | 0.91543829 | 2.7512951 | 3.7708285 | KO Fib up (group c) |
| Afap1l2 | 0.53307625 | 0.9328629 | 2.2121894 | 3.9653708 | KO Fib up (group c) |
| LOC630285 | 0.93245522 | 0.87173244 | 2.8500051 | 3.6740713 | KO Fib up (group c) |
| Fam18a | 1.2658776 | 1.3789244 | 3.2151775 | 4.1624187 | KO Fib up (group c) |
| ENSMUST00000111365 | 0.79668885 | 0.68443416 | 2.8594426 | 3.3574585 | KO Fib up (group c) |
| Ube2l6 | 0.43167333 | 0.42940028 | 2.7599626 | 2.858136 | KO Fib up (group c) |
| H2-Q2 | 0.73306332 | 0.44481436 | 2.8017411 | 3.1415036 | KO Fib up (group c) |
| LOC677644 | 0.78508526 | 0.76070897 | 3.1757566 | 3.148534 | KO Fib up (group c) |
| LOC100047388 | 0.72041019 | 0.5284374 | 2.9480478 | 3.0831028 | KO Fib up (group c) |
| Ctss | −1.4035841 | −1.3083946 | 0.6517325 | 1.4199001 | KO Fib up (group c) |
| Chi3l1 | 0.82637389 | 0.82637389 | 2.3851971 | 4.0538998 | KO Fib up (group c) |
| Ifi47 | 0.99632245 | 0.8027684 | 3.0027301 | 3.5832878 | KO Fib up (group c) |
| ENSMUST00000106892 | 0.0527031 | −0.2895674 | 2.1329326 | 2.4279536 | KO Fib up (group c) |
| 1700008P20Rik | 0.02833295 | 0.11176487 | 1.897621 | 3.0448226 | KO Fib up (group c) |
| Zfp275 | 2.35275814 | 2.69321531 | 4.400863 | 5.4516206 | KO Fib up (group c) |
| H2-M2 | −0.1901311 | −0.2166642 | 1.6783504 | 2.7541724 | KO Fib up (group c) |
| Muc13 | −0.08669 | −0.1705189 | 1.8849313 | 2.7129027 | KO Fib up (group c) |
| Als2cl | 1.53472253 | 1.63206977 | 3.6272541 | 4.3955384 | KO Fib up (group c) |
| Igf2bp3 | 1.94845899 | 1.44093215 | 3.5551592 | 4.6925709 | KO Fib up (group c) |
| LOC639910 | −0.2056317 | 0.0465388 | 2.5816513 | 2.1290521 | KO Fib up (group c) |
| Ifi27l2a | 4.10539897 | 4.06104973 | 6.272263 | 6.7718364 | KO Fib up (group c) |
| Pde3b | 0.54375789 | 0.61035564 | 2.307011 | 3.7508434 | KO Fib up (group c) |
| LOC547349 | 1.15202664 | 0.31822107 | 3.4163555 | 2.9633998 | KO Fib up (group c) |
| Celsr1 | −0.5546504 | −0.1812086 | 1.6214148 | 2.5562319 | KO Fib up (group c) |
| Sh3rf2 | 0.86022087 | 0.98640348 | 2.9039714 | 3.8630119 | KO Fib up (group c) |
| Gm10406 | 0.57705089 | 0.53841641 | 2.5366908 | 3.509161 | KO Fib up (group c) |
| Hoxd9 | −0.6578158 | −0.9742058 | 1.1327423 | 2.1785226 | KO Fib up (group c) |
| Robo1 | 0.25215562 | 0.44776027 | 2.2423746 | 3.4109947 | KO Fib up (group c) |
| Gata3 | 0.94515014 | 0.81633418 | 3.1058499 | 3.6247992 | KO Fib up (group c) |
| Gm567 | −0.4747343 | −0.4613635 | 1.4645659 | 2.5740494 | KO Fib up (group c) |
| AA684185 | −0.8976477 | 0.16757311 | 2.5230099 | 1.7217792 | KO Fib up (group c) |
| Oas1e | 0.27303467 | 0.45865835 | 2.9120844 | 2.8012558 | KO Fib up (group c) |
| Lamb3 | 1.03672261 | 1.03484938 | 3.2689983 | 3.8032572 | KO Fib up (group c) |
| Vgll2 | 0.11408281 | −0.0522436 | 2.0919345 | 2.9795488 | KO Fib up (group c) |
| Cxcl17 | 0.3403726 | 0.45376126 | 2.1569664 | 3.6657764 | KO Fib up (group c) |
| Isg15 | 1.7240267 | 1.75525904 | 4.087428 | 4.4247383 | KO Fib up (group c) |
| Smpdl3a | 2.65834993 | 2.86999787 | 4.4162523 | 6.158415 | KO Fib up (group c) |
| Zscan4e | −0.4144297 | −0.1833771 | 2.9271708 | 1.5344671 | KO Fib up (group c) |
| Mndal | 2.50508141 | 2.55199064 | 5.0546437 | 5.0717377 | KO Fib up (group c) |
| LOC100046632 | 0.00016994 | 0.31191825 | 2.7637973 | 2.6194729 | KO Fib up (group c) |
| Arhgap36 | 0.37050223 | −0.070503 | 2.6444079 | 2.744027 | KO Fib up (group c) |
| Il3ra | 0.02716805 | −0.1365444 | 1.9155433 | 3.0659264 | KO Fib up (group c) |
| ENSMUST00000116010 | 1.24143831 | 0.88803269 | 3.488447 | 3.7418631 | KO Fib up (group c) |
| Rab3b | 0.25072956 | 0.35180133 | 2.111801 | 3.6431658 | KO Fib up (group c) |
| Slc38a1 | 0.93029219 | 1.06615963 | 3.1467947 | 4.0185762 | KO Fib up (group c) |
| Chmp4c | −0.1086449 | 0.22903428 | 2.2322708 | 3.1007035 | KO Fib up (group c) |
| Hsh2d | 0.37674103 | 0.95324195 | 3.0810694 | 3.4703667 | KO Fib up (group c) |
| Fgf5 | −0.0081795 | −0.0451061 | 2.2142129 | 2.9699544 | KO Fib up (group c) |
| Oas1c | 0.9726625 | 0.83421498 | 3.4179404 | 3.6277904 | KO Fib up (group c) |
| Gm16525 | 0.53177683 | 0.39974134 | 2.5834567 | 3.6009845 | KO Fib up (group c) |
| Lgals3bp | 1.64300638 | 1.49630026 | 4.0990438 | 4.2983961 | KO Fib up (group c) |
| Ccl27a | 1.05016294 | 1.12175555 | 3.2045545 | 4.2460516 | KO Fib up (group c) |
| BC006779 | 0.44678139 | 0.64813235 | 2.8772945 | 3.4999613 | KO Fib up (group c) |
| Lce3c | 0.15030878 | −0.3395692 | 2.561388 | 2.543928 | KO Fib up (group c) |
| ENSMUSG00000068790 | 0.53062893 | 0.46523633 | 2.7182127 | 3.5733703 | KO Fib up (group c) |
| Cited1 | 0.33530016 | 0.19548717 | 2.2216292 | 3.6239863 | KO Fib up (group c) |
| Cmpk2 | −0.0442289 | 0.16297937 | 2.0460806 | 3.3997059 | KO Fib up (group c) |
| Mybpc2 | 0.21130495 | 0.09560972 | 2.280261 | 3.3673571 | KO Fib up (group c) |
| Tor3a | −0.0832033 | 0.03156465 | 2.5466528 | 2.7458977 | KO Fib up (group c) |
| BC094916 | −0.1209462 | 0.06708519 | 2.1319186 | 3.1623284 | KO Fib up (group c) |
| ENSMUST00000105041 | 0.82341671 | 0.1677689 | 3.0692932 | 3.2704514 | KO Fib up (group c) |
| Ifih1 | 1.01468982 | 0.96189347 | 3.0219373 | 4.3089162 | KO Fib up (group c) |
| Clic5 | 0.21712874 | 0.17882485 | 2.438452 | 3.3184221 | KO Fib up (group c) |
| chr6: 57583950-57624216_F | 0.37088437 | 0.60988928 | 2.8972206 | 3.4450617 | KO Fib up (group c) |
| C3 | 1.43298458 | 0.94409466 | 3.1780952 | 4.5694965 | KO Fib up (group c) |
| Oasl1 | 2.62796905 | 2.36307086 | 5.1083866 | 5.2678074 | KO Fib up (group c) |
| Lypd6b | 0.1453015 | 0.39152801 | 2.392835 | 3.5564822 | KO Fib up (group c) |
| Uba7 | 3.33120749 | 3.07795235 | 5.8268835 | 6.0094539 | KO Fib up (group c) |
| Selp | −0.1432106 | −0.2045388 | 2.2019322 | 2.8805911 | KO Fib up (group c) |
| Hoxd13 | 3.43926435 | 3.60487538 | 5.6231361 | 6.8908514 | KO Fib up (group c) |
| Rbm47 | −0.2984204 | −0.2688806 | 1.9350434 | 2.974471 | KO Fib up (group c) |
| LOC676689 | 0.90044099 | 0.50553787 | 3.3301761 | 3.5584612 | KO Fib up (group c) |
| Acox2 | 0.12891346 | 0.63457384 | 2.612899 | 3.6603328 | KO Fib up (group c) |
| ENSMUST00000077662 | 0.93909112 | 0.48138335 | 3.3022582 | 3.6374815 | KO Fib up (group c) |
| ENSMUST00000093902 | −0.2163373 | 0.06735305 | 2.2290932 | 3.1504471 | KO Fib up (group c) |
| LOC638189 | −0.1961775 | −0.2115938 | 2.5696954 | 2.5591358 | KO Fib up (group c) |
| Ccl5 | 4.2595558 | 4.16163716 | 6.5476072 | 7.4570647 | KO Fib up (group c) |
| Shroom2 | −0.5096148 | −0.404856 | 2.0652026 | 2.61931 | KO Fib up (group c) |
| Stat2 | 1.36384719 | 1.27440861 | 3.8801521 | 4.3640868 | KO Fib up (group c) |
| Crabp2 | 0.70712376 | 0.19853344 | 2.7103494 | 3.8155288 | KO Fib up (group c) |
| Tnfsf18 | 0.05273689 | −0.1908201 | 2.2943434 | 3.1924317 | KO Fib up (group c) |
| Gm4841 | 0.06244453 | −0.1152698 | 2.2312182 | 3.3416491 | KO Fib up (group c) |
| H2-Ab1 | −0.0049702 | −0.1051286 | 1.8543966 | 3.7276041 | KO Fib up (group c) |
| ENSMUST00000112728 | 0.2805107 | 0.09934872 | 2.6905648 | 3.3862607 | KO Fib up (group c) |
| LOC100038937 | −0.2605106 | −0.227596 | 2.7746356 | 2.4777926 | KO Fib up (group c) |
| Inpp5j | 1.00798018 | 1.25720183 | 3.4763295 | 4.5477457 | KO Fib up (group c) |
| Gm14446 | 0.10508822 | 0.22736915 | 2.3396626 | 3.7618892 | KO Fib up (group c) |
| Itgb7 | −1.1997525 | −2.3204482 | 1.4132274 | 0.8382074 | KO Fib up (group c) |
| Saa3 | 1.69037286 | 1.54528337 | 4.1875175 | 4.8331205 | KO Fib up (group c) |
| Rnf39 | 1.1178757 | 1.19315865 | 3.5331232 | 4.5718543 | KO Fib up (group c) |
| Csprs | 0.43762474 | 0.0877901 | 2.8393488 | 3.5109822 | KO Fib up (group c) |
| ENSMUST00000090647 | 0.60151867 | 0.89502445 | 3.165631 | 4.2155541 | KO Fib up (group c) |
| Gm5797 | 0.71080491 | 0.30158424 | 2.9534222 | 4.0449964 | KO Fib up (group c) |
| Neurl3 | −0.4736199 | −0.2464201 | 2.1980121 | 3.103179 | KO Fib up (group c) |
| Rtp4 | 2.69357671 | 2.63078485 | 5.5297243 | 5.8242581 | KO Fib up (group c) |
| Hck | −0.4450682 | −0.4874107 | 2.0063509 | 3.0925146 | KO Fib up (group c) |
| Fam176a | 1.87840025 | 1.62774324 | 3.9508462 | 5.6150159 | KO Fib up (group c) |
| Dhx58 | 1.69706268 | 1.71017523 | 4.7171373 | 4.7668961 | KO Fib up (group c) |
| Gm9706 | 1.71203979 | 1.0357745 | 4.7512567 | 4.1166882 | KO Fib up (group c) |
| LOC100048720 | 1.31535136 | 1.80902137 | 4.3045859 | 4.953289 | KO Fib up (group c) |
| Gbp1 | 2.21358523 | 2.22574529 | 5.007251 | 5.5820258 | KO Fib up (group c) |
| Tap1 | 1.12655862 | 1.00604382 | 3.9923375 | 4.3281816 | KO Fib up (group c) |
| Abcc3 | 0.32594912 | 0.65869711 | 2.8323077 | 4.3511366 | KO Fib up (group c) |
| Psmb8 | 1.94332126 | 1.74185172 | 4.619844 | 5.3447311 | KO Fib up (group c) |
| Papss2 | 1.85081691 | 1.5602113 | 4.4504486 | 5.2543604 | KO Fib up (group c) |
| Mlana | 2.22276351 | −0.3990529 | 3.4252005 | 4.7164664 | KO Fib up (group c) |
| Parm1 | 2.16522603 | 1.87201327 | 4.5128162 | 5.8634474 | KO Fib up (group c) |
| Gm3411 | 0.11225984 | 0.25078536 | 2.7068297 | 4.0101794 | KO Fib up (group c) |
| Gm13043 | −0.4160307 | −0.7956807 | 3.016718 | 2.1813119 | KO Fib up (group c) |
| Irgm2 | 2.48990663 | 2.60873861 | 5.4161656 | 6.1426143 | KO Fib up (group c) |
| Cxcl10 | 2.01487523 | 2.06372557 | 4.8409299 | 5.7194929 | KO Fib up (group c) |
| Gbp2 | 2.01952146 | 1.95111806 | 4.8912365 | 5.5998185 | KO Fib up (group c) |
| ligp1 | 0.36478425 | 0.56435707 | 3.2072498 | 4.2598603 | KO Fib up (group c) |
| Il28ra | −0.030967 | −0.1109727 | 2.769267 | 3.6293406 | KO Fib up (group c) |
| Tgtp1 | −0.0442231 | −0.4198176 | 2.737194 | 3.3550676 | KO Fib up (group c) |
| Klhl13 | 0.29468847 | −0.1523642 | 2.7823884 | 3.9429186 | KO Fib up (group c) |
| H2-T24 | 0.32766315 | 0.55418969 | 3.8828796 | 3.656121 | KO Fib up (group c) |
| chr1: 85304494-85305390_R | 0.00014321 | −0.2120119 | 2.9479637 | 3.5983206 | KO Fib up (group c) |
| Gbp5 | −0.0275462 | −0.0108029 | 3.000819 | 3.7872898 | KO Fib up (group c) |
| ENSMUST00000089689 | 2.89047308 | 2.48104682 | 5.7807496 | 6.4221844 | KO Fib up (group c) |
| Bst2 | 1.1817683 | 1.41160797 | 4.7835117 | 4.6437663 | KO Fib up (group c) |
| Ifit1 | 3.73861466 | 3.87992228 | 7.0728045 | 7.3856528 | KO Fib up (group c) |
| Tbx5 | 0.27895742 | −0.0057721 | 2.8770192 | 4.2396222 | KO Fib up (group c) |
| Igtp | 1.55993554 | 1.46776697 | 4.7442757 | 5.1758745 | KO Fib up (group c) |
| 4930506M07Rik | −0.010178 | 0.1349343 | 3.0380115 | 3.9983126 | KO Fib up (group c) |
| AI607873 | 1.37394082 | 1.44770099 | 4.4080786 | 5.3312929 | KO Fib up (group c) |
| Bex1 | −0.0451455 | 0.1036499 | 3.0588932 | 3.9891983 | KO Fib up (group c) |
| Ccl2 | 4.52815765 | 4.0164947 | 6.9700928 | 8.5795893 | KO Fib up (group c) |
| Ly6c1 | 3.30748398 | 2.81697289 | 6.0348825 | 7.1655791 | KO Fib up (group c) |
| Psmb9 | 2.09879747 | 1.92814508 | 5.2911794 | 5.8123484 | KO Fib up (group c) |
| chr1: 85036677-85038530_R | 0.57156116 | 0.73846038 | 3.9652169 | 4.4385901 | KO Fib up (group c) |
| Cdkn1c | 1.14793585 | 1.51980243 | 4.3156107 | 5.4629525 | KO Fib up (group c) |
| Ano1 | 0.83102647 | 1.02565883 | 4.0190746 | 5.0199684 | KO Fib up (group c) |
| Myom2 | 0.67550549 | 0.12284179 | 3.7833364 | 4.2170368 | KO Fib up (group c) |
| 9030619P08Rik | 2.42641924 | 1.88582992 | 5.5782937 | 5.9808921 | KO Fib up (group c) |
| Oasl2 | 3.59962113 | 3.47167599 | 6.9830387 | 7.3491526 | KO Fib up (group c) |
| Gm7609 | 0.40344343 | 0.39199341 | 3.6567631 | 4.4431644 | KO Fib up (group c) |
| Ly6e | 0.85368054 | 0.88256831 | 4.5088563 | 4.5547648 | KO Fib up (group c) |
| Gm1966 | 0.50723141 | 0.77241268 | 3.7523859 | 4.8939622 | KO Fib up (group c) |
| Gbp6 | 1.73896386 | 1.74714471 | 5.2650684 | 5.6146291 | KO Fib up (group c) |
| Defb1 | 0.18956814 | 0.16303507 | 3.5864167 | 4.3042235 | KO Fib up (group c) |
| Ly6a | 4.15621551 | 3.76960831 | 7.3151511 | 8.1716363 | KO Fib up (group c) |
| Gm4902 | 1.71855053 | 1.34431043 | 4.9197598 | 5.7643882 | KO Fib up (group c) |
| Mpa2l | 0.75539065 | 1.01959 | 4.3796511 | 5.0199532 | KO Fib up (group c) |
| Slc35f2 | 0.58454066 | 0.50069652 | 3.8726123 | 4.8502022 | KO Fib up (group c) |
| Cd74 | 2.24401705 | 1.99872923 | 5.1682995 | 6.7303573 | KO Fib up (group c) |
| Xaf1 | 2.97241781 | 2.88836249 | 6.3706816 | 7.1799302 | KO Fib up (group c) |
| Tgtp2 | 1.40974686 | 1.45977855 | 4.9055848 | 5.767482 | KO Fib up (group c) |
| Casp4 | 2.12836828 | 2.00605058 | 5.394012 | 6.5737857 | KO Fib up (group c) |
| Zfp612 | 0.13178549 | 0.39778739 | 3.7179027 | 4.7082653 | KO Fib up (group c) |
| Susd4 | 0.03493046 | 0.23761521 | 3.5243935 | 4.7543097 | KO Fib up (group c) |
| Ccl7 | 5.47021136 | 4.98307494 | 8.5399286 | 9.9624738 | KO Fib up (group c) |
| Rnf128 | 0.32717906 | 0.21145277 | 3.797551 | 4.7976929 | KO Fib up (group c) |
| Parp14 | 2.52085083 | 2.59762236 | 6.3135573 | 7.0271613 | KO Fib up (group c) |
| chr7: 111526943-111545093_R | 0.82184162 | 0.73851324 | 4.5839694 | 5.2244622 | KO Fib up (group c) |
| Slfn2 | −0.1285938 | 0.33857206 | 3.8383973 | 4.7361273 | KO Fib up (group c) |
| Trim79 | 0.69801438 | 0.84829022 | 4.6857983 | 5.2495424 | KO Fib up (group c) |
| H28 | −0.2826466 | −0.0126845 | 3.6626176 | 4.5021496 | KO Fib up (group c) |
| Gm3667 | 1.49311569 | 1.42389046 | 5.0783826 | 6.375782 | KO Fib up (group c) |
| ENSMUST00000098144 | 0.9090934 | 1.08365214 | 4.7810775 | 5.7899654 | KO Fib up (group c) |
| Phf11 | 1.51340364 | 1.22137028 | 5.6892794 | 5.6687095 | KO Fib up (group c) |
| D14Ertd668e | 1.423754 | 1.25608766 | 5.4458482 | 5.8871924 | KO Fib up (group c) |
| I830012O16Rik | 1.93549805 | 2.01765646 | 6.3873015 | 6.2932481 | KO Fib up (group c) |
| Lsr | 0.10159782 | 0.24367988 | 3.9404579 | 5.2051424 | KO Fib up (group c) |
| Mnda | 0.63951236 | 0.53813534 | 4.7624217 | 5.2217243 | KO Fib up (group c) |
| Fam174b | 0.20163846 | 0.20835294 | 4.197426 | 5.0363092 | KO Fib up (group c) |
| Gm3252 | 1.46419213 | 1.269865 | 5.2678018 | 6.3834638 | KO Fib up (group c) |
| Trim30 | 1.13843167 | 1.50427781 | 5.3799364 | 6.4171145 | KO Fib up (group c) |
| Gbp3 | 3.3233646 | 3.3437592 | 7.7057081 | 8.166102 | KO Fib up (group c) |
| Gvin1 | 0.97099915 | 1.26992856 | 5.0840005 | 6.3814919 | KO Fib up (group c) |
| Gm4951 | 0.13990147 | 0.32061527 | 4.3916869 | 5.3134984 | KO Fib up (group c) |
| Cfb | 1.31484816 | 1.56850698 | 5.7954206 | 6.3821432 | KO Fib up (group c) |
| Oas1f | 2.81710175 | 1.63931646 | 7.3311324 | 6.6145602 | KO Fib up (group c) |
| LOC100041903 | 0.64667947 | 1.014583 | 5.0486967 | 6.1263289 | KO Fib up (group c) |
| Oas1a | 2.41429805 | 2.35130304 | 7.0601626 | 7.2419222 | KO Fib up (group c) |
| Usp18 | 4.22532728 | 4.06105969 | 8.9306995 | 8.9267065 | KO Fib up (group c) |
| Ubd | 0.16448724 | 0.09153723 | 4.2710208 | 5.6424205 | KO Fib up (group c) |
| LOC100041068 | 0.3654865 | 0.34414297 | 4.8483889 | 5.6484809 | KO Fib up (group c) |
| Mx2 | 3.65919728 | 3.68448967 | 8.3554187 | 8.8363411 | KO Fib up (group c) |
| Ifit3 | 3.34174579 | 3.36100981 | 7.9814516 | 8.6029488 | KO Fib up (group c) |
| Zbp1 | 2.4861459 | 2.45493843 | 7.3453201 | 7.5848111 | KO Fib up (group c) |
| Mx1 | 2.21963052 | 2.04684751 | 6.8500287 | 7.6942127 | KO Fib up (group c) |
| Oas3 | 0.35148729 | 0.59655392 | 5.5374869 | 5.7442938 | KO Fib up (group c) |
| Ripk4 | −0.5165847 | −0.3534195 | 4.2283543 | 5.4328469 | KO Fib up (group c) |
| Ifi44 | 4.29015824 | 4.08043585 | 9.4962253 | 9.7341433 | KO Fib up (group c) |
| ENSMUST00000113743 | −0.0600719 | 0.07467749 | 4.8101326 | 6.2123003 | KO Fib up (group c) |
| Krt19 | 0.6253784 | 0.79412757 | 5.8550539 | 6.9212477 | KO Fib up (group c) |
| Rsad2 | 0.38906246 | 0.72396612 | 5.6966717 | 6.8141603 | KO Fib up (group c) |
| Irf7 | 2.81782042 | 2.78114467 | 8.2513862 | 8.7902233 | KO Fib up (group c) |
| Oas2 | 3.23734173 | 2.81549938 | 9.2810681 | 9.5827361 | KO Fib up (group c) |

We claim:

1. A method of assessing the prognosis of a subject in need of prognosis for a tumor: comprising: measuring the level of HSF1 expression and/or activation in a sample comprising tumor-associated stromal cells obtained from the tumor; and comparing the level of HSF1 expression and/or activation in the tumor-associated stromal cells with a control level of HSF1 expression and/or activation of HSF1, wherein an increased level of HSF1 expression and/or activation in tumor-associated stromal cells of a tumor as compared to a control level of HSF1 expression and/or activation is correlated with poor outcome, thereby assessing the prognosis of the subject.

2. The method of claim 1, wherein a higher level of HSF1 expression and/or activation in tumor-associated stromal cells of a tumor as compared to a control level indicates that the prognosis of the subject is poor, and wherein a lower or similar level of HSF1 expression and/or activation in tumor-associated stromal cells of a tumor as compared to a control level indicates that the prognosis of the subject is more favorable.

3. The method of claim 1 or claim 2, wherein the level of HSF1 expression and/or activation is measured specifically in tumor-associated stromal cells.

4. The method of any of claims 1-3, wherein the level of HSF1 expression and/or activation as compared with a control level is measured specifically in tumor-associated stromal cells, and wherein the method further comprises: measuring the level of HSF1 expression and/or activation specifically in cancer cells in the sample; comparing the level of HSF1 expression and/or HSF1 activation in the cancer cells with a control level of HSF1 expression and/or activation, wherein a lower or similar level of HSF1 expression and/or activation in cancer cells of the tumor as compared to a control level is indicative of a better prognosis than if the level of HSF1 expression and/or activation in cancer cells of the tumor is higher than the control level; and refining the prognosis based on the results of the comparison.

5. The method of any of claims 1-4, wherein the prognosis is for overall survival.

6. The method of any of claims 1-4, wherein the prognosis is for disease-free survival.

7. The method of any of claims 1-4, wherein the prognosis is for progression-free survival.

8. The method of any of claims 1-7, wherein the tumor-associated stromal cells comprise cancer-associated fibroblasts.

9. The method of any of claims 1-8, further comprising: selecting a treatment regimen for the subject based at least in part on the prognosis; and subjecting the subject to the selected treatment regimen.

10. The method of claim 9, wherein the method comprises determining that the subject has a poor prognosis and the method further comprises subjecting the subject to a relatively intensive treatment regimen based at least in part on the prognosis.

11. The method of claim 9 or claim 10, wherein the treatment regimen comprises administering an anticancer agent or radiotherapy to the subject.

12. The method of claim 9 or claim 10, wherein the treatment regimen comprises administering adjuvant chemotherapy or radiotherapy to the subject based at least in part on the prognosis.

13. A method of diagnosing a tumor in a subject comprising: measuring the level of HSF1 expression and/or activation in a sample comprising stromal cells obtained from a location in the subject's body that is suspected of harboring a tumor; and comparing the level of HSF1 expression and/or activation in the stromal cells with a control level of HSF expression and/or activation, wherein a higher level of HSF1 expression and/or activation in the stromal cells as compared to a control level of HSF1 expression and/or activation is indicative of the presence of a tumor.

14. The method of claim 13, wherein the stromal cells comprise fibroblasts.

15. A method for providing treatment-specific predictive information relating to a tumor, the method comprising: measuring the level of HSF1 expression and/or activation in a sample comprising tumor-associated stromal cells obtained from the tumor; and comparing the level of HSF1 expression and/or activation in the tumor-associated stromal cells with a control level of HSF1 expression and/or activation, wherein a higher level of HSF1 expression and/or activation in tumor-associated stromal cells of a tumor as compared to a control level of HSF1 expression and/or activation is correlated with tumor sensitivity or resistance to a treatment, thereby providing treatment-specific predictive information.

16. The method of claim 15, wherein a higher level of HSF1 expression and/or activation in tumor-associated stromal cells as compared to a control level indicates that the tumor has an increased likelihood of being sensitive to HSF1 inhibition.

17. A method of determining whether a subject with a tumor is a suitable candidate for treatment with an HSF1 inhibitor comprising: measuring the level of HSF1 expression and/or activation in a tumor sample comprising tumor-associated stromal cells obtained from the tumor; and comparing the level of HSF1 expression and/or activation in the tumor-associated stromal cells with a control level of HSF1 expression and/or activation of HSF1, wherein a higher level of HSF1 expression and/or activation in tumor-associated stromal cells of a tumor as compared to a control level of HSF1 expression and/or activation is indicative that the subject is a suitable candidate for treatment with an HSF1 inhibitor.

18. A method of predicting the likelihood that a tumor will be sensitive to an HSF1 inhibitor, the method comprising: measuring the level of HSF1 expression and/or activation in a tumor sample comprising tumor-associated stromal cells obtained from the tumor; and comparing the level of HSF1 expression and/or activation in the tumor-associated stromal cells with a control level of HSF1 expression and/or activation of HSF1, wherein a higher level of HSF1 expression and/or activation in tumor-associated stromal cells of a tumor as compared to a control level of HSF1 expression and/or activation is indicative that the tumor has an increased likelihood of being sensitive to the HSF inhibitor.

19. A method for tumor diagnosis, prognosis, treatment-specific prediction, or treatment selection comprising: measuring the level of HSF1 expression and/or activation in a sample comprising tumor-associated stromal cells obtained from a subject in need of diagnosis, prognosis, treatment-specific prediction, or treatment selection for a tumor; and scoring the sample based on the level of HSF1 expression and/or activation in the tumor-associated stromal cells, wherein the score provides diagnostic, prognostic, treatment-specific predictive, or treatment selection information.

20. The method of any of claims 15-19, wherein the level of HSF1 expression and/or activation is measured specifically in tumor-associated stromal cells.

21. The method of any of claims 15-19, wherein the level of HSF expression and/or activation is measured specifically in tumor-associated stromal cells, and wherein the method further comprises: measuring the level of HSF1 expression and/or activation specifically in cancer cells in the sample; comparing the level of HSF1 expression and/or HSF1 activation in the cancer cells with a control level of HSF1 expression and/or activation; and refining the prediction or score based on the result of the comparison.

22. The method of any of claims 15-21, wherein the tumor-associated stromal cells comprise cancer-associated fibroblasts.

23. The method of any of claims 1-22, wherein the tumor is a carcinoma.

24. The method of any of claims 1-22, wherein the tumor is an adenocarcinoma.

25. The method of any of claims 1-24, wherein the tumor is a Stage I tumor.

26. The method of any of claims 1-25, wherein the tumor is a breast, lung, skin, esophageal, colon, gastric, or prostate tumor.

27. The method of any of claims 1-26, wherein the tumor is a breast tumor.

28. The method of any of claims 1-26, wherein the tumor is a lung tumor, optionally wherein the lung tumor is a KRAS mutant lung tumor.

29. The method of any of claims 1-25, wherein the tumor is a Stage I lung adenocarcinoma, optionally wherein the lung adenocarcinoma is a KRAS mutant lung adenocarcinoma.

30. The method of any of claims 1-26, wherein the tumor is a human epidermal growth factor 2 (HER2) positive breast tumor.

31. The method of any of claims 1-30, wherein measuring the level of HSF1 expression comprises determining the level of an HSF1 gene product.

32. The method of any of claims 1-30, wherein measuring the level of HSF1 expression comprises determining the level of HSF1 mRNA.

33. The method of any of claims 1-30, wherein measuring the level of HSF1 expression comprises determining the level of HSF1 polypeptide.

34. The method of any of claims 1-30, wherein measuring the level of HSF1 expression and/or activation comprises detecting HSF1 polypeptide using an antibody that binds to HSF1 polypeptide.

35. The method of any of claims 1-30, wherein the method comprises: contacting a sample comprising tumor-associated stromal cells or tumor stromal tissue obtained from the subject with an antibody that binds specifically to HSF1; and detecting the level of antibody binding to the sample thereby measuring the level of HSF1 expression.

36. The method of any of claims 1-30, wherein the method comprises: contacting a sample comprising tumor-associated stromal cells or tumor stromal tissue obtained from the subject with an antibody that binds specifically to HSF1; and detecting the level of antibody binding to cell nuclei in the sample thereby measuring the level of HSF1 activation.

37. The method of any of claims 1-30, wherein the method comprises contacting a sample comprising tumor-associated stromal cells or tumor stromal tissue obtained from the subject with a first antibody that binds specifically to HSF1 and a second antibody that binds to tumor-associated stromal cells; and detecting binding of the first antibody to cells in the sample to which the second antibody binds, thereby detecting HSF1 specifically in tumor-associated stromal cells.

38. The method of any of claims 1-30, wherein the sample comprises tumor stromal tissue, and determining the level of expression of HSF1 comprises performing immunohistochemistry (IHC) on the tissue sample.

39. The method of any of claims 1-30, wherein determining the level of HSF1 activation comprises determining the localization of HSF1 polypeptide in cells, wherein nuclear localization is indicative of HSF1 activation.

40. The method of any of claims 1-30, wherein measuring the level of HSF1 expression and/or activation comprises measuring the expression of one or more genes that are regulated by HSF1 in tumor-associated stromal cells; and comparing the level of expression of the one or more genes with a control level, wherein an increased level of expression of the one or more genes is indicative of increased HSF1 expression and/or activation.

41. A method of identifying a candidate anti-cancer agent comprising: contacting tumor-associated stromal cells with a test agent; measuring HSF1 expression and/or activation in the tumor-associated stromal cells; comparing the level of HSF1 expression and/or activation with a control level; and identifying the test agent as a candidate anti-cancer agent if the level of HSF1 expression and/or activation measured is lower than the control level.

42. The method of claim 41, wherein the tumor-associated stromal cells are contacted with the test agent in vitro.

43. The method of claim 41, wherein the tumor-associated stromal cells are contacted with the test agent by administering the test agent to a subject having a tumor.

44. The method of any of claims 41-43, wherein the control level is a level of HSF1 expression and/or activation in tumor-associated stromal cells not contacted with the test agent.

45. The method of any of claims 41-44, wherein the tumor-associated stromal cells comprise cancer-associated fibroblasts.

46. The method of any of claims 41-45, wherein measuring the level of HSF1 expression comprises determining the level of an HSF1 gene product.

47. The method of any of claims 41-46, wherein measuring the level of HSF1 expression comprises determining the level of HSF1 mRNA.

48. The method of any of claims 41-46, wherein measuring the level of HSF1 expression comprises determining the level of HSF1 polypeptide.

49. The method of any of claims 41-46 or 48, wherein measuring the level of HSF1 expression and/or activation comprises detecting HSF1 polypeptide using an antibody that binds to HSF1 polypeptide.

50. The method of any of claims 41-46 or 48, wherein the method comprises: contacting the tumor-associated stromal cells with an antibody that binds specifically to HSF1; and detecting the level of antibody binding to the tumor-associated stromal cells, thereby measuring the level of HSF1 expression.

51. The method of any of claims 41-46 or 48, wherein the method comprises: contacting tumor-associated stromal cells or with an antibody that binds specifically to HSF1; and detecting the level of antibody binding to cell nuclei, thereby measuring the level of HSF1 activation.

52. The method of any of claims 41-46 or 48, wherein the method comprises: contacting tumor-associated stromal cells with a first antibody that binds specifically to HSF1 and a second antibody that binds to tumor-associated stromal cells; and detecting binding of the first antibody to cells to which the second antibody binds, thereby detecting HSF1 specifically in tumor-associated stromal cells.

53. The method of any of claims 41-46 or 48, wherein measuring the level of expression of HSF1 comprises performing immunohistochemistry (IHC) on a sample comprising tumor-associated stromal cells.

54. The method of any of claims 41-46 or 48, wherein determining the level of HSF1 activation comprises determining the localization of HSF1 polypeptide in cells, wherein nuclear localization is indicative of HSF1 activation.

55. The method of any of claims 41-46 or 48, wherein measuring the level of HSF1 expression and/or activation comprises measuring the expression of one or more genes that are regulated by HSF1 in tumor-associated stromal cells; and comparing the level of expression of the one or more genes with a control level, wherein an increased level of expression of the one or more genes is indicative of increased HSF1 expression and/or activation.

56. The method of any of claims 41-55, wherein the test agent is a small molecule.

57. The method of any of claims 41-56, further comprising measuring the effect of a candidate anti-cancer agent on cancer cell proliferation, tumor growth, tumor progression, tumor metastasis, tumor-associated mortality, or any combination thereof.
